(19) United States
(12) Patent Application Publication
(10) Pub. No.: US 2005/0209181 A1
(43) Pub. Date: Sep. 22, 2005

(54) Compositions and methods for diagnosing and treating mental disorders

(76) Inventors: Huda Akil, Ann Arbor, MI (US); Mary Atz, Tustin, CA (US); William E. Bunney JR., Laguna Beach, CA (US); Prabhakara V. Choudary, Davis, CA (US); Simon J. Evans, Milan, MI (US); Edward G. Jones, Winters, CA (US); Jun Li, Palo Alto, CA (US); Juan F. Lopez, Ann Arbor, MI (US); Richard M. Myers, Stanford, CA (US); Robert C. Thompson, Ann Arbor, MI (US); Hiroaki Tomita, Irvine, CA (US); Marquis P. Vawter, Niguel, CA (US); Stanley Watson, Ann Arbor, MI (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/982,556

(22) Filed: Nov. 4, 2004

Related U.S. Application Data
(60) Provisional application No. 60/517,751, filed on Nov. 5, 2003.

(51) Int. Cl. C12Q 1/68; A61K 48/00; A61K 31/519

(52) U.S. Cl. 514/44; 435/6; 514/220; 514/259.41; 514/469

(57) ABSTRACT

The present invention provides methods for diagnosing mental disorders (e.g., psychotic disorders such as schizophrenia). The invention also provides methods of identifying modulators of such mental disorders as well as methods of using these modulators to treat patients suffering from such mental disorders.

### CROSS-REFERENCES TO RELATED APPLICATIONS

The present application claims priority to U.S. Ser. No. 60/517,751, filed Nov. 5, 2003, herein incorporated by reference in it entirety.

### STATEMENT AS TO RIGHTS TO INVENTIONS MADE UNDER FEDERALLY SPONSORED RESEARCH AND DEVELOPMENT

Not applicable.

### BACKGROUND OF THE INVENTION

Schizophrenia and other mental disorders are a major public health problem, affecting a significant portion of the adult population of the United States each year. While it has been hypothesized that mental disorders, including psychotic disorders such as schizophrenia as well as mood disorders such as major depression and bipolar disorder have genetic roots, little progress has been made in identifying gene sequences and gene products that play a role in causing these disorders, as is true for many diseases with a complex genetic origin (see, e.g., Burmeister, Biol. Psychiatry 45:522-532 (1999)). Relying on the discovery that certain genes expressed in particular brain pathways and regions are likely involved in the development of mental disorders, the present invention provides methods for diagnosis and treatment of mental disorders such as schizophrenia, as well as methods for identifying compounds effective in treating mental disorders.

### BRIEF SUMMARY OF THE INVENTION

In order to further understand the neurobiology of psychotic disorders such as schizophrenia, the inventors of the present application have used DNA microarrays to study expression profiles of human post-mortem brains from patients diagnosed with schizophrenia. The work has focused on ten brain regions that are pathways or circuits involved in schizophrenia: anterior cingulate cortex (AnCg), dorsolateral prefrontal cortex (DLPFC), amygdala (AMY), cerebellar cortex (CB), entorhinal cortex (ERC), superior temporal gyrtus (STG), parietal cortex (PC), nucleus accumbens (nAcc), ventral thalamus (VThal), medial thalamus (MThal) and/or the hippocampus (HC).

The present invention demonstrates, for the first time, differential expression of genes in selected regions of brains of patients suffering from psychotic disorders, such as schizophrenia, in comparison with normal control subjects. These genes include the 1021 nucleic acids listed in Table 1; the genes listed in Table 22 which are differentially expressed in the DLPFC, AnCg and amygdala using Affymetrix chips and using brains with no agonal factors (904 genes); the genes listed in Table 23 which are differentially expressed in the DLPFC, AnCg and amygdala using Affymetrix chips and using brains with agonal factors (“affymetrix based on AFS postive”) (231 genes); and the genes listed in Table 24 wich are from the DLFC are are significantly different using the Codelink platform (89 genes).

In addition, the present invention identifies genes involved in psychotic disorders, where the proteins encoded by the nucleic acids listed in Tables 2-21 are components of biochemical pathways that play a role in mental disorders, e.g., psychotic disorders such as schizophrenia. Finally, Table 25 lists biochemical pathways involved in psychotic disorders, e.g., schizophrenia.

Genes that are differentially expressed in schizophrenia and by gender are useful in diagnosing psychotic disorders, e.g., providing SNPs, biomarkers, diagnostic probe sets for PCR and chip assays, and antigens and antibodies for immunoassays such as ELISA and immunohistochemical assays. Differential expression by brain region similarly is a useful diagnostic and therapeutic tool, as psychotic disorders such as schizophrenia primarily affect certain brain regions that are part of circuits or pathways involved in schizophrenia. The identification of genes, proteins, and biochemical assays involved in psychotic disorders also provides the means for drug discovery for anti-psychotic therapeutics.

This invention thus provides methods for determining whether a subject has or is predisposed for a mental disorder such as schizophrenia. The invention also provides methods of providing a prognosis and for monitoring disease progression and treatment. Furthermore, the present invention provides nucleic acid and protein targets for assays for drugs for the treatment of mental disorders such as schizophrenia.

In one aspect, the methods comprise the steps of: (i) obtaining a biological sample from a subject; (ii) contacting the sample with a reagent that selectively associates with a polynucleotide or polypeptide encoded by a nucleic acid that hybridizes under stringent conditions to a nucleotide sequence listed in Tables 1 and 22-24; and (iii) detecting the level of reagent that selectively associates with the sample, thereby determining whether the subject has or is predisposed for a mental disorder.

In some embodiments, the reagent is an antibody. In some embodiments, the reagent is a nucleic acid. In some embodiments, the reagent associates with a polynucleotide. In some embodiments, the reagent associates with a polypeptide. In some embodiments, the polynucleotide comprises a nucleotide sequence listed in Tables 1 and 22-24. In some embodiment, the polypeptide comprises an amino acid sequence of a gene listed in Tables 1 and 22-24. In some embodiments, the level of reagent that associates with the sample is different (i.e., higher or lower) from a level associated with humans without a mental disorder. In some embodiments, the biological sample is obtained from amniotic fluid, spinal fluid, or saliva. In some embodiments, the mental disorder is a mood disorder. In some embodiments, the mental disorder is a psychotic disorder such as schizophrenia.

The invention also provides methods of identifying a compound for treatment of a mental disorder. In some embodiments, the methods comprises the steps of: (i) contacting the compound with a polypeptide, which is encoded by a polynucleotide that hybridizes under stringent conditions to a nucleic acid comprising a nucleotide sequence of Tables 1 and 22-24; and (ii) determining the functional effect of the compound upon the polypeptide, thereby identifying a compound for treatment of a mental disorder.

In some embodiments, the contacting step is performed in vitro. In some embodiment, the polypeptide comprises an amino acid sequence of a gene listed in Tables 1 and 22-24. In some embodiments, the polypeptide is expressed in a cell or biological sample, and the cell or biological sample is contacted with the compound. In some embodiments, the mental disorder is a mood disorder or psychotic disorder. In some embodiments, the mood disorder is selected from the group consisting of bipolar disorder I and II and major depression. In some embodiments, the psychotic disorder is schizophrenia. In some embodiments, the methods further comprise administering the compound to an animal and determining the effect on the animal, e.g., an invertebrate, a vertebrate, or a mammal. In some embodiments, the determining step comprises testing the animal's mental function.

In some embodiments, the methods comprise the steps of (i) contacting the compound to a cell, the cell comprising a polynucleotide that hybridizes under stringent conditions to a nucleotide sequence of Tables 1 and 22-24; and (ii) selecting a compound that modulates expression of the polynucleotide, thereby identifying a compound for treatment of a mental disorder. In some embodiments, the polynucleotide comprises a nucleotide sequence listed in Tables 1 and 22-24. In some embodiment, the expression of the polynucleotide is enhanced. In some embodiments, the expression of the polynucleotide is decreased. In some embodiments, the methods further comprise administering the compound to an animal and determining the effect on the animal. In some embodiments, the determining step comprises testing the animal's mental function. In some embodiments, the mental disorder is a mood disorder or a psychotic disorder. In some embodiments, the mood disorder is selected from the group consisting of bipolar disorder I and II and major depression. In some embodiments, the psychotic disorder is schizophrenia.

The invention also provides methods of treating a mental disorder in a subject. In some embodiments, the methods comprise the step of administering to the subject a therapeutically effective amount of a compound identified using the methods described above. In some embodiments, the mental disorder is a mood disorder or a psychotic disorder. In some embodiments, the mood disorder is selected from the group consisting of bipolar disorder I and II and major depression. In some embodiments, the psychotic disorder is schizophrenia. In some embodiments, the compound is a small organic molecule, an antibody, an antisense molecule, an aptamer, an siRNA molecule, or a peptide.

The invention also provides methods of treating mental disorder in a subject, comprising the step of administering to the subject a therapeutically effective amount of a polypeptide, which is encoded by a polynucleotide that hybridizes under stringent conditions to a nucleic acid of Tables 1 and 22-24. In some embodiments, the polypeptide comprises an amino acid sequence encoded by a gene listed in Tables 1 and 22-24. In some embodiments, the mental disorder is a mood disorder or a psychotic disorder. In some embodiments, the psychotic disorder is schizophrenia. In some embodiments, the mood disorder is a bipolar disorder I or II or major depression.

The invention also provides methods of treating mental disorder in a subject, comprising the step of administering to the subject a therapeutically effective amount of a polynucleotide, which hybridizes under stringent conditions to a nucleic acid of Tables 1 and 22-24. In some embodiments, the mental disorder is a mood disorder or a psychotic disorder. In some embodiments, the psychotic disorder is schizophrenia. In some embodiments, the mood disorder is a bipolar disorder or major depression.

### BRIEF DESCRIPTION OF THE DRAWINGS

Table 1: Table 1 lists genes differentially expressed in mental disorder subjects suffering from schizophrenia. The table gives the ratio of expression as compared to normal controls. Thus, a gene that is over-expressed in subjects suffering from schizophrenia will have a value greater than one, while those that are underexpressed will have a value less than one.

Table 2: Table 2 lists neurofilament genes differentially expressed in all brain regions assayed.

Table 3: Table 3 lists developmental genes differentially expressed in all brain regions assayed.

Table 4: Table 4 lists extracellular genes differentially expressed in all brain regions assayed.

Table 5: Table 5 lists cell to cell signaling genes differentially expressed in all brain regions assayed.

Table 6: Table 6 lists synaptic transmission genes differentially expressed in all brain regions assayed.

Table 7: Table 7 lists organogenesis genes differentially expressed in all brain regions assayed.

Table 8: Table 8 lists cytoplasmic genes differentially expressed in VThal.

Table 9: Table 9 lists synaptic transmission genes differentially expressed in VThal.

Table 10: Table 10 lists 26S proteasome genes differentially expressed in VThal.

Table 11: Table 11 lists macromolecule biosynthesis genes differentially expressed in VThal.

Table 12: Table 12 lists neurofilament genes differentially expressed in MThal.

Table 13: Table 13 lists extracellular genes differentially expressed in HC.

Table 14: Table 14 lists proteasome complex genes differentially expressed in AnCg.

Table 15: Table 15 lists sterol biosynthesis genes differentially expressed in PC.

Table 16 Table 16 lists 26S proteasome genes differentially expressed in nAcc.

Table 17: Table 17 lists cytoplasmic genes differentially expressed in nAcc.

Table 18: Table 18 lists biotic stimulation genes differentially expressed in HC.

Table 19: Table 19 lists ribosomal genes differentially expressed in DLPFC.

Table 20: Table 20 lists protein targeting genes differentially expressed in AnCg.

Table 21: Table 21 lists endoplasmic reticulum (ER) genes differentially expressed in AnCg.

Table 22: Table 22 lists selected genes (i.e., synaptic transmission, ribosomal genes, cation homeostasis genes, and heat shock protein genes) differentially expressed by at least 1.2 fold in any brain region.

Table 22: Table 22 provides a list of genes differentially expressed in the DLPFC, AnCg and amygdala using Affymetrix chips and using brains with no agonal factors (904 genes).

Table 23: Table 23 provides a list of genes differentially expressed in the DLPFC, AnCg and amygdala using Affymetrix chips and using brains with agonal factors (“affymetrix based on AFS postive”) (231 genes).

Table 24: Table 24 provides a list of genes from the DLFC that were significantly different using the Codelink platform (89 genes).

Table 25: Table 25 lists biochemical pathways involved in psychotic disorders.

### Definitions

A “mental disorder” or “mental illness” or “mental disease” or “psychiatric or neuropsychiatric disease or illness or disorder” refers to mood disorders (e.g., major depression, mania, and bipolar disorders), psychotic disorders (e.g., schizophrenia, schizoaffective disorder, schizophreniform disorder, delusional disorder, brief psychotic disorder, and shared psychotic disorder), personality disorders, anxiety disorders (e.g., obsessive-compulsive disorder) as well as other mental disorders such as substance-related disorders, childhood disorders, dementia, autistic disorder, adjustment disorder, delirium, multi-infarct dementia, and Tourette's disorder as described in Diagnostic and Statistical Manual of Mental Disorders, Fourth Edition, (DSM IV). Typically, such disorders have a complex genetic and/or a biochemical component.

“A psychotic disorder” refers to a condition that affects the mind, resulting in at least some loss of contact with reality. Symptoms of a psychotic disorder include, e.g., hallucinations, changed behavior that is not based on reality, delusions and the like. See, e.g., DSM IV. Schizophrenia, schizoaffective disorder, schizophreniform disorder, delusional disorder, brief psychotic disorder, substance-induced psychotic disorder, and shared psychotic disorder are examples of psychotic disorders.

“Schizophrenia” refers to a psychotic disorder involving a withdrawal from reality by an individual. Symptoms comprise for at least a part of a month two or more of the following symptoms: delusions (only one symptom is required if a delusion is bizarre, such as being abducted in a space ship from the sun); hallucinations (only one symptom is required if hallucinations are of at least two voices talking to one another or of a voice that keeps up a running commentary on the patient's thoughts or actions); disorganized speech (e.g., frequent derailment or incoherence); grossly disorganized or catatonic behavior; or negative symptoms, i.e., affective flattening, alogia, or avolition. Schizophrenia encompasses disorders such as, e.g., schizoaffective disorders. Diagnosis of schizophrenia is described in, e.g., DSM IV. Types of schizophrenia include, e.g., paranoid, disorganized, catatonic, undifferentiated, and residual.

A “mood disorder” refers to disruption of feeling tone or emotional state experienced by an individual for an extensive period of time. Mood disorders include major depression disorder (i.e., unipolar disorder), mania, dysphoria, bipolar disorder, dysthymia, cyclothymia and many others. See, e.g., Diagnostic and Statistical Manual of Mental Disorders, Fourth Edition, (DSM IV).

“Major depression disorder,” “major depressive disorder,” or “unipolar disorder” refers to a mood disorder involving any of the following symptoms: persistent sad, anxious, or “empty” mood; feelings of hopelessness or pessimism; feelings of guilt, worthlessness, or helplessness; loss of interest or pleasure in hobbies and activities that were once enjoyed, including sex; decreased energy, fatigue, being “slowed down”; difficulty concentrating, remembering, or making decisions; insonmia, early-morning awakening, or oversleeping; appetite and/or weight loss or overeating and weight gain; thoughts of death or suicide or suicide attempts; restlessness or irritability; or persistent physical symptoms that do not respond to treatment, such as headaches, digestive disorders, and chronic pain. Various subtypes of depression are described in, e.g., DSM IV.

“Bipolar disorder” is a mood disorder characterized by alternating periods of extreme moods. A person with bipolar disorder experiences cycling of moods that usually swing from being overly elated or irritable (mania) to sad and hopeless (depression) and then back again, with periods of normal mood in between. Diagnosis of bipolar disorder is described in, e.g., DSM IV. Bipolar disorders include bipolar disorder I (mania with or without major depression) and bipolar disorder II (hypomania with major depression), see, e.g., DSM IV.

An “agonist” refers to an agent that binds to a polypeptide or polynucleotide of the invention, stimulates, increases, activates, facilitates, enhances activation, sensitizes or up regulates the activity or expression of a polypeptide or polynucleotide of the invention.

An “antagonist” refers to an agent that inhibits expression of a polypeptide or polynucleotide of the invention or binds to, partially or totally blocks stimulation, decreases, prevents, delays activation, inactivates, desensitizes, or down regulates the activity of a polypeptide or polynucleotide of the invention.

“Inhibitors,” “activators,” and “modulators” of expression or of activity are used to refer to inhibitory, activating, or modulating molecules, respectively, identified using in vitro and in vivo assays for expression or activity, e.g., ligands, agonists, antagonists, and their homologs and mimetics. The term “modulator” includes inhibitors and activators. Inhibitors are agents that, e.g., inhibit expression of a polypeptide or polynucleotide of the invention or bind to, partially or totally block stimulation or enzymatic activity, decrease, prevent, delay activation, inactivate, desensitize, or down regulate the activity of a polypeptide or polynucleotide of the invention, e.g., antagonists. Activators are agents that, e.g., induce or activate the expression of a polypeptide or polynucleotide of the invention or bind to, stimulate, increase, open, activate, facilitate, enhance activation or enzymatic activity, sensitize or up regulate the activity of a polypeptide or polynucleotide of the invention, e.g., agonists. Modulators include naturally occurring and synthetic ligands, antagonists, agonists, small chemical molecules and the like. Assays to identify inhibitors and activators include, e.g., applying putative modulator compounds to cells, in the presence or absence of a polypeptide or polynucleotide of the invention and then determining the functional effects on a polypeptide or polynucleotide of the invention activity. Samples or assays comprising a polypeptide or polynucleotide of the invention that are treated with a potential activator, inhibitor, or modulator are compared to control samples without the inhibitor, activator, or modulator to examine the extent of effect. Control samples (untreated with modulators) are assigned a relative activity value of 100%. Inhibition is achieved when the activity value of a polypeptide or polynucleotide of the invention relative to the control is about 80%, optionally 50% or 25-1%. Activation is achieved when the activity value of a polypeptide or polynucleotide of the invention relative to the control is 110%, optionally 150%, optionally 200-500%, or 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, RNAi, oligonucleotide, etc. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 Daltons and less than about 2500 Daltons, preferably less than about 2000 Daltons, preferably between about 100 to about 1000 Daltons, more preferably between about 200 to about 500 Daltons. An “siRNA” or “RNAi” refers to a nucleic acid that forms a double stranded RNA, which double stranded RNA has the ability to reduce or inhibit expression of a gene or target gene when the siRNA expressed in the same cell as the gene or target gene. “siRNA” or “RNAi” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 or about 24-29 nucleotides in length, e.g., 20, 21, 22, 23, 24, 25, 26, 27, 28, 29, or 30 nucleotides in length.

“Determining the functional effect” refers to assaying for a compound that increases or decreases a parameter that is indirectly or directly under the influence of a polynucleotide or polypeptide of the invention (such as a polynucleotide of Tables 1 and 22-24 or a polypeptide encoded by a gene of Tables 1 and 22-24), e.g., measuring physical and chemical or phenotypic effects. Such functional effects can be measured by any means known to those skilled in the art, e.g., changes in spectroscopic (e.g., fluorescence, absorbance, refractive index), hydrodynamic (e.g., shape), chromatographic, or solubility properties for the protein; measuring inducible markers or transcriptional activation of the protein; measuring binding activity or binding assays, e.g. binding to antibodies; measuring changes in ligand binding affinity; measurement of calcium influx; measurement of the accumulation of an enzymatic product of a polypeptide of the invention or depletion of an substrate; measurement of changes in protein levels of a polypeptide of the invention;
measurement of RNA stability; G-protein binding; GPCR phosphorylation or dephosphorylation; signal transduction, e.g., receptor-ligand interactions, second messenger concentrations (e.g., cAMP, EP3, or intracellular Ca²⁺); identification of downstream or reporter gene expression (CAT, luciferase, β-gal, GFP and the like), e.g., via chemiluminescence, fluorescence, calorimetric reactions, antibody binding, inducible markers, and ligand binding assays.

Samples or assays comprising a nucleic acid or protein disclosed herein that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition is achieved when the activity value relative to the control is about 80%, preferably 50%, more preferably 25-0%. Activation is achieved when the activity value relative to the control (untreated with activators) is 110%, more preferably 150%, more preferably 200-500% (i.e., two to five fold higher relative to the control), more preferably 1000-3000% higher.

“Biological sample” includes sections of tissues such as biopsy and autopsy samples, and frozen sections taken for histologic purposes. Such samples include blood, spinal fluid, sputum, tissue, lysed cells, brain biopsy, cultured cells, e.g., primary cultures, explants, and transformed cells, stool, urine, etc. A biological sample is typically obtained from a eukaryotic organism, most preferably a mammal such as a primate, e.g., chimpanzee or human; cow; dog; cat; a rodent, e.g., guinea pig, rat, mouse; rabbit; or a bird; reptile; or fish.

“Antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies (e.g., single chain Fv).

The terms “peptidomimetic” and “mimetic” refer to a synthetic chemical compound that has substantially the same structural and functional characteristics of the polynucleotides, polypeptides, antagonists or agonists of the invention. Peptide analogs are commonly used in the pharmaceutical industry as non-peptide drugs with properties analogous to those of the template peptide. These types of non-peptide compound are termed “peptide mimetics” or “peptidomimetics” (Fauchere, Adv. Drug Res. 15:29 (1986); Veber and Freidinger TINS p. 392 (1985); and Evans et al., J. Med. Chem. 30:1229 (1987), which are incorporated herein by reference). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., a polypeptide that has a biological or pharmacological activity), such as a CCX CKR, but have one or more peptide linkages optionally replaced by a linkage selected from the group consisting of, e.g., —CH₂NH—, —CH₂S—, —CH₂—CH₂—, —CH═CH— (cis and trans), —COCH₂—, —CH(OH)CH₂—, and —CH₂SO—. The mimetic can be either entirely composedof synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. For example, a mimetic composition is within the scope of the invention if it is capable of carrying out the binding or enzymatic activities of a polypeptide or polynucleotide of the invention or inhibiting or increasing the enzymatic activity or expression of a polypeptide or polynucleotide of the invention.

The term “gene” means the segment of DNA involved in producing a polypeptide chain; it includes regions preceding and following the coding region (leader and trailer) as well as intervening sequences (introns) between individual coding segments (exons).

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, haplotypes, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)). The term nucleic acid is used interchangeably with gene, cDNA, and mRNA encoded by a gene.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or 5×SSC, 1% SDS, incubating at 65° C., with wash in 0.2×SSC, and 0.1% SDS at 65° C. Such washes can be performed for 5, 15, 30, 60, 120, or more minutes. Nucleic acids that hybridize to the genes listed in Tables 1-22 are encompassed by the invention.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. Such washes can be performed for 5, 15, 30, 60, 120, or more minutes. A positive hybridization is at least twice background. Those of ordinary skill will readily recognize that alternative hybridization and wash conditions can be utilized to provide conditions of similar stringency.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al., PCR Protocols, A Guide to Methods and Applications (1990).

The phrase “a nucleic acid sequence encoding” refers to a nucleic acid that contains sequence information for a structural RNA such as rRNA, a tRNA, or the primary amino acid sequence of a specific protein or peptide, or a binding site for a trans-acting regulatory agent. This phrase specifically encompasses degenerate codons (i.e., different codons which encode a single amino acid) of the native sequence or sequences which may be introduced to conform with codon preference in a specific host cell.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under-expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “specifically (or selectively) binds to an antibody” or “specifically (or selectively) immunoreactive with”, when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised against a protein having an amino acid sequence encoded by any of the polynucleotides of the invention can be selected to obtain antibodies specifically immunoreactive with that protein and not with other proteins, except for polymorphic variants. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See, Harlow and Lane Antibodies, A Laboratory Manual, Cold Spring Harbor Publications, NY (1988) for a description of immunoassay formats and conditions that can be used to determine specific immunoreactivity. Typically, a specific or selective reaction will be at least twice the background signal or noise and more typically more than 10 to 100 times background.

One who is “predisposed for a mental disorder” as used herein means a person who has an inclination or a higher likelihood of developing a mental disorder when compared to an average person in the general population.

### DETAILED DESCRIPTION OF THE INVENTION

I. Introduction

To understand the complex genetic basis of mental disorders, the present invention provides studies that have been conducted to investigate the expression patterns of genes that are differentially expressed specifically in central nervous system of subjects with psychotic disorders. The large spectrum of symptoms associated with mental disorders is a reflection of the complex genetic basis and complex gene expression patterns in patients with mental disorders. Different combinations of the genes disclosed herein can be responsible for one or more mental disorders. Furthermore, brain pathways or circuits as well as subcellular pathways are important for understanding the development and diagnosis of mental disorders. The selected brain regions described herein (anterior cingulate cortex (AnCg), dorsolateral prefrontal cortex (DLPFC), cerebellar cortex (CB), entorhinal cortex (ERC), superior temporal gyrus (STG), parietal cortex (PC), nucleus accumbens (nAcc), ventral thalamus (VThal), medial thalamus (MThal) and/or the hippocampus (HC)) are implicated in the clinical symptoms of mental disorders such as psychotic disorders, e.g., schizophrenia. Brain imaging studies focusing on particular brain regions, cytoarchitectural changes in brain regions, expression of key neurotransmittors or related molecules in brain regions, and subcellular pathways in brain regions all contribute to the development of mental disorders, and thus are an important consideration in the diagnosis and therapeutic uses described herein.

The present invention demonstrates the altered expression (either higher or lower) of the genes of Tables 1-25 at the mRNA level in the brains of patients with mental disorders (e.g., schizophrenia) in comparison with normal individuals. This invention thus provides methods for diagnosis of mental disorders such as mood disorders (e.g., bipolar disorder, major depression, and the like), psychotic disorders (e.g., schizophrenia, and the like), and other mental disorders by detecting the level of a transcript or translation product of the genes listed in Tables 1-25 as well as their corresponding biochemical pathways. The chromosomal location of such genes can be used to discover other genes in the region that are linked to development of a particular disorder.

The invention further provides methods of identifying a compound useful for the treatment of such disorders by selecting compounds that modulates the functional effect of the translation products or the expression of the transcripts described herein. The invention also provides for methods of treating patients with such mental disorders, e.g., by administering the compounds of the invention or by gene therapy.

The genes and the polypeptides that they encode, which are associated with psychotic disorders such as schizophrenia, are useful for facilitating the design and development of various molecular diagnostic tools such as GeneChips™ containing probe sets specific for all or selected mental disorders, including but not limited to psychotic disorders, and as an ante- and/or post-natal diagnostic tool for screening newborns in concert with genetic counseling. Other diagnostic applications include evaluation of disease susceptibility, prognosis, and monitoring of disease or treatment process, as well as providing individualized medicine via predictive drug profiling systems, e.g., by correlating specific genomic motifs with the clinical response of a patient to individual drugs. In addition, the present invention is useful for multiplex SNP or haplotype profiling, including but not limited to the identification of pharmacogenetic targets at the gene, mRNA, protein, and pathway level. Profiling of splice variants is also useful for diagnostic and therapeutic applications.

The genes and the polypeptides that they encode, described herein, as also useful as drug targets for the development of therapeutic drugs for the treatment or prevention of mental disorders, including but not limited to psychotic disorders such as schizophrenia. Mental disorders have a high co-morbidity with other neurological disorders, such as Parkinson's disease or Alzheimeres. Therefore, the present invention can be used for diagnosis and treatment of patients with multiple disease states that include a mental disorder such as a psychotic disorder.

Antipsychotic medicines are in general equally effect for the treatment of schizophrenia, but act by different mechanisms. The similar effectiveness of the drugs for treatment of schizophrenia suggests that they act through a yet as unidentified common pathway. As demonstrated by the results shown herein, these drugs regulate a common gene, and/or a common group of genes as well as a unique set of genes.

II. General Recombinant Nucleic Acid Methods for use with the Invention

In numerous embodiments of the present invention, polynucleotides of the invention will be isolated and cloned using recombinant methods. Such polynucleotides include, e.g., those listed in Tables 1-22, which can be used for, e.g., protein expression or during the generation of variants, derivatives, expression cassettes, to monitor gene expression, for the isolation or detection of sequences of the invention in different species, for diagnostic purposes in a patient, e.g., to detect mutations or to detect expression levels of nucleic acids or polypeptides of the invention. In some embodiments, the sequences of the invention are operably linked to a heterologous promoter. In one embodiment, the nucleic acids of the invention are from any mammal, including, in particular, e.g., a human, a mouse, a rat, a primate, etc.

A. General Recombinant Nucleic Acids Methods

This invention relies on routine techniques in the field of recombinant genetics. Basic texts disclosing the general methods of use in this invention include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

B. Cloning Methods for the Isolation of Nucleotide Sequences Encoding Desired Proteins

In general, the nucleic acids encoding the subject proteins are cloned from DNA sequence libraries that are made to encode cDNA or genomic DNA. The particular sequences can be located by hybridizing with an oligonucleotide probe, the sequence of which can be derived from the sequences of the genes listed in Tables 1-22, which provide a reference for PCR primers and defines suitable regions for isolating specific probes. Alternatively, where the sequence is cloned into an expression library, the expressed recombinant protein can be detected immunologically with antisera or purified antibodies made against a polypeptide comprising an amino acid sequence encoded by a gene listed in Tables 1-25.

Methods for making and screening genomic and cDNA libraries are well known to those of skill in the art (see, e.g., Gubler and Hoffman Gene 25:263-269 (1983); Benton and Davis Science, 196:180-182 (1977); and Sambrook, supra). Brain cells are an example of suitable cells to isolate RNA and cDNA sequences of the invention.

Briefly, to make the cDNA library, one should choose a source that is rich in mRNA. The mRNA can then be made into cDNA, ligated into a recombinant vector, and transfected into a recombinant host for propagation, screening and cloning. For a genomic library, the DNA is extracted from a suitable tissue and either mechanically sheared or enzymatically digested to yield fragments of preferably about 5-100 kb. The fragments are then separated by gradient centrifugation from undesired sizes and are constructed in bacteriophage lambda vectors. These vectors and phage are packaged in vitro, and the recombinant phages are analyzed by plaque hybridization. Colony hybridization is carried out as generally described in Grunstein et al., Proc. Natl. Acad. Sci. USA., 72:3961-3965 (1975).

An alternative method combines the use of synthetic oligonucleotide primers with polymerase extension on an mRNA or DNA template. Suitable primers can be designed from specific sequences of the invention. This polymerase chain reaction (PCR) method amplifies the nucleic acids encoding the protein of interest directly from mRNA, cDNA, genomic libraries or cDNA libraries. Restriction endonuclease sites can be incorporated into the primers. Polymerase chain reaction or other in vitro amplification methods may also be useful, for example, to clone nucleic acids encoding specific proteins and express said proteins, to synthesize nucleic acids that will be used as probes for detecting the presence of mRNA encoding a polypeptide of the invention in physiological samples, for nucleic acid sequencing, or for other purposes (see, U.S. Pat. Nos. 4,683,195 and 4,683,202). Genes amplified by a PCR reaction can be purified from agarose gels and cloned into an appropriate vector.

Appropriate primers and probes for identifying polynucleotides of the invention from mammalian tissues can be derived from the sequences provided herein. For a general overview of PCR, see, Innis et al. PCR Protocols: A Guide to Methods and Applications, Academic Press, San Diego (1990).

Synthetic oligonucleotides can be used to construct genes. This is done using a series of overlapping oligonucleotides, usually 40-120 bp in length, representing both the sense and anti-sense strands of the gene. These DNA fragments are then annealed, ligated and cloned.

A gene encoding a polypeptide of the invention can be cloned using intermediate vectors before transformation into mammalian cells for expression. These intermediate vectors are typically prokaryote vectors or shuttle vectors. The proteins can be expressed in either prokaryotes, using standard methods well known to those of skill in the art, or eukaryotes as described infra.

III. Purification of Proteins of the Invention

Either naturally occurring or recombinant polypeptides of the invention can be purified for use in functional assays. Naturally occurring polypeptides, e.g., polypeptides encoded by genes listed in Tables 1-22, can be purified, for example, from mouse or human tissue such as brain or any other source of an ortholog. Recombinant polypeptides can be purified from any suitable expression system.

The polypeptides of the invention may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant polypeptides are purified. For example, proteins having established molecular adhesion properties can be reversible fused to polypeptides of the invention. With the appropriate ligand, the polypeptides can be selectively adsorbed to a purification column and then freed from the column in a relatively pure form. The fuised protein is then removed by enzymatic activity. Finally the polypeptide can be purified using immunoaffinity columns.

A. Purification of Proteins from Recombinant Bacteria

When recombinant proteins are expressed by the transformed bacteria in large amounts, typically after promoter induction, although expression can be constitutive, the proteins may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells typically, but not limited to, by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al. and Sambrook et al., both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques.

Alternatively, it is possible to purify proteins from bacteria periplasm. Where the protein is exported into the periplasm of the bacteria, the periplasmic fraction of the bacteria can be isolated by cold osmotic shock in addition to other methods known to those of skill in the art (see, Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Standard Protein Separation Techniques For Purifying Proteins

1. Solubility Fractionation

Often as an initial step, and if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

2. Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

3. Column Chromatography

The proteins of interest can also be separated from other proteins on the basis of their size, net surface charge, hydrophobicity and affinity for ligands. In addition, antibodies raised against proteins can be conjugated to column matrices and the proteins immunopurified. All of these methods are well known in the art.

It will be apparent to one of skill that chromatographic techniques can be performed at any scale and using equipment from many different manufacturers (e.g., Pharmacia Biotech).

IV. Detection of Gene Expression

Those of skill in the art will recognize that detection of expression of polynucleotides of the invention has many uses. For example, as discussed herein, detection of the level of polypeptides or polynucleotides of the invention in a patient is useful for diagnosing mood disorders or psychotic disorder or a predisposition for a mood disorder or psychotic disorder. Moreover, detection of gene expression is useful to identify modulators of expression of the polypeptides or polynucleotides of the invention.

A variety of methods of specific DNA and RNA measurement using nucleic acid hybridization techniques are known to those of skill in the art (see, Sambrook, supra). Some methods involve an electrophoretic separation (e.g., Southern blot for detecting DNA, and Northern blot for detecting RNA), but measurement of DNA and RNA can also be carried out in the absence of electrophoretic separation (e.g., by dot blot). Southern blot of genomic DNA (e.g., from a human) can be used for screening for restriction fragment length polymorphism (RFLP) to detect the presence of a genetic disorder affecting a polypeptide of the invention.

The selection of a nucleic acid hybridization format is not critical. A variety of nucleic acid hybridization formats are known to those skilled in the art. For example, common formats include sandwich assays and competition or displacement assays. Hybridization techniques are generally described in Hames and Higgins Nucleic Acid Hybridization, A Practical Approach, IRL Press (1985); Gall and Pardue, Proc. Natl. Acad. Sci. U.S.A., 63:378-383 (1969); and John et al. Nature, 223:582-587 (1969).

Detection of a hybridization complex may require the binding of a signal-generating complex to a duplex of target and probe polynucleotides or nucleic acids. Typically, such binding occurs through ligand and anti-ligand interactions as between a ligand-conjugated probe and an anti-ligand conjugated with a signal. The binding of the signal generation complex is also readily amenable to accelerations by exposure to ultrasonic energy.

The label may also allow indirect detection of the hybridization complex. For example, where the label is a hapten or antigen, the sample can be detected by using antibodies. In these systems, a signal is generated by attaching fluorescent or enzyme molecules to the antibodies or in some cases, by attachment to a radioactive label (see, e.g., Tijssen, “Practice and Theory of Enzyme Immunoassays,” Laboratory Techniques in Biochemistry and Molecular Biology, Burdon and van Knippenberg Eds., Elsevier (1985), pp. 9-20).

The probes are typically labeled either directly, as with isotopes, chromophores, lumiphores, chromogens, or indirectly, such as with biotin, to which a streptavidin complex may later bind. Thus, the detectable labels used in the assays of the present invention can be primary labels (where the label comprises an element that is detected directly or that produces a directly detectable element) or secondary labels (where the detected label binds to a primary label, e.g., as is common in immunological labeling). Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labeled probes or the like.

Other labels include, e.g., ligands that bind to labeled antibodies, fluorophores, chemiluminescent agents, enzymes, and antibodies which can serve as specific binding pair members for a labeled ligand. An introduction to labels, labeling procedures and detection of labels is found in Polak and Van Noorden Introduction to Immunocytochemistry, 2nd ed., Springer Verlag, NY (1997); and in Haugland Handbook of Fluorescent Probes and Research Chemicals, a combined handbook and catalogue Published by Molecular Probes, Inc. (1996).

In general, a detector which monitors a particular probe or probe combination is used to detect the detection reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill in the art. Commonly, an optical image of a substrate comprising bound labeling moieties is digitized for subsequent computer analysis.

Most typically, the amount of RNA is measured by quantifying the amount of label fixed to the solid support by binding of the detection reagent. Typically, the presence of a modulator during incubation will increase or decrease the amount of label fixed to the solid support relative to a control incubation which does not comprise the modulator, or as compared to a baseline established for a particular reaction type. Means of detecting and quantifying labels are well known to those of skill in the art.

In preferred embodiments, the target nucleic acid or the probe is immobilized on a solid support. Solid supports suitable for use in the assays of the invention are known to those of skill in the art. As used herein, a solid support is a matrix of material in a substantially fixed arrangement.

A variety of automated solid-phase assay techniques are also appropriate. For instance, very large scale immobilized polymer arrays (VLSIPS™), available from Affymetrix, Inc. (Santa Clara, Calif.) can be used to detect changes in expression levels of a plurality of genes involved in the same regulatory pathways simultaneously. See, Tijssen, supra., Fodor et al. (1991) Science, 251: 767-777; Sheldon et al. (1993) Clinical Chemistry 39(4): 718-719, and Kozal et al. (1996) Nature Medicine 2(7): 753-759.

Detection can be accomplished, for example, by using a labeled detection moiety that binds specifically to duplex nucleic acids (e.g., an antibody that is specific for RNA-DNA duplexes). One preferred example uses an antibody that recognizes DNA-RNA heteroduplexes in which the antibody is linked to an enzyme (typically by recombinant or covalent chemical bonding). The antibody is detected when the enzyme reacts with its substrate, producing a detectable product. Coutlee et al. (1989) Analytical Biochemistry 181:153-162; Bogulavski (1986) et al. J. Immunol. Methods 89:123-130; Prooijen-Knegt (1982) Exp. Cell Res. 141:397-407; Rudkin (1976) Nature 265:472-473, Stollar (1970) Proc. Nat'l Acad. Sci. USA 65:993-1000; Ballard (1982) Mol. Immunol. 19:793-799; Pisetsky and Caster (1982) Mol. Immunol. 19:645-650; Viscidi et al. (1988) J. Clin. Microbial. 41:199-209; and Kiney et al. (1989) J. Clin. Microbiol. 27:6-12 describe antibodies to RNA duplexes, including homo and heteroduplexes. Kits comprising antibodies specific for DNA:RNA hybrids are available, e.g., from Digene Diagnostics, Inc. (Beltsville, Md.).

In addition to available antibodies, one of skill in the art can easily make antibodies specific for nucleic acid duplexes using existing techniques, or modify those antibodies that are commercially or publicly available. In addition to the art referenced above, general methods for producing polyclonal and monoclonal antibodies are known to those of skill in the art (see, e.g., Paul (3rd ed.) Fundamental Immunology Raven Press, Ltd., NY (1993); Coligan Current Protocols in Immunology Wiley/Greene, NY (1991); Harlow and Lane Antibodies: A Laboratory Manual Cold Spring Harbor Press, NY (1988); Stites et al. (eds.) Basic and Clinical Immunology (4th ed.) Lange Medical Publications, Los Altos, Calif., and references cited therein; Goding Monoclonal Antibodies: Principles and Practice (2d ed.) Academic Press, New York, N.Y., (1986); and Kohler and Milstein Nature 256: 495-497 (1975)). Other suitable techniques for antibody preparation include selection of libraries of recombinant antibodies in phage or similar vectors (see, Huse et al. Science 246:1275-1281 (1989); and Ward et al. Nature 341:544-546 (1989)). Specific monoclonal and polyclonal antibodies and antisera will usually bind with a K_{D} of at least about 0.1 μM, preferably at least about 0.01 μM or better, and most typically and preferably, 0.001 μM or better.

The nucleic acids used in this invention can be either positive or negative probes. Positive probes bind to their targets and the presence of duplex formation is evidence of the presence of the target. Negative probes fail to bind to the suspect target and the absence of duplex formation is evidence of the presence of the target. For example, the use of a wild type specific nucleic acid probe or PCR primers may serve as a negative probe in an assay sample where only the nucleotide sequence of interest is present.

The sensitivity of the hybridization assays may be enhanced through use of a nucleic acid amplification system that multiplies the target nucleic acid being detected. Examples of such systems include the polymerase chain reaction (PCR) system, in particular RT-PCR or real time PCR, and the ligase chain reaction (LCR) system. Other methods recently described in the art are the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation.

An alternative means for determining the level of expression of the nucleic acids of the present invention is in situ hybridization. In situ hybridization assays are well known and are generally described in Angerer et al., Methods Enzymol. 152:649-660 (1987). In an in situ hybridization assay, cells or tissue, preferentially human cells or tissue from a selected brain region, are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled with radioisotopes or fluorescent reporters.

V. Immunological Detection of the Polypeptides of the Invention

In addition to the detection of polynucleotide expression using nucleic acid hybridization technology, one can also use immunoassays to detect polypeptides of the invention. Immunoassays can be used to qualitatively or quantitatively analyze polypeptides. A general overview of the applicable technology can be found in Harlow & Lane, Antibodies: A Laboratory Manual (1988).

A. Antibodies to Target Polypeptides or other Immunogens

Methods for producing polyclonal and monoclonal antibodies that react specifically with a protein of interest or other immunogen are known to those of skill in the art (see, e.g., Coligan, supra; and Harlow and Lane, supra; Stites et al., supra and references cited therein; Goding, supra; and Kohler and Milstein Nature, 256:495-497 (1975)). Such techniques include antibody preparation by selection of antibodies from libraries of recombinant antibodies in phage or similar vectors (see, Huse et al., supra; and Ward et al., supra). For example, in order to produce antisera for use in an immunoassay, the protein of interest or an antigenic fragment thereof, is isolated as described herein. For example, a recombinant protein is produced in a transformed cell line. An inbred strain of mice or rabbits is immunized with the protein using a standard adjuvant, such as Freund's adjuvant, and a standard immunization protocol. Alternatively, a synthetic peptide derived from the sequences disclosed herein and conjugated to a carrier protein can be used as an immunogen.

Polyclonal sera are collected and titered against the immunogen in an immunoassay, for example, a solid phase immunoassay with the immunogen immobilized on a solid support. Polyclonal antisera with a titer of 10⁴ or greater are selected and tested for their cross-reactivity against unrelated proteins or even other homologous proteins from other organisms, using a competitive binding immunoassay. Specific monoclonal and polyclonal antibodies and antisera will usually bind with a K_{D} of at least about 0.1 mM, more usually at least about 1 μM, preferably at least about 0.1 μM or better, and most preferably, 0.01 μM or better.

A number of proteins of the invention comprising immunogens may be used to produce antibodies specifically or selectively reactive with the proteins of interest. Recombinant protein is the preferred immunogen for the production of monoclonal or polyclonal antibodies. Naturally occurring protein, such as one comprising an amino acid sequence encoded by a gene listed in Table 1-25 may also be used either in pure or impure form. Synthetic peptides made using the protein sequences described herein may also be used as an immunogen for the production of antibodies to the protein. Recombinant protein can be expressed in eukaryotic or prokaryotic cells and purified as generally described supra. The product is then injected into an animal capable of producing antibodies. Either monoclonal or polyclonal antibodies may be generated for subsequent use in immunoassays to measure the protein.

Methods of production of polyclonal antibodies are known to those of skill in the art. In brief, an immunogen, preferably a purified protein, is mixed with an adjuvant and animals are immunized. The animal's immune response to the immunogen preparation is monitored by taking test bleeds and determining the titer of reactivity to the polypeptide of interest. When appropriately high titers of antibody to the immunogen are obtained, blood is collected from the animal and antisera are prepared. Further fractionation of the antisera to enrich for antibodies reactive to the protein can be done if desired (see, Harlow and Lane, supra).

Monoclonal antibodies may be obtained using various techniques familiar to those of skill in the art. Typically, spleen cells from an animal immunized with a desired antigen are immortalized, commonly by fusion with a myeloma cell (see, Kohler and Milstein, Eur. J. Immunol. 6:511-519 (1976)). Alternative methods of immortalization include, e.g., transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., supra.

Once target protein specific antibodies are available, the protein can be measured by a variety of immunoassay methods with qualitative and quantitative results available to the clinician. For a review of immunological and immunoassay procedures in general see, Stites, supra. Moreover, the immunoassays of the present invention can be performed in any of several configurations, which are reviewed extensively in Maggio Enzyme Immunoassay, CRC Press, Boca Raton, Fla. (1980); Tijssen, supra; and Harlow and Lane, supra.

Immunoassays to measure target proteins in a human sample may use a polyclonal antiserum that was raised to the protein (e.g., one has an amino acid sequence encoded by a gene listed in Table 1-25) or a fragment thereof. This antiserum is selected to have low cross-reactivity against different proteins and any such cross-reactivity is removed by immunoabsorption prior to use in the immunoassay.

B. Immunological Binding Assays

In a preferred embodiment, a protein of interest is detected and/or quantified using any of a number of well-known immunological binding assays (see, e.g., U.S. Pat. Nos. 4,366,241; 4,376,110; 4,517,288; and 4,837,168). For a review of the general immunoassays, see also Asai Methods in Cell Biology Volume 37: Antibodies in Cell Biology, Academic Press, Inc. NY (1993); Stites, supra. Immunological binding assays (or immunoassays) typically utilize a “capture agent” to specifically bind to and often immobilize the analyte (in this case a polypeptide of the present invention or antigenic subsequences thereof). The capture agent is a moiety that specifically binds to the analyte. In a preferred embodiment, the capture agent is an antibody that specifically binds, for example, a polypeptide of the invention. The antibody may be produced by any of a number of means well known to those of skill in the art and as described above.

Immunoassays also often utilize a labeling agent to specifically bind to and label the binding complex formed by the capture agent and the analyte. The labeling agent may itself be one of the moieties comprising the antibody/analyte complex. Alternatively, the labeling agent may be a third moiety, such as another antibody, that specifically binds to the antibody/protein complex.

In a preferred embodiment, the labeling agent is a second antibody bearing a label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents. These proteins are normal constituents of the cell walls of streptococcal bacteria. They exhibit a strong non-immunogenic reactivity with immunoglobulin constant regions from a variety of species (see, generally, Kronval, et al. J. Immunol., 111:1401-1406 (1973); and Akerstrom, et al. J. Immunol., 135:2589-2542 (1985)).

Throughout the assays, incubation and/or washing steps may be required after each combination of reagents. Incubation steps can vary from about 5 seconds to several hours, preferably from about 5 minutes to about 24 hours. The incubation time will depend upon the assay format, analyte, volume of solution, concentrations, and the like. Usually, the assays will be carried out at ambient temperature, although they can be conducted over a range of temperatures, such as 10° C. to 40° C.

1. Non-Competitive Assay Formats

Immunoassays for detecting proteins of interest from tissue samples may be either competitive or noncompetitive. Noncompetitive immunoassays are assays in which the amount of captured analyte (in this case the protein) is directly measured. In one preferred “sandwich” assay, for example, the capture agent (e.g., antibodies specific for a polypeptide encoded by a gene listed in Table 1-25) can be bound directly to a solid substrate where it is immobilized. These immobilized antibodies then capture the polypeptide present in the test sample. The polypeptide thus immobilized is then bound by a labeling agent, such as a second antibody bearing a label. Alternatively, the second antibody may lack a label, but it may, in turn, be bound by a labeled third antibody specific to antibodies of the species from which the second antibody is derived. The second can be modified with a detectable moiety, such as biotin, to which a third labeled molecule can specifically bind, such as enzyme-labeled streptavidin.

2. Competitive Assay Formats

In competitive assays, the amount of analyte (such as a polypeptide encoded by a gene listed in Table 1-25) present in the sample is measured indirectly by measuring the amount of an added (exogenous) analyte displaced (or competed away) from a capture agent (e.g., an antibody specific for the analyte) by the analyte present in the sample. In one competitive assay, a known amount of, in this case, the protein of interest is added to the sample and the sample is then contacted with a capture agent, in this case an antibody that specifically binds to a polypeptide of the invention. The amount of immunogen bound to the antibody is inversely proportional to the concentration of immunogen present in the sample. In a particularly preferred embodiment, the antibody is immobilized on a solid substrate. For example, the amount of the polypeptide bound to the antibody may be determined either by measuring the amount of subject protein present in a protein/antibody complex or, alternatively, by measuring the amount of remaining uncomplexed protein. The amount of protein may be detected by providing a labeled protein molecule.

Immunoassays in the competitive binding format can be used for cross-reactivity determinations. For example, a protein of interest can be immobilized on a solid support. Proteins are added to the assay which compete with the binding of the antisera to the immobilized antigen. The ability of the above proteins to compete with the binding of the antisera to the immobilized protein is compared to that of the protein of interest. The percent cross-reactivity for the above proteins is calculated, using standard calculations. Those antisera with less than 10% cross-reactivity with each of the proteins listed above are selected and pooled. The cross-reacting antibodies are optionally removed from the pooled antisera by immunoabsorption with the considered proteins, e.g., distantly related homologs.

The immunoabsorbed and pooled antisera are then used in a competitive binding immunoassay as described above to compare a second protein, thought to be perhaps a protein of the present invention, to the immunogen protein. In order to make this comparison, the two proteins are each assayed at a wide range of concentrations and the amount of each protein required to inhibit 50% of the binding of the antisera to the immobilized protein is determined. If the amount of the second protein required is less than 10 times the amount of the protein partially encoded by a sequence herein that is required, then the second protein is said to specifically bind to an antibody generated to an immunogen consisting of the target protein.

3. Other Assay Formats

In a particularly preferred embodiment, western blot (immunoblot) analysis is used to detect and quantify the presence of a polypeptide of the invention in the sample. The technique generally comprises separating sample proteins by gel electrophoresis on the basis of molecular weight, transferring the separated proteins to a suitable solid support (such as, e.g., a nitrocellulose filter, a nylon filter, or a derivatized nylon filter) and incubating the sample with the antibodies that specifically bind the protein of interest. For example, the antibodies specifically bind to a polypeptide of interest on the solid support. These antibodies may be directly labeled or alternatively may be subsequently detected using labeled antibodies (e.g., labeled sheep anti-mouse antibodies) that specifically bind to the antibodies against the protein of interest.

Other assay formats include liposome immunoassays (LIA), which use liposomes designed to bind specific molecules (e.g., antibodies) and release encapsulated reagents or markers. The released chemicals are then detected according to standard techniques (see, Monroe et al. (1986) Amer. Clin. Prod. Rev. 5:34-41).

4. Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well developed in the field of immunoassays and, in general, most labels useful in such methods can be applied to the present invention. Thus, a label is any composition detectable by spectroscopic, photochemical, biochemical, immunochemical, electrical, optical or chemical means. Useful labels in the present invention include magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

The label may be coupled directly or indirectly to the desired component of the assay according to methods well known in the art. As indicated above, a wide variety of labels may be used, with the choice of label depending on the sensitivity required, the ease of conjugation with the compound, stability requirements, available instrumentation, and disposal provisions.

Non-radioactive labels are often attached by indirect means. The molecules can also be conjugated directly to signal generating compounds, e.g., by conjugation with an enzyme or fluorescent compound. A variety of enzymes and fluorescent compounds can be used with the methods of the present invention and are well-known to those of skill in the art (for a review of various labeling or signal producing systems which may be used, see, e.g., U.S. Pat. No. 4,391,904).

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge-coupled devices (CCDS) or photomultipliers and the like. Similarly, enzymatic labels may be detected by providing the appropriate substrates for the enzyme and detecting the resulting reaction product. Finally simple colorimetric labels may be detected directly by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need to be labeled and the presence of the target antibody is detected by simple visual inspection.

VI. Screening for Modulators of Polypeptides and Polynucleotides of the Invention

Modulators of polypeptides or polynucleotides of the invention, i.e. agonists or antagonists of their activity or modulators of polypeptide or polynucleotide expression, are useful for treating a number of human diseases, including mood disorders or psychotic disorders. Administration of agonists, antagonists or other agents that modulate expression of the polynucleotides or polypeptides of the invention can be used to treat patients with mood disorders or psychotic disorders.

A. Screening Methods

A number of different screening protocols can be utilized to identify agents that modulate the level of expression or activity of polypeptides and polynucleotides of the invention in cells, particularly mammalian cells, and especially human cells. In general terms, the screening methods involve screening a plurality of agents to identify an agent that modulates the polypeptide activity by binding to a polypeptide of the invention, modulating inhibitor binding to the polypeptide or activating expression of the polypeptide or polynucleotide, for example.

1. Binding Assays

Preliminary screens can be conducted by screening for agents capable of binding to a polypeptide of the invention, as at least some of the agents so identified are likely modulators of polypeptide activity. The binding assays usually involve contacting a polypeptide of the invention with one or more test agents and allowing sufficient time for the protein and test agents to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots (see, e.g., Bennet and Yamamura, (1985) “Neurotransmitter, Hormone or Drug Receptor Binding Methods,” in Neurotransmitter Receptor Binding (Yamamura, H. I., et al., eds.), pp. 61-89. The protein utilized in such assays can be naturally expressed, cloned or synthesized.

Binding assays are also useful, e.g., for identifying endogenous proteins that interact with a polypeptide of the invention. For example, antibodies, receptors or other molecules that bind a polypeptide of the invention can be identified in binding assays.

2. Expression Assays

Certain screening methods involve screening for a compound that up or down-regulates the expression of a polypeptide or polynucleotide of the invention. Such methods generally involve conducting cell-based assays in which test compounds are contacted with one or more cells expressing a polypeptide or polynucleotide of the invention and then detecting an increase or decrease in expression (either transcript, translation product, or catalytic product). Some assays are performed with peripheral cells, or other cells, that express an endogenous polypeptide or polynucleotide of the invention.

Polypeptide or polynucleotide expression can be detected in a number of different ways. As described infra, the expression level of a polynucleotide of the invention in a cell can be determined by probing the mRNA expressed in a cell with a probe that specifically hybridizes with a transcript (or complementary nucleic acid derived therefrom) of a polynucleotide of the invention. Probing can be conducted by lysing the cells and conducting Northern blots or without lysing the cells using in situ-hybridization techniques. Alternatively, a polypeptide of the invention can be detected using immunological methods in which a cell lysate is probed with antibodies that specifically bind to a polypeptide of the invention.

Other cell-based assays are reporter assays conducted with cells that do not express a polypeptide or polynucleotide of the invention. Certain of these assays are conducted with a heterologous nucleic acid construct that includes a promoter of a polynucleotide of the invention that is operably linked to a reporter gene that encodes a detectable product. A number of different reporter genes can be utilized. Some reporters are inherently detectable. An example of such a reporter is green fluorescent protein that emits fluorescence that can be detected with a fluorescence detector. Other reporters generate a detectable product. Often such reporters are enzymes. Exemplary enzyme reporters include, but are not limited to, β-glucuronidase, chloramphenicol acetyl transferase (CAT); Alton and Vapnek (1979) Nature 282:864-869), luciferase, β-galactosidase, green fluorescent protein (GFP) and alkaline phosphatase (Toh, et al. (1980) Eur. J. Biochem. 182:231-238; and Hall et al. (1983) J. Mol. Appl. Gen. 2:101).

In these assays, cells harboring the reporter construct are contacted with a test compound. A test compound that either activates the promoter by binding to it or triggers a cascade that produces a molecule that activates the promoter causes expression of the detectable reporter. Certain other reporter assays are conducted with cells that harbor a heterologous construct that includes a transcriptional control element that activates expression of a polynucleotide of the invention and a reporter operably linked thereto. Here, too, an agent that binds to the transcriptional control element to activate expression of the reporter or that triggers the formation of an agent that binds to the transcriptional control element to activate reporter expression, can be identified by the generation of signal associated with reporter expression.

The level of expression or activity can be compared to a baseline value. As indicated above, the baseline value can be a value for a control sample or a statistical value that is representative of expression levels for a control population (e.g., healthy individuals not having or at risk for mood disorders or psychotic disorders). Expression levels can also be determined for cells that do not express a polynucleotide of the invention as a negative control. Such cells generally are otherwise substantially genetically the same as the test cells.

A variety of different types of cells can be utilized in the reporter assays. Cells that express an endogenous polypeptide or polynucleotide of the invention include, e.g., brain cells, including cells from the cerebellum, anterior cingulate cortex, or dorsolateral prefrontal cortex. Cells that do not endogenously express polynucleotides of the invention can be prokaryotic, but are preferably eukaryotic. The eukaryotic cells can be any of the cells typically utilized in generating cells that harbor recombinant nucleic acid constructs. Exemplary eukaryotic cells include, but are not limited to, yeast, and various higher eukaryotic cells such as the COS, CHO and HeLa cell lines and stem cells, e.g., neural stem cells.

Various controls can be conducted to ensure that an observed activity is authentic including running parallel reactions with cells that lack the reporter construct or by not contacting a cell harboring the reporter construct with test compound. Compounds can also be further validated as described below.

3. Catalytic Activity

Catalytic activity of polypeptides of the invention can be determined by measuring the production of enzymatic products or by measuring the consumption of substrates. Activity refers to either the rate of catalysis or the ability to the polypeptide to bind (Kₘ) the substrate or release the catalytic product (K_{d}).

Analysis of the activity of polypeptides of the invention are performed according to general biochemical analyses. Such assays include cell-based assays as well as in vitro assays involving purified or partially purified polypeptides or crude cell lysates. The assays generally involve providing a known quantity of substrate and quantifying product as a function of time.

4. Validation

Agents that are initially identified by any of the foregoing screening methods can be further tested to validate the apparent activity. Preferably such studies are conducted with suitable animal models. The basic format of such methods involves administering a lead compound identified during an initial screen to an animal that serves as a model for humans and then determining if expression or activity of a polynucleotide or polypeptide of the invention is in fact upregulated. The animal models utilized in validation studies generally are mammals of any kind. Specific examples of suitable animals include, but are not limited to, primates, mice, and rats.

5. Animal models

Animal models of mental disorders also find use in screening for modulators. In one embodiment, rat models of schizophrenia or other mental disorder, such as depression, are used for screening. In one embodiment, invertebrate models such as Drosophila models can be used, screening for modulators of Drosophila orthologs of the human genes disclosed herein. In another embodiment, transgenic animal technology including gene knockout technology, for example as a result of homologous recombination with an appropriate gene targeting vector, or gene overexpression, will result in the absence, decreased or increased expression of a polynucleotide or polypeptide of the invention. The same technology can also be applied to make knockout cells. When desired, tissue-specific expression or knockout of a polynucleotide or polypeptide of the invention may be necessary. Transgenic animals generated by such methods find use as animal models of mental disorder and are useful in screening for modulators of mental disorder.

Knockout cells and transgenic mice can be made by insertion of a marker gene or other heterologous gene into an endogenous gene site in the mouse genome via homologous recombination. Such mice can also be made by substituting an endogenous polynucleotide of the invention with a mutated version of the polynucleotide, or by mutating an endogenous polynucleotide, e.g., by exposure to carcinogens.

For development of appropriate stem cells, a DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987).

B. Modulators of Polypeptides or Polynucleotides of the Invention

The agents tested as modulators of the polypeptides or polynucleotides of the invention can be any small chemical compound, or a biological entity, such as a protein, sugar, nucleic acid or lipid. Alternatively, modulators can be genetically altered versions of a polypeptide or polynucleotide of the invention. Typically, test compounds will be small chemical molecules and peptides. Essentially any chemical compound can be used as a potential modulator or ligand in the assays of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like. Modulators also include agents designed to reduce the level of mRNA of the invention (e.g. antisense molecules, ribozymes, DNAzymes and the like) or the level of translation from an mRNA.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.). In addition, numerous combinatorial libraries are themselves commercially available (see, e.g., ComGenex, Princeton, N.J.; Tripos, Inc., St. Louis, Mo.; 3D Pharmaceuticals, Exton, Pa.; Martek Biosciences, Columbia, Md., etc.).

C. Solid State and Soluble High Throughput Assays

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 different compounds are possible using the integrated systems of the invention. More recently, microfluidic approaches to reagent manipulation have been developed.

The molecule of interest can be bound to the solid state component, directly or indirectly, via covalent or non-covalent linkage, e.g., via a tag. The tag can be any of a variety of components. In general, a molecule that binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.). Antibodies to molecules with natural binders such as biotin are also widely available and appropriate tag binders (see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs, such as agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I (1993)). Similarly, toxins and venoms, viral epitopes, hormones (e.g., opiates, steroids, etc.), intracellular receptors (e.g., which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-Gly sequences of between about 5 and 200 amino acids. Such flexible linkers are known to those of skill in the art. For example, poly(ethelyne glycol) linkers are available from Shearwater Polymers, Inc., Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature (see, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank and Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

The invention provides in vitro assays for identifying, in a high throughput format, compounds that can modulate the expression or activity of the polynucleotides or polypeptides of the invention. In a preferred embodiment, the methods of the invention include such a control reaction. For each of the assay formats described, “no modulator” control reactions that do not include a modulator provide a background level of binding activity.

In some assays it will be desirable to have positive controls to ensure that the components of the assays are working properly. At least two types of positive controls are appropriate. First, a known activator of a polynucleotide or polypeptide of the invention can be incubated with one sample of the assay, and the resulting increase in signal resulting from an increased expression level or activity of polynucleotide or polypeptide determined according to the methods herein. Second, a known inhibitor of a polynucleotide or polypeptide of the invention can be added, and the resulting decrease in signal for the expression or activity can be similarly detected.

D. Computer-Based Assays

Yet another assay for compounds that modulate the activity of a polypeptide or polynucleotide of the invention involves computer assisted drug design, in which a computer system is used to generate a three-dimensional structure of the polypeptide or polynucleotide based on the structural information encoded by its amino acid or nucleotide sequence. The input sequence interacts directly and actively with a pre-established algorithm in a computer program to yield secondary, tertiary, and quaternary structural models of the molecule. Similar analyses can be performed on potential receptors or binding partners of the polypeptides or polynucleotides of the invention. The models of the protein or nucleotide structure are then examined to identify regions of the structure that have the ability to bind, e.g., a polypeptide or polynucleotide of the invention. These regions are then used to identify polypeptides that bind to a polypeptide or polynucleotide of the invention.

The three-dimensional structural model of a protein is generated by entering protein amino acid sequences of at least 10 amino acid residues or corresponding nucleic acid sequences encoding a potential receptor into the computer system. The amino acid sequences encoded by the nucleic acid sequences provided herein represent the primary sequences or subsequences of the proteins, which encode the structural information of the proteins. At least 10 residues of an amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art.

The amino acid sequence represents a primary structure that encodes the information necessary to form the secondary, tertiary, and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential ligand binding regions are identified by the computer system. Three-dimensional structures for potential ligands are generated by entering amino acid or nucleotide sequences or chemical formulas of compounds, as described above. The three-dimensional structure of the potential ligand is then compared to that of a polypeptide or polynucleotide of the invention to identify binding sites of the polypeptide or polynucleotide of the invention. Binding affinity between the protein and ligands is determined using energy terms to determine which ligands have an enhanced probability of binding to the protein.

Computer systems are also used to screen for mutations, polymorphic variants, alleles and interspecies homologs of genes encoding a polypeptide or polynucleotide of the invention. Such mutations can be associated with disease states or genetic traits and can be used for diagnosis. As described above, GeneChip™ and related technology can also be used to screen for mutations, polymorphic variants, alleles and interspecies homologs. Once the variants are identified, diagnostic assays can be used to identify patients having such mutated genes. Identification of the mutated a polypeptide or polynucleotide of the invention involves receiving input of a first amino acid sequence of a polypeptide of the invention (or of a first nucleic acid sequence encoding a polypeptide of the invention), e.g., any amino acid sequence having at least 60%, optionally at least 70% or 85%, identity with the amino acid sequence of interest, or conservatively modified versions thereof. The sequence is entered into the computer system as described above. The first nucleic acid or amino acid sequence is then compared to a second nucleic acid or amino acid sequence that has substantial identity to the first sequence. The second sequence is entered into the computer system in the manner described above. Once the first and second sequences are compared, nucleotide or amino acid differences between the sequences are identified. Such sequences can represent allelic differences in various polynucleotides, including SNPs and/or haplotypes, of the invention, and mutations associated with disease states and genetic traits.

VII. Compositions, Kits and Integrated Systems

The invention provides compositions, kits and integrated systems for practicing the assays described herein using polypeptides or polynucleotides of the invention, antibodies specific for polypeptides or polynucleotides of the invention, etc.

The invention provides assay compositions for use in solid phase assays; such compositions can include, for example, one or more polynucleotides or polypeptides of the invention immobilized on a solid support, and a labeling reagent. In each case, the assay compositions can also include additional reagents that are desirable for hybridization. Modulators of expression or activity of polynucleotides or polypeptides of the invention can also be included in the assay compositions.

The invention also provides kits for carrying out the therapeutic and diagnostic assays of the invention. The kits typically include a probe that comprises an antibody that specifically binds to polypeptides or polynucleotides of the invention, and a label for detecting the presence of the probe. The kits may include several polynucleotide sequences encoding polypeptides of the invention. Kits can include any of the compositions noted above, and optionally further include additional components such as instructions to practice a high-throughput method of assaying for an effect on expression of the genes encoding the polypeptides of the invention, or on activity of the polypeptides of the invention, one or more containers or compartments (e.g., to hold the probe, labels, or the like), a control modulator of the expression or activity of polypeptides of the invention, a robotic armature for mixing kit components or the like.

The invention also provides integrated systems for high-throughput screening of potential modulators for an effect on the expression or activity of the polypeptides of the invention. The systems typically include a robotic armature which transfers fluid from a source to a destination, a controller which controls the robotic armature, a label detector, a data storage unit which records label detection, and an assay component such as a microtiter dish comprising a well having a reaction mixture or a substrate comprising a fixed nucleic acid or immobilization moiety.

A number of robotic fluid transfer systems are available, or can easily be made from existing components. For example, a Zymate XP (Zymark Corporation; Hopkinton, Mass.) automated robot using a Microlab 2200 (Hamilton; Reno, Nev.) pipetting station can be used to transfer parallel samples to 96 well microtiter plates to set up several parallel simultaneous STAT binding assays.

Optical images viewed (and, optionally, recorded) by a camera or other recording device (e.g., a photodiode and data storage device) are optionally further processed in any of the embodiments herein, e.g., by digitizing the image and storing and analyzing the image on a computer. A variety of commercially available peripheral equipment and software is available for digitizing, storing and analyzing a digitized video or digitized optical image, e.g., using PC, MACINTOSH®, or UNIX® based (e.g., SUN® work station) computers.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. The apparatus and methods of the invention are easily used for viewing any sample, e.g., by fluorescent or dark field microscopic techniques. Lasar based systems can also be used.

VIII. Administration and Pharmaceutical compositions

Modulators of the polynucleotides or polypeptides of the invention (e.g., antagonists or agonists) can be administered directly to a mammalian subject for modulation of activity of those molecules in vivo. Administration is by any of the routes normally used for introducing a modulator compound into ultimate contact with the tissue to be treated and is well known to those of skill in the art. Although more than one route can be used to administer a particular composition, a particular route can often provide a more immediate and more effective reaction than another route.

Diseases that can be treated include the following, which include the corresponding reference number from Morrison, DSM-IV Made Easy, 1995: Schizophrenia, Catatonic, Subchronic, (295.21); Schizophrenia, Catatonic, Chronic (295.22); Schizophrenia, Catatonic, Subchronic with Acute Exacerbation (295.23); Schizophrenia, Catatonic, Chronic with Acute Exacerbation (295.24); Schizophrenia, Catatonic, in Remission (295.55); Schizophrenia, Catatonic, Unspecified (295.20); Schizophrenia, Disorganized, Subchronic (295.11); Schizophrenia, Disorganized, Chronic (295.12); Schizophrenia, Disorganized, Subchronic with Acute Exacerbation (295.13); Schizophrenia, Disorganized, Chronic with Acute Exacerbation (295.14); Schizophrenia, Disorganized, in Remission (295.15); Schizophrenia, Disorganized, Unspecified (295.10); Schizophrenia, Paranoid, Subchronic (295.31); Schizophrenia, Paranoid, Chronic (295.32); Schizophrenia, Paranoid, Subchronic with Acute Exacerbation (295.33); Schizophrenia, Paranoid, Chronic with Acute Exacerbation (295.34); Schizophrenia, Paranoid, in Remission (295.35); Schizophrenia, Paranoid, Unspecified (295.30); Schizophrenia, Undifferentiated, Subchronic (295.91); Schizophrenia, Undifferentiated, Chronic (295.92); Schizophrenia, Undifferentiated, Subchronic with Acute Exacerbation (295.93); Schizophrenia, Undifferentiated, Chronic with Acute Exacerbation (295.94); Schizophrenia, Undifferentiated, in Remission (295.95); Schizophrenia, Undifferentiated, Unspecified (295.90); Schizophrenia, Residual, Subchronic (295.61); Schizophrenia, Residual, Chronic (295.62); Schizophrenia, Residual, Subchronic with Acute Exacerbation (295.63); Schizophrenia, Residual, Chronic with Acute Exacerbation (295.94); Schizophrenia, Residual, in Remission (295.65); Schizophrenia, Residual, Unspecified (295.60); Delusional (Paranoid) Disorder (297.10); Brief Reactive Psychosis (298.80); Schizophreniform Disorder (295.40); Schizoaffective Disorder (295.70); Induced Psychotic Disorder (297.30); Psychotic Disorder NOS (Atypical Psychosis) (298.90); Personality Disorders, Paranoid (301.00); Personality Disorders, Schizoid (301.20); Personality Disorders, Schizotypal (301.22); Personality Disorders, Antisocial (301.70); Personality Disorders, Borderline (301.83) and bipolar disorders, maniac, hypomaniac, dysthymic or cyclothymic disorders, substance-induced major depression, psychotic disorder, including schizophrenia (paranoid, catatonic, delusional) having schizoaffective disorder, and substance-induced psychotic disorder.

In some embodiments, modulators of polynucleotides or polypeptides of the invention can be combined with other drugs useful for treating mental disorders including psychotic disorders, e.g., schizophrenia; and mood disorders, e.g., bipolar disorders, or major depression. In some preferred embodiments, pharmaceutical compositions of the invention comprise a modulator of a polypeptide of polynucleotide of the invention combined with at least one of the compounds useful for treating schizophrenia, bipolar disorder, or major depression, e.g., such as those described in U.S. Pat. Nos. 6,297,262; 6,284,760; 6,284,771; 6,232,326; 6,187,752; 6,117,890; 6,239,162 or 6,166,008.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17^{th} ed. 1985)).

The modulators (e.g., agonists or antagonists) of the expression or activity of the a polypeptide or polynucleotide of the invention, alone or in combination with other suitable components, can be made into aerosol formulations (i.e., they can be “nebulized”) to be administered via inhalation or in compositions useful for injection. Aerosol formulations can be placed into pressurized acceptable propellants, such as dichlorodifluoromethane, propane, nitrogen, and the like.

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, nasally, topically, intravenously, intraperitoneally, or intrathecally. The formulations of compounds can be presented in unit-dose or multi-dose sealed containers, such as ampoules and vials. Solutions and suspensions can be prepared from sterile powders, granules, and tablets of the kind previously described. The modulators can also be administered as part of a prepared food or drug.

The dose administered to a patient, in the context of the present invention should be sufficient to effect a beneficial response in the subject over time. The optimal dose level for any patient will depend on a variety of factors including the efficacy of the specific modulator employed, the age, body weight, physical activity, and diet of the patient, on a possible combination with other drugs, and on the severity of the mental disorder. The size of the dose also will be determined by the existence, nature, and extent of any adverse side effects that accompany the administration of a particular compound or vector in a particular subject.

In determining the effective amount of the modulator to be administered a physician may evaluate circulating plasma levels of the modulator, modulator toxicity, and the production of anti-modulator antibodies. In general, the dose equivalent of a modulator is from about 1 ng/kg to 10 mg/kg for a typical subject.

For administration, modulators of the present invention can be administered at a rate determined by the LD-50 of the modulator, and the side effects of the modulator at various concentrations, as applied to the mass and overall health of the subject. Administration can be accomplished via single or divided doses.

IX. Gene Therapy Applications

A variety of human diseases can be treated by therapeutic approaches that involve stably introducing a gene into a human cell such that the gene is transcribed and the gene product is produced in the cell. Diseases amenable to treatment by this approach include inherited diseases, including those in which the defect is in a single or multiple genes. Gene therapy is also useful for treatment of acquired diseases and other conditions. For discussions on the application of gene therapy towards the treatment of genetic as well as acquired diseases, see, Miller, Nature 357:455-460 (1992); and Mulligan, Science 260:926-932 (1993).

In the context of the present invention, gene therapy can be used for treating a variety of disorders and/or diseases in which the polynucleotides and polypeptides of the invention has been implicated. For example, compounds, including polynucleotides, can be identified by the methods of the present invention as effective in treating a mental disorder. Introduction by gene therapy of these polynucleotides can then be used to treat, e.g., mental disorders including mood disorders or psychotic disorders (e.g., schizophrenia).

A. Vectors for Gene Delivery

For delivery to a cell or organism, the polynucleotides of the invention can be incorporated into a vector. Examples of vectors used for such purposes include expression plasmids capable of directing the expression of the nucleic acids in the target cell. In other instances, the vector is a viral vector system wherein the nucleic acids are incorporated into a viral genome that is capable of transfecting the target cell. In a preferred embodiment, the polynucleotides can be operably linked to expression and control sequences that can direct expression of the gene in the desired target host cells. Thus, one can achieve expression of the nucleic acid under appropriate conditions in the target cell.

B. Gene Delivery Systems

Viral vector systems useful in the expression of the nucleic acids include, for example, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including but not limited to Rous sarcoma virus), and MoMLV. Typically, the genes of interest are inserted into such vectors to allow packaging of the gene construct, typically with accompanying viral DNA, followed by infection of a sensitive host cell and expression of the gene of interest.

As used herein, “gene delivery system” refers to any means for the delivery of a nucleic acid of the invention to a target cell. In some embodiments of the invention, nucleic acids are conjugated to a cell receptor ligand for facilitated uptake (e.g., invagination of coated pits and internalization of the endosome) through an appropriate linking moiety, such as a DNA linking moiety (Wu et al., J. Biol. Chem. 263:14621-14624 (1988); WO 92/06180). For example, nucleic acids can be linked through a polylysine moiety to asialo-oromucocid, which is a ligand for the asialoglycoprotein receptor of hepatocytes.

Similarly, viral envelopes used for packaging gene constructs that include the nucleic acids of the invention can be modified by the addition of receptor ligands or antibodies specific for a receptor to permit receptor-mediated endocytosis into specific cells (see, e.g., WO 93/20221, WO 93/14188, and WO 94/06923). In some embodiments of the invention, the DNA constructs of the invention are linked to viral proteins, such as adenovirus particles, to facilitate endocytosis (Curiel et al., Proc. Natl. Acad. Sci. U.S.A. 88:8850-8854 (1991)). In other embodiments, molecular conjugates of the instant invention can include microtubule inhibitors (WO/9406922), synthetic peptides mimicking influenza virus hemagglutinin (Plank et al., J. Biol. Chem. 269:12918-12924 (1994)), and nuclear localization signals such as SV40 T antigen (WO93/19768).

Retroviral vectors are also useful for introducing the nucleic acids of the invention into target cells or organisms. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site) (see, Mulligan, In: Experimental Manipulation of Gene Expression, Inouye (ed), 155-173 (1983); Mann et al., Cell 33:153-159 (1983); Cone and Mulligan, Proceedings of the National Academy of Sciences, U.S.A., 81:6349-6353 (1984)).

The design of retroviral vectors is well known to those of ordinary skill in the art. In brief, if the sequences necessary for encapsidation (or packaging of retroviral RNA into infectious virions) are missing from the viral genome, the result is a cis-acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including, e.g., European Patent Application EPA 0 178 220; U.S. Pat. No. 4,405,712, Gilboa Biotechniques 4:504-512 (1986); Mann et al., Cell 33:153-159 (1983); Cone and Mulligan Proc. Natl. Acad. Sci. USA 81:6349-6353 (1984); Eglitis et al. Biotechniques 6:608-614 (1988); Miller et al. Biotechniques 7:981-990 (1989); Miller (1992) supra; Mulligan (1993), supra; and WO 92/07943.

The retroviral vector particles are prepared by recombinantly inserting the desired nucleotide sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence. As a result, the patient is capable of producing, for example, a polypeptide or polynucleotide of the invention and thus restore the cells to a normal phenotype.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include Crip, GPE86, PA317 and PG13 (see Miller et al., J. Virol. 65:2220-2224 (1991)). Examples of other packaging cell lines are described in Cone and Mulligan Proceedings of the National Academy of Sciences, USA, 81:6349-6353 (1984); Danos and Mulligan Proceedings of the National Academy of Sciences, USA, 85:6460-6464 (1988); Eglitis et al. (1988), supra; and Miller (1990), supra.

Packaging cell lines capable of producing retroviral vector particles with chimeric envelope proteins may be used. Alternatively, amphotropic or xenotropic envelope proteins, such as those produced by PA317 and GPX packaging cell lines may be used to package the retroviral vectors.

In some embodiments of the invention, an antisense polynucleotide is administered which hybridizes to a gene encoding a polypeptide of the invention. The antisense polypeptide can be provided as an antisense oligonucleotide (see, e.g., Murayama et al., Antisense Nucleic Acid Drug Dev. 7:109-114 (1997)). Genes encoding an antisense nucleic acid can also be provided; such genes can be introduced into cells by methods known to those of skill in the art. For example, one can introduce an antisense nucleotide sequence in a viral vector, such as, for example, in hepatitis B virus (see, e.g., Ji et al., J. Viral Hepat. 4:167-173 (1997)), in adeno-associated virus (see, e.g., Xiao et al., Brain Res. 756:76-83 (1997)), or in other systems including, but not limited, to an HVJ (Sendai virus)-liposome gene delivery system (see, e.g., Kaneda et al., Ann. NY Acad. Sci. 811:299-308 (1997)), a “peptide vector” (see, e.g., Vidal et al., CR Acad. Sci III 32:279-287 (1997)), as a gene in an episomal or plasmid vector (see, e.g., Cooper et al., Proc. Natl. Acad. Sci. U.S.A. 94:6450-6455 (1997), Yew et al. Hum Gene Ther. 8:575-584 (1997)), as a gene in a peptide-DNA aggregate (see, e.g., Niidome et al., J. Biol. Chem. 272:15307-15312 (1997)), as “naked DNA” (see, e.g., U.S. Pat. Nos. 5,580,859 and 5,589,466), in lipidic vector systems (see, e.g., Lee et al., Crit Rev Ther Drug Carrier Syst. 14:173-206 (1997)), polymer coated liposomes (U.S. Pat. Nos. 5,213,804 and 5,013,556), cationic liposomes (Epand et al., U.S. Pat. Nos. 5,283,185; 5,578,475; 5,279,833; and 5,334,761), gas filled microspheres (U.S. Pat. No. 5,542,935), ligand-targeted encapsulated macromolecules (U.S. Pat. Nos. 5,108,921; 5,521,291; 5,554,386; and 5,166,320).

In another embodiment, conditional expression systems, such as those typified by the tet-regulated systems and the RU-486 system, can be used (see, e.g., Gossen & Bujard, PNAS 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)). These systems impart small molecule control on the expression of the target gene(s) of interest.

C. Pharmaceutical Formulations

When used for pharmaceutical purposes, the vectors used for gene therapy are formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. Biochemistry 5:467 (1966).

The compositions can additionally include a stabilizer, enhancer, or other pharmaceutically acceptable carriers or vehicles. A pharmaceutically acceptable carrier can contain a physiologically acceptable compound that acts, for example, to stabilize the nucleic acids of the invention and any associated vector. A physiologically acceptable compound can include, for example, carbohydrates, such as glucose, sucrose or dextrans; antioxidants, such as ascorbic acid or glutathione; chelating agents; low molecular weight proteins or other stabilizers or excipients. Other physiologically acceptable compounds include wetting agents, emulsifying agents, dispersing agents, or preservatives, which are particularly useful for preventing the growth or action of microorganisms. Various preservatives are well known and include, for example, phenol and ascorbic acid. Examples of carriers, stabilizers, or adjuvants can be found in Remington's Pharmaceutical Sciences, Mack Publishing Company, Philadelphia, Pa., 17th ed. (1985).

D. Administration of Formulations

The formulations of the invention can be delivered to any tissue or organ using any delivery method known to the ordinarily skilled artisan. In some embodiments of the invention, the nucleic acids of the invention are formulated in mucosal, topical, and/or buccal formulations, particularly mucoadhesive gel and topical gel formulations. Exemplary permeation enhancing compositions, polymer matrices, and mucoadhesive gel preparations for transdermal delivery are disclosed in U.S. Pat. No. 5,346,701.

E. Methods of Treatment

The gene therapy formulations of the invention are typically administered to a cell. The cell can be provided as part of a tissue, such as an epithelial membrane, or as an isolated cell, such as in tissue culture. The cell can be provided in vivo, ex vivo, or in vitro.

The formulations can be introduced into the tissue of interest in vivo or ex vivo by a variety of methods. In some embodiments of the invention, the nucleic acids of the invention are introduced into cells by such methods as microinjection, calcium phosphate precipitation, liposome fusion, or biolistics. In further embodiments, the nucleic acids are taken up directly by the tissue of interest.

In some embodiments of the invention, the nucleic acids of the invention are administered ex vivo to cells or tissues explanted from a patient, then returned to the patient. Examples of ex vivo administration of therapeutic gene constructs include Nolta et al., Proc Natl. Acad. Sci. USA 93(6):2414-9 (1996); Koc et al., Seminars in Oncology 23 (1):46-65 (1996); Raper et al., Annals of Surgery 223(2):116-26 (1996); Dalesandro et al., J. Thorac. Cardi. Surg., 11(2):416-22 (1996); and Makarov et al., Proc. Natl. Acad. Sci. USA 93(1):402-6 (1996).

X. Diagnosis of Mood Disorders and Psychotic Disorders

The present invention also provides methods of diagnosing mood disorders (such as major depression or bipolar disorder), psychotic disorders (such as schizophrenia). In one preferred embodiment, the disease state encompasses psychotic disorders. Diagnosis involves determining the level of a polypeptide or polynucleotide of the invention in a patient and then comparing the level to a baseline or range. Typically, the baseline value is representative of a polypeptide or polynucleotide of the invention in a healthy person not suffering from a mood disorder or psychotic disorder or under the effects of medication or other drugs. Variation of levels of a polypeptide or polynucleotide of the invention from the baseline range (either up or down) indicates that the patient has a mood disorder or psychotic disorder or at risk of developing at least some aspects of a mood disorder or psychotic disorder. In some embodiments, the level of a polypeptide or polynucleotide of the invention are measured by taking a blood, urine or tissue sample from a patient and measuring the amount of a polypeptide or polynucleotide of the invention in the sample using any number of detection methods, such as those discussed herein, e.g., SNPs or haplotypes associated with this genes. The genes provided herein also can be used to develop probe sets for PCR and chip assays.

Single nucleotide polymorphism (SNP) analysis is also useful for detecting differences between alleles of the polynucleotides (e.g., genes) of the invention. SNPs linked to genes encoding polypeptides of the invention are useful, for instance, for diagnosis of diseases (e.g., mood disorders such as bipolar disease, major depression, and schizophrenia disorders) whose occurrence is linked to the gene sequences of the invention. For example, if an individual carries at least one SNP linked to a disease-associated allele of the gene sequences of the invention, the individual is likely predisposed for one or more of those diseases. If the individual is homozygous for a disease-linked SNP, the individual is particularly predisposed for occurrence of that disease. In some embodiments, the SNP associated with the gene sequences of the invention is located within 300,000; 200,000; 100,000; 75,000; 50,000; or 10,000 base pairs from the gene sequence.

Various real-time PCR methods can be used to detect SNPs, including, e.g., Taqman or molecular beacon-based assays (e.g., U.S. Pat. Nos. 5,210,015; 5,487,972; Tyagi et al., Nature Biotechnology 14:303 (1996); and PCT WO 95/13399 are useful to monitor for the presence of absence of a SNP. Additional SNP detection methods include, e.g., DNA sequencing, sequencing by hybridization, dot blotting, oligonucleotide array (DNA Chip) hybridization analysis, or are described in, e.g., U.S. Pat. No. 6,177,249; Landegren et al., Genome Research, 8:769-776 (1998); Botstein et al., Am J Human Genetics 32:314-331 (1980); Meyers et al., Methods in Enzymology 155:501-527 (1987); Keen et al., Trends in Genetics 7:5 (1991); Myers et al., Science 230:1242-1246 (1985); and Kwok et al., Genomics 23:138-144 (1994).

In some embodiments, the level of the enzymatic product of a polypeptide or polynucleotide of the invention is measured and compared to a baseline value of a healthy person or persons. Modulated levels of the product compared to the baseline indicates that the patient has a mood disorder or psychotic disorder or is at risk of developing at least some aspects of a mood disorder or psychotic disorder. Patient samples, for example, can be blood, PBS, lymphocytes, saliva, CSF, urine or tissue samples.

Immunoassays using antigens and antibodies for genes differentially expressed in psychotic disorders are also useful for immunoassays such as ELISA and immunohistochemical assays. The genes described herein are also useful for making differential diagnoses for psychiatric disorders.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

### Identification of Genes Dysregulated in Psychotic Disorders

Post mortem mental disorder brains (i.e. from schizophrenia patients) and control brains were used in this study. Each brain pair (case and control) was matched on the basis of gender, age, and postmortem interval. Ten brain regions, anterior cingulate cortex (AnCg), dorsolateral prefrontal cortex (DLPFC), cerebellar cortex (CB), entorhinal cortex (ERC), superior temporal gyrus (STG), parietal cortex (PC), nucleus accumbens (nAcc), ventral thalamus (VThal), medial thalamus (MThal) and/or the hippocampus (HC) were extracted for RNA and subjected to microarray analysis using Affymetrix oligonucleotide GeneChips™. Each RNA sample was subjected to two independent analyses. The results were analyzed using multiple statistical tools and algorithms with various stringencies. The patient's particular conditions in their terminal phase (agonal factors, e.g., seizure, coma, hypoxia, dehydration, and pyrexia) and the conditions of the brain tissue after death (postmortem factors, e.g., postmortem interval, and freezer interval) are two major influences on RNA preservation in postmortem brain tissue. Brain pH has been evaluated as an indicator for agonal status, and as an indicator of RNA preservation. Subjects with agonal factors and low pH samples, in which RNA quality was found to be compromised were eliminated from the study. The genes identified using this study are listed in Table 1.

Genes differentially expressed in mental disorders and their gene ontologies are listed in Tables 2-22. Each gene ontology (GO) term is listed with accompanying gene list of differentially expressed genes that belong to the given GO term. A separate table is given for each term either within specific brain regions or across a union of regions as indicated. An annotated table showing the enrichment of synaptic transmission, neurogenesis, ribosomal, cation homeostasis, and heat shock protein is included for genes 1.2 fold in any brain region. The current invention establishes a strong association between schizophrenia and genes in chromosomes 2, 5, 6 and 12.

Within genes differentially expressed in both Bipolar disorders (BP) and Schizophrenia (SZ) in either the AnCg or DLPFC, the direction of change in the disease state compared to controls is typically the same. Conversely, genes commonly differentially expressed in both SZ and Mayor depression (MD) are often disregulated in opposing directions. For example, genes disregulated in both SZ and MD are typically increased in SZ and decreased in MD. This is true for both the AnCg and DLPFC but is most striking in the DLPFC.

### Example 1

### Identification of Genes Dysregulated in Psychotic Disorders

The genes listed in Tables 22-25 are from the analysis of 3 brain regions, Dorsolateral Prefrontal Cortex (DLFC), Anterior Cingulate (AnCg) and Amigdala (AMY). The analysis was based on a new cohort of 10 schizophrenics and 7 controls. The criteria for selecting the brains were agonal factor (AFS=0), pH (>6.4), and ratio 28S/18S. Data was analyzed by GCRMA. Duplicated experimental data was averaged. Student t test was applied for statistical significance. The criteria of p<0.05, and fold change>1.2 or <0.83333 were used for significant criteria.

### Example 3

### Variation in GRM3 Affects Cognition, Prefrontal Glutamate, and Risk for Schizophrenia

As described in Egan et al., Proc. Nat'l Acad. Sci. USA 101:12604-12609 (2004) (herein incorporated by reference in its entirety), the metabotropic glutamate receptor GRM3 is involved in schizophrenia. A common GRM3 haplotype is strongly associated with schizophrenia. Within this hapltotype, the A allele of a single-nucleotdite polymorphism (SNP) 4 (hCV11245618) in intron 2 is overtransmitted.

The above examples are provided to illustrate the invention but not to limit its scope. Other variants of the invention will be readily apparent to one of ordinary skill in the art and are encompassed by the appended claims. All publications, databases, Genbank sequences, GO terms, patents, and patent applications cited herein are hereby incorporated by reference.

TABLE 1

| GenBank | | | | | | | | | | | | |
|---|---|---|---|---|---|---|---|---|---|---|---|---|
| Accession | | | | | | | | | | | | |
| Nos. | Gene Name | Chromosome | AnCg | CB | DLPFC | ERC | HC | nAcc | PC | STG | Mthal | Vthal |
| U48705 | | | | | 1.39 | | | | | | 1.27 | |
| NM_000991.1 | ribosomal protein L28 | \|19\|19q\|19q13\| | | | 1.23 | | | 1.24 | | | | 1.25 |
| NM_003753.1 | eukaryotic translation initiation factor | \|22\|22q\|22q13\| | | | | | | | | | | 1.25 |
| | 3, subunit 7 zeta, 66/67 kDa | | | | | | | | | | | |
| NM_003753.1 | eukaryotic translation initiation factor | \|22\|22q\|22q13\| | | | | | | | | | | 1.25 |
| | 3, subunit 7 zeta, 66/67 kDa | | | | | | | | | | | |
| NM_004500.1 | heterogeneous nuclear | \|14\|14q\|14q11\| | 1.31 | | 1.23 | | | | | | | |
| | ribonucleoprotein C (C1/C2) | | | | | | | | | | | |
| NM_004404.1 | neural precursor cell expressed, | \|2\|2q\| | 1.25 | 1.20 | 1.47 | | | | | | 1.21 | |
| | developmentally down-regulated 5 | | | | | | | | | | | |
| NM_003754.1 | eukaryotic translation initiation factor | \|11\| | | | 1.20 | | | | | | | 1.22 |
| | 3, subunit 5 epsilon, 47 kDa | | | | | | | | | | | |
| NM_007104.2 | ribosomal protein L10a | \|6\|6p\|6p21\| | | | | | 1.21 | 1.23 | | | | |
| NM_001344.1 | defender against cell death 1 | \|14\|14q\|14q11\| | | 1.23 | 1.29 | 1.32 | | | 1.25 | | | 1.27 |
| L05095.1 | hypothetical protein FLJ22875 | \|15\|15q\|15q22\| | | | 1.23 | | | | | | | |
| U16738.1 | ribosomal protein L14 | \|3\|3p\|3p22\| | | | | | 1.22 | | | | | |
| BE869922 | H3 histone, family 3A | \|1\|1q\| | 1.26 | | 1.34 | | | | | | | |
| AK024976.1 | coated vesicle membrane protein | \|12\|12q\|12q24\| | 1.24 | | | | | | | | | 1.25 |
| BG168896 | farnesyltransferase, CAAX box, alpha | \|8\|8p\|8p22\| | | | 1.36 | | | | 1.25 | | | 1.39 |
| N32864 | histidine triad nucleotide binding | \|5\|5q\|5q31\| | 1.20 | | | | | 1.23 | | | | |
| | protein 1 | | | | | | | | | | | |
| AI862255 | ATPase, H+ transporting, lysosomal | \|5\|5q\|5q35\| | | 1.24 | 1.42 | 1.23 | | | 1.27 | | 1.22 | 1.21 |
| | 9 kDa, V0 subunit e | | | | | | | | | | | |
| NM_002444.1 | moesin | \|X\|Xq\|Xq11\| | | 1.21 | 1.38 | | | | 1.39 | | | |
| NM_006265.1 | RAD21 homolog (S. pombe) | \|8\|8q\| | | | | | | | | | 1.22 | |
| NM_004068.1 | adaptor-related protein complex 2, | \|3\|3q\| | | | | | | 1.25 | | 1.26 | | |
| | mu 1 subunit | | | | | | | | | | | |
| NM_004872.1 | chromosome 1 open reading frame 8 | \|1\|1p\|1p36\| | | | | 1.22 | | | | | | 1.35 |
| NM_004184.2 | adaptor-related protein complex 1, | \|19\|19p\|19p13\| | 1.24 | | | | | 1.22 | | 1.35 | 1.23 | 1.42 |
| | mu 1 subunit | | | | | | | | | | | |
| NM_005022.1 | profilin 1 | \|17\|17p\|17p13\| | | | | | | | | | 1.21 | 1.31 |
| BC003623.1 | tyrosine 3- | \|8\|8q\|8q23\| | | | 0.80 | | 0.74 | | | | | |
| | monooxygenase/tryptophan 5- | | | | | | | | | | | |
| | monooxygenase activation protein, | | | | | | | | | | | |
| | zeta polpeptide | | | | | | | | | | | |
| U28964.1 | tyrosine 3- | \|8\|8q\|8q23\| | | 0.79 | 0.73 | | 0.69 | | | | | |
| | monooxygenase/tryptophan 5- | | | | | | | | | | | |
| | monooxygenase activation protein, | | | | | | | | | | | |
| | zeta polypeptide | | | | | | | | | | | |
| NM_000454.1 | superoxide dismutase 1, soluble | \|21\|21q\|21q22\| | | | | | | | | | | 1.23 |
| | (amyotrophic lateral sclerosis 1 | | | | | | | | | | | |
| | (adult)) | | | | | | | | | | | |
| NM_023009.1 | macrophage myristoylated alanine- | \|1\|1p\|1p34\| | | | 1.27 | | | | | | | |
| | rich C kinase substrate | | | | | | | | | | | |
| NM_005566.1 | lactate dehydrogenase A | \|11\|11p\|11p15\| | 1.31 | | | | | 1.25 | | 1.29 | 1.29 | |
| J02783.1 | procollagen-proline, 2-oxoglutarate 4- | \|17\|17q\| | | | | | | | | | | 1.21 |
| | dioxygenase (proline 4-hydroxylase), | | | | | | | | | | | |
| | beta polypeptide (protein disulfide | | | | | | | | | | | |
| | isomerase; thyroid hormone binding | | | | | | | | | | | |
| | protein p55) | | | | | | | | | | | |
| NM_014765.1 | translocase of outer mitochondrial | \|1\|1q\| | 1.23 | | | | | 1.20 | | 1.22 | | |
| | membrane 20 (yeast) homolog | | | | | | | | | | | |
| NM_003118.1 | secreted protein, acidic, cysteine-rich | \|5\|5q\|5q31\| | | 1.73 | | | | | | | 1.47 | |
| | (osteonectin) | | | | | | | | | | | |
| NM_006145.1 | DnaJ (Hsp40) homolog, subfmaily B, | \|19\|19p\|19p13\| | | | | 1.31 | | | | | | |
| | member 1 | | | | | | | | | | | |
| NM_004339.2 | pituitary tumor-transforming 1 | \|21\|21q\|21q22\| | 1.39 | 1.29 | 1.52 | 1.40 | 1.20 | | 1.24 | | 1.26 | 1.25 |
| | interacting protein | | | | | | | | | | | |
| NM_006708.1 | glyoxalase I | \|6\|6p\|6p21\| | 1.20 | | | | | | | | | |
| BC000478.1 | heat shock 70 kDa protein 9B | \|5\|5q\|5q31\| | | | | | | | | | | 1.23 |
| | (mortalin-2) | | | | | | | | | | | |
| NM_006826.1 | tyrosine 3- | \|2\| | | | | | | | | | 1.30 | 1.26 |
| | monooxygenase/tryptophan 5- | | | | | | | | | | | |
| | monooxygenase activation protein, | | | | | | | | | | | |
| | theta polypeptide | | | | | | | | | | | |
| NM_000177.1 | gelsolin (amyloidosis, Finnish type) | \|9\|9q\| | | | | | | | 1.25 | | | 1.43 |
| NM_003746.1 | dynein, cytoplasmic, light polypeptide 1 | \|12\|12q\|12q24\| | | | | | | | | | | |
| AB034747.1 | LPS-induced TNF-alpha factor | \|16\|16p\|16p13\| | | | 1.51 | 1.30 | 1.20 | | 1.53 | | 1.35 | 1.63 |
| NM_006013.1 | ribosomal protein L10 | \|X\|Xq\| | | | 1.22 | | | | | | | |
| AA699583 | ARP2 actin-related protein 2 homolog | \|2\|2p\| | 1.27 | | | | | 1.20 | | 1.25 | | |
| | (yeast) | | | | | | | | | | | |
| NM_000291.1 | phosphoglycerate kinase 1 | \|X\|Xq\| | 1.34 | | | | | | | | | |
| NM_000291.1 | phosphoglycerate kinase 1 | \|X\|Xq\| | 1.21 | | | | | | | 1.21 | | |
| BG231932 | ceroid-lipofuscinosis, neuronal 2, late | \|11\|11P\| | | | | 1.36 | | | 1.22 | | 1.27 | 127 |
| | infantile (Jansky-Bielschowsky | | | | | | | | | | | |
| | disease) | | | | | | | | | | | |
| BF112006 | RAN, member RAS oncogene family | \|6\|6p\| | 1.24 | | | | | | | | | |
| AF054183.1 | RAN, member RAS oncogene family | \|6\|6p\| | 1.26 | | | | | 1.23 | | | | |
| N92494 | vitamin A responsive; cytoskeleton | \|3\|3p\| | | | | 1.26 | | 1.27 | | 1.34 | 1.24 | |
| | related | | | | | | | | | | | |
| NM_001003.1 | ribosomal protein, large, P1 | \|15\|15q\| | | | 1.22 | | | | | | | |
| NM_001903.1 | catenin (cadherin-associated protein), | \|5\|5q\| | | 1.24 | 1.38 | 1.39 | | | | | 1.30 | 1.39 |
| | alpha 1 (102 kDa | | | | | | | | | | | |
| BF686442 | prothymosin, alpha (gene sequence | \|2\|2q\|2q35\| | | | 1.35 | | | | | | | 1.35 |
| | 28) | | | | | | | | | | | |
| NM_001019.1 | ribosomal protein S15a | \|16\|16p\| | | | 1.25 | | | | | | | |
| NM_002539.1 | ornithine decarboxylase 1 | \|2\|2p\| | 1.28 | | | | | | | 1.28 | 1.20 | 1.25 |
| NM_005345.3 | heat shock 70 kDa protein 1A | \|6\|6p\|6p21\| | | | 1.33 | 1.52 | | | 1.22 | | | |
| NM_005345.3 | heat shock 70 kDa protein 1A | \|6\|6p\|6p21\| | | | 1.37 | 1.55 | | | 1.22 | 1.30 | | |
| NM_006513.1 | seryl-tRNA synthetase | \|1\|1p\|1p13\| | | | | | | | | 1.20 | | 1.24 |
| NM_003217.1 | testis enhanced gene transcript (BAX | \|12\|12q\|12q12\| | | | 1.26 | 1.24 | | | | | 1.20 | |
| | inhibitor 1) | | | | | | | | | | | |
| BE256479 | heat shock 60 kDa protein 1 | \|12\|12q\| | 1.33 | | | 1.47 | | | | | | |
| | (chaperonin) | | | | | | | | | | | |
| NM_002156.1 | heat shock 60 kDa protein 1 | \|12\|12q\| | | | | 1.25 | | | | | | |
| | (chaperonin) | | | | | | | | | | | |
| NM_006429.1 | chaperonin containing TCP1, subunit | \|2\|2p\| | | | | | | 1.23 | | | | |
| | 7 (eta) | | | | | | | | | | | |
| NM_002812.1 | proteasome (prosome, macropain) | \|19\|19q\|19q13\| | 1.20 | | | | | | | | | |
| | 26S subunit, non-ATPase, 8 | | | | | | | | | | | |
| NM_013995.1 | lysosomal-associated membrane | \|X\|Xq\| | | 1.20 | 1.77 | | | | 1.48 | | 1.36 | 1.74 |
| | protein 2 | | | | | | | | | | | |
| NM_000992.1 | ribosomal protein L29 | \|3\|3p\|p21\| | | 1.20 | 1.20 | | | 1.21 | | | 1.24 | |
| NM_002808.1 | proteasome (prosome, macropain) | \|3\|3q\|3q27\| | | | | | | | | | | 1.22 |
| | 26S subunit, non-ATPase, 2 | | | | | | | | | | | |
| AB032261.1 | stearoyl-CoA desaturase (delta-9- | \|10\|10q\|10q23\| | | | | | | | | 0.59 | | |
| | desaturase) | | | | | | | | | | | |
| NM_005745.3 | accessory protein BAP31 | \|X\|Xq\| | | | | | | | | | | 1.26 |
| NM_005548.1 | lysyl-tRNA synthetase | \|16\|16q\|16q23\| | 1.36 | | | | | 1.20 | 1.26 | 1.29 | | 1.27 |
| BE869583 | anti-oxidant protein 2 (non-selenium | \|1\|1q\|1q23\| | 1.21 | | 1.26 | 1.20 | | | | | | |
| | glutathione peroxidase, acidic | | | | | | | | | | | |
| | calcium-independent phospholipase | | | | | | | | | | | |
| | A2) | | | | | | | | | | | |
| NM_004905.1 | anti-oxidant protein 2 (non-selenium | \|1\|1q\|1q23\| | | | 1.30 | | | | | | | |
| | glutathione peroxidase, acidic | | | | | | | | | | | |
| | calcium-independent phospholipase | | | | | | | | | | | |
| | A2) | | | | | | | | | | | |
| NM_016127.1 | hypothetical protein MGC8721 | \|8\|8p\| | 1.24 | | | | | | | | | |
| AA479488 | S-adenosylhomocysteine hydrolase- | \|1\|1p\| | 1.64 | 1.28 | 1.45 | 1.53 | 1.26 | | 1.28 | | 1.35 | |
| | like 1 | | | | | | | | | | | |
| AA479488 | S-adenosylhomocysteine hydrolase- | \|1\|1p\| | 1.32 | 1.22 | 1.43 | 1.28 | | | | | 1.24 | |
| | like 1 | | | | | | | | | | | |
| NM_006621.1 | S-adenosylhomocysteine hydrolase- | \|1\|1p\| | 1.45 | 1.20 | 1.43 | 1.35 | 1.22 | | | 1.25 | 1.20 | |
| | like 1 | | | | | | | | | | | |
| NM_002106.1 | H2A histone family, member Z | \|4\|4q\| | 1.28 | | | 1.27 | | 1.26 | | 1.24 | | |
| NM_001012.1 | ribosomal protein S8 | \|1\|1p\|1p34\| | | | 1.26 | | | | | | | |
| NM_014762.1 | 24-dehydrocholesterol reductase | \|1\|1p\|1p33\| | | | | | | 1.27 | 1.20 | | | |
| NM_000980.1 | ribosomal protein L18a | \|19\|19p\| | | 1.30 | 1.30 | | | 1.41 | | | | 1.24 |
| NM_002778.1 | prosaposin (variant Gaucher disease | \|10\|10q\|10q21\| | 1.21 | | | | | | | 1.22 | | 1.20 |
| | and variant metachromatic | | | | | | | | | | | |
| | leukodystrophy) | | | | | | | | | | | |
| NM_006585.1 | chaperonin containing TCP1, subunit | \|21\|21q\|21q22\| | 1.31 | | | | | 1.23 | 1.27 | 132 | | |
| | 8 (theta) | | | | | | | | | | | |
| NM_002793.1 | proteasome (prosome, macropain) | \|6\|6q\| | | | | | | | | | | 1.27 |
| | subunit, beta type, 1 | | | | | | | | | | | |
| NM_006430.1 | chaperonin containing TCP1, subunit | \|2\|2p\| | 1.20 | | | 1.24 | | | | 1.32 | | 1.29 |
| | 4 (delta) | | | | | | | | | | | |
| AL534104 | DnaJ (Hsp40) homolog, subfamily A, | \|9\|9p\|9p13\| | 1.27 | | | | | | | | | |
| | member 1 | | | | | | | | | | | |
| NM_001539.1 | DnaJ (Hsp40) homolog, subfamily A, | \|9\|9p\|9p13\| | | | | | | | | | 1.26 | |
| | member 1 | | | | | | | | | | | |
| NM_003366.1 | ubiquinol-cytochrome c reductase | \|16\|16p\| | 1.36 | | | | | 1.24 | | 1.29 | | |
| | core protein II | | | | | | | | | | | |
| NM_016081.1 | palladin | \|4\|4q\|4q32\| | 1.29 | | 1.32 | 1.22 | 1.31 | | | | | |
| NM_012215.1 | meningioma expressed antigen 5 | \|10\|10q\|10q24\| | 1.23 | | | | | | | | | |
| | (hyaluronidase) | | | | | | | | | | | |
| NM_001004.1 | ribosomal protein, large P2 | \|11\|11p\|11p15\| | | | 1.22 | | | | | | | |
| NM_005998.1 | chaperonin containing TCP1, subunit | \|1\|1q\| | 1.20 | | | | | 1.24 | 1.20 | | | 1.23 |
| | 3 (gamma) | | | | | | | | | | | |
| NM_000973.1 | ribosomal protein L8 | \|8\|8q\|8q24\| | | | | | | | | | | 1.23 |
| NM_005625.1 | syndecan binding protein (syntenin) | \|8\|8q\| | 1.23 | | | | | | | | | |
| AI348010 | Homo sapiens cDNA FLJ36224 fis, | | | | 1.21 | 1.29 | | 1.23 | 1.25 | 1.33 | 1.23 | |
| | clone THYMU2000990 | | | | | | | | | | | |
| NM_000942.1 | peptidylprolyl isomerase B | \|15\|15q\|15q21\| | | | | | | | | | | 1.26 |
| | (cyclophilin B) | | | | | | | | | | | |
| BG107676 | stress-associated endoplasmic | \|3\|3q\|3q25\| | 1.33 | | 1.21 | | | | | | | |
| | reticulum protein 1; ribosome | | | | | | | | | | | |
| | associated membrane protein 4 | | | | | | | | | | | |
| AL136807.1 | stress-associated endoplasmic | \|3\|3q\|3q25\| | 1.20 | | | | | | | | | |
| | reticulum protein 1; ribosome | | | | | | | | | | | |
| | associated membrane protein 4 | | | | | | | | | | | |
| AF090891.1 | Tax1 (human T-cell leukemia virus | \|7\|7p\| | 1.25 | | | | | | | | | |
| | type I) binding protein 1 | | | | | | | | | | | |
| NM_000611.1 | CD59 antigen p18-20 (antigen | \|11\|11p\| | 1.36 | | | 1.41 | | 1.30 | 1.23 | 1.21 | 1.42 | 1.46 |
| | identified by monoclonal antibodies | | | | | | | | | | | |
| | 16.3A5, EJ16, EJ30, EL32 and G344) | | | | | | | | | | | |
| NM_001769.1 | CD9 antigen (p24) | \|12\|12p\|12p13\| | | | 1.50 | | | | | | 1.31 | 1.47 |
| NM_005642.1 | TAF7 RNA polymerase II, TATA box | \|5\|5q\| | 1.25 | | | | | | | | | 1.26 |
| | binding protein (TBP)-associated | | | | | | | | | | | |
| | factor, 55 kDa | | | | | | | | | | | |
| NM_002414.1 | antigen identified by monoclonal | \|X\|Xp\|Xp22\| | 1.52 | 1.31 | 1.67 | 1.43 | | | 1.31 | 1.26 | 1.44 | |
| | antibodies 12E7, F21 and O13 | | | | | | | | | | | |
| BE545756 | adducin 3 (gamma) | \|10\|10q\|10q24\| | 1.33 | | 1.44 | | | | | | | |
| NM_004417.2 | dual specificity phosphatase 1 | \|5\|5q\| | | | | | | 0.66 | 0.72 | | | |
| NM_022551.1 | ribosomal protein S18 | \|6\|6p\|6p21\| | | 1.24 | 1.25 | | 1.22 | | | | | |
| BE966599 | heterogeneous nuclear | \|5\|5q\| | 1.23 | | | | | | | | | |
| | ribonucleoprotein A0 | | | | | | | | | | | |
| NM_006097.1 | myosin, light polypeptide 9, | \|20\|20q\|20q11\| | | | 0.71 | 0.77 | | | 0.80 | | | |
| | regulatory | | | | | | | | | | | |
| NM_002901.1 | reticulocalbin 1, EF-hand calcium | \|11\|11p\| | | | | | | 1.26 | | | | |
| | binding domain | | | | | | | | | | | |
| NM_004082.2 | dynactin 1 (p150, glued homolog, | \|2\|2p\| | | | | | | 1.22 | | | | |
| | Drosophila) | | | | | | | | | | | |
| NM_002266.1 | karyopherin alpha 2 (RAG cohort 1, | \|17\|17q\|17q23\| | 1.28 | | | | | | | | | |
| | importin alpha 1) | | | | | | | | | | | |
| BE299495 | hypothetical protein FLJ20719 | \|1\|1p\| | | | 1.20 | | | | | | | |
| NM_002305.2 | lectin, galactoside-binding, soluble, 1 | \|22\|22q\|22q13\| | | 1.29 | | | | | | | | |
| | (galectin 1) | | | | | | | | | | | |
| AF053641.1 | CSE1 chromosome segregation 1- | \|20\|20q\| | 1.43 | | 1.26 | 1.43 | | 1.31 | 1.31 | 1.33 | 1.32 | 1.34 |
| | like (yeast) | | | | | | | | | | | |
| NM_001873.1 | carboxypeptidase E | \|4\|4q\|4q32\| | | | | | | | | | | |
| NM_001970.1 | eukaryotic translation initiation factor | \|17\|17p\|17p13\| | | 1.61 | | 1.69 | | 1.46 | 1.67 | 2.38 | | |
| | 5A | | | | | | | | | | | |
| NM_019597.1 | heterogeneous nuclear | \|X\|Xq\| | 1.22 | | | | | | | | | |
| | ribonucleoprotein H2 (H′) | | | | | | | | | | | |
| NM_006164.1 | nuclear factor (erythroid-derived 2)- | \|2\|2q\| | | | 1.36 | 1.23 | | | 1.25 | | | |
| | like 2 | | | | | | | | | | | |
| NM_021079.1 | N-myristoyltransferase 1 | \|17\|17q\|17q21\| | | | | | | | | 1.26 | | 1.22 |
| AL556190 | cold shock domain protein A | \|12\|12p\|12p13\| | | | | | | | | 1.50 | | |
| NM_001553.1 | insulin-like growth factor binding | \|4\|4q\| | 1.20 | | 1.24 | | | | | | 1.26 | |
| | protein 7 | | | | | | | | | | | |
| NM_001553.1 | insulin-like growth factor binding | \|4\|4q\| | 1.33 | 1.38 | 1.35 | | | | | | 1.31 | |
| | protein 7 | | | | | | | | | | | |
| NM_003945.1 | ATPase, H+ transporting, lysosomal | \|5\|5q\|5q35\| | 1.27 | 1.20 | 1.35 | 1.20 | | | 1.26 | | | |
| | 9 kDa, V0 subunit e | | | | | | | | | | | |
| NM_002775.1 | protease, serine, 11 (IGF binding) | \|10\|10q\|10q26\| | | 1.22 | 1.49 | | | | 1.23 | | | |
| AB018009.1 | solute carrier family 7 (cationic amino | \|16\|16q\|16q24\| | | | | | | | | | | 1.51 |
| | acid transporter, y+ system), member 5 | | | | | | | | | | | |
| AI860431 | proteasome (prosome, macropain) | \|2\|2q\|2q36\| | 1.27 | | | | | 1.21 | | 1.24 | | |
| | 26S subunit, non-ATPase, 1 | | | | | | | | | | | |
| NM_002592.1 | proliferating cell nuclear antigen | \|20\|20p\|20pter\| | | | | | | | | | | 1.32 |
| NM_001428.1 | enolase 1, (alpha) | \|1\|1p\|1p36\| | 1.27 | | | | | | | | | |
| NM_002817.1 | proteasome (prosome, macropain) | \|11\|11p\|11p15\| | 1.25 | | | | | 1.22 | 1.21 | | | 1.29 |
| | 26S subunit, non-ATPase, 13 | | | | | | | | | | | |
| NM_017670.1 | hypothetical protein FLJ20113 | \|11\|11q\|11q13\| | | | | | | | | | | |
| NM_001020.1 | ribosomal protein S16 | \|19\|19q\|19q13\| | | 1.23 | 1.24 | | | | | | | 1.21 |
| AI768845 | synaptophysin-like protein | \|7\|7q\|7q11\| | | | | | | | | | | 1.38 |
| NM_006754.1 | synaptophysin-like protein | \|7\|7q\|7q11\| | | | | | | | | | | 1.31 |
| NM_001175.1 | Rho GDP dissociation inhibitor (GDI) | \|12\|12p\|12p12\| | | 1.25 | | | 1.29 | | | | | |
| | beta | | | | | | | | | | | |
| NM_015626.1 | SOCS box-containing WD protein | \|17\|17q\|17q11\| | | | | | | | | | | |
| | SWiP-1 | | | | | | | | | | | |
| NM_000311.1 | prion protein (p27-30) (Creutzfeld- | \|20\|20p\|20pter\| | 1.24 | | | | | | | | | |
| | Jakob disease, Gerstmann-Strausler- | | | | | | | | | | | |
| | Scheinker syndrome, fatal familial | | | | | | | | | | | |
| | insomnia) | | | | | | | | | | | |
| NM_001975.1 | enolase 2, (gamma, neuronal) | \|12\|12p\| | | | | | | 1.21 | | 1.31 | | |
| NM_006435.1 | interferon induced transmembrane | \|11\|11p\|11p15\| | 1.41 | 1.33 | 1.42 | 1.44 | 1.42 | 1.44 | 1.34 | 1.59 | 1.49 | 1.53 |
| | protein 2 (1-8D) | | | | | | | | | | | |
| NM_002787.1 | proteasome (prosome, macropain) | \|7\|7p\|7p15\| | | | | | | | 1.21 | | | 12.2 |
| | subunit, alpha type, 2 | | | | | | | | | | | |
| NM_001423.1 | epithelial membrane protein 1 | \|12\|12p\|12p12\| | 1.51 | | | | | | | | | |
| BC003570.1 | vesicle-associated membrane protein | \|1\|1p\|1p36\| | | | 1.40 | 1.28 | | | | | | 1.33 |
| | 3 (cellubrevin) | | | | | | | | | | | |
| NM_003633.1 | ectodermal-neural cortex (with BTB- | \|5\|5q\|5q12\| | | | 0.83 | | 0.80 | | | | | |
| | like domain) | | | | | | | | | | | |
| NM_024551.1 | hypothetical protein FLJ21432 | \|12\|12p\|12p13\| | | | 1.35 | | | | 1.26 | | | 1.33 |
| NM_002084.2 | glutathione peroxidase 3 (plasma) | \|5\|5q\| | | | | | 0.64 | | | 1.54 | 1.45 | |
| AI356398 | zinc finger protein 36, C3H type-like 2 | \|2\|2p\|2p22\| | | | 1.39 | | | | | | | |
| NM_006623.1 | phosphoglycerate dehydrogenase | \|1\|1p\| | | | 1.38 | 1.20 | | | 1.26 | | | |
| BC000687.1 | translocating chain-associating | \|8\|8q\|8q13\| | | | 1.36 | | | | | | 1.35 | 1.33 |
| | membrane protein | | | | | | | | | | | |
| NM_002795.1 | proteasome (prosome, macropain) | \|17\|17q\| | | | | | | 1.21 | | | | 1.20 |
| | subunit, beta type, 3 | | | | | | | | | | | |
| NM_003107.1 | SRY (sex determining region Y)-box 4 | \|6\|6p\|6p22\| | | | | | | | 0.68 | 0.66 | | 0.63 |
| NM_005410.1 | selenoprotein P, plasma, 1 | \|5\|5q\| | | 1.24 | 1.47 | | | | 1.38 | | | 1.42 |
| NM_001387.1 | dihydropyrimidinase-like 3 | \|5\|5q\| | | | | | | | | | 1.38 | |
| NM_001752.1 | catalase | \|11\|11p\| | | | 1.39 | 1.31 | | | 1.23 | | 1.22 | 1.23 |
| AF248966.1 | ATPase, H+ transporting, lysosomal | \|X\|Xq\| | 1.40 | | | | | 1.27 | | 1.32 | | |
| | interacting protein 2 | | | | | | | | | | | |
| NM_005765.1 | ATPase, H+ transporting, lysosomal | \|X\|Xq\| | 1.22 | | | | | | | | | |
| | interacting protein 2 | | | | | | | | | | | |
| NM_001839.1 | calponin 3, acidic | \|1\|1p\|1p22\| | 1.85 | 1.51 | 1.74 | 1.81 | 1.48 | | 1.69 | 1.53 | 1.97 | |
| NM_006310.1 | aminopeptidase puromycin sensitive | \|17\|17q\| | 1.21 | | | | | | | | | |
| NM_006310.1 | aminopeptidase puromycin sensitive | \|17\|17q\| | 1.26 | | | | | | | | | |
| NM_006755.1 | transaldolase 1 | \|11\|11p\|11p15\| | | | 1.26 | 1.26 | | | 1.21 | | | 1.48 |
| NM_004832.1 | glutathione-S-transferase like; | \|10\|10q\|10q25\| | 1.21 | | | | | 1.25 | | | | 1.25 |
| | glutathione transferase omega | | | | | | | | | | | |
| BC005020.1 | peptidylprolyl isomerase F | \|10\|10q\|10q22\| | | | | | | | | | 1.25 | 1.35 |
| | (cyclophilin F) | | | | | | | | | | | |
| AI889739 | myosin, heavy polypeptide 11, | \|16\|16p\|16p13\| | | | 0.73 | | | | | | | |
| | smooth muscle | | | | | | | | | | | |
| NM_022844.1 | myosin, heavy polypeptide 11, | \|16\|16p\|16p13\| | | | 0.76 | | | | | | | |
| | smooth muscle | | | | | | | | | | | |
| AI078167 | nuclear factor of kappa light | \|14\|14q\| | | 1.22 | 1.25 | 1.35 | | | | 1.30 | | |
| | polypeptide gene enhancer in B-cells | | | | | | | | | | | |
| | inhibitor, alpha | | | | | | | | | | | |
| BG500067 | Ras-GTPase-activating protein SH3- | \|5\|5q\|5q33\| | 1.35 | 1.21 | 1.26 | | | | 1.33 | 1.21 | 1.33 | 1.24 |
| | domain-binding protein | | | | | | | | | | | |
| BG398414 | replication protein A1, 70 kDa | \|17\|17p\|17p13\| | 1.31 | | 1.29 | 1.43 | | | 1.34 | 1.41 | 1.27 | 1.33 |
| NM_002945.1 | replication protein A1, 70 kDa | \|17\|17p\|17p13\| | 1.24 | | | 1.29 | | | | 1.20 | 1.24 | |
| NM_002788.1 | proteasome (prosome, macropain) | \|14\|14p\| | 1.23 | | | | | 1.20 | | 1.26 | | 1.22 |
| | subunit, alpha type, 3 | | | | | | | | | | | |
| NM_004238.1 | thyroid hormone receptor interactor | \|2\|2q\|2q36\| | | | | 1.22 | | | | 1.20 | | 1.25 |
| | 12 | | | | | | | | | | | |
| J03263.1 | lysosomal-associated membrane | \|13\|13q\| | | | | 1.29 | | | | | | 1.31 |
| | protein 1 | | | | | | | | | | | |
| NM_005561.2 | lysosomal-associated membrane | \|13\|13q\| | | | 1.32 | | | | 1.33 | | 1.34 | 1.67 |
| | protein 1 | | | | | | | | | | | |
| NM_013943.1 | chloride intracellular channel 4 | \|1\|1p\|1p36\| | | 1.36 | 1.61 | | | | | | 1.27 | 1.26 |
| NM_004039.1 | annexin A2 | \|15\|15q\|15q21\| | | | | | | | | 1.29 | 1.60 | 1.30 |
| NM_007184.1 | nischarin | \|3\|3p\|3p21\| | | 1.28 | | | | | | | | |
| NM_003641.1 | interferon induced transmembrane | \|11\| | 1.43 | 1.39 | 1.37 | | 1.46 | 1.40 | 1.21 | 1.47 | 1.39 | 1.51 |
| | protein 1 (9-27) | | | | | | | | | | | |
| NM_000696.1 | aldehyde dehydrogenase 9 family, | \|1\|1q\|1q22\| | | | 1.21 | | | | | | | |
| | member A1 | | | | | | | | | | | |
| NM_001349.1 | aspartyl-tRNA synthetase | \|2\|2q\|2q14\| | | | | | | | | | | |
| NM_005506.1 | scavenger receptor class B, member 2 | \|4\|4q\|14q21\| | | | | 1.32 | | | | | | 1.41 |
| NM_006837.1 | COP9 constitutive photomorphogenic | \|8\|8q\|8q12\| | 1.21 | | | | | | | | | |
| | homolog subunit 5 (Arabidopsis) | | | | | | | | | | | |
| NM_005776.1 | cornichon-like | \|14\|14q\|14q22\| | 1.35 | | | | | | | 1.30 | | 1.21 |
| NM_000210.1 | integrin, alpha 6 | \|2\|2q\|2q31\| | 1.33 | | 1.26 | | | | 1.25 | | 1.20 | 1.34 |
| AL525798 | fatty-acid-Coenzyme A ligase, long- | \|2\|2q\|2q34\| | 1.22 | | | | | | | 1.25 | | |
| | chain 3 | | | | | | | | | | | |
| NM_004457.2 | fatty-acid-Coenzyme A ligase, long- | \|2\|2q\|2q34\| | 1.28 | | | | | | | 1.28 | | |
| | chain 3 | | | | | | | | | | | |
| NM_000165.2 | gap junction protein, alpha 1, 43 kDa | \|6\|6q\|6q21\| | 1.52 | | 1.93 | | | | | | | |
| | (connexin 43) | | | | | | | | | | | |
| NM_002356.4 | myristoylated alanine-rich protein | \|6\|6q\|6q22\| | | | | 1.26 | | | | | | |
| | kinase C subtrate | | | | | | | | | | | |
| NM_001964.1 | early growth response 1 | \|5\|5q\|5q31\| | 0.77 | 0.77 | 0.66 | 0.73 | | | 0.71 | 0.68 | | |
| NM_002806.1 | proteasome (prosome, macropain) | \|14\|14q\|14q22\| | 1.29 | | | | | 1.24 | 1.28 | 1.33 | | 1.29 |
| | 26S subunit, ATPase, 6 | | | | | | | | | | | |
| D42063.1 | RAN binding protein 2 | \|2\|2q\|2q12\| | 1.32 | | | 1.44 | | | 1.22 | 1.41 | | |
| AI589086 | Lysosomal-associated multispanning | \|1\|1p\| | | | | | | 1.26 | | | | |
| | membrane protein-5 | | | | | | | | | | | |
| NM_005627.1 | serum/glucocorticoid regulated | \|6\|6q\| | | | 1.72 | 1.64 | | 1.30 | 1.34 | | 1.29 | 1.58 |
| | kinase | | | | | | | | | | | |
| NM_002822.1 | protein tyrosine kinase 9 | \|12\|12p\|12p11\| | | | 1.30 | | | | | | | |
| AI763123 | adducin 3 (gamma) | \|10\|10q\|10q24\| | | | 1.33 | 1.23 | | | | | | |
| NM_019903.1 | adducin 3 (gamma) | \|10\|10q\|10q24\| | | | 1.39 | | | | | | | |
| NM_004552.1 | NADH dehydrogenase (ubiquinone) | \|1\|1p\|1p34\| | | | | | | 1.22 | | | | |
| | Fe—S protein 5, 15 kDa (NADH- | | | | | | | | | | | |
| | coenzyme Q reductase) | | | | | | | | | | | |
| NM_006636.2 | methylene tetrahydrofolate | \|2\|2p\| | 1.38 | | | | | | | | | 1.36 |
| | dehydrogenase (NAD+ dependent), | | | | | | | | | | | |
| | methenyltetrahydrofolate | | | | | | | | | | | |
| | cyclohydrolase | | | | | | | | | | | |
| NM_007282.1 | ring finger protein 13 | \|3\|3q\|3q25\| | | | | | | | 1.43 | | | 1.56 |
| NM_002933.1 | ribonuclease, RNase A family, 1 | \|14\|14q\|14q11\| | | | | | | | | | 1.26 | 1.50 |
| | (pancreatic) | | | | | | | | | | | |
| AW150953 | 7-dehydrocholesterol reductase | \|11\|11q\|11q13\| | | | 1.26 | | | | | | 1.21 | 1.22 |
| NM_001360.1 | 7-dehydrocholesterol reductase | \|11\|11q\|11q13\| | | | 136 | | | | 1.29 | | 1.35 | |
| NM_001540.2 | heat shock 27 kDa protein 1 | \|7\|7p\|7p12\| | 1.55 | 1.35 | 1.59 | 1.62 | | 1.22 | | 1.65 | | 1.32 |
| AI826799 | EGF-containing fibulin-like | \|2\|2p\| | | | | | 0.63 | 0.62 | 0.74 | 0.67 | | |
| | extracellular matrix protein 1 | | | | | | | | | | | |
| NM_004105.2 | EGF-containing fibulin-like | \|2\|2p\| | | | | | 0.68 | 0.57 | | 0.71 | | |
| | extracellular matrix protein 1 | | | | | | | | | | | |
| AB029551.1 | RING1 and YY1 binding protein | \|3\|3p\| | 1.35 | | 1.26 | | | | | | 1.24 | |
| NM_00235.1 | lipase A, lysosomal acid, cholesterol | \|10\|10q\|10q23\| | | | 1.37 | 1.53 | | 1.21 | 1.31 | 1.34 | 1.26 | 1.58 |
| | esterase (Wolman disease) | | | | | | | | | | | |
| NM_000305.1 | paraoxonase 2 | \|7\|7q\|7q21\| | | 1.28 | 1.57 | | | | | 0.81 | | |
| NM_004048.1 | beta-2-microglobulin | \|15\|15q\|15q21\| | | 1.34 | 1.34 | | | | 1.20 | | | |
| NM_003365.1 | ubiquinol-cytochrome c reductase | \|3\|3p\|3p21\| | | | | | | | | | | 1.22 |
| | core protein I | | | | | | | | | | | |
| NM_006431.1 | chaperonin containing TCP1, subunit | \|12\|12q\|12q13\| | 1.21 | | | | | | | | | |
| | 2 (beta) | | | | | | | | | | | |
| NM_005720.1 | actin related protein 2/3 complex, | \|7\|7q\|7q11\| | | | | | 1.25 | | | | | 1.29 |
| | subunit 1B, 41 kDa | | | | | | | | | | | |
| NM_021122.2 | fatty-acid-Coenzyme A ligase, long- | \|4\|4q\|4q34\| | 1.64 | | 1.73 | | | 1.38 | 1.72 | 1.56 | | 1.76 |
| | chain 2 | | | | | | | | | | | |
| NM_001690.1 | ATPase, H+ transporting, lysosomal | \|3\|3q\|3q13\| | | | | | | 1.30 | | | | |
| | 70 kDa, V1 subunit A, isoform 1 | | | | | | | | | | | |
| NM_012334.1 | myosin X | \|5\|5p\|5p15\| | | | 1.32 | | | 0.83 | | 0.73 | | |
| AW157070 | epidermal growth factor receptor | \|7\|7p\| | | 1.20 | | | | | | | 0.77 | 0.76 |
| | (erythroblastic leukemia viral (v-erb- | | | | | | | | | | | |
| | b) oncogene homolog, avian) | | | | | | | | | | | |
| AI743792 | sialyltransferase 1 (beta-galactoside | \|3\|3q\|3q27\| | 1.28 | | | | | 1.22 | | | | 1.27 |
| | alpha-2,6-sialytransferase) | | | | | | | | | | | |
| NM_006519.1 | t-complex-associated-testis- | \|6\|6q\|6q25\| | 1.36 | 1.21 | 1.40 | 1.33 | | | 1.28 | | 1.24 | 1.28 |
| | expressed 1-like 1 | | | | | | | | | | | |
| NM_003257.1 | tight junction protein 1 (zona | \|15\|15q\| | | | 1.26 | | | | | | | |
| | occludens 1) | | | | | | | | | | | |
| AF083441.1 | putative translation initiation factor | \|17\| | | | | | | | | | | 1.23 |
| BC000603.1 | ribosomal protein L38 | \|17\|17q\|17q23\| | | | | | 1.23 | | | | | |
| NM_003012.2 | secreted frizzled-related protein 1 | \|8\|8p\|8p12\| | | | | | 0.65 | | | | | |
| NM_004788.1 | ubiquitination factor E4A (UFD2 | \|11\|11q\|11q23\| | 1.21 | | | | | | | 1.34 | | 1.24 |
| | homolog, yeast) | | | | | | | | | | | |
| NM_000527.2 | low density lipoprotein receptor | \|19\|19p\|19p13\| | | | 1.35 | | | | 1.34 | 1.23 | 1.34 | 1.27 |
| | (familial hypercholesterolemia) | | | | | | | | | | | |
| NM_005003.1 | NADH dehydrogenase (ubiquinone) | \|16\|16p\|16p11\| | | | | | | 1.22 | | | | |
| | 1, alpha/beta subcomplex, 1, 8 kDa | | | | | | | | | | | |
| NM_003003.1 | SEC14-like 1 (S. cerevisiae) | 17\|17q\|17q25\| | | | | | | | | | | 1.25 |
| NM_004817.1 | tight junction protein 2 (zona | \|9\|9q\|9q13\| | 1.56 | 1.39 | 1.66 | 1.42 | | | | | 1.46 | |
| | occludens 2) | | | | | | | | | | | |
| AI635449 | LIV-1 protein, estrogen regulated | \|18\|18q\|18q12\| | | | | | | | 1.22 | | | |
| AF043453.1 | sorting nexin 2 | \|5\|5q\| | 1.27 | | | | | | | 1.29 | 1.24 | |
| AA541758 | copine III | \|8\|8q\|8q21\| | 1.38 | | 1.26 | 1.34 | | | | | | |
| AW006290 | sudD suppressor of bimD6 homolog | \|18\|18q\|18q11\| | | | 1.21 | | | | | 1.31 | | 1.26 |
| | (A. nidulans) | | | | | | | | | | | |
| AA081084 | transcriptional co-activator with PDZ- | \|3\|3q\|3q23\| | | | 1.21 | | | | | | | |
| | binding motif (TAZ) | | | | | | | | | | | |
| AA747426 | interferon-related developmental | \|7\|7q\|7q22\| | | | | | | | | | | |
| | regulator 1 | | | | | | | | | | | |
| NM_007146.1 | zinc finger protein 161 | \|17\|17q\|17q23\| | | | | | | | | | | |
| NM_017458.1 | major vault protein | \|16\|16p\|16p13\| | | | | | | 1.21 | | | | 1.23 |
| AL117354 | CGI-100 protein | \|1\|1p\|1pter\| | 1.35 | | 1.24 | 1.33 | | | | | 1.21 | |
| NM_005629.1 | solute carrier family 6 | \|X\|Xq\| | | | 1.29 | | | | | | 1.25 | 1.26 |
| | (neurotransmitter transporter, | | | | | | | | | | | |
| | creatine), member 8 | | | | | | | | | | | |
| NM_006387.2 | calcium homeostasis endoplasmic | \|19\|19p\|19p13\| | | | | | | | | | | |
| | reticulum protein | | | | | | | | | | | |
| NM_002796.1 | proteasome (prosome, macropain) | \|1\|1q\| | 1.21 | | | 1.22 | | 1.27 | | | | 1.28 |
| | subunit, beta type, 4 | | | | | | | | | | | |
| BE561596 | metastasis associated 1 | \|14\|14q\|14q32\| | | | | | | | | | | |
| NM_003165.1 | syntaxin binding protein 1 | \|9\|9q\|9q34\| | | | | | | 1.21 | | 1.21 | | |
| NM_005180.1 | hypothetical protein MGC12685 | \|10\|10p\|10p11\| | | | | 1.25 | | | | | | |
| NM_004894.1 | chromosome 14 open reading frame 2 | \|14\|14q\|14q32\| | | 1.21 | | | | | | | | |
| NM_002615.1 | serine (or cysteine) proteinase | \|17\|17p\|17p13\| | | | | | 0.56 | | | 1.23 | 1.20 | |
| | inhibitor, clade F (alpha-2 | | | | | | | | | | | |
| | antiplasmin, pigment epithelium | | | | | | | | | | | |
| | derived factor) member 1 | | | | | | | | | | | |
| NM_007033.1 | similar to S. cerevisiae RER1 | \|1\|1p\|1pter\| | | | | | | | | | | 1.21 |
| NM_000786.1 | cytochrome P450, 51 (lanosterol 14- | \|7\|7q\|7q21\| | | | | | | | 130 | | | |
| | alpha-demethylase) | | | | | | | | | | | |
| NM_002135.1 | nuclear receptor subfamily 4, group | \|12\|12q\| | | | 0.78 | 0.79 | 0.82 | 0.79 | 0.74 | 0.75 | | |
| | A, member 1 | | | | | | | | | | | |
| NM_001444.1 | fatty acid binding protein 5 (psoriasis- | \|8\|8q\|8q21\| | 1.43 | | 1.34 | | | | 1.50 | 1.23 | 1.51 | 1.41 |
| | associated) | | | | | | | | | | | |
| AI093579 | integrin, alpha V (vitronectin receptor, | \|2\|2q\|2q31\| | | | 1.35 | 1.29 | | | 1.24 | | | |
| | alpha polypeptide, antigen CD51) | | | | | | | | | | | |
| AF231124.1 | sparc/osteonectin, cwcv and kazal- | \|5\|5q\| | | | | | | 1.24 | | | | |
| | like domains proteoglycan (testican) | | | | | | | | | | | |
| NM_001085.2 | serine (or cysteine) proteinase | \|14\|14q\|14q32\| | 1.97 | | 1.50 | 1.61 | 1.52 | 1.41 | 1.55 | 1.72 | 1.96 | 1.65 |
| | inhibitor, clade A (alpha-1 | | | | | | | | | | | |
| | antiproteinase, antitrypsin), member 3 | | | | | | | | | | | |
| AW026535 | leptin receptor gene-related protein | \|1\| | | | 1.36 | | | | 1.23 | | | |
| NM_017526.1 | leptin receptor gene-related protein | \|1\| | | | 1.23 | | | | | | | |
| NM_006178.1 | N-ethylmaleimide-sensitive factor | \|17\|17q\| | | | | | | 1.28 | | 1.41 | | |
| NM_005532.1 | interferon, alpha-inducible protein 27 | \|14\|14q\| | | 1.31 | 1.20 | | 1.39 | 1.23 | 1.26 | | 1.36 | 1.55 |
| NM_000104.2 | cytochrome P450, subfamily I (dioxin- | \|2\|2p\| | | | | | 1.31 | 1.22 | | 1.46 | | 1.32 |
| | inducible), polypeptide 1 (glaucoma | | | | | | | | | | | |
| | 3, primary infantile) | | | | | | | | | | | |
| NM_000104.2 | cytochrome P450, subfamily I (dioxin- | \|2\|2p\| | 1.66 | | | | 1.68 | 1.40 | | 1.68 | | 1.51 |
| | inducible), polypeptide 1 (glaucoma | | | | | | | | | | | |
| | 3, primary infantile) | | | | | | | | | | | |
| BF346014 | Homo sapiens mRNA; cDNA | | | | 0.70 | | | | | | | |
| | DKFZp434G012 (from clone | | | | | | | | | | | |
| | DKFZp434G012) | | | | | | | | | | | |
| NM_004236.1 | thyroid receptor interacting protein 15 | \|15\|15q\|15q21\| | 1.28 | | | 1.25 | | 1.21 | | 1.30 | | 1.30 |
| BC002637.1 | GS3955 protein | \|2\|2p\|2p25\| | 0.80 | | 0.77 | | | | 0.79 | 0.79 | | |
| NM_003640.1 | inhibitor of kappa light polypeptide | \|9\|9q\| | 1.26 | | | | | 1.20 | | 1.30 | 1.24 | 1.21 |
| | gene enhancer in B-cells, kinase | | | | | | | | | | | |
| | complex-associated protein | | | | | | | | | | | |
| NM_006931.1 | solute carrier family 2 (facilitated | \|12\|12p\|12p13\| | 1.35 | | | | | | | | | 1.23 |
| | glucose transporter), member 3 | | | | | | | | | | | |
| NM_006736.1 | DnaJ (Hsp40) homolog, subfamily B, | \|2\|2q\|2q32\| | | | 1.26 | 1.24 | | | 1.24 | 1.20 | 1.29 | 1.25 |
| | member 2 | | | | | | | | | | | |
| NM_001313.1 | collapsin response mediator protein 1 | \|4\|4p\|4p16\| | | | | 0.83 | 0.83 | | | | | |
| AL518627 | 3-hydroxy-3-methylglutaryl- | \|5\|5q\|5q13\| | 1.41 | | | | | 1.34 | | | | |
| | Coenzyme A reductase | | | | | | | | | | | |
| NM_004757.1 | small inducible cytokine subfamily E, | \|4\|4q\| | | | 1.20 | | | | | | | 1.23 |
| | member 1 (endothelial monocyte- | | | | | | | | | | | |
| | activating) | | | | | | | | | | | |
| NM_016441.1 | cysteine-rich motor neuron 1 | \|2\|2p\| | 1.26 | | | | | | | | | 1.22 |
| NM_005965.1 | myosin, light polypeptide kinase | \|3\|3q\| | | | 1.34 | | | | | | | 1.35 |
| AL718418 | stress 70 protein chaperone, | \|21\|21q\| | 1.36 | | | | | | | | | |
| | microsome-associated, 60 kDa | | | | | | | | | | | |
| NM_015607.1 | DKFZP547E1010 protein | \|1\|1q\|1q21\| | | | 1.24 | 1.25 | | | | | | 1.24 |
| NM_014902.1 | KIAA0964 protein | \|20\|20q\|20q11\| | | | | 0.81 | | | | | | |
| NM_005346.2 | heat shock 70 kDa protein 1B | \|6\|6p\|6p21\| | | | 1.54 | | | | 1.45 | 1.48 | | 0.71 |
| BC000436.1 | endosulfine alpha | \|1\|1q\|1q21\| | | | 0.83 | 0.80 | | | | 0.81 | | |
| NM_003489.1 | nuclear receptor interacting protein 1 | \|21\|21q\|21q11\| | 1.23 | | | | | | | | | |
| NM_004447.1 | epidermal growth factor receptor | \|12\|12q\|12q23\| | | 1.20. | 1.25 | | | | | | | |
| | pathway substrate 8 | | | | | | | | | | | |
| NM_000935.1 | procollagen-lysine, 2-oxoglutarate 5- | \|3\|3q\|3q23\| | 1.35 | 1.25 | | | | | 1.26 | | | |
| | dioxygenase (lysine hydroxylase) 2 | | | | | | | | | | | |
| AI005043 | Homo sapiens mRNA; cDNA | | 1.67 | | 1.44 | | 1.29 | | 1.59 | | | 1.74 |
| | DKFZp667A0918 (from clone | | | | | | | | | | | |
| | DKFZp667A0918) | | | | | | | | | | | |
| NM_003859.1 | dolichyl-phosphate | \|20\|20q\|20q13\| | 1.28 | | | | | 1.20 | | 1.31 | | |
| | mannosyltransferase polypeptide 1, | | | | | | | | | | | |
| | catalytic subunit | | | | | | | | | | | |
| NM_000271.1 | Niemann-Pick disease, type C1 | \|18\|18q\|18q11\| | | | 1.49 | | | | 1.27 | | | 1.58 |
| AA675892 | transducer of ERBB2, 1 | \|17\|17q\| | 1.32 | | 1.26 | | 0.83 | | | | | |
| NM_002015.2 | forkhead box O1A | \|13\|13q\|13q14\| | | | 1.22 | | | | | | | |
| | (rhabdomyosarcoma) | | | | | | | | | | | |
| NM_014814.1 | KIAA0107 gene product | \|3\|3p\|3p14\| | 1.21 | | | | | | | | | 1.20 |
| NM_007373.1 | soc-2 suppressor of clear homolog | \|10\|10q\| | 1.22 | | | | | | | | | |
| | (C. elegans) | | | | | | | | | | | |
| NM_000436.1 | 3-oxoacid CoA transferase | \|5\|5p\| | 1.22 | | | | | | | | | |
| NM_014016.1 | SAC1 suppressor of actin mutations | \|3\|3p\|3p21\| | 1.21 | | | | | | | | | |
| | 1-like (yeast) | | | | | | | | | | | |
| NM_006323.1 | SEC24 related gene family, member | \|4\|4q\| | 1.29 | | | 1.33 | | | | 1.33 | | |
| | B (S. cerevisiae) | | | | | | | | | | | |
| NM_004172.1 | solute carrier family 1 (glial high | \|5\|5p\| | 1.45 | 1.35 | 1.76 | 1.31 | | | | | | |
| | affinity glutamate transporter), | | | | | | | | | | | |
| | member 3 | | | | | | | | | | | |
| NM_001151.1 | solute carrier family 25 (mitochondrial | \|4\|4q\| | | | | | | 1.21 | | | | |
| | carrier; adenine nucleotide | | | | | | | | | | | |
| | translocator), member 4 | | | | | | | | | | | |
| NM_000295.1 | serine (or cysteine) proteinase | \|14\|14q\|14q32\| | | | | | 1.23 | | | | | |
| | inhibitor, clade A (alpha-1 | | | | | | | | | | | |
| | antiproteinase, antitrypsin), member 1 | | | | | | | | | | | |
| NM_000029.1 | angiotensinogen (serine (or cysteine) | \|1\|1q\|1q42\| | | 1.21 | 1.74 | 1.23 | | | 1.22 | | | |
| | proteinase inhibitor, clade A (alpha-1 | | | | | | | | | | | |
| | antiproteinase, antitrypsin), member | | | | | | | | | | | |
| | 8) | | | | | | | | | | | |
| AL080081.1 | DnaJ (Hsp40) homolog, subfamily B, | \|7\|7q\| | 1.28 | | | | | | | 1.23 | | |
| | member 9 | | | | | | | | | | | |
| NM_002858.2 | ATP-binding cassette, sub-family D | \|1\|1p\|1p22\| | | | 1.22 | | | | | | | |
| | (ALD), member 3 | | | | | | | | | | | |
| NM_000194.1 | hypoxanthine | \|X\|Xq\|Xq26\| | | | | | | 1.22 | | | | |
| | phosphoribosyltransferase 1 (Lesch- | | | | | | | | | | | |
| | Nyhan syndrome) | | | | | | | | | | | |
| NM_004762.1 | pleckstrin homology, Sec7 and | \|17\|17q\| | | | | | | | | | | 1.25 |
| | coiled/coil domains 1(cytohesin 1) | | | | | | | | | | | |
| NM_019058.1 | HIF-1 responsive RTP801 | \|10\|10p\|10pter\| | 1.37 | 1.41 | 1.63 | 1.51 | 1.31 | | 1.43 | 1.50 | | |
| T62571 | microtubule-associated protein 7 | \|6\|6q\|6q23\| | 1.47 | | 1.36 | | | | | | | 1.38 |
| BC002642.1 | cathepsin S | \|1\|1q\| | | | | | 1.21 | | | | | |
| NM_006005.2 | Wolfram syndrome 1 (wolframin) | \|4\|4p\| | 1.20 | | 1.20 | | | 1.24 | | | | |
| BF726212 | Homo sapiens, clone | | 1.28 | | | | | | | | | |
| | IMAGE: 4246029, mRNA | | | | | | | | | | | |
| NM_003850.1 | succinate-CoA ligase, ADP-forming, | \|13\|13q\|13q12\| | 1.28 | | | | | 1.22 | | | | |
| | beta subunit | | | | | | | | | | | |
| NM_005433.1 | v-yes-1 Yamaguchi sarcoma viral | \|18\|18p\|18p11\| | | 1.21 | 1.29 | | | | | | | |
| | oncogene homolog 1 | | | | | | | | | | | |
| NM_005433.1 | v-yes-1 Yamaguchi sarcoma viral | \|18\|18p\|18p11\| | | | 1.26 | | | | | | | |
| | oncogene homolog 1 | | | | | | | | | | | |
| AI382146 | SRY (sex determining region Y)-box | \|17\|17q\|17q24\| | 1.26 | 1.30 | 1.60 | 1.24 | | | | | | |
| | 9 (campomelic dysplasia, autosomal | | | | | | | | | | | |
| | sex-reversal) | | | | | | | | | | | |
| NM_000346.1 | SRY (sex determining region, Y)-box | \|17\|17q\|17q24\| | 1.43 | 1.36 | 1.56 | 1.32 | | | | | | |
| | 9 (campomelic dysplasia, autosomal | | | | | | | | | | | |
| | sex-reversal) | | | | | | | | | | | |
| NM_000877.1 | interleukin 1 receptor, type I | \|2\|2q\| | | | | | 1.25 | | | 1.24 | | |
| NM_000491.2 | complement component 1, q | \|1\|1p\|1p36\| | 1.58 | 1.39 | | | 1.49 | 1.32 | 1.36 | | 1.37 | |
| | subcomponent, beta polypeptide | | | | | | | | | | | |
| NM_004889.1 | ATP synthase, H+ transporting, | \|7\|7q\|7q11\| | 1.21 | | | | | | | | | |
| | mitochondrial F0 complex, subunit f, | | | | | | | | | | | |
| | isoform 2 | | | | | | | | | | | |
| N21138 | Rho-related BTB domain containing 3 | \|5\|5q\|5q21\| | 1.30 | 1.20 | 1.64 | 1.41 | 1.36 | 1.22 | | | | |
| NM_014899.1 | Rho-related BTB domain containing 3 | \|5\|5q\|5q21\| | 1.32 | 1.27 | 1.61 | 1.27 | 1.43 | | 1.33 | | | |
| NM_007029.1 | stathmin-like 2 | \|8\|8q\|8q13\| | | | | | | 1.40 | | 1.22 | | |
| NM_005539.1 | inositol polyphosphate-5- | \|10\|10q\|10q26\| | | 0.79 | | | | 1.22 | | | | |
| | phosphatase, 40 kDa | | | | | | | | | | | |
| NM_005581.1 | Lutheran blood group (Auberger b | \|19\|19q\|19q13\| | 0.80 | | | | | | | | | |
| | antigen included) | | | | | | | | | | | |
| NM_000990.1 | ribosomal protein L27a | \|11\|11p\| | | | 1.26 | | 1.23 | | | | | |
| NM_002844.1 | protein tyrosine phosphatase, | \|6\|6q\|6q22\| | | | | | | | | 1.28 | | 1.34 |
| | receptor type, K | | | | | | | | | | | |
| J04183.1 | lysosomal-associated membrane | \|X\|Xq\| | | | 1.59 | 1.65 | | 1.32 | 1.47 | | 1.43 | 1.86 |
| | protein 2 | | | | | | | | | | | |
| NM_002294.1 | lysosomal-associated membrane | \|X\|Xq\| | | | 1.51 | | | | 1.43 | | 1.31 | 1.65 |
| | protein 2 | | | | | | | | | | | |
| NM_014849.1 | synaptic vesicle glycoprotein 2 | \|1\|1q\|1q21\| | | | | | 0.80 | | | | | |
| NM_004636.1 | sema domain, immunoglobulin | \|3\|3p\|3p21\| | | | 1.34 | | | | | | | 1.25 |
| | domain (Ig), short basic domain, | | | | | | | | | | | |
| | secreted, (semaphorin) 3B | | | | | | | | | | | |
| NM_013450.1 | bromodomain adjacent to zinc finger | \|2\|2q\|2q23\| | | | 1.29 | | | | | | | |
| | domain, 2B | | | | | | | | | | | |
| NM_005211.1 | colony stimulating factor 1 receptor, | \|5\|5q\|5q33\| | | | | | 1.24 | | | | | |
| | formerly McDonough feline sarcoma | | | | | | | | | | | |
| | viral (v-fms) oncogene homolog | | | | | | | | | | | |
| NM_005426.1 | tumor protein p53 binding protein, 2 | \|1\|1q\|1q42\| | 1.27 | | 1.47 | 1.29 | 1.23 | | 1.23 | | | |
| NM_006206.1 | platelet-derived growth factor | \|4\|4q\|4q11\| | | | | | | 0.74 | 0.74 | 0.55 | 0.77 | 0.75 |
| | receptor, alpha polypeptide | | | | | | | | | | | |
| NM_003642.1 | histone acetyltransferase 1 | \|2\|2q\|2q31\| | 1.38 | | 1.30 | 1.23 | | | 1.23 | | | |
| NM_001706.1 | B-cell CLL/lymphoma 6 (zinc finger | \|3\|3q\| | | 1.30 | | | 1.28 | | | 1.43 | | |
| | protein 51) | | | | | | | | | | | |
| AB020645.1 | glutaminase | \|2\|2q\|2q32\| | | | 0.82 | | | | | | | |
| AF097493.1 | glutaminase | \|2\|2q\|2q32\| | | | 0.72 | | | | | | | |
| NM_001482.1 | glycine amidinotransferase (L- | \|15\|15q\| | | | 1.26 | | | | | | | |
| | arginine: glycine amidinotransferase) | | | | | | | | | | | |
| NM_014737.1 | Ras association (RalGDS/AF-6) | \|20\|20p\|20pter\| | | 1.25 | | | | | | | | |
| | domain family 2 | | | | | | | | | | | |
| NM_006876.1 | UDP-GIcNAc: betaGal beta-1,3-N- | \|11\|11q\|11q12\| | 1.30 | | | | | 1.25 | | 1.35 | | |
| | acetylglucosaminyltransferase 6 | | | | | | | | | | | |
| NM_004999.1 | myosin VI | \|6\|6q\| | | | 1.42 | | | | | | | |
| AW235612 | spinocerebellar ataxia 1 | \|6\|6p\| | | | 0.78 | | 0.75 | | 0.59 | 0.72 | | 0.64 |
| | (olivopontocerebellar ataxia 1, | | | | | | | | | | | |
| | autosomal dominant, ataxin 1) | | | | | | | | | | | |
| NM_006457.1 | LIM protein (similar to rat protein | \|4\|4q\| | | 1.28 | | | | | | | | |
| | kinase C-binding enigma) | | | | | | | | | | | |
| AA810268 | mitogen-activated protein kinase | \|17\|17p\|17p11\| | | | 0.80 | | | | | | | |
| | kinase 4 | | | | | | | | | | | |
| AW440492 | ATPase, Na+/K+ transporting, alpha | \|1\|1q\|1q21\| | | 1.24 | 1.32 | | 1.29 | | | | | |
| | 2 (+) polypeptide | | | | | | | | | | | |
| NM_000702.1 | ATPase, Na+/K+ transporting, alpha | \|1\|1q\|1q21\| | 1.29 | 1.41 | 1.43 | | 1.28 | | | | | |
| | 2 (+) polypeptide | | | | | | | | | | | |
| NM_004973.2 | jumonji homolog (mouse) | \|6\|6p\|6p24\| | | | | | | | | | | 1.28 |
| NM_007269.1 | syntaxin binding protein 3 | \|1\|1p\|1p13\| | | | 1.29 | | | | 1.21 | | | |
| NM_014970.1 | kinesin-associated protein 3 | \|1\|1q\| | 1.26 | | | | | | | 1.30 | | |
| NM_004454.1 | ets variant gene 5 (ets-related | \|3\|3q\| | | | 0.80 | | | | 0.80 | 0.73 | | |
| | molecule) | | | | | | | | | | | |
| AW117368 | ADP-ribosylation factor guanine | \|8\|8p\|8pter\| | | | 1.22 | | | 1.34 | | | | |
| | nucleotide factor 6 | | | | | | | | | | | |
| NM_015310.1 | ADP-ribosylation factor guanine | \|8\|8p\|8pter\| | 1.31 | | | | | | | | | |
| | nucleotide factor 6 | | | | | | | | | | | |
| N33009 | apolipoprotein E | \|19\|19q\|19q13\| | 1.41 | 1.24 | 1.66 | 1.33 | | | | | | |
| NM_000041.1 | apolipoprotein E | \|19\|19q\|19q13\| | | 1.24 | 1.57 | | | | | 0.81 | | |
| BE973687 | hairy homolog (Drosophila) | \|3\|3q\|3q28\| | | | | | | 0.83 | | | | |
| NM_002789.1 | proteasome (prosome, macropain) | \|15\|15q\|15q24\| | 1.21 | | | | | 1.23 | | | | 1.28 |
| | subunit, alpha type, 4 | | | | | | | | | | | |
| NM_001063.1 | transferrin | \|3\|3q\| | | | | | | | 1.44 | | 1.20 | 1.98 |
| NM_002765.1 | phosphoribosyl pyrophosphate | \|X\|Xp\|Xp22\| | 1.28 | | | | | | | | | |
| | synthetase 2 | | | | | | | | | | | |
| NM_000560.1 | CD53 antigen | \|1\|1p\| | | | | | 1.26 | | | | | |
| NM_014034.1 | DKFZP547E2110 protein | \|6\|6q\|6q22\| | | | 1.25 | 1.26 | | | | | 1.23 | |
| NM_004045.1 | ATX1 antioxidant protein 1 homolog | \|5\|5q\| | | | | | | | | | | 1.22 |
| | (yeast) | | | | | | | | | | | |
| NM_002970.1 | spermidine/spermine N1- | \|X\|Xp\|Xp22\| | | | | | 1.20 | | | | | |
| | acetyltransferase | | | | | | | | | | | |
| NM_014302.1 | Sec61 gamma | \|7\|7p\|7p14\| | 1.22 | | | | | 1.23 | 1.25 | | | |
| NM_005822.1 | Down syndrome critical region gene | \|6\|6p\|6p12\| | | | | | | 1.24 | | | | |
| | 1-like 1 | | | | | | | | | | | |
| NM_006378.1 | sema domain, immunoglobulin | \|9\|9q\|9q22\| | | | | 1.26 | | | | | | 1.28 |
| | domain (Ig), transmembrane domain | | | | | | | | | | | |
| | (TM) and short cytoplasmic domain, | | | | | | | | | | | |
| | (semaphorin) 4D | | | | | | | | | | | |
| NM_002055.1 | glial fibrillary acidic protein | \|17\|17q\| | | | 1.59 | | | | | | | |
| NM_001206.1 | basic transcription element binding | \|9\|9q\| | 1.40 | | | 1.26 | | | | 1.40 | 1.52 | 1.27 |
| | protein 1 | | | | | | | | | | | |
| BF672975 | lipoprotein lipase | \|8\|8p\| | | 0.81 | | | | 1.28 | | | | |
| NM_000237.1 | lipoprotein lipase | \|8\|8p\| | | 0.77 | | | | | | | | |
| AW298170 | mitogen-activated protein kinase | \|14\|14q\|14q11\| | | | | | | | | | | |
| | kinase kinase kinase 5 | | | | | | | | | | | |
| NM_006575.1 | mitogen-activated protein kinase | \|14\|14q\|14q11\| | | | | 1.51 | | | | | | |
| | kinase kinase kinase 5 | | | | | | | | | | | |
| NM_005103.2 | fasciculation and elongation protein | \|11\|11q\|11q24\| | | | | | | | | | | 1.28 |
| | zeta 1 (zygin I) | | | | | | | | | | | |
| NM_014795.1 | zinc finger homeobox 1b | \|2\|2q\| | | | | | 1.21 | | | 0.76 | | |
| NM_003136.1 | signal recognition particle 54 kDa | \|14\|14q\| | 1.35 | | | | | | | | 1.28 | 1.26 |
| NM_004553.1 | NADH dehydrogenase (ubiquinone) | \|5\|5p\|5p15\| | | | | | | 1.20 | | | | 1.25 |
| | Fe—S protein 6, 13 kDa (NADH- | | | | | | | | | | | |
| | coenzyme Q reductase) | | | | | | | | | | | |
| NM_016235.1 | G protein-coupled receptor, family C, | \|16\|16p\| | | | 1.59 | | | | 1.36 | | 1.23 | 1.44 |
| | group 5, member B | | | | | | | | | | | |
| NM_022969.1 | fibroblast growth factor receptor 2 | \|10\|10q\| | | 1.34 | 1.35 | | | | | 0.83 | | 1.25 |
| | (bacteria-expressed kinase, | | | | | | | | | | | |
| | keratinocyte growth factor receptor, | | | | | | | | | | | |
| | craniofacial dysostosis 1, Crouzon | | | | | | | | | | | |
| | syndrome, Pfeiffer syndrome, | | | | | | | | | | | |
| | Jackson-Weiss syndrome) | | | | | | | | | | | |
| U91903.1 | frizzled-related protein | \|2\|2q\| | 0.80 | | 071 | 0.52 | | 0.79 | 0.66 | | 0.73 | 0.77 |
| NM_001463.1 | frizzled-related protein | \|2\|2q\| | | | | 0.66 | | | 0.83 | | 0.78 | 0.81 |
| NM_013989.1 | deiodinase, iodothyronine, type II | \|14\|14q\|14q24\| | | | | | | | | 0.57 | 0.83 | |
| NM_003748.1 | aldehyde dehydrogenase 4 family, | \|1\|1p\| | | | 1.25 | | | | | | | |
| | member A1 | | | | | | | | | | | |
| NM_002221.1 | inositol 1,4,5-trisphosphate 3-kinase B | \|1\|1q\|1q41\| | 1.32 | | 1.63 | 1.31 | | | 1.29 | | | 1.23 |
| NM_012198.1 | grancalcin, EF-hand calcium binding | \|2\|2q\|2q24\| | | | 1.28 | | | 1.20 | 1.21 | 1.35 | 1.39 | 1.35 |
| | protein | | | | | | | | | | | |
| NM_006379.1 | sema domain, immunoglobulin | \|7\|7q\|7q21\| | | | | | | | 1.22 | | | 0.72 |
| | domain (Ig), short basic domain, | | | | | | | | | | | |
| | secreted, (semaphorin) 3C | | | | | | | | | | | |
| NM_006107.1 | acid-inducible phosphoprotein | \|17\| | | | | | | | | | | |
| BG252490 | DnaJ (Hsp40) homolog, subfamily B, | \|1\|1p\|1p31\| | | | | | | | | | 1.22 | |
| | member 4 | | | | | | | | | | | |
| AV727449 | p300/CBP-associated factor | \|3\|3p\| | 1.42 | | 1.50 | 1.32 | 1.21 | | 1.22 | | | |
| NM_015833.1 | adenosine deaminase, RNA-specific, | \|21\|21q\|21q22\| | | | 0.81 | | | | | | | |
| | B1 (RED1 homolog rat) | | | | | | | | | | | |
| NM_022832.1 | hypothetical protein FLJ12552 | \|4\|4p\| | 1.24 | | | | | | | | | |
| NM_022832.1 | hypothetical protein FLJ12552 | \|4\|4P\| | 1.21 | | | | | | | | | |
| NM_005694.1 | COX17 homolog, cytochrome c | \|3\|3q\|3q13\| | | | | | | | | | | 1.25 |
| | oxidase assembly protein (yeast) | | | | | | | | | | | |
| NM_014999.1 | RAB21, member RAS oncogene | \|12\|12q\|12q13\| | 1.24 | | | 1.30 | | | 1.21 | 1.36 | 1.27 | 1.36 |
| | family | | | | | | | | | | | |
| NM_014799.1 | hephaestin | \|X\|Xq\|Xq11\| | | | 1.42 | 1.29 | 1.23 | | 1.30 | | | |
| NM_006359.1 | solute carrier family 9 | \|X\|Xq\|Xq26\| | | | | | | | | 1.25 | | |
| | (sodium/hydrogen exchanger), | | | | | | | | | | | |
| | isoform 6 | | | | | | | | | | | |
| NM_000784.1 | cytochrome P450, subfamily XXVIIA | \|2\|2q\|2q33\| | | | | | | | | | | 1.22 |
| | (steroid 27-hydroxylase, | | | | | | | | | | | |
| | cerebrotendinous xanthomatosis), | | | | | | | | | | | |
| | polypeptide 1 | | | | | | | | | | | |
| NM_004480.1 | fucosyltransferase 8 (alpha (1,6) | \|14\|14q\|14q24\| | | | | | | | | | | |
| | fucosyltransferase) | | | | | | | | | | | |
| NM_005639.1 | synaptotagmin I | \|12\| | | 0.83 | 0.76 | | | 1.31 | | | | 0.52 |
| NM_000570.1 | Fc fragment of IgG, low affinity IIIb, | \|1\|1q\| | | | | | 1.31 | 1.23 | | | | |
| | receptor for (CD16) | | | | | | | | | | | |
| NM_014575.1 | schwannomin interacting protein 1 | \|3\|3q\|3q25\| | 1.28 | | | 1.24 | | | | | 1.32 | 1.45 |
| NM_013279.1 | chromosome 11 open reading frame 9 | \|11\|11q\|11q12\| | | | | | | | | | 1.24 | 1.64 |
| NM_003595.1 | tyrosylprotein sulfotransferase 2 | \|22\|22q\|22q12\| | | | | | | | 1.23 | 1.22 | | 1.24 |
| NM_006176.1 | neurogranin (protein kinase C | \|11\|11q\| | | | 0.78 | | | | | | 0.82 | |
| | substrate, RC3) | | | | | | | | | | | |
| NM_003332.1 | TYRO protein tyrosine kinase binding | \|19\|19q\|19q13\| | | 1.23 | 1.38 | | 1.28 | | 1.28 | | | |
| | protein | | | | | | | | | | | |
| NM_016013.1 | CGI-65 protein | \|15\|15q\|15q11\| | | | | | | | | | | 1.24 |
| NM_003272.1 | transmembrane 7 superfamily | \|1\|1q\|1q42\| | | | 1.29 | 1.21 | | | | | | |
| | member 1 (upregulated in kidney) | | | | | | | | | | | |
| NM_003596.1 | tyrosylprotein sulfotransferase 1 | \|7\|7q\|7q11\| | 1.35 | | | | | | | 1.37 | 1.29 | 1.31 |
| AA044154 | KIAA0999 protein | \|11\|11q\|11q23\| | | | | | | | | | | |
| AW194947 | ectonucleotide | \|6\|6p\|6p12\| | 1.49 | | 1.29 | 1.52 | | 1.23 | 1.21 | 1.43 | 1.36 | 1.61 |
| | pyrophosphatase/phosphodiesterase | | | | | | | | | | | |
| | 4 (putative function) | | | | | | | | | | | |
| NM_000097.1 | coproporphyrinogen oxidase | \|3\|3q\| | | | | | | | | | | 1.32 |
| | (coproporphyria, harderoporphyria) | | | | | | | | | | | |
| NM_001860.1 | solute carrier family 31 (copper | \|9\|9q\|9q31\| | | | | | | | 1.30 | | | 1.53 |
| | transporters), member 2 | | | | | | | | | | | |
| NM_005619.1 | reticulon 2 | \|19\|19q\|19q13\| | | | | | | | | | | |
| NM_020309.1 | Homo sapiens cDNA FLJ33742 fis, | | | 0.81 | 0.77 | | 0.74 | | | | | |
| | clone BRAWH2019053, highly similar | | | | | | | | | | | |
| | to Homo sapiens BNPI mRNA for | | | | | | | | | | | |
| | brain-specific Na-dependent | | | | | | | | | | | |
| | inorganic phosphate cotransporter | | | | | | | | | | | |
| NM_004106.1 | Fc fragment of IgE, high affinity I, | \|1\|1q\| | | | | | 1.20 | | | | | |
| | receptor for; gamma polypeptide | | | | | | | | | | | |
| NM_005386.1 | neuronatin | \|20\|20q\|20q11\| | | | 0.79 | 0.61 | 0.65 | | 0.81 | 0.69 | | |
| NM_000115.1 | endothelin receptor type B | \|13\|13q\| | 1.43 | | 1.70 | | | | | | | |
| BF002254 | golgi phosphoprotein 4 | \|3\|3q\| | 0.80 | | | | | | | | | |
| NM_002450.1 | metallothionein 1L | \|16\|16q\| | | 1.43 | 1.50 | 1.47 | 1.39 | | | 1.38 | | 1.35 |
| NM_021107.1 | mitochondrial ribosomal protein S12 | \|19\|19q\|19q13\| | | | | | | | | 1.28 | | 1.23 |
| NM_005613.2 | regulator of G-protein signalling 4 | \|1q\|1q23\| | | | 0.68 | | | | | | | 0.79 |
| NM_003930.1 | src family associated phosphoprotein 2 | \|7\|7p\|7p21\| | 1.36 | | | | | 1.22 | 1.23 | 1.36 | | |
| NM_001993.2 | coagulation factor III (thromboplastin, | \|1\|1p\|1p22\| | 1.48 | | 1.60 | 1.37 | 1.39 | | | | 1.35 | |
| | tissue factor) | | | | | | | | | | | |
| NM_014810.1 | centrosome-associated protein 350 | \|1\|1p\|1p36\| | 1.34 | | | 1.36 | | 1.26 | | | | |
| NM_003657.1 | breast carcinoma amplified sequence 1 | \|20\|20q\|20q13\| | | | | | | | | 0.65 | | |
| NM_000142.2 | fibroblast growth factor receptor 3 | \|4\|4p\|4p16\| | | | 1.28 | | | | | 0.73 | | |
| | (achondroplasia, thanatophoric | | | | | | | | | | | |
| | dwarfism) | | | | | | | | | | | |
| NM_005864.1 | signal transduction protein (SH3 | \|14\|14q\|14q11\| | | 1.30 | | | | | 1.36 | 0.77 | | 1.33 |
| | containing) | | | | | | | | | | | |
| BC005248.1 | eukaryotic translation initiation factor | \|Y\|Yq\|Yq11\| | 1.44 | 1.28 | 1.44 | 1.34 | 1.25 | | 1.31 | | 1.32 | 1.30 |
| | 1A, Y chromosome | | | | | | | | | | | |
| NM_021076.1 | neurofilament, heavy polypeptide | \|22\|22q\|22q12\| | | | 0.76 | | | | | | 0.64 | |
| | 200 kDa | | | | | | | | | | | |
| NM_000153.1 | galactosylceramidase (Krabbe | \|14\|14q\| | | | 1.29 | 1.28 | | | 1.24 | | 1.23 | 1.22 |
| | disease) | | | | | | | | | | | |
| M27968.1 | fibroblast growth factor 2 (basic) | \|4\|4q\|4q26\| | | | | 1.26 | | | | | | |
| NM_002006.1 | fibroblast growth factor 2 (basic) | \|4\|4q\|4q26\| | 1.60 | | 1.44 | | 1.24 | | | | | |
| NM_016725.1 | folate receptor 1 (adult) | \|11\|11q\|11q13\| | | | | | 0.59 | 0.82 | | 0.83 | | |
| NM_000698.1 | arachidonate 5-lipoxygenase | \|10\|10q\|10q11\| | | | | | 1.34 | | 1.29 | | | |
| BG260394 | synuclein, alpha (non A4 component | \|4\|4q\| | | | | | | 1.26 | | | 1.32 | |
| | of amyloid precursor) | | | | | | | | | | | |
| NM_002851.1 | protein tyrosine phosphatase, | \|7\|7q\|7q31\| | | | 1.21 | | | | | 0.72 | | 0.82 |
| | receptor-type, Z polypeptide 1 | | | | | | | | | | | |
| NM_005261.1 | GTP binding protein overexpressed | \|8\|8q\|8q13\| | | | | | 0.83 | | | | | |
| | in skeletal muscle | | | | | | | | | | | |
| NM_024112.1 | chromosome 9 open reading frame | \|9\|9q\|9q34\| | | | | 0.82 | 0.79 | | | | 0.81 | |
| | 16 | | | | | | | | | | | |
| NM_015993.1 | transmembrane 4 superfamily | \|16\|16q\| | | | | | | | | | 1.20 | 1.53 |
| | member 11 (plasmolipin) | | | | | | | | | | | |
| NM_001958.1 | eukaryotic translation elongation | \|20\|20q\|20q13\| | | | | | | 1.27 | | | | |
| | factor 1 alpha 2 | | | | | | | | | | | |
| NM_006822.1 | RAB40B, member RAS oncogene | \|17\|17q\|17q25\| | | | 1.21 | 1.22 | | | | | 1.25 | 1.58 |
| | family | | | | | | | | | | | |
| NM_004508.1 | isopentenyl-diphosphate delta | \|10\|10p\|10p15\| | | | 1.20 | | | | 1.20 | | 1.29 | |
| | isomerase | | | | | | | | | | | |
| NM_006002.1 | ubiquitin carboxyl-terminal esterase | \|13\|13q\|13q21\| | | | | | | 1.32 | | | | |
| | L3 (ubiquitin thiolesterase) | | | | | | | | | | | |
| NM_004385.1 | chondroitin sulfate proteoglycan 2 | \|5\|5q\|5q14\| | | | 1.24 | 1.27 | | | | | | |
| | (versican) | | | | | | | | | | | |
| NM_006186.1 | nuclear receptor subfamily 4, group | \|2\|2q\|2q22\| | | | 0.82 | 0.81 | | 0.78 | 0.71 | | | |
| | A, member 2 | | | | | | | | | | | |
| AF074393.1 | ribosomal protein S6 kinase, 90 kDa, | \|14\|14q\|14q31\| | | | 1.20 | 1.53 | | 1.22 | | 1.37 | | |
| | polypeptide 5 | | | | | | | | | | | |
| NM_012294.1 | guanine nucleotide exchange factor | \|7\|7p\|7p21\| | 1.38 | | | | | | | 1.48 | | 1.86 |
| | for Rap1; M-Ras-regulated GEF | | | | | | | | | | | |
| NM_007191.1 | WNT inhibitory factor 1 | \|12\|12q\|12q13\| | | 1.57 | | | | 0.78 | | 0.43 | | |
| NM_000130.2 | coagulation factor V (proaccelerin, | \|1\|1q\| | | | | 0.80 | 0.63 | | | | | |
| | labile factor) | | | | | | | | | | | |
| NM_004445.1 | EphB6 | \|7\|7q\|7q33\| | | | 0.82 | | | | | | | |
| NM_007168.1 | ATP-binding cassette, sub-family A | \|17\|17q\| | | | 1.86 | 1.58 | 1.48 | 1.27 | 1.39 | | 1.31 | 1.99 |
| | (ABC1), member 8 | | | | | | | | | | | |
| NM_014747.1 | KIAA0237 gene product | \|1\|1p\|1pter\| | | | 0.83 | | | | | | | |
| NM_002774.1 | kallikrein 6 (neurosin, zyme) | \|19\|19q\|19q13\| | | | | | | | 1.30 | | 1.24 | 1.60 |
| NM_005950.1 | metallothionein 1G | \|16\|16q\| | | | 1.20 | | | | | | 1.23 | |
| BE670563 | guanine nucleotide binding protein (G | \|16\|16q\| | | | | | | 1.21 | | | | |
| | protein), alpha activating activity | | | | | | | | | | | |
| | polypeptide O | | | | | | | | | | | |
| NM_014210.1 | ecotropic viral integration site 2A | \|17\|17q\|17q11\| | | | 1.67 | | 1.24 | | 1.59 | | 1.30 | 2.03 |
| NM_002371.2 | mal, T-cell differentiation protein | \|2\| | | 1.23 | 1.81 | 1.43 | 1.21 | | 1.52 | | 1.34 | 1.64 |
| NM_004434.1 | echinoderm microtubule associated | \|14\|4q\| | | | | | | 1.23 | | | | |
| | protein like 1 | | | | | | | | | | | |
| NM_000096.1 | ceruloplasmin (ferroxidase) | \|3\|3q\|3q23\| | | | | | | | | | 1.34 | |
| AF040254.1 | doublecortex; lissencephaly, X-linked | \|X\|Xq\|Xq22\| | | | 0.83 | | | | 0.77 | | | |
| | (doublecortin) | | | | | | | | | | | |
| NM_002594.1 | proprotein convertase subtilisin/kexin | \|20\|20p\|20p11\| | | | 0.79 | | 0.75 | | | | | |
| | type 2 | | | | | | | | | | | |
| NM_003358.1 | UDP-glucose ceramide | \|9\|9q\| | 1.30 | | | | | 1.23 | | 1.32 | | |
| | glucosyltransferase | | | | | | | | | | | |
| NM_000166.1 | gap junction protein, beta 1, 32 kDa | \|X\|Xq\|Xq13\| | | | | | | | | | | 1.27 |
| | (connexin 32, Charcot-Marie-Tooth | | | | | | | | | | | |
| | neuropathy, X-linked) | | | | | | | | | | | |
| AA988241 | RAB3A, member RAS oncogene | \|19\|19p\|19p13\| | | | | 0.78 | | | | | | |
| | family | | | | | | | | | | | |
| NM_004660.2 | DEAD/H (Asp-Glu-Ala-Asp/His) box | \|Y\|Yq\| | 1.42 | 1.31 | | 1.57 | | | | 1.43 | | |
| | polypeptide, Y chromosome | | | | | | | | | | | |
| NM_001446.1 | fatty acid binding protein 7, brain | \|6\|6q\|6q22\| | | | | | | | | 0.80 | | |
| NM_003832.1 | phosphoserine phosphatase-like | \|7\|7q\|7q11\| | | | | 1.52 | | | | | | |
| NM_000222.1 | v-kit Hardy-Zuckerman 4 feline | \|4\|4q\|4q11\| | | | | | | | | | | 0.59 |
| | sarcoma viral oncogene homolog | | | | | | | | | | | |
| NM_002643.1 | phosphatidylinositol glycan, class F | \|2\|2p\|2p21\| | | | 1.20 | 1.20 | | | 1.20 | | | 1.22 |
| NM_002643.1 | phosphatidylinositol glycan, class F | \|2\|2p\|2p21\| | 1.26 | | 1.22 | 1.24 | | 1.23 | 1.23 | | 1.22 | |
| NM_006365.1 | Homo sapiens cDNA FLJ37174 fis, | | | 1.23 | | | | | | 0.68 | | |
| | clone BRACE2028406 | | | | | | | | | | | |
| NM_002372.1 | mannosidase, alpha, class 2A, | \|5\|5q\|5q21\| | | | | | | | | | | 1.35 |
| | member 1 | | | | | | | | | | | |
| NM_000384.1 | apolipoprotein B (including Ag(x) | \|2\|2p\|2p24\| | 0.78 | | | | | | | 0.82 | | |
| | antigen) | | | | | | | | | | | |
| NM_005382.1 | neurofilament 3 (150 kDa medium) | \|8\|8p\| | | | 0.82 | | | 1.27 | | 1.58 | 0.59 | |
| NM_002983.1 | chemokine (C—C motif) ligand 3 | \|17\|17q\|17q11\| | | | | | 0.83 | 0.82 | | 0.79 | | |
| NM_002029.1 | formyl peptide receptor 1 | \|19\|19q\|19q13\| | | | | | 1.25 | | | | | |
| U29586.1 | sarcoglycan, beta (43 kDa dystrophin- | \|4\|4q\| | | | | | | | | | | 1.25 |
| | associated glycoprotein) | | | | | | | | | | | |
| BF439316 | transmembrane protein with EGF-like | \|9\|9q\| | | | | | | | | | | |
| | and two follistatin-like domains 1 | | | | | | | | | | | |
| NM_002157.1 | heat shock 10 kDa protein 1 | \|2\|2q\|2q33\| | | | 1.21 | 1.24 | | | 1.30 | | | 1.27 |
| | (chaperonin 10) | | | | | | | | | | | |
| AV724192 | KIAA0644 gene product | \|7\|7p\|7p21\| | | | 1.37 | | | 0.78 | | 0.76 | | |
| AI003579 | solute carrier family 6 | \|3\|3p\|3p25\| | | | | | | | | 0.75 | 0.80 | 0.73 |
| | (neurotransmitter transporter, GABA), | | | | | | | | | | | |
| | member 1 | | | | | | | | | | | |
| NM_006946.1 | spectrin, beta, non-erythrocytic 2 | \|11\|11q\| | | | | 0.74 | | | | 0.79 | | |
| NM_000168.2 | GLI-Kruppel family member GL13 | \|7\|7p\| | 0.83 | | | | | | | 0.82 | | |
| | (Greig cephalopolysyndactyly | | | | | | | | | | | |
| | syndrome) | | | | | | | | | | | |
| NM_005389.1 | protein-L-isoaspartate (D-aspartate) | \|6\|6q\|6q24\| | 1.30 | | | | | | | | | |
| | O-methyltransferase | | | | | | | | | | | |
| NM_000216.1 | Kallmann syndrome 1 sequence | \|X\|Xp\|Xp22\| | 1.41 | | 1.32 | | | | | | 1.46 | |
| NM_022817.1 | period homolog 2 (Drosophila) | \|2\|2q\|2q37\| | | | 0.76 | | | | 0.77 | 0.80 | | |
| NM_001635.1 | amphiphysin (Stiff-Man syndrome | \|7\|7p\|7p14\| | | | | | | | | 1.34 | | |
| | with breast cancer 128 kDa | | | | | | | | | | | |
| | autoantigen) | | | | | | | | | | | |
| NM_000901.1 | nuclear receptor subfamily 3, group | \|4\|4q\|4q31\| | | 1.26 | | 1.34 | | | | | | |
| | C, member 2 | | | | | | | | | | | |
| NM_000817.1 | glutamate decarboxylase 1 (brain, | \|2\|2q\| | | | 0.82 | 0.83 | | | | | 0.78 | 0.66 |
| | 67 kDa) | | | | | | | | | | | |
| NM_000824.1 | glycine receptor, beta | \|4\|4q\|4q31\| | | | | | 0.77 | | | | | |
| AB017120.1 | BAI1-associated protein 2 | \|17\|17q\| | | | | | | 1.24 | | | | |
| NM_005856.1 | receptor (calcitonin) activity modifying | \|7\|7p\|7p13\| | | | | | | | 1.23 | 1.22 | | 1.34 |
| | protein 3 | | | | | | | | | | | |
| NM_006984.1 | claudin 10 | \|13\|13q\|13q31\| | | 1.21 | 1.27 | | | | | 0.74 | 0.74 | |
| NM_002854.1 | parvalbumin | \|22\|22q\|22q13\| | 0.69 | | 0.71 | 0.75 | | | | | 0.64 | |
| NM_004411.1 | dynein, cytoplasmic, intermediate | \|7\|7q\|7q21\| | 1.22 | | | | | 1.22 | | 1.32 | | |
| | polypeptide 1 | | | | | | | | | | | |
| NM_005025.1 | serine (or cysteine) proteinase | \|3\|3q\|3q26\| | | 1.20 | | | | 1.20 | | 1.27 | 1.28 | |
| | inhibitor, clade I (neuroserpin), | | | | | | | | | | | |
| | member 1 | | | | | | | | | | | |
| NM_003986.1 | butyrobetaine (gamma), 2- | \|11\|11p\| | 1.32 | | 1.34 | | | | | | | |
| | oxoglutarate dioxygenase (gamma- | | | | | | | | | | | |
| | butyrobetaine hydroxylase) 1 | | | | | | | | | | | |
| NM_001918.1 | dihydrolipoamide branched chain | \|1\|1p\| | | | | | | | | | | |
| | transacylase (E2 component of | | | | | | | | | | | |
| | branched chain keto acid | | | | | | | | | | | |
| | dehydrogenase complex; maple | | | | | | | | | | | |
| | syrup urine disease) | | | | | | | | | | | |
| NM_015831.1 | acetylcholinesterase (YT blood | \|7\|7q\| | | | | | | | | | 1.34 | |
| | group) | | | | | | | | | | | |
| NM_015642.1 | zinc finger protein 288 | \|3\|3q\|3q13\| | | | 1.30 | | | | | | | |
| NM_004166.1 | chemokine (C—C motif) ligand 14 | \|17\|17q\|17q11\| | 0.81 | | | | | | | | | |
| NM_004734.1 | doublecortin and CaM kinase-like 1 | \|13\|13q\|13q13\| | | | | | | 1.45 | | | | |
| NM_005525.1 | hydroxysteroid (11-beta) | \|1\|1q\|1q32\| | | 1.37 | | | | | 1.32 | | 1.24 | 1.51 |
| | dehydrogenase 1 | | | | | | | | | | | |
| NM_001740.2 | calbindin 2, 29 kDa (calretinin) | \|16\|16q\|16q22\| | | | | | | | 0.80 | | 1.38 | |
| NM_000055.1 | butyrylcholinesterase | \|3\|3q\|3q26\| | | | | | | | | 0.75 | | |
| NM_021647.1 | KIAA0626 gene product | \|4\|4q\|4q32\| | | | 1.32 | | | | 1.21 | | | 1.31 |
| BC001777.1 | hippocalcin | \|1\|1p\|1p35\| | | | 0.81 | 0.81 | 0.80 | | 0.83 | 0.82 | | 0.75 |
| NM_007281.1 | scrapie responsive protein 1 | \|4\|4q\|4q31\| | 1.30 | 1.36 | 1.44 | 1.44 | 1.39 | | 1.36 | | | |
| NM_001888.1 | crystallin, mu | \|16\|16p\|16p13\| | | | 0.81 | | | | | | | |
| NM_001037.1 | sodium channel, voltage-gated, type | \|19\|19q\|19q13\| | | | 0.81 | | | | | | 0.75 | |
| | I, beta polypeptide | | | | | | | | | | | |
| NM_003186.2 | transgelin | \|11\|11q\|11q23\| | | | | 0.65 | 1.29 | 1.27 | | 1.50 | 1.49 | 1.38 |
| NM_006198.1 | Purkinje cell protein 4 | \|21\|21q\|21q22\| | | 0.74 | 0.79 | 0.72 | 0.65 | | | | | |
| NM_005739.2 | RAS guanyl releasing protein 1 | \|15\|15q\| | | | | | | | | | 0.66 | 1.36 |
| | calcium and DAG-regulated) | | | | | | | | | | | |
| NM_014897.1 | KIAA0924 protein | \|17\|17q\| | | | | | 1.22 | 1.28 | | | | |
| NM_000950.1 | proline-rich Gla (G-carboxyglutamic | \|X\|Xp\|Xp21\| | | | | | | | 1.31 | | 1.28 | 1.46 |
| | acid) polypeptide 1 | | | | | | | | | | | |
| AW014927 | calbindin 1, 28 kDa | \|8\|8q\|8q21\| | | 0.61 | | | | | | | | |
| NM_004929.2 | calbindin 1, 28 kDa | \|8\|8q\|8q21\| | | 0.70 | | | | | | | | |
| BC002599.1 | corticotropin releasing hormone | \|8\|8q\| | | | 0.83 | | | | | 0.82 | | |
| NM_000756.1 | corticotropin releasing hormone | \|8\|8q\| | | | 0.72 | 0.70 | | | 0.69 | 0.63 | | |
| NM_003558.1 | phosphatidylinositol-4-phosphate 5- | \|9\|9q\| | | | | | 0.66 | | | | | |
| | kinase, type I, beta | | | | | | | | | | | |
| NM_007023.1 | cAMP-regulated guanine nucleotide | \|2\|2q\|2q31\| | | | | 0.74 | | | | 0.61 | | 0.69 |
| | exchange facter II | | | | | | | | | | | |
| NM_006998.1 | secretagogin, EF-hand calcium | \|6\|6p\|6p22\| | | 1.29 | | | | | | | | |
| | binding protein | | | | | | | | | | | |
| AL022718 | odz, odd Oz/ten-m homolog | \|X\|Xq\| | | | | | | | | | | 0.78 |
| | 1(Drosophila) | | | | | | | | | | | |
| NM_004102.2 | fatty acid binding protein 3, muscle | \|1\|1p\|1p33\| | | | | 0.73 | | | | | | |
| | and heart (mammary-derived growth | | | | | | | | | | | |
| | inhibitor) | | | | | | | | | | | |
| NM_001392.1 | dystrobrevin, alpha | \|18\|18q\| | 1.89 | 1.41 | 1.73 | 1.75 | 1.75 | 1.56 | 1.86 | 1.57 | 1.52 | 1.53 |
| NM_004352.1 | cerebellin 1 precursor | \|16\|16q\|16q12\| | | 0.81 | | | | | | | | |
| NM_003026.1 | SH3-domain GRB2-like 2 | \|9\|9p\| | | | | | | 1.21 | | 1.36 | | |
| NM_000840.1 | glutamate receptor, metabotropic 3 | \|7\|7q\|7q21\| | | | | | | | | | 1.24 | 1.35 |
| NM_000729.2 | cholecystokinin | \|3\|3p\|3p22\| | | | | | | | | | 1.69 | |
| NM_004271.1 | MD-1, RP105-associated | \|6\|6p\|6p24\| | | | | | 1.20 | | | | | |
| NM_004476.1 | folate hydrolase (prostate-specific | \|11\|11p\|11p11\| | | | | | | | | | | 1.79 |
| | membrane antigen) 1 | | | | | | | | | | | |
| AI818488 | adducin 3 (gamma) | \|10\|10q\|10q24\| | | | 1.23 | | | | | | | |
| NM_003914.1 | cyclin A1 | \|13\|13q\|13q12\| | | | | | | | | | | 0.83 |
| NM_002612.1 | pyruvate dehydrogenase kinase, | \|7\|7q\|7q21\| | | | 1.24 | 1.60 | | | | | 1.27 | |
| | isoenzyme 4 | | | | | | | | | | | |
| NM_005954.1 | metallothionein 3 (growth inhibitory | \|16\|16q\| | 1.24 | 1.43 | 1.34 | | 1.27 | | 1.28 | | | |
| | factor (neurotrophic)) | | | | | | | | | | | |
| NM_004795.1 | klotho | \|13\|13q\| | | | | | 0.55 | 1.20 | | | | |
| NM_002433.1 | myelin oligodendrocyte glycoprotein | \|6\|6p\|6p22\| | | | 1.69 | | | 1.39 | 1.54 | | 1.27 | 1.93 |
| NM_000905.1 | neuropeptide Y | \|7\|7p\|7p15\| | | | | 0.59 | 0.71 | | | | | |
| NM_000266.1 | Norrie disease (pseudoglioma) | \|X\|Xp\|Xp11\| | 1.33 | | 1.23 | 1.26 | | | 1.33 | | | |
| NM_006681.1 | neuromedin U | \|4\|4q\| | | | | | | | | | 1.57 | |
| NM_000049.1 | aspartoacylase (aminoacylase 2, | \|17\|17p\|17pter\| | 1.55 | | 1.45 | 1.53 | | 1.21 | | | 1.27 | 1.77 |
| | Canavan disease) | | | | | | | | | | | |
| NM_004794.1 | RAB33A, member RAS oncogene | \|X\|Xq\|Xq26\| | | | | 1.30 | | | | | 1.32 | 1.37 |
| | family | | | | | | | | | | | |
| NM_003430.1 | zinc finger protein 91 (HPF7, HTF10) | \|19\|19p\|19p13\| | 0.77 | | 0.77 | 0.79 | | | | 0.72 | 0.68 | 0.75 |
| NM_006157.1 | NEL-like 1 (chicken) | \|11\|11p\|11p15\| | | | | | | | | | | 0.69 |
| NM_014682.1 | zinc finger protein 387 | \|8\|8q\|8q11\| | | | | | | | | | | 1.71 |
| NM_003180.1 | synaptotagmin V | \|19\|19q\| | | | | 0.83 | 0.80 | | | | | |
| NM_005584.1 | mab-21-like 1 (C. elegans) | \|13\|13q\| | | | | | | | | | 0.78 | 0.68 |
| NM_003551.1 | non-metastatic cells 5, protein | \|5\|5q\| | | | | | 0.82 | | | | | |
| | expressed in (nucleoside- | | | | | | | | | | | |
| | diphosphate kinase) | | | | | | | | | | | |
| NM_002500.1 | neurogenic differentiation 1 | \|2\|2q\| | | | | | 0.77 | | | | | |
| NM_001036.1 | ryanodine receptor 3 | \|15\|15q\|15q14\| | 1.41 | | 1.41 | 1.34 | | | 1.24 | | | 1.35 |
| NM_004291.1 | cocaine- and amphetamine-regulated | \|5\|5q\|5q13\| | | | | | | | | 1.42 | 2.00 | |
| | transcript | | | | | | | | | | | |
| NM_006123.1 | iduronate 2-sulfatase (Hunter | \|X\|Xq\| | | | | | | | | | | |
| | syndrome) | | | | | | | | | | | |
| NM_005097.1 | leucine-rich, glioma inactivated 1 | \|10\|10q\| | | | | | | | | | | 1.24 |
| NM_003412.1 | Zic family member 1 (odd-paired | \|3\|3q\| | 1.34 | | 1.30 | | | | | | | |
| | homolog, Drosophila) | | | | | | | | | | | |
| NM_018057.1 | homolog of rat orphan transporter v7-3 | \|12\|12q\|12q21\| | 1.57 | | | | | 1.20 | | 1.36 | 1.35 | 1.26 |
| NM_020987.1 | ankyrin 3, node of Ranvier (ankyrin | \|10\|10q\| | | | | | | 1.20 | | | | |
| | G) | | | | | | | | | | | |
| NM_014717.1 | KIAA0390 gene product | \|19\|19q\|19q13\| | | | 1.47 | 1.32 | | | | | | 1.63 |
| NM_005951.1 | metallothionein 1H | \|16\|16q\| | | | 1.22 | | | | | | | |
| NM_004746.1 | discs, large (Drosophila) homolog- | \|18\|18p\|18p11\| | | | 0.80 | | | | 0.81 | | | |
| | associated protein 1 | | | | | | | | | | | |
| NM_006240.1 | protien phosphatase, EF hand | \|X\|Xp\|Xp22\| | | | | 0.65 | | | | | | |
| | calcium-binding domain 1 | | | | | | | | | | | |
| NM_003182.1 | tachykinin, precursor 1 (substance K, | \|7\|7q\|7q21\| | | | 0.66 | 0.53 | 0.49 | | | 0.55 | | |
| | substabce P, neurokinin 1, neurokinin | | | | | | | | | | | |
| | 2, neuromedin L, neurokinin alpha, | | | | | | | | | | | |
| | neuropeptide K, neuropeptide | | | | | | | | | | | |
| | gamma) | | | | | | | | | | | |
| NM_015364.1 | MD-2 protein | \|8\|8q\|8q13\| | | | | | 1.23 | | 1.32 | | | |
| NM_004654.2 | ubiquitin specific protease 9, Y | \|Y\|Yq\|Yq11\| | 1.30 | | | | | | | 1.34 | | |
| | chromosome (fat facets-like | | | | | | | | | | | |
| | Drosophila) | | | | | | | | | | | |
| NM_000079.1 | cholinergic receptor, nicotinic, alpha | \|2\|2q\|2q24\| | | | | 0.79 | | | | | | |
| | polypeptide 1 (muscle) | | | | | | | | | | | |
| NM_005413.1 | sine oculis homeobox homolog 3 | \|2\|2p\|2p16\| | 0.78 | | | | | | | | | |
| | (Drosophila) | | | | | | | | | | | |
| NM_000407.3 | glycoprotein lb (platelet), beta | \|22\|22q\|22q11\| | | | 0.80 | 0.75 | | | | 0.64 | | |
| | polypeptide | | | | | | | | | | | |
| NM_013445.1 | glutamate decarboxylase 1 (brain, | \|2\|2q\| | | | 0.76 | | | | 0.77 | | 0.79 | 0.68 |
| | 67 kDa) | | | | | | | | | | | |
| NM_000806.2 | gamma-aminobutyric acid (GABA) A | \|5\|5q\|5q34\| | | | 0.81 | | | | 0.73 | | 0.71 | |
| | receptor, alpha 1 | | | | | | | | | | | |
| NM_001163.1 | amyloid beta (A4) precursor protein- | \|9\|9q\|9q13\| | 0.79 | | | | | | | | | |
| | binding, family A, member 1 (X11) | | | | | | | | | | | |
| NM_003991.1 | endothelin receptor type B | \|13\|13q\| | | | | 1.25 | | | | 0.78 | | |
| NM_014927.1 | connector enhancer of KSR2 | \|X\|Xp\|Xp22\| | | | | | | 1.35 | | 1.30 | | |
| NM_014926.1 | KIAA0848 protein | \|3\|3q\|3q26\| | | | 0.81 | | | | | | | |
| AF153820.1 | potassium inwardly-rectifying | \|17\|17q\|17q23\| | | | | 1.35 | | | | | | 1.47 |
| | channel, subfamily J, member 2 | | | | | | | | | | | |
| NM_002347.1 | lymphocyte antigen 6 complex, locus H | \|8\|8q\|8q24\| | | | | | 0.76 | | | | | |
| NM_000496.1 | crystallin, beta B2 | \|22\|22q\|22q11\| | 0.80 | | | 0.76 | | | | | 0.79 | |
| NM_000818.1 | glutamate decarboxylase 2 | \|10\|10p\|10p11\| | | | 0.81 | 0.80 | | | | | 0.75 | 0.66 |
| | (pancreatic islets and brian, 65 kDa) | | | | | | | | | | | |
| NM_024411.1 | prodynorphin | \|20\|20p\|20pter\| | | | | 0.73 | | | | | | |
| NM_002677.1 | peripheral myelin protein 2 | \|8\|8q\|8q21\| | | | 1.44 | | | | | 0.64 | | |
| NM_000816.1 | gamma-aminobutyric acid (GABA) A | \|5\|5q\|5q31\| | | | 0.74 | | | | | | | |
| | receptor, gamma 2 | | | | | | | | | | | |
| AL138761 | Ste20-related serine/threonine kinase | \|10\|10q\|10q25\| | 1.21 | | | 1.22 | | 1.34 | | 1.26 | | |
| NM_005071.1 | solute carrier family 1 (high affinity | \|19\|19p\|19p13\| | | 0.74 | | | | | | | | |
| | aspartate/glutamate transporter), | | | | | | | | | | | |
| | member 6 | | | | | | | | | | | |
| U82532.1 | Rho GDP dissociation inhibitor (GDI) | \|16\|16p\|16p13\| | | | 0.79 | 0.79 | | | 0.81 | | | |
| | gamma | | | | | | | | | | | |
| NM_012344.1 | neurotensin receptor 2 | \|2\|2p\|2p25\| | | | 1.31 | | | | | | | |
| NM_002509.1 | NK2 transcription factor homolog B | \|20\|20p\|20pter\| | | 1.21 | | | | | 1.25 | | | 1.35 |
| | (Drosophila) | | | | | | | | | | | |
| NM_002590.2 | protocadherin 8 | \|13\|13q\|13q14\| | | | 0.64 | | | | | | | |
| NM_006644.1 | heat shock 105 kD | \|13\|13q\|13q12\| | 1.24 | | | 1.36 | 0.83 | 1.22 | | 1.43 | | |
| NM_002930.1 | Ras-like without CAAX 2 | \|18\|18q\|18q12\| | | | | | | 1.24 | | | | |
| NM_000812.2 | gamma-aminobutyric acid (GABA) A | \|4\|4p\| | | 1.25 | | | | | | | 1.28 | |
| | receptor, beta 1 | | | | | | | | | | | |
| NM_000807.1 | gamma-aminobutyric acid (GABA) A | \|4\|4p\| | | 1.40 | | | | | | | | |
| | receptor, alpha 2 | | | | | | | | | | | |
| NM_001321.1 | cysteine and glycine-rich protein 2 | \|12\|12q\|12q21\| | 1.54 | 1.25 | 1.54 | 1.43 | 1.27 | | 1.34 | | | 1.29 |
| NM_002650.1 | phosphatidylinositol 4-kinase, | \|22\|22q\|22q11\| | | | 0.82 | | | | | | | |
| | catalytic, alpha polypeptide | | | | | | | | | | | |
| NM_002562.1 | purinergic receptor P2X, ligand-gated | \|12\|12q\| | 1.33 | | 1.35 | 1.64 | | 1.27 | | | 1.25 | 1.33 |
| | ion channel, 7 | | | | | | | | | | | |
| NM_000621.1 | 5-hydroxytryptamine (serotonin) | \|13\|13q\|13q\|14\| | | | | | | | | | | 0.79 |
| | receptor 2A | | | | | | | | | | | |
| NM_006352.1 | zinc finger protein 238 | \|1\|1q\|1q44\| | | 0.71 | 0.77 | | | | | | | |
| NM_001954.2 | discoidin domain receptor family, | \|6\|6p\|6p21\| | | | 1.47 | 1.28 | | | | | 1.31 | |
| | member 1 | | | | | | | | | | | |
| NM_004466.2 | glypican 5 | \|13\|13q\| | | | 1.30 | | | | | 0.80 | | |
| NM_000811.1 | gamma-aminobutyric acid (GABA) A | \|5\|5q\| | | 0.83 | | | | | | | | |
| | receptor, alpha 6 | | | | | | | | | | | |
| NM_004459.2 | fetal Alzheimer antigen | \|17\|17q\| | | | | | | | | | | |
| NM_014461.1 | contactin 6 | \|3\|3p\|3p26\| | | | | | | | | | 0.80 | |
| NM_004065.1 | cerebellar degeneration-related | \|X\|Xq\|Xq27\| | | | | | | 1.54 | | | | |
| | protein 1, 34 kDa | | | | | | | | | | | |
| NM_000838.2 | glutamate receptor, metabotropic 1 | \|6\|6q\| | | | | | | | | 1.25 | | |
| NM_000868.1 | 5-hydroxytryptamine (serotonin) | \|X\|Xq\| | | | | | 0.33 | | | | | 0.76 |
| | receptor 2C | | | | | | | | | | | |
| NM_012090.1 | microtubule-actin crosslinking factor 1 | \|1\|1p\|1p32\| | | | | | | | | | | |
| NM_000864.1 | 5-hydroxytryptamine (serotonin) | \|1\|1p\|1p36\| | 0.83 | | | | | | | | | |
| | receptor 1D | | | | | | | | | | | |
| NM_002570.1 | paired basic amino acid cleaving | \|15\|15q\| | | | | | | | | | | 1.58 |
| | system 4 | | | | | | | | | | | |
| AF220532.1 | nuclear receptor subfamily 2, group | \|6\|6q\| | | | | | | 0.83 | | | | |
| | E, member 1 | | | | | | | | | | | |
| NM_020149.1 | Meis1, myeloid ecotropic viral | \|15\|15q\|15q13\| | 1.22 | | | | | | 1.21 | | | 0.65 |
| | integration site 1 homolog 2 (mouse) | | | | | | | | | | | |
| NM_000385.2 | aquaporin 1 (channel-forming integral | \|7\|7p\| | 1.87 | | 1.56 | 1.73 | | 1.32 | | 1.61 | 1.96 | |
| | protein, 28 kDa) | | | | | | | | | | | |
| NM_007177.1 | TU3A protein | \|3\|3p\|3p21\| | | 1.27 | 1.42 | 1.25 | 1.24 | | | | 1.32 | |
| NM_001939.1 | dystrophin related protein 2 | \|X\|Xq\| | 0.81 | | | | | | | | | |
| NM_006501.1 | myelin-associated oligodendrocyte | \|3\|3p\|3p21\| | | | | | 1.21 | | | 0.68 | | |
| | basic protein | | | | | | | | | | | |
| NM_013436.1 | NCK-associated protein 1 | \|2\|2q\| | 1.27 | | | | | | | | | |
| NM_0.14033.1 | DKFZP586A0522 protein | \|12\|12q\| | 1.44 | 1.40 | 1.74 | 1.30 | | | 1.30 | | | |
| NM_001965.1 | early growth response 4 | \|2\|2p\| | | 0.79 | 0.62 | 0.57 | 0.70 | | 0.63 | 0.69 | | |
| NM_015874.1 | H-2K binding factor-2 | \|9\| | 1.37 | | 1.33 | 1.36 | | | 1.20 | | | 1.27 |
| NM_004161.1 | RAB1A, member RAS oncogene | \|2\|2p\| | 1.30 | | | | | | | | | |
| | family | | | | | | | | | | | |
| NM_000313.1 | protein S (alpha) | \|3\|3p\|3p11\| | | | | | 0.59 | | | | 1.33 | |
| NM_015530.1 | golgi reassembly stacking protein 2, | \|2\|2p\|2p24\| | | | | 1.29 | | | | 1.26 | 1.24 | 1.26 |
| | 55 kDa | | | | | | | | | | | |
| L36675.1 | synuclein, alpha (non A4 component | \|4\|4q\| | 1.37 | | | | | | | | 1.23 | |
| | of amyloid precursor) | | | | | | | | | | | |
| NM_003085.2 | synuclein, beta | \|5\|5q\| | | | | | | | | | | |
| NM_004902.1 | RNA-binding region (RNP1, RRM) | \|20\|20q\|20q11\| | 1.30 | | 1.21 | 1.29 | | | | | 1.23 | |
| | containing 2 | | | | | | | | | | | |
| NM_006930.1 | S-phase kinase-associated protein | \|5\|5q\|5q22\| | | | | | | | | | | |
| | 1A (p19A) | | | | | | | | | | | |
| NM_007274.1 | brain acyl-CoA hydrolase | \|1\|1p\|1p36\| | | | | | | 1.26 | | | | |
| NM_018407.1 | | | 1.24 | | | 1.39 | | | | | | 1.24 |
| NM_021575.1 | adaptor-related protein complex 2, | \|19\|19q\|19q13\| | | | | | | 1.21 | | | | |
| | sigma 1 subunit | | | | | | | | | | | |
| NM_002848.2 | | | 1.31 | | | | | 1.27 | | | | |
| NM_003471.1 | potassium voltage-gated channel, | \|3\|3q\|3q26\| | | 0.83 | | | 0.82 | 1.47 | | | | |
| | shaker-related subfamily, beta | | | | | | | | | | | |
| | member 1 | | | | | | | | | | | |
| M87771.1 | fibroblast growth factor receptor 2 | \|10\|10q\| | | 1.40 | 1.46 | | 0.81 | | | | | 1.36 |
| | (bacteria-expressed kinase, | | | | | | | | | | | |
| | keratinocyte growth factor receptor, | | | | | | | | | | | |
| | craniofacial dysostosis 1, Crouzon | | | | | | | | | | | |
| | syndrome, Pfeiffer syndrome, | | | | | | | | | | | |
| | Jackson-Weiss syndrome) | | | | | | | | | | | |
| NM_17618.1 | hypothetical protein FLJ20006 | \|16\|16q\|16q23\| | | | | | | | | | | |
| NM_000175.1 | glucose phosphate isomerase | \|19\|19q\|19q13\| | | | | | | 1.21 | | 1.23 | | |
| NM_003360.1 | UDP glycosyltransferase 8 (UDP- | \|4\|4q\| | | | | | | | | | | 1.74 |
| | galactose ceramide | | | | | | | | | | | |
| | galactosyltransferase) | | | | | | | | | | | |
| NM_004171.1 | solute carrier family 1 (glial high | \|11\|11p\|11p13\| | | | | | | | | 0.61 | 0.76 | |
| | affinity glutamate transporter), | | | | | | | | | | | |
| | member 2 | | | | | | | | | | | |
| NM_006016.1 | CD164 antigen, sialomucin | \|6\|6q\| | | | | | | | | | | |
| NM_000457.1 | hepatocyte nuclear factor 4, alpha | \|20\|20q\|20q12\| | 0.79 | | | | | | | | | |
| NM_000592.2 | complement component 4B | \|6\|6p\|6p21\| | 1.45 | | 1.43 | 1.37 | | | 1.37 | 1.48 | 1.77 | 1.38 |
| NM_000815.1 | gamma-aminobutyric acid (GABA) A | \|1\|1p\|1p36\| | | | 0.75 | | | | 0.78 | | | |
| | receptor, delta | | | | | | | | | | | |
| NM_006161.1 | neurogenin 1 | \|5\|5q\|5q23\| | | | | | | | | | | 0.83 |
| NM_005952.1 | metallothionein 1X | \|16\|16q\| | | 1.38 | 1.43 | 1.35 | 1.27 | | | | | |
| J05021.1 | villin 2 (ezrin) | \|6\|6q\|6q25\| | | | 1.28 | | | 0.79 | | 0.64 | | |
| BC003576.1 | actinin, alpha 1 | \|14\|14q\| | | | | | | 1.27 | | 1.28 | | |
| AA872727 | farnesyl-diphosphate | \|8\|8p\|8p23\| | 1.28 | | 1.30 | 1.27 | | 1.24 | 1.32 | | 1.38 | 1.33 |
| | farnesyltransferase 1 | | | | | | | | | | | |
| BG327863 | CD24 antigen (small cell lung | \|6\|6q\| | | | | | 0.73 | 0.82 | | | | |
| | carcinoma cluster 4 antigen) | | | | | | | | | | | |
| BC004443.1 | ATPase, H+ transporting, lysosomal | \|22\|22q\|22q11\| | 1.22 | | | | | | | 1.26 | | 1.24 |
| | 31 kDa, V1 subunit E isoform 1 | | | | | | | | | | | |
| L19184.1 | peroxiredoxin 1 | \|1\|1p\|1p34\| | | | 1.22 | 1.22 | | | | | | |
| M23254.1 | calpain 2, (m/II) large subunit | \|1\|1q\|1q41\| | | | 1.20 | 1.29 | | | | | | |
| BC001188.1 | transferrin receptor (p90, CD71) | \|3\|3q\|3q26\| | | | | | 1.25 | | | | | 1.41 |
| U14990.1 | ribosomal protein S3 | \|11\|11q\|11q13\| | | | 1.27 | | 1.24 | | | | 1.30 | |
| D30658.1 | glycyl-tRNA synthetase | \|7\|7p\| | | | | | | | | | | 1.25 |
| BC001019.1 | ribosomal protein L39 | \|X\|Xq\|Xq22\| | | | 1.25 | | | | | | | |
| AL080102.1 | eukaryotic translation initiation factor 5 | \|14\|14q\|14q32\| | 1.24 | | | | | 1.20 | 1.21 | 1.26 | | 1.31 |
| AF092131.1 | NADH dehydrogenase (ubiquinone) | \|11\|11q\| | | | | | | 1.26 | | | | 1.25 |
| | flavoprotein 1, 51 kDa | | | | | | | | | | | |
| U59321.1 | DEAD/H (Asp-Glu-Ala-Asp/His) box | \|22\|22q\|22q13\| | | 0.58 | | | | | | | | 0.70 |
| | polypeptide 17, 72 kDa | | | | | | | | | | | |
| BC000905.1 | RAB1A, member RAS oncogene | \|2\|2p\| | 1.23 | | | | | | | | | |
| | family | | | | | | | | | | | |
| BC000461.1 | eukaryotic translation initiation factor | \|20\|20p\|20pter\| | | | | 1.28 | | 1.21 | | 1.26 | 1.24 | 1.42 |
| | 2, subunit 2 beta, 38 kDa | | | | | | | | | | | |
| D83043.1 | major histocompatibility complex, | \|6\|6p\|6p21\| | 1.38 | | 1.28 | | 1.27 | 1.33 | | 1.32 | 1.44 | 1.39 |
| | class I, B | | | | | | | | | | | |
| NM_002865.1 | RAB2, member RAS oncogene family | \|8\|8q\|8q11\| | | | | | | | | | | 0.79 |
| NM_002865.1 | RAB2, member RAS oncogene family | \|8\|8q\|8q11\| | 1.21 | | | | | | | | | |
| AF070655.1 | ATP synthase, H+ transporting, | \|3\|3q\| | 1.24 | | | | | 1.24 | | | | |
| | mitochondrial F0 complex, subunit g | | | | | | | | | | | |
| M18767.1 | complement component 1, s | \|12\|12p\| | 1.22 | | | | | | | 1.27 | | |
| | subcomponent | | | | | | | | | | | |
| AA580004 | ADP-ribosylation factor 1 | \|1\|1q\| | 1.22 | | | | | 1.31 | | 1.30 | | |
| D89976.1 | 5-aminoimidazole-4-carboxamide | \|2\|2q\| | | | 1.21 | 1.25 | | | 1.24 | 1.23 | 1.23 | 1.28 |
| | ribonucleotide formyltransferase/IMP | | | | | | | | | | | |
| | cyclohydrolase | | | | | | | | | | | |
| D13119.1 | ATP synthase, H+ transporting, | \|12\|12q\| | | | | | | 1.24 | | | | 1.22 |
| | mitochondrial F0 complex, subunit c | | | | | | | | | | | |
| | (subunit 9), isoform 2 | | | | | | | | | | | |
| D89729.1 | exportin 1 (CRM1 homolog, yeast) | \|2\|2p\| | 1.30 | | | 1.37 | | | | | 1.20 | |
| L20817.1 | discoidin domain receptor family, | \|6\|6p\|6p21\| | | | 1.40 | | | | 1.20 | | 1.36 | |
| | member 1 | | | | | | | | | | | |
| AF154847.1 | VAMP (vesicle-associated membrane | \|18\|18\|18p11\| | | | | | | | | | | 1.21 |
| | protein)-associated protein A, 33 kDa | | | | | | | | | | | |
| AL136969.1 | homolog of yeast long chain | \|6\|6p\|6p21\| | | | 1.70 | 1.42 | 1.26 | | 1.52 | | 1.26 | 1.34 |
| | polyunsaturated fatty acid elongation | | | | | | | | | | | |
| | enzyme 2 | | | | | | | | | | | |
| M25915.1 | clusterin (complement lysis inhibitor, | \|8\|8p\|8p21\| | 1.41 | | 1.32 | 1.36 | 1.22 | | 1.22 | 1.20 | | |
| | SP-40,40, sulfated glycoprotein 2, | | | | | | | | | | | |
| | testosterone-repressed prostate | | | | | | | | | | | |
| | message 2, apolipoprotein J) | | | | | | | | | | | |
| M25915.1 | clusterin (complement lysis inhibitor, | \|8\|8p\|8p21\| | 1.50 | 1.23 | 1.44 | 1.38 | 1.20 | | 1.21 | 1.25 | 1.21 | |
| | SP-40,40, sulfated glycoprotein 2, | | | | | | | | | | | |
| | testosterone-repressed prostate | | | | | | | | | | | |
| | message 2, apolipoprotein J) | | | | | | | | | | | |
| NM_006947.1 | signal recognition particle 72 kDa | \|4\|4q\| | 1.23 | | | | | | | | | |
| NM_006947.1 | signal recognition particle 72 kDa | \|4\|4q\| | 1.25 | | | | | | | | | |
| BC002979.1 | proteasome (prosome, macropain) | \|14\|14q\| | 1.24 | | | | | 1.20 | | | | 1.22 |
| | subunit, alpha type, 6 | | | | | | | | | | | |
| BC000903.1 | high-mobility group box 2 | \|4\|4q\| | 1.61 | 1.42 | 1.48 | | 1.34 | | 1.43 | 1.39 | | |
| BC004489.1 | major histocompatibility complex, | \|6\|6p\|6p21\| | 1.41 | 1.32 | 1.40 | | 1.32 | 1.30 | 1.39 | 1.40 | 1.34 | 1.51 |
| | class I, C | | | | | | | | | | | |
| BC000419.1 | catechol-O-methyltransferase | \|22\|22q\|22q11\| | | | | | | | | 0.79 | | |
| AL037339 | PTK2 protein tyrosine kinase 2 | \|8\|8q\|8q24\| | | | | | | | | | | 1.21 |
| AB014560.1 | Ras-GTPase activating protein SH3 | \|4\|4q\|4q21\| | 1.30 | | | | | 1.23 | | 1.28 | | |
| | domain-binding protein 2 | | | | | | | | | | | |
| BC005247.1 | isopentenyl-diphosphate delta | \|10\|10p\|10p15\| | | | 1.20 | | | | 1.25 | | 1.35 | |
| | isomerase | | | | | | | | | | | |
| BC003143.1 | dual specificity phosphatase 6 | \|12\|12q\|12q22\| | | | 0.77 | | | | | | | 1.24 |
| BC001362.1 | 2′,3′-cyclic nucleotide 3′ | \|17\|17q\| | | | | | | | 1.25 | | 1.24 | 1.46 |
| | phosphodiesterase | | | | | | | | | | | |
| AV752215 | sorcin | \|7\|7q\|7q21\| | 1.25 | 1.21 | 1.26 | | | | | | | |
| L12387.1 | sorcin | \|7\|7q\|7q21\| | 1.23 | 1.25 | 1.22 | 1.20 | | | | | | |
| AF161522.1 | chromosome 3 open reading fram 4 | \|3\|3p\|3p11\| | | | 1.54 | 1.40 | | | | | 1.32 | 1.54 |
| BC004954.1 | ribosomal protein L13 | \|16\|16q\|16q24\| | | | 1.21 | | | | | | | |
| BC002515.1 | aldehyde dehydrogenase 7 family, | \|5\|5q\| | | | 1.43 | 1.59 | 1.23 | | 1.25 | | | |
| | member A1 | | | | | | | | | | | |
| U62891.1 | dUTP pyrophosphatase | \|15\|15q\|15q15\| | 1.23 | | 1.22 | | | | | | | |
| BE540552 | fatty acid desaturase 1 | \|11\|11q\|11q12\| | | | 1.31 | | | | | | | |
| BE540552 | fatty acid desaturase 1 | \|11\|11q\|11q12\| | | | 1.29 | | | | | | | |
| AL512760.1 | fatty acid desaturase 1 | \|11\|11q\|11q12\| | | | 1.38 | | | | | | 1.26 | |
| BC002719.1 | eukaryotic translation initiation factor | \|15\|15q\| | | | | | 1.20 | | | | | 1.22 |
| | 3, subunit 1 alpha, 35 kDa | | | | | | | | | | | |
| AL559478 | transcription factor 12 (HTF4, helix- | \|15\|15q\| | | | 1.33 | | | | | | | |
| | loop-helix transcription factors 4) | | | | | | | | | | | |
| BC001329.1 | KIAA0202 protein | \|5\|5q\| | | | | | | | 1.27 | | | 1.31 |
| AK026678.1 | stromal antigen 2 | \|X\|Xq\| | | | 1.35 | | | | | | | |
| AF141349.1 | hypothetical protein DKFZp434N0650 | \|21\|21q\|21q22\| | | | | | | 1.22 | | | 1.22 | |
| BF673013 | spectrin SH3 domain binding protein 1 | \|10\|10p\|10p11\| | 1.33 | | | | | | | | 1.20 | |
| AF006516.1 | spectrin SH3 domain binding protein 1 | \|10\|10p\|10p11\| | 1.26 | | | | | | | | | |
| D86550.1 | dual-specificity tyrosine-(Y)- | \|21\|21q\|21q22\| | 1.22 | | | | | | | | | |
| | phosphorylation regulated kinase 1A | | | | | | | | | | | |
| U17496.1 | proteasome (prosome, macropain) | \|6\|6p\|6p21\| | | | | | | | | | | 1.21 |
| | subunit, beta type, 8 (large | | | | | | | | | | | |
| | multifunctional protease 7) | | | | | | | | | | | |
| AL518391 | aquaporin 1 (channel-forming integral | \|7\|7p\| | 1.97 | | 1.94 | 1.98 | | 1.34 | 1.38 | 1.66 | 2.49 | |
| | protein, 28 kDa) | | | | | | | | | | | |
| AL050264.1 | TU3A protein | \|3\|3p\|3p21\| | | 1.26 | 1.44 | 1.30 | | | 1.20 | | 1.35 | |
| AL049597 | SH3-domain GRB2-like endophilin B1 | \|1\|1p\| | | | 1.24 | 1.25 | | | | | | |
| AF061730.1 | CGI-150 protein | \|17\|17p\|17p13\| | 1.34 | | | | | | | 1.26 | | |
| AF141347.1 | tubulin, alpha 3 | \|12\|12q\|12q12\| | | | | | | | | | | 1.25 |
| AF289489.1 | aspartate beta-hydroxylase | \|8\|8q\|8q12\| | 1.53 | | 1.49 | | 1.31 | | 1.22 | | | |
| AV704962 | sterol-C4-methyl oxidase-like | \|4\|4q\|4q32\| | 1.25 | | 1.47 | | | 1.22 | 1.38 | | 1.41 | 1.28 |
| AF234997.1 | Tax interaction protein 1 | \|17\|17p\| | 1.48 | 1.34 | 1.68 | 1.37 | 1.39 | 1.31 | 1.73 | 1.36 | 1.32 | 1.40 |
| AF016004.1 | glycoprotein M6B | \|X\|Xp\|Xp22\| | | | 1.40 | | | | | 0.83 | | 1.21 |
| AF016004.1 | Homo sapiens cDNA FLJ38338 fis, | | 1.21 | | 1.24 | | | | | | 1.21 | 1.23 |
| | clone FCBBF3027104, highly similar | | | | | | | | | | | |
| | to Mus musculus proteolipid M6B | | | | | | | | | | | |
| | isoform alpha-beta-TMD-omega | | | | | | | | | | | |
| | (M6B) mRNA | | | | | | | | | | | |
| AF016004.1 | glycoprotein M6B | \|X\|Xp\|Xp22\| | | | 1.43 | | | | | | | 1.20 |
| AW516932 | down-regulator of transcription 1, | \|1\|1p\|1p22\| | 1.25 | | | | | | | | 1.21 | |
| | TBP-binding (negative cofactor 2) | | | | | | | | | | | |
| BC004490.1 | v-fos FBJ murine osteosarcoma viral | \|14\|14q\|14q24\| | | | 0.71 | | 0.74 | 0.61 | 0.65 | | | |
| | oncogene homolog | | | | | | | | | | | |
| BC004291.1 | hypothetical protein MGC17226 | \|1\|1q\|1q21\| | | | | | | | | | | 1.32 |
| N22468 | MADS box transcription enhancer | \|5\|5q\| | | | | | 1.30 | | | 1.29 | | |
| | factor 2, polypeptide C (myocyte | | | | | | | | | | | |
| | enhancer factor 2C) | | | | | | | | | | | |
| AW469573 | mitogen inducible 2 | \|14\|14q\|14q22\| | 1.31 | | | 1.37 | | | | | | |
| Z24725.1 | mitogen inducible 2 | \|14\|14q\|14q22\| | 1.47 | 1.35 | 1.64 | 1.31 | 1.23 | | | | | |
| BC002511.1 | carbonyl reductase 1 | \|21\|21q\|21q22\| | | | 1.41 | 1.38 | | | 1.20 | | 1.25 | 1.40 |
| AF098865.1 | squalene epoxidase | \|8\|8q\|8q24\| | | | | | | | 1.29 | | 1.26 | |
| U72069.1 | karyopherin (importin) beta 2 | \|5\|5q\|5q13\| | | | | | | 1.23 | | | | |
| AF070560.1 | O-linked N-acetylglucosamine | \|X\|Xq\| | 1.22 | | | 1.31 | | | | 1.25 | | |
| | (GlcNAc) transferase (UDP-N- | | | | | | | | | | | |
| | acetylglucosamine: polypeptide-N- | | | | | | | | | | | |
| | acetylglucosaminyl transferase) | | | | | | | | | | | |
| BC000961.2 | degenerative spermatocyte homolog, | \|1\|1q\| | 1.24 | | | | | | | | | |
| | lipid desaturase (Drosophila) | | | | | | | | | | | |
| AF020043.1 | chondroitin sulfate proteoglycan 6 | \|10\|10q\| | 1.28 | | 1.22 | | | | 1.20 | | | |
| | (bamacan) | | | | | | | | | | | |
| BC002675.1 | hypothetical protein MGC4276 similar | \|9\|9q\|9q22\| | 1.26 | | | | | | | | | |
| | to CG8198 | | | | | | | | | | | |
| AF007162.1 | crystallin, alpha B | \|11\|11q\|11q22\| | | 1.31 | 1.80 | 1.57 | 1.31 | 1.28 | 1.35 | | 1.31 | 1.75 |
| NM_001546.1 | inhibitor of DNA binding 4, dominant | \|6\|6p\|6p22\| | 1.46 | | 1.48 | | | | | | | |
| | negative helix-loop-helix protein | | | | | | | | | | | |
| NM_001546.1 | inhibitor of DNA binding 4, dominant | \|6\|6p\|6p22\| | 1.37 | | | | | 0.75 | | | | |
| | negative helix-loop-helix protein | | | | | | | | | | | |
| M36532.1 | carbonic anhydrase II | \|8\|8q\| | | | | 1.36 | 0.69 | | 1.54 | | | 1.59 |
| U65585.1 | major histocompatibility complex, | \|6\|6p\|6p21\| | | | 1.27 | | | | 1.23 | | | |
| | class II, DR beta I | | | | | | | | | | | |
| BC003525.1 | MAX protein | \|14\|14q\| | | 1.25 | | | | | | | | |
| AF063020.1 | PC4 and SFRS1 interacting protein 2 | \|9\|9p\|9p22\| | 1.25 | | 1.21 | 1.20 | | | | | 1.31 | |
| BC002449.1 | hypothetical protein FLJ13612 | \|2\|2q\|2q36\| | | | 1.52 | 1.31 | | | 1.21 | | 1.23 | 1.29 |
| AB000889.1 | phosphatidic acid phosphatase type | \|1\|1p\|1pter\| | | 1.31 | 1.70 | 1.30 | | | | 0.74 | | |
| | 2B | | | | | | | | | | | |
| L35594.1 | ectonucleotide | \|8\|8q\|8q24\| | | | | | 0.63 | 1.25 | 1.39 | | 1.27 | 1.68 |
| | pyrophosphatase/phosphodiesterase | | | | | | | | | | | |
| | 2 (autotaxin) | | | | | | | | | | | |
| M80927.1 | chitinase 3-like 1 (cartilage | \|1\|1q\|1q31\| | 1.87 | | 1.60 | 1.53 | 1.74 | 1.52 | 1.32 | 1.45 | | 1.43 |
| | glycoprotein-39) | | | | | | | | | | | |
| M80927.1 | chitinase 3-like 1 (cartilage | \|1\|1q\|1q31\| | 1.49 | | | 1.49 | 1.56 | | | | | |
| | glycoprotein-39) | | | | | | | | | | | |
| AL109965 | chromosome 20 open reading frame | \|20\|20q\|20q11\| | 1.21 | | | | | | | | | |
| | 104 | | | | | | | | | | | |
| U48437.1 | amyloid beta (A4) precursor-like | \|19\|19q\|19q13\| | | | | | | | | | 1.27 | 1.45 |
| | protein 1 | | | | | | | | | | | |
| AL565812 | pleiotrophin (heparin binding growth | \|7\|7q\|7q33\| | | | | 0.76 | | | | 0.64 | | |
| | factor 8, neurite growth-promoting | | | | | | | | | | | |
| | factor 1) | | | | | | | | | | | |
| M57399.1 | pleiotrophin (heparin binding growth | \|7\|7q\|7q33\| | | | | | | | | 0.69 | 0.79 | 0.79 |
| | factor 8, neurite growth-promoting | | | | | | | | | | | |
| | factor 1) | | | | | | | | | | | |
| D49958.1 | glycoprotein M6A | \|4\|4q\| | | | | | | | | | | 0.75 |
| NM_000618.1 | insulin-like growth factor 1 | \|12\|12q\|12q22\| | | | | 0.83 | | | | | 0.79 | 0.65 |
| | (somatomedin C) | | | | | | | | | | | |
| BC001745.1 | DNA segment on chromosome 4 | \|4\|4p\|4p16\| | | | 0.82 | | | 1.23 | | | | |
| | (unique) 234 expressed sequence | | | | | | | | | | | |
| AL049933.1 | guanine nucleotide binding protein (G | \|7\|7q\| | | | | 1.38 | | | | | 1.40 | 1.70 |
| | protein), alpha inhibiting activity | | | | | | | | | | | |
| | polypeptide 1 | | | | | | | | | | | |
| AF114784.1 | methyl-CpG binding domain protein 4 | \|3\|3q\|3q21\| | 1.23 | | | | | | | | | |
| BC001387.1 | HRAS-like suppressor 3 | \|11\|11q\|11q13\| | | 1.24 | 1.56 | 1.34 | | 1.20 | 1.32 | | | 1.63 |
| AL530874 | EphB2 | \|1\|1p\|1p36\| | 0.82 | | | | | | | | | |
| U51120.1 | p21/Cdc42/Rac1-activated kinase 1 | \|11\|11q\|11q13\| | | | 0.78 | | | | | | | |
| | (STE20 homolog, yeast) | | | | | | | | | | | |
| U87460.1 | G protein-coupled receptor 37 | \|7\|7q\| | | | | 1.67 | | | 1.37 | | 1.30 | 1.80 |
| | (endothelin receptor type B-like) | | | | | | | | | | | |
| AI803181 | brain cell membrane protein 1 | \|X\|Xp\|Xp11\| | 1.32 | | 1.26 | 1.22 | | | | | | |
| AL136550.1 | brain cell membrane protein 1 | \|X\|Xp\|Xp11\| | 1.51 | | 1.36 | 1.21 | | | | | | |
| M65217.1 | heat shock transcription factor 2 | \|6\|6q\|6q22\| | | | | 1.26 | | | | | | |
| AF162690.1 | transthyretin (prealbumin, | \|18\|18q\|18q12\| | | | | 0.53 | 0.13 | 0.81 | | | 0.45 | 0.38 |
| | amyloidosis type I) | | | | | | | | | | | |
| BC005383.1 | centrin, EF-hand protein, 3 (CDC31 | \|5\|5q\|5q14\| | 1.42 | 1.22 | 1.42 | 1.27 | | | 1.35 | | | |
| | homolog, yeast) | | | | | | | | | | | |
| AL136924.1 | Ras and Rab interactor 2 | | | | 1.46 | 1.24 | | | 1.20 | | | |
| M13975.1 | protein kinase C, beta 1 | \|16\|16p\|16p11\| | | | 0.83 | | | | | | | |
| BC001766.1 | S100 calcium binding protein, beta | \|21\|21q\|21q22\| | | 1.35 | 1.50 | 1.26 | 1.24 | | 1.35 | | | |
| | (neural) | | | | | | | | | | | |
| U19495.1 | chemokine (C—X—C motif) ligand 12 | \|10\|10q\|10q11\| | | | | | 0.80 | | | 1.21 | | |
| | (stromal cell-derived factor 1) | | | | | | | | | | | |
| BC004492.1 | DKFZP586A0522 protein | \|12\|12q\| | | | | | | | | | | |
| L20860.1 | glycoprotein lb (platelet), beta | \|22\|22q\|22q11\| | | | | | | | | | | |
| | polypeptide | | | | | | | | | | | |
| AL049569 | peptidyl arginine deiminase, type II | \|1\|1p\|1p35\| | | | 1.25 | | | | | | | |
| AL567302 | glutamate receptor, ionotropic, AMPA 1 | \|5\|5q\|5q31\| | | 1.34 | | | 0.75 | 1.24 | | | | |
| AF001294.1 | tumor suppressing subtransferable | \|11\|11p\|11p15\| | | | 0.78 | | | | | | | |
| | candidate 3 | | | | | | | | | | | |
| U43148.1 | patched homolog (Drosophila) | \|9\|9q\|9q22\| | | | 1.22 | | | | | | | |
| AB002384.1 | chromosome 6 open reading frame | \|6\|6p\|6p22\| | | | | | 0.66 | | | | | |
| | 32 | | | | | | | | | | | |
| J04543.1 | annexin A7 | \|10\|10q\|10q21\| | 1.30 | | | 1.30 | | | | 1.30 | | |
| M83248.1 | secreted phosphoprotein 1 | \|4\|4q\|4q21\| | | 1.40 | 1.88 | 1.95 | 1.44 | 1.35 | 1.96 | 1.94 | 1.27 | 2.05 |
| | (osteopontin, bone sialoprotein I, | | | | | | | | | | | |
| | early T-lymphocyte activation 1) | | | | | | | | | | | |
| AF055585.1 | slit homolog 2 (Drosophila) | \|4\|4p\|4p15\| | | | | | | 1.20 | | | | 0.81 |
| AL162079.1 | solute carrier family 16 | \|1\|1p\| | 1.51 | | 1.32 | | | | 1.39 | | 1.27 | 1.49 |
| | (monocarboxylic acid transporters), | | | | | | | | | | | |
| | member 1 | | | | | | | | | | | |
| U90223.1 | dUTP pyrophosphatase | \|15\|15q\|15q15\| | | | 1.21 | | | | | 1.23 | | |
| AF225981.1 | ATPase, Ca++ transporting, type 2C, | \|3\|3q\|3q21\| | 1.30 | | | | | | | | | |
| | member 1 | | | | | | | | | | | |
| D14826.1 | cAMP responsive element modulator | \|10\|10p\|10p12\| | 1.26 | | | | | | 1.20 | | | |
| AF069755.1 | G protein-coupled receptor 51 | \|9\|9q\|9q22\| | | | 0.79 | | | | 0.78 | | | |
| AF079363.1 | sperm associated antigen 6 | \|10\|10p\|10p12\| | | | | | 0.65 | 0.73 | | | | |
| D63412.1 | aquaporin 4 | \|18\|18q\|18q11\| | 2.03 | | 1.62 | 1.76 | 1.31 | | | | 1.33 | |
| D63412.1 | aquaporin 4 | \|18\|18q\|18q11\| | 1.82 | | 1.62 | 1.69 | 1.25 | | | | | |
| U63622.1 | aguaporin 4 | \|18\|18q\|18q11\| | 1.77 | | 2.09 | 1.84 | 1.45 | 1.23 | 1.40 | | | |
| U46745.1 | dystrobrevin, alpha | \|18\|18q\| | 1.58 | | | 1.37 | 1.39 | 1.52 | | 1.37 | | 1.38 |
| D49372.1 | chemokine (C—C motif) ligand 11 | \|17\|17q\|17q21\| | | | | | | | | | | |
| AW070431 | myelin basic protein | \|18\|18q\| | | | 1.71 | | 1.44 | 1.37 | | | | 1.96 |
| AF074979.1 | regulator of G-protein signalling 20 | \|8\|8q\|8q12\| | | 1.31 | 1.29 | 1.25 | | | | 0.76 | 1.21 | 1.24 |
| L03203.1 | peripheral myelin protein 22 | \|17\|17p\|17p12\| | | 1.27 | 1.58 | 1.39 | | | 1.55 | | | 1.68 |
| D25547.1 | protein-L-isoaspartate (D-aspartate) | \|6\|6q\|6q24\| | 1.26 | | | | | | | | | |
| | O-methyltransferase | | | | | | | | | | | |
| AB007880.1 | KIAA0420 gene product | \|16\|16p\|16p13\| | | | | | | | | | | 1.57 |
| AJ007557.1 | potassium inwardly-rectifying | \|2\|2q\| | | | | 0.80 | 0.66 | | | | | |
| | channel, subfamily J, member 13 | | | | | | | | | | | |
| AF169148.1 | calcium binding protein 1 (calbrain) | \|12\|12q\|12q24\| | | | 0.78 | 1.26 | | | | 1.37 | | |
| AB000263.1 | cortistatin | \|1\|1p\|1p36\| | | | | 0.81 | | | | | | |
| D28114.1 | myelin-associated oligodendrocyte | \|3\|3p\|3p21\| | | | | | | | | 0.65 | | |
| | basic protein | | | | | | | | | | | |
| BC002665.1 | proteolipid protein 1 (Pelizaeus- | \|X\|Xq\| | | | | | | | | | | |
| | Merzbacher disease, spastic | | | | | | | | | | | |
| | paraplegia 2, uncomplicated) | | | | | | | | | | | |
| AF001383.1 | bridging integrator 1 | \|2\|2q\| | | | 1.31 | | | | | | 1.36 | 1.39 |
| U87558.1 | bridging integrator 1 | \|2\|2q\| | | | 1.35 | 1.28 | | 1.23 | | | 1.38 | 1.38 |
| AF028832.1 | heat shock 90 kDa protein 1, alpha | \|14\|14q\|14q32\| | | | | | | | | 1.23 | | |
| BC000513.1 | cholinergic receptor, nicotinic, alpha | \|15\|15q\| | | | | 0.72 | | | | | 0.69 | |
| | polypeptide 3 | | | | | | | | | | | |
| BC000314.1 | reticulon 1 | \|14\|14q\|14q21\| | | | | | | 1.22 | | 1.31 | | |
| AF120274.1 | artemin | \|1\|1p\|1p33\| | 0.82 | | | | | | | | | |
| AW139618 | synapsin II | \|3\|3p\| | | | | | | | | | | 0.77 |
| AF112221.1 | KIAA0871 protein | \|4\|4q\|4q13\| | 1.31 | | | 1.44 | | 1.26 | | | | |
| U28936.1 | tyrosine 3- | \|17\|17p\|17p13\| | | | | | | | | | | |
| | monooxygenase/tryptophan 5- | | | | | | | | | | | |
| | monooxygenase activation protein, | | | | | | | | | | | |
| | epsilon polypeptide | | | | | | | | | | | |
| AB034951.1 | heat shock 70 kDa protein 8 | \|11\|11q\|11q23\| | 1.23 | | | | | | | | | |
| BC003092.1 | retinoblastoma binding protein 4 | \|1\|1p\|1p34\| | 1.26 | | | | | | | | 1.26 | |
| BC000740.1 | cholecystokinin B receptor | \|11\|11p\|11p15\| | | | 0.79 | | | | | 0.81 | | |
| AB009288.1 | copine VI (neuronal) | \|14\|14q\|14q11\| | | | | | 0.76 | 1.20 | | | | |
| AF164622.1 | golgin-67 | \|15\|15q\|15q11\| | | 0.73 | | | | | | | | |
| BC001388.1 | annexin A2 | \|15\|15q\|15q21\| | | | | | | | | 1.26 | 1.61 | 1.28 |
| AF225986.1 | sodium channel, voltage-gated, type | \|2\|2q\| | | | | | | | 0.81 | | | 0.66 |
| | III, alpha polypeptide | | | | | | | | | | | |
| AB033605.1 | proteasome (prosome, macropain) | \|1\|1q\|1q21\| | | | | | | 1.27 | | | | 1.26 |
| | 26S subunit, non-ATPase, 4 | | | | | | | | | | | |
| M37712.1 | cell division cycle 2-like 2 | \|1\|1p\|1p36\| | | | 1.30 | | | | | | | |
| D82346.1 | potassium voltage-gated channel, | \|20\|20q\|20q13\| | 0.80 | | | | | | | | | |
| | KQT-like subfamily, member 2 | | | | | | | | | | | |
| AF022375.1 | vascular endothelial growth factor | \|6\|6p\| | | | | 0.70 | 0.81 | 0.69 | | 0.63 | 0.68 | 0.70 |
| BC000906.1 | NAD(P)H dehydrogenase, quinone 1 | \|16\|16q\|16q22\| | | | | 1.28 | | | 1.20 | | | |
| U26744.1 | dystrobrevin, alpha | \|18\|18q\| | 1.72 | | | 1.70 | 1.54 | 1.67 | 1.44 | 1.33 | 1.30 | 1.34 |
| AF028825.1 | discs, large (Drosophila) homolog 4 | \|17\|17p\|17p13\| | | 0.82 | | | | | | | | |
| AF011390.1 | solute carrier family 4, sodium | \|4\|4q\| | | | | | | | | 0.80 | | |
| | bicarbonate cotransporter, member 4 | | | | | | | | | | | |
| L11315.1 | discoidin domain receptor family, | \|6\|6p\|6p21\| | | | 1.37 | | | | | | 1.28 | |
| | member 1 | | | | | | | | | | | |
| AF053640.1 | CSE1 chromosome segregation 1- | \|20\|20q\| | | | | | | | | | | 1.28 |
| | like (yeast) | | | | | | | | | | | |
| L05666.1 | glutamate receptor, ionotropic, N- | \|9\|9q\|9q34\| | | | | | | | | | | |
| | methyl D-aspartate 1 | | | | | | | | | | | |
| M81590.1 | 5-hydroxytryptamine (serotonin) | \|6\|6q\| | 0.80 | | | | | | | | | |
| | receptor 1B | | | | | | | | | | | |
| AF001602.1 | paraoxonase 2 | \|7\|7q\|7q21\| | | 1.41 | 1.56 | 1.36 | | | | 0.81 | | |
| D45421.1 | ectonucleotide | \|8\|8q\|8q24\| | | | | | 0.58 | | | | 1.27 | 1.63 |
| | pyrophosphatase/phosphodiesterase | | | | | | | | | | | |
| | 2 (autotaxin) | | | | | | | | | | | |
| D14705.1 | catenin (cadherin-associated protein), | \|5\|5q\| | | | 1.36 | 1.25 | | | | | 1.25 | 1.34 |
| | alpha 1 (102 kDa | | | | | | | | | | | |
| AF135593.1 | vacuolar protein sorting 41 (yeast) | \|7\|7p\|7p14\| | | | | | | | | | | |
| U34846.1 | aquaporin 4 | \|18\|18q\|18q11\| | 1.63 | | 1.89 | 1.55 | 1.39 | | | | | |
| BC003169.1 | calpain 3, (p94) | \|15\|15q\|15q15\| | | | | | | | | | | 1.61 |
| Z24727.1 | tropomyosin 1 (alpha) | \|15\|15q\|15q22\| | | | | | | | | | | 1.23 |
| L12723.1 | heat shock 70 kDa protein 4 | \|5\|5q\|5q31\| | | | | 1.20 | | | | | | |
| BC006259.1 | cytoplasmic linker 2 | \|7\|7q\|7q11\| | | | | | | | | | 1.31 | 1.21 |
| Z25521.1 | | | | | | 1.56 | | | 1.30 | 1.33 | | 1.34 |
| AF015730.1 | glutamate receptor, ionotropic, N- | \|9\|9q\|9q34\| | | | 0.81 | | | | | | | |
| | methyl D-aspartate 1 | | | | | | | | | | | |
| L38019.1 | inositol 1,4,5-triphosphate receptor, | \|3\|3p\|3p26\| | | 0.65 | 0.82 | | | | | | | |
| | type 1 | | | | | | | | | | | |
| D50855.1 | calcium-sensing receptor | \|3\|3q\|3q21\| | | | | | | | | | | 0.82 |
| | (hypocalciuric hypercalcemia 1, | | | | | | | | | | | |
| | severe neonatal | | | | | | | | | | | |
| | hyperparathyroidism) | | | | | | | | | | | |
| AB013889.1 | potassium inwardly-rectifying | \|2\|2q\| | | | | | 0.60 | | | | | |
| | channel, subfamily J, member 13 | | | | | | | | | | | |
| M81778.1 | 5-hydroxytryptamine (serotonin) | \|X\|Xq\| | | | | | 0.65 | 1.30 | | | | |
| | receptor 2C | | | | | | | | | | | |
| AF112206.1 | RAB14, member RAS oncogene | \|9\|9q\|9q32\| | | | | 1.27 | | 1.28 | | | | |
| | family | | | | | | | | | | | |
| U56725.1 | heat shock 70 kDa protein 2 | \|14\|14q\|14q24\| | | | 1.78 | 1.67 | | | 1.56 | 1.35 | 1.26 | 1.75 |
| AF020340.1 | guanylate cyclase 1, soluble, beta 3 | \|4\|4q\|4q31\| | | | | | | | | | | 0.77 |
| M97260.1 | ATPase, Ca++ transporting, plasma | \|3\|3p\|3p26\| | | 0.78 | 0.73 | | | | 0.79 | | | |
| | membrane 2 | | | | | | | | | | | |
| AB050468.1 | leucine-rich repeats and | | 1.31 | | 1.55 | 1.36 | 1.27 | | 1.26 | | | |
| | immunoglobulin-like domains 1 | | | | | | | | | | | |
| U20489.1 | | | 0.69 | 0.83 | 0.73 | 0.83 | 0.83 | 0.77 | 078 | 0.75 | 0.73 | 0.71 |
| M92439.1 | leucine-rich PPR-motif containing | \|2\|2p\|2p22\| | 1.20 | | | 1.22 | | 1.23 | | 1.33 | | |
| M61900.1 | | | | | 1.29 | | | | | | | |
| AF250307.1 | dynein, cytoplasmic, intermediate | \|2\|2q\| | | | | | | | | | | 1.23 |
| | polypeptide 2 | | | | | | | | | | | |
| AF349571.1 | hemoglobin, alpha 1 | \|16\|16p\|16p13\| | | | | | | 1.51 | | | | |
| AF353990.1 | beta-amyloid binding protein | \|1\|1p\|1p32\| | 1.26 | | | | | | | | | |
| | precursor | | | | | | | | | | | |
| BC005916.1 | pleiotrophin (heparin binding growth | \|7\|7q\|7q33\| | | | | 0.82 | | | | 0.66 | 0.78 | 0.78 |
| | factor 8, neurite growth-promoting | | | | | | | | | | | |
| | factor 1) | | | | | | | | | | | |
| BC005931.1 | hemoglobin, alpha 2 | \|16\|16p\|16p13\| | | | | | | 1.51 | | | | |
| BC005932.1 | proteasome (prosome, macropain) | \|11\|11p\|11p15\| | 1.23 | | | | | 1.21 | | 1.22 | | |
| | subunit, alpha type, 1 | | | | | | | | | | | |
| BC005939.1 | prostaglandin D2 synthase 21 kDa | \|9\|9q\|9q34\| | | | 1.35 | | | | | | | |
| | (brain) | | | | | | | | | | | |
| BC005954.1 | NADH dehydrogenase (ubiquinone) | \|19\|19p\| | | | | | | | | | | 1.30 |
| | Fe—S protein 7, 20 kDa (NADH- | | | | | | | | | | | |
| | coenzyme Q reductase) | | | | | | | | | | | |
| BC005961.1 | parathyroid hormone-like hormone | \|12\|12p\|12p12\| | | | | 0.65 | | | | | | |
| AF043179.1 | T cell receptor beta locus | \|7\|7q\| | | | | | | | | | 0.62 | |
| AF172268.1 | KIAA0551 protein | \|3\|3q\|3q26\| | | | | | | | | | | 0.83 |
| U18800.1 | myelin oligodendrocyte glycoprotein | \|6\|6p\|6p22\| | | | | | | | | | 1.39 | 1.66 |
| AF073745.2 | phosphodiesterase 4A, cAMP- | \|19\|19p\|19p13\| | | | | | | | | | | |
| | specific (phosphodiesterase E2 | | | | | | | | | | | |
| | dunce homolog, Drosophila) | | | | | | | | | | | |
| L07950.1 | | | 1.37 | 1.25 | 1.38 | 1.34 | 1.32 | 1.34 | | 1.43 | 1.61 | 1.49 |
| AF348514.1 | prothymosin, alpha (gene sequence | \|2\|2q\|2q35\| | | | 1.36 | | | | | | | 1.36 |
| | 28) | | | | | | | | | | | |
| AY028632.1 | catalase | \|11\|11p\| | | | 1.29 | 1.33 | | | | | 1.25 | 1.21 |
| AF216292.1 | heat shock 70 kDa protein 5 (glucose- | \|9\|9q\|9q33\| | | | | | | | | | | 1.22 |
| | regulated protein, 78 kDa) | | | | | | | | | | | |
| BG500301 | ESTs, Highly similar to | | | | | | | | | | | |
| | ITB1_HUMAN Integrin | | | | | | | | | | | |
| | beta-1 precursor (Fibronectin | | | | | | | | | | | |
| | receptor beta subunit) (CD29) | | | | | | | | | | | |
| | (Integrin VLA-4 beta subunit) | | | | | | | | | | | |
| | [H. sapiens] | | | | | | | | | | | |
| NM_002271.1 | karyopherin (importin) beta 3 | \|13\|13q\|13q32\| | 1.26 | | | 1.27 | | | | | 1.28 | 1.23 |
| BF246436 | putative translation initiation factor | \|17\| | 1.26 | | 1.21 | 1.24 | | | | | | 1.33 |
| L27560.1 | Human insulin-like growth factor | | 1.43 | | 1.35 | | | | | | | |
| | binding protein 5 (IGFBP5) mRNA | | | | | | | | | | | |
| AK000826.1 | RAB7, member RAS oncogene family | \|3\|3q\|3q22\| | | | | | | | | | | 1.20 |
| X79067.1 | zinc finger protein 36, C3H type-like 1 | \|14\|14q\|14q22\| | 1.45 | 1.30 | 1.47 | 1.27 | | | | | 1.38 | |
| AL516350 | actin related protein 2/3 complex, | \|1\|1q\|1q24\| | | | | | | | | | 1.22 | 1.31 |
| | subunit 5, 16 kDa | | | | | | | | | | | |
| BG538627 | pro-oncosis receptor inducing | \|11\|11q\|11q22\| | | | 1.34 | | | | | | | |
| | membrane injury gene | | | | | | | | | | | |
| BG287862 | Homo sapiens cDNA FLJ33834 fis, | | | | 1.20 | | | | | | | |
| | clone CTONG2004264, moderately | | | | | | | | | | | |
| | similar to NEUROBLAST | | | | | | | | | | | |
| | DIFFERENTIATION ASSOCIATED | | | | | | | | | | | |
| | PROTEIN AHNAK | | | | | | | | | | | |
| NM_001068.1 | topoisomerase (DNA) II beta 180 kDa | \|3\|3p\| | 1.20 | | | | | | | | | |
| AL567820 | actin, gamma 1 | \|17\|17q\| | | | | | | 1.22 | | | | |
| AK025647.1 | hypothetical protein H41 | \|3\|3q\|3q22\| | 1.25 | | | | | | | | | |
| AK025557.1 | Homo sapiens cDNA: FLJ21904 fis, | | | | | | | 1.38 | | | | |
| | clone HEP03585 | | | | | | | | | | | |
| BE903880 | CD44 antigen (homing function and | \|11\|11p\| | 2.09 | | 1.80 | 1.57 | 1.36 | | 1.37 | 1.43 | 2.47 | 2.02 |
| | Indian blood group system) | | | | | | | | | | | |
| BE734356 | myosin, light polypeptide 6, alkali, | \|12\|12q\| | | | | | | 1.21 | | | | |
| | smooth muscle and non-muscle | | | | | | | | | | | |
| BE858180 | paternally expressed 10 | \|7\|7q\| | 1.22 | | | | 0.79 | | | | | |
| AL096842.1 | AT2 receptor-interacting protein 1 | \|8\|8p\| | | | 1.66 | 1.48 | 1.22 | 1.24 | 1.42 | | 1.23 | 1.57 |
| AW517686 | ESTs | | 1.30 | | | | | | | | | |
| NM_005953.1 | metallothionein 2A | \|16\|16q\| | | 1.26 | 1.33 | 1.26 | | | | | | |
| NM_000954.1 | prostaglandin D2 synthase 21 kDa | \|9\|9q\|9q34\| | | | 1.37 | | | | | | | |
| | (brain) | | | | | | | | | | | |
| AL541302 | serine (or cysteine) proteinase | \|2\|2q\|2q33\| | 1.20 | 1.31 | | | | | | | | |
| | inhibitor, clade E (nexin, plasminogen | | | | | | | | | | | |
| | activator inhibitor type 1), member 2 | | | | | | | | | | | |
| AF052169.1 | hypothetical protein BC013764 | \|13\|13q\|13q22\| | 1.75 | | 1.38 | | | 1.30 | | 1.42 | 1.25 | |
| AL049265.1 | Homo sapiens mRNA; cDNA | | 1.32 | | 1.38 | 1.31 | 1.25 | | | | | |
| | DKFZp564F053 (from clone | | | | | | | | | | | |
| | DKFZp564F053) | | | | | | | | | | | |
| BF338947 | interferon induced transmembrane | \|11\| | 1.51 | 1.38 | 1.52 | 1.52 | 1.46 | 1.51 | 1.37 | 1.64 | 1.55 | 1.54 |
| | protein 3 (1-8U) | | | | | | | | | | | |
| AF132733.1 | DKFZP564G2022 protein | \|15\|15q\| | | | 1.20 | | | | | | | 1.33 |
| AL576654 | phosphatidic acid phosphatase type | \|1\|1p\|1pter\| | | | 1.55 | | | 0.81 | | 0.72 | | |
| | 2B | | | | | | | | | | | |
| AL576654 | phosphatidic acid phosphatase type | \|1\|1p\|1pter\| | 1.28 | 1.34 | 1.62 | | | 0.80 | | | | |
| | 2B | | | | | | | | | | | |
| Z19574 | keratin 17 | \|17\|17q\|17q12\| | | | | 0.60 | | | | | | |
| AL565074 | tubulin, alpha 1 (testis specific) | \|2\|2q\|2q36\| | 1.36 | | | | | 1.45 | | 1.39 | | 1.37 |
| AI972475 | Homo sapiens cDNA FLJ20738 fis, | | 1.36 | | | 1.32 | | 1.21 | 1.28 | 1.25 | 1.27 | 1.41 |
| | clone HEP08257 | | | | | | | | | | | |
| AF142419.1 | homolog of mouse quaking QKI (KH | \|6\|6q\|6q26\| | | | | | | | | 0.78 | | |
| | domain RNA binding protein) | | | | | | | | | | | |
| AF142419.1 | homolog of mouse quaking QKI (KH | \|6\|6q\|6q26\| | | | | | | | | | | |
| | domain RNA binding protein) | | | | | | | | | | | |
| AA195999 | mitogen-activated protein kinase 1 | \|22\|22q\|22q11\| | | | | | | 1.24 | | | | |
| AB011126.1 | formin-binding protein 17 | \|9\|9q\| | | | 1.37 | | | | | | | 1.30 |
| BE620457 | neuropilin 1 | \|10\|10p\| | | | | | | | 1.22 | | | |
| AW167793 | glucosamine (N-acetyl)-6-sulfatase | \|12\|12q\| | | | | | 0.82 | | | | | 1.29 |
| | (Sanfilippo disease IIID) | | | | | | | | | | | |
| AA621962 | myosin ID | \|17\|17q\|17q11\| | | | | | | | | | | 1.36 |
| BE780075 | transmembrane trafficking protein | \|14\|14q\|14q24\| | 1.29 | | 1.24 | 1.31 | | | | | 1.33 | 1.30 |
| AW043713 | sulfatase FP | \|8\|8q\|8q13\| | 1.27 | | | | | | | | 1.53 | |
| AL137751.1 | radixin | \|11\|11q\| | | | 1.53 | | | | | | 1.23 | 1.37 |
| AL137751.1 | radixin | \|11\|11q\| | | | | | | | | | | |
| AV715767 | Homo sapiens mRNA; cDNA | | | | 1.28 | | | | | | | |
| | DKFZp564A072 (from clone | | | | | | | | | | | |
| | DKFZp564A072 | | | | | | | | | | | |
| AL049949.1 | Homo sapiens, similar to | | | 1.27 | 1.25 | | | | | | | |
| | Y43E12A.2.p, clone MGC: 33537 | | | | | | | | | | | |
| | IMAGE: 4821347, mRNA, complete | | | | | | | | | | | |
| | cds | | | | | | | | | | | |
| AL049949.1 | Homo sapiens, similar to | | | 1.28 | 1.27 | | 1.20 | | | | | |
| | Y43E12A.2.p, clone MGC: 33537 | | | | | | | | | | | |
| | IMAGE: 4821347, mRNA, complete | | | | | | | | | | | |
| | cds | | | | | | | | | | | |
| AA630314 | ribosomal protein S2 | \|16\|16p\|16p13\| | | | 1.24 | | | | | | | 1.22 |
| AB033105.1 | DKFZP586B0923 protein | \|10\|10q\|10q22\| | 1.24 | | | | | | | 1.21 | | |
| D26069.1 | centaurin, beta 2 | \|3\|3q\| | | | | 1.27 | | | | | | |
| AL135735 | phosphatidylinositol binding clathrin | \|11\|11q\| | 1.53 | | 1.40 | 1.40 | | 1.26 | 1.35 | 1.32 | | 1.54 |
| | assembly protein | | | | | | | | | | | |
| BE568219 | phosphodiesterase 8A | \|15\|15q\|15q25\| | | | | | | | | | | 1.31 |
| AL080111.1 | NIMA (never in mitosis gene a)- | \|1\|1q\|1q31\| | | 1.27 | 1.25 | | | | | | | 0.82 |
| | related kinase 7 | | | | | | | | | | | |
| AU144066 | Homo sapiens cDNA FLJ11904 fis, | | | | 1.23 | | | | | | | |
| | clone HEMBB1000048 | | | | | | | | | | | |
| AL576253 | zizimin1 | \|13\|13q\|13q32\| | | | | | | | | | 1.26 | 1.31 |
| BE617588 | hippocalcin-like 1 | \|2\|2p\|2p25\| | | | | | | | | | 0.79 | |
| AL573201 | KIAA0830 protein | \|11\|11q | | | 1.24 | 1.35 | | | 1.30 | | 1.21 | 1.58 |
| BF026595 | ribosomal protein S17 | \|15\|15q\| | | | 1.23 | | | | | | | |
| AB040884.1 | oxysterol binding protein-like 8 | \|12\|12q\| | | | 0.81 | | 0.72 | | 0.56 | | | 0.75 |
| AW298092 | KIAA0776 protein | \|6\|6q\|6q16\| | | | 1.30 | | | | | | | |
| AL031781 | homolog of mouse quaking QKI (KH | \|6\|6q\|6q26\| | | 1.26 | 1.45 | | | | 1.21 | | | |
| | domain RNA binding protein) | | | | | | | | | | | |
| AL581768 | tubulin, alpha 3 | \|12\|12q\|12q12\| | | | | | | 1.26 | | | | 1.22 |
| D42043.1 | KIAA0084 protein | \|3\|3p\|3p24\| | | 1.44 | | | | | | | 1.20 | |
| AL575922 | myosin IF | \|19\|19p\|19p13\| | | 1.78 | | | | | | | 1.63 | |
| BF966021 | ESTs, Highly similar to T08670 cell | | | | 1.46 | | | | | | | 1.38 |
| | division control protein homolog | | | | | | | | | | | |
| | DKFZp564M1416.1 - human | | | | | | | | | | | |
| | (fragment) [H. sapiens] | | | | | | | | | | | |
| AB011178.1 | SCN Circadian Oscillatory Protein | \|18\|18q\|18q21\| | | | 1.31 | | | | | | 1.22 | |
| | (SCOP) | | | | | | | | | | | |
| U79297.1 | Homo sapiens mRNA; cDNA | | 1.22 | | | | | | | 1.20 | | |
| | DKFZp667C0525 (from clone | | | | | | | | | | | |
| | DKFZp667C0525) | | | | | | | | | | | |
| AA923354 | monoamine oxidase A | \|X\|Xp\|Xp11\| | | | 1.22 | 1.20 | | | | | | |
| AL050144.1 | zinc finger protein 363 | \|4\|4q\|4q21\| | 1.29 | 1.20 | | 1.40 | 1.22 | 1.20 | 1.21 | 1.21 | 1.24 | |
| BG165094 | translocase of outer mitochondrial | \|1\|1q\| | | | | | | 1.49 | | 1.27 | | |
| | membrane 20 (yeast) homolog | | | | | | | | | | | |
| AB020684.1 | KIAA0877 protein | \|7\|7p\|7P15\| | | | | | | | 1.21 | | | 1.30 |
| AK001389.1 | hypothetical protein DKFZp564O043 | \|7\|7p\| | 1.23 | | | 1.30 | | | | 1.27 | | |
| BE742268 | sortilin 1 | \|1\|1p\|1p21\| | | | | 1.31 | | | | | | 1.38 |
| AI700633 | Homo sapiens cDNA: FLJ22642 fis, | | | | | | | | | | | |
| | clone HSI06970 | | | | | | | | | | | |
| AA149644 | junctional adhesion molecule 3 | \|11\|11q\| | | | | | | | | | | 1.52 |
| AK023253.1 | DnaJ (Hsp40) homolog, subfamily B, | \|9\|9p\|9p11\| | | | | 0.80 | | | | 0.79 | | |
| | member 5 | | | | | | | | | | | |
| BE878277 | Homo sapiens cDNA FLJ13267 fis, | | | 1.23 | 1.51 | 1.27 | 1.25 | 1.20 | 1.23 | | | 1.55 |
| | clone OVARC1000964 | | | | | | | | | | | |
| AA584297 | low density lipoprotein receptor- | \|11\|11p\|11p11\| | | | 1.35 | 1.22 | 1.22 | | 1.23 | | | |
| | related protein 4 | | | | | | | | | | | |
| AK024044.1 | Sjogren syndrome antigen A2 | \|1\|1q\| | | | 1.34 | | | | | | | |
| | (60 kDa, ribonucleoprotein | | | | | | | | | | | |
| | autoantigen SS-A/Ro) | | | | | | | | | | | |
| BG231551 | activated RNA polymerase II | \|5\|5p\|5p13\| | 1.20 | | | | | | | | | |
| | transcription cofactor 4 | | | | | | | | | | | |
| AI628605 | son of sevenless homolog 2 | \|14\|14q\| | | | | | | | | | | |
| | (Drosophila) | | | | | | | | | | | |
| AI753659 | Sec23 homolog A (S. cerevisiae) | \|14\|14q\|14q13\| | 1.37 | | | | | 1.23 | | | | |
| BG109746 | Homo sapiens cDNA FLJ33775 fis, | | | | 1.26 | | | | | | | |
| | clone BRSSN2000498 | | | | | | | | | | | |
| AI992251 | Homo sapiens cDNA FLJ14821 fis, | | | | 1.30 | | 1.39 | 1.20 | 1.32 | | 1.23 | 1.51 |
| | clone OVARC1000556, highly similar | | | | | | | | | | | |
| | to RIBOSOMAL PROTEIN S6 | | | | | | | | | | | |
| | KINASE II ALPHA 2 (EC 2.7.1.—) | | | | | | | | | | | |
| BE251303 | calreticulin | \|19\|19p\|19p13\| | | | | | | 1.21 | | | | |
| AI769685 | cysteinyl-tRNA synthetase | \|11\|11p\|11p15\| | | | | | | | | | 1.24 | 1.26 |
| BF791738 | KIAA0738 gene product | \|7\|7q\| | | | | | | | | | | |
| BF115739 | Homo sapiens mRNA; cDNA | | | | | | | | | | | 1.27 |
| | DKFZp434E033 (from clone | | | | | | | | | | | |
| | DKFZp434E033) | | | | | | | | | | | |
| AI377497 | mob protein | \|10\|10q\| | | | | | | | 1.20 | | | |
| AB020717.1 | synaptojanin 1 | \|21\|21q\|21q22\| | | | | | | 1.22 | | | | |
| AA114166 | ESTs, Weakly similar to S26689 | | | | 1.35 | | 1.20 | | 1.24 | | | |
| | hypothetical protein hc1 - mouse | | | | | | | | | | | |
| | (fragment) [M. musculus] | | | | | | | | | | | |
| BF347326 | myristoylated alanine-rich protein | \|6\|6q\|6q22\| | | | | | | | 0.82 | | | 0.83 |
| | kinase C substrate | | | | | | | | | | | |
| BF448315 | Homo sapiens clone 24630 mRNA | | | | 1.33 | 1.22 | | | | | | |
| | sequence | | | | | | | | | | | |
| BF448315 | Homo sapiens clone 24630 mRNA | | | | 1.43 | 1.52 | | 1.22 | | | | |
| | sequence | | | | | | | | | | | |
| AU146655 | Homo sapiens cDNA FLJ11968 fis, | | | | 1.25 | | | | | | | |
| | clone HEMBB1001133 | | | | | | | | | | | |
| AL008583 | neuronal pentraxin receptor | \|22\|22q\|22q13\| | | | | | | | | 0.79 | | |
| AU145019 | KIAA1013 protein | \|3\|3p\|3p14\| | | | | | | | | | | 1.31 |
| AV682436 | ribosomal protein L35a | \|3\|3q\|3q29\| | | | 1.25 | 1.22 | | 0.75 | | 0.73 | | |
| AW052179 | collagen, type IV, alpha 5 (Alport | \|X\|Xq\| | | | | | | | | | | 1.30 |
| | syndrome) | | | | | | | | | | | |
| BE908931 | ESTs, Highly similar to GCHUH | | | | 1.26 | | | | | 0.78 | | |
| | glycine cleavage system protein H | | | | | | | | | | | |
| | precursor - human [H. sapiens] | | | | | | | | | | | |
| R38389 | olfactomedin 1 | \|9\|9q\|9q34\| | | | 0.79 | | | | | | | |
| AL049423.1 | Homo sapiens, clone | | | | 1.36 | | | 0.79 | | | | |
| | IMAGE: 4182947, mRNA | | | | | | | | | | | |
| AL049423.1 | Homo sapiens, clone | | | | 1.39 | | | | | | | |
| | IMAGE: 4182947, mRNA | | | | | | | | | | | |
| AU145005 | Sp3 transcription factor | \|2\|2q\| | | | | | | | | | | |
| BG538564 | ferritin, light polypeptide | \|19\|19q\|19q13\| | | 1.38 | 1.43 | 1.32 | 1.29 | 1.22 | | | | |
| U93305 | synaptophysin | \|X\|Xp\|Xp11\| | | | | | | | | | | |
| AJ271832.1 | protein phosphatase 1B (formerly | \|2\|2p\|2p22\| | | | | | | | | | | |
| | 2C), magnesium-dependent, beta | | | | | | | | | | | |
| | isoform | | | | | | | | | | | |
| BF590131 | Dicer1, Dcr-1 homolog (Drosophila) | \|14\|14q\|14q32\| | | | 1.27 | | | | | | | |
| AI922519 | rab6 GTPase activating protein (GAP | \|9\|9q\|9q34\| | 1.23 | | | | | | | 1.25 | | 1.21 |
| | and centrosome-associated) | | | | | | | | | | | |
| AU150319 | TAP binding protein related | \|12\|12p\|12p13\| | | | | | 1.28 | 1.20 | 1.21 | 1.70 | | 1.52 |
| BF062629 | Ras-induced senescence 1 | \|3\|3p\|3p21\| | | | 0.82 | | | | | 0.80 | 1.56 | |
| N33167 | cyclin-dependent kinase inhibitor 1C | \|11\|11p\|11p15\| | | | 1.55 | 1.43 | | | 1.62 | | | 1.95 |
| | (p57, Kip2) | | | | | | | | | | | |
| AI799007 | ribosomal protein S12 | \|6\|6q\|6q23\| | | | 1.21 | | | | | | | |
| AW070229 | Homo sapiens unknown mRNA | | | | 1.25 | | | | | | | |
| AL022327 | megalencephalic | \|22\|22q\|22q13\| | | | 1.31 | | | | | | | |
| | leukoencephalopathy with subcortical | | | | | | | | | | | |
| | cysts 1 | | | | | | | | | | | |
| AI560720 | ribophorin II | \|20\|20q\|20q12\| | 1.24 | | | | | | | | | 1.22 |
| BE259729 | ribosomal protein S19 | \|19\|19q\|19q13\| | | | 1.28 | | | | | | | |
| U73304 | cannabinoid receptor 1(brain) | \|6\|6q\|6q14\| | | 0.80 | | | 0.66 | | | | | |
| BF718769 | protein phosphatase 1, regulatory | \|2\|2q\|2q37\| | | | | | | 1.22 | | | | |
| | subunit 7 | | | | | | | | | | | |
| AL565749 | tubulin, beta, 4 | \|16\|16q\|16q24\| | 1.25 | | | | | 1.24 | | 1.22 | | |
| U26662.1 | neuronal pentraxin II | \|7\|7q\|7q21\| | | | | | 0.73 | | | 0.67 | | 1.61 |
| BE908217 | annexin A2 | \|15\|15q\|15q21\| | | | | | | | | 1.23 | 1.62 | 1.28 |
| M98528 | DNA segment on chromosome 4 | \|4\|4p\|4p16\| | | | 0.82 | | | | | | | |
| | (unique) 234 expressed sequence | | | | | | | | | | | |
| BE962615 | sorting nexin 3 | \|6\|6q\|6q22\| | 1.22 | | | | | | | | | |
| AB011131.1 | piccolo (presynaptic cytomatrix | \|7\|7q\|7q11\| | | | | | | 1.25 | | 1.21 | | |
| | protein) | | | | | | | | | | | |
| AI554300 | serine (or cysteine) proteinase | \|6\|6p\| | | | 1.25 | | | | | | 1.23 | 1.24 |
| | inhibitor, clade B (ovalbumin), | | | | | | | | | | | |
| | member 1 | | | | | | | | | | | |
| AB011152.1 | centaurin, delta 1 | \|4\|4p\|4p15\| | | | 1.24 | 1.46 | 1.21 | | | | | |
| NM_007054.1 | kinesin family member 3A | \|5\|5q\| | | | | | | 1.21 | | | | |
| AW235061 | solute carrier family 1 | \|9\|9p\| | | | | | | 1.24 | | | | |
| | (neuronal/epithelial high affinity | | | | | | | | | | | |
| | glutamate transporter, system Xag), | | | | | | | | | | | |
| | member 1 | | | | | | | | | | | |
| AA854017 | tyrosine 3- | \|2\| | 1.24 | | | | | | | | 1.22 | 1.31 |
| | monooxygenase/tryptophan 5- | | | | | | | | | | | |
| | monooxygenase activation protein, | | | | | | | | | | | |
| | theta polypeptide | | | | | | | | | | | |
| BG260658 | CS box-containing WD protein | | | | | | 1.20 | | | | | |
| AI557312 | cytochrome c oxidase subunit Vb | \|2\| | | | | | | | | | | 1.21 |
| AW241752 | splicing factor, arginine/serine-rich 11 | \|1\|1p\| | | | | | | | | | | |
| AW950513 | achaete-scute complex-like 1 | \|12\|12q\|12q22\| | 0.83 | | | | | | | | | |
| | (Drosophila) | | | | | | | | | | | |
| BE550452 | Homer, neuronal immediate early | \|5\|5q\|5q14\| | | | 0.71 | | 0.81 | | | | | |
| | gene, 1B | | | | | | | | | | | |
| BE674466 | Homo sapiens clone 23688 mRNA | | | | 0.81 | | | 1.29 | | 1.53 | | |
| | sequence | | | | | | | | | | | |
| AI200589 | ribosomal protein S16 | \|19\|19q\|19q13\| | | | 1.21 | | | | | | | |
| BF718636 | H2A histone family, member Z | \|4\|4q\| | 1.30 | | | | | 1.20 | 1.20 | 1.25 | | |
| NM_001048.1 | somatostatin | \|3\|3q\| | | | | 0.67 | 0.58 | 1.27 | 0.72 | 0.65 | | |
| AL134612 | Homo sapiens clone 23798 and | | | | 0.82 | | 0.81 | | 0.83 | 0.62 | | |
| | 23825 mRNA sequence | | | | | | | | | | | |
| AI885290 | spondin 1, (f-spondin) extracellular | \|11\|11p\|11p14\| | | | 1.37 | 1.28 | | | | | | |
| | matrix protein | | | | | | | | | | | |
| AA749101 | interferon induced transmembrane | \|11\| | | 1.27 | 1.31 | | 1.47 | 1.23 | 1.31 | | 1.21 | 1.40 |
| | protein 1 (9-27) | | | | | | | | | | | |
| AL533838 | tubulin, beta polypeptide | \|6\|6p\|6p21\| | 1.29 | 1.42 | 1.52 | 1.23 | 1.27 | | 1.38 | | | |
| BE857772 | ribosomal protein L37a | \|2\|2q\| | | 1.22 | | | | | | | | |
| AA017721 | Homo sapiens mRNA; cDNA | | 1.34 | | | 1.30 | | | | | | |
| | DKFZp686F1844 (from clone | | | | | | | | | | | |
| | DKFZp686F1844) | | | | | | | | | | | |
| AI073407 | transferrin | \|3\|3q\| | | | | | | | | | | 1.94 |
| BE043477 | ATPase, H+ transporting, lysosomal | \|5\|5q\|5q35\| | | 1.24 | 1.34 | | | | 1.22 | | | |
| | 9 kDa, V0 subunit e | | | | | | | | | | | |
| AA555113 | ribosomal protein, large, P0 | \|12\|12q\|12q24\| | | | | | | | | | 1.23 | |
| AA602532 | ceroid-lipofuscinosis, neuronal 2, late | \|11\|11p\| | | | | 1.25 | | | | 1.26 | | |
| | infantile (Jansky-Bielschowsky | | | | | | | | | | | |
| | disease) | | | | | | | | | | | |
| AA083483 | ferritin, heavy polypeptide 1 | \|11\|11q\| | | 1.34 | 1.39 | 1.22 | 1.35 | | 1.27 | | | 1.23 |
| AL122077.1 | dynein, axonemal, heavy polypeptide | \|17\|17q\| | | | | | | | | | | 1.34 |
| | 17 | | | | | | | | | | | |
| W86293 | proteasome (prosome, macropain) | \|6\|6q\| | 1.21 | | | | | | | | | 1.25 |
| | subunit, beta type, 1 | | | | | | | | | | | |
| AI348935 | calreticulin | \|19\|19p\|19p13\| | 1.32 | | | | | | | | 1.32 | |
| AI218219 | heat shock 90 kDa protein 1, beta | \|6\|6p\| | 1.24 | | | | | | | | | |
| BF063121 | testis intracellular mediator protein | \|6\|6p\|6p21\| | | | | | | | | | | |
| NM_000703.1 | hypothetical protein MGC13276 | \|19\|19q\|19q13\| | | | 0.77 | 0.79 | 0.74 | | | | | |
| AF127764.1 | calpain 3, (p94) | \|15\|15q\|15q15\| | | | | | | | | | | 1.54 |
| L03419.1 | Fc fragment of IgG, high affinity la, | \|1\|1q\|1q21\| | | | | | 1.25 | | | | | |
| | receptor for (CD64) | | | | | | | | | | | |
| NM_006713.1 | activated RNA polymerase II | \|5\|5p\|5p13\| | 1.32 | | | 1.33 | | | | | | |
| | transcription cofactor 4 | | | | | | | | | | | |
| AF065484.1 | sorting nexin 1 | \|15\|15q\|15q22\| | | | | | | | | | | 1.31 |
| T16257 | Homo sapiens cDNA FLJ38058 fis, | | | | | | | | | | 1.37 | 1.53 |
| | clone CTONG2014898 | | | | | | | | | | | |
| AL119322 | fibroblast growth factor 12 | \|3\|3q\| | | | 0.73 | | | | | | | |
| BG252666 | ATPase, Class I, type 8B, member 1 | \|18\|18q\|18q21\| | | | | | | | | | | |
| BG034080 | bridging integrator 1 | \|2\|2q\| | | | | | | | | | 1.25 | |
| AL050328 | | | | | 1.56 | 1.53 | | 1.23 | 1.40 | | 1.26 | 1.86 |
| AU134977 | multiple endocrine neoplasia I | \|11\|11q\| | | 0.65 | | | | | | | | |
| BF674712 | neurotrophic tyrosine kinase, | \|9\|9q\|9q22\| | 1.34 | | 1.39 | | | | | | | |
| | receptor, type 2 | | | | | | | | | | | |
| AI251890 | CDC-like kinase 1 | \|2\|2q\| | | 1.27 | 1.28 | | | | | | | |
| AK024789.1 | KRAB zinc finger protein KR18 | \|19\|19q\|19q13\| | | | | | | | | | | |
| AW516297 | ESTs, Weakly similar to hypothetical | | 1.25 | | 1.30 | | 1.22 | | | | | |
| | protein FLJ20378 [Homo sapiens] | | | | | | | | | | | |
| | [H. sapiens] | | | | | | | | | | | |
| AK025457.1 | golgi apparatus protein 1 | \|16\|16q\|16q22\| | | | | 1.24 | | 1.27 | | | | |
| AL162013.1 | plexin A1 | \|3\|3q\|3q21\| | | | | | | | 0.82 | 0.72 | | |
| AB007958.1 | KIAA0489 protein | | | | | | | | | | | |
| AW449813 | KIAA0918 protein | \|13\|13q\|13q31\| | | | | | | | | | | 0.80 |
| AA769818 | calcium channel, voltage-dependent, | \|19\|19p\|19p13\| | | | 0.83 | | | | | | | |
| | P/Q type, alpha 1A subunit | | | | | | | | | | | |
| X06989.1 | amyloid beta (A4) precursor protein | \|21\|21q\|21q21\| | | | | | 0.83 | | | | | |
| | (protease nexin-II, Alzheimer | | | | | | | | | | | |
| | disease) | | | | | | | | | | | |
| AL117593.1 | fasciculation and elongation protein | \|2\|2p\| | 1.23 | | | 1.23 | | | | 1.26 | | 1.32 |
| | zeta 2 (zygin II) | | | | | | | | | | | |
| AL161952.1 | glutamate-ammonia ligase (glutamine | \|1\|1q\| | | 1.34 | 1.33 | | | | | | | |
| | synthase) | | | | | | | | | | | |
| AB002438.1 | sema domain, transmembrane | \|5\|5q\|5q23\| | 1.59 | | 1.47 | | | | | | | 1.39 |
| | domain (TM), and cytoplasmic | | | | | | | | | | | |
| | domain, (semaphorin) 6A | | | | | | | | | | | |
| BC004145.1 | trinucleotide repeat containing 4 | \|1\|1q\| | | | | 0.83 | | | | 0.73 | | |
| AF035321.1 | dynamin 1 | \|9\|9q\| | | | | | 0.80 | 1.21 | | | | |
| AA679705 | eukaryotic translation initiation factor | \|16\|16p\|16p11\| | 1.43 | 1.22 | 1.25 | 1.34 | | 1.38 | 1.28 | 1.40 | 1.35 | 1.42 |
| | 3, subunit 8, 110 kDa | | | | | | | | | | | |
| AV721177 | phosphatidylinositol binding clathrin | \|11\|11q\| | | | | 1.29 | | | | 1.33 | | 1.45 |
| | assembly protein | | | | | | | | | | | |
| AL109722.1 | Homo sapiens cDNA FLJ33753 fis, | | 1.25 | | | | | | | | | |
| | clone BRCAN2000383 | | | | | | | | | | | |
| AW168915 | Homo sapiens prostate-specific | | | | | 1.43 | | | | | | 1.77 |
| | membrane antigen PSM mRNA, exon | | | | | | | | | | | |
| | 6 alternative splice variant, partial cds | | | | | | | | | | | |
| AK000270.1 | A kinase (PRKA) anchor protein | \|7\|7q\|7q21\| | | 1.28 | | | | | | | | |
| | (yotiao) 9 | | | | | | | | | | | |
| AK021882.1 | ras homolog gene family, member I | \|1\|1p\| | | | | | | | | | 1.76 | |
| BF966183 | gamma-aminobutyric acid (GABA) A | \|15\|15q\|15q11\| | | | | | | | | | | |
| | receptor, alpha 5 | | | | | | | | | | | |
| AV693216 | plexin B1 | \|3\|3p\|3p21\| | | | 1.31 | 1.25 | | | | | | |
| AA131826 | Homo sapiens cDNA FLJ36627 fis, | | | | | | | | | 0.69 | | 0.81 |
| | clone TRACH2017965, highly similar | | | | | | | | | | | |
| | to SPECTRIN BETA CHAIN, BRAIN | | | | | | | | | | | |
| AK027146.1 | ribosomal protein L5 | \|1\|1p\|1p22\| | | | | | | | | 1.26 | | 1.26 |
| AB028977.1 | KIAA1054 protein | \|5\|5q\|5q13\| | | | | | | | | | 0.43 | |
| X63575.1 | ATPase, Ca++ transporting, plasma | \|3\|3p\|3p26\| | | 0.74 | 0.71 | | | | | | 0.75 | |
| | membrane 2 | | | | | | | | | | | |
| AW188940 | beta-2-microglobulin | \|15\|15q\|15q21\| | 1.29 | 1.23 | 1.22 | | | | | | | 1.22 |
| s77154.1 | nuclear receptor subfamily 4, group | \|2\|2q\|2q22\| | | | | | | 0.73 | 0.65 | | | |
| | A, member 2 | | | | | | | | | | | |
| AB018277.1 | BAI1-associated protein 3 | \|16\|16p\|16p13 | | | | | 0.80 | 083 | | | | |
| S69738.1 | chemokine (C—C motif) ligand 2 | \|17\|17q\|17q11\| | | | | | 1.21 | | | | | |
| X98405.1 | myelin associated glycoprotein | \|19\|19q\|19q13\| | | | | | | | | | 1.47 | 1.81 |
| X86401.1 | glycine amidinotransferase (L- | \|15\|15q\| | | | 1.28 | | | | | | | |
| | arginine: glycine amidinotransferase) | | | | | | | | | | | |
| AC003999 | | | 1.20 | | | | | | | | | |
| U23850.1 | inositol 1,4,5-triphosphate receptor, | \|3\|3p\|3p26\| | | 0.65 | | 1.32 | | | | 1.29 | | |
| | type 1 | | | | | | | | | | | |
| AF279774.1 | growth associated protein 43 | \|3\|3q\|3q13\| | | | 0.82 | 0.80 | | | | | | |
| AF095723.1 | | | | | 0.79 | | | | 0.79 | | | |
| AF217990.1 | homocysteine-inducible, endoplasmic | \|16\|16q\|16q12\| | | | | | | | | 1.43 | | 1.24 |
| | reticulum stress-inducible, ubiquitin- | | | | | | | | | | | |
| | like domain member 1 | | | | | | | | | | | |
| U88968.1 | enolase 1, (alpha) | \|1\|1p\|1p36\| | 1.22 | | | | | | | | | |
| M22094.1 | neural cell adhesion molecule 1 | \|11\|11q\|11q23\| | | | | 1.46 | | | | | 1.30 | 1.43 |
| AL050361.1 | mitochondrial ribosomal protein S18B | \|6\|6p\|6p21\| | | | 1.21 | | | | 1.20 | | | |
| NM_021103.1 | thymosin, beta 10 | \|2\|2p\|2p11\| | | | | | | 1.25 | 1.22 | | 1.27 | 1.42 |
| NM_000972.1 | ribosomal protein L7a | | | | | | | | | | | 1.20 |
| NM_001013.1 | ribosomal protein S9 | \|19\|19q\|19q13\| | | | | | | 1.25 | | | | 1.26 |
| NM_001029.1 | ribosomal protein S26 | \|12\|12q\| | | 1.33 | 1.35 | 1.37 | 1.23 | 1.27 | 1.25 | | 1.39 | 1.31 |
| NM_018269.1 | SIPL protein | \|2\|2p\|2p25\| | 1.41 | 1.31 | 1.52 | 1.29 | | | 1.60 | | 1.32 | 1.30 |
| BE789881 | RAB31, member RAS oncogene | \|18\|18p\|18p11\| | | 1.30 | 1.34 | 1.22 | 1.32 | | 1.29 | | | |
| | family | | | | | | | | | | | |
| NM_006868.1 | RAB31, member RAS oncogene | \|18\|18p\|18p11\| | | 1.27 | 1.32 | 1.25 | 1.34 | | | | | |
| | family | | | | | | | | | | | |
| AF183421.1 | RAB31, member RAS oncogene | \|18\|18p\|18p11\| | | 1.28 | 1.31 | 1.20 | 1.27 | | | | | |
| | family | | | | | | | | | | | |
| NM_006555.1 | SNARE protein Ykt6 | \|7\|7p\|7p15\| | 0.81 | | | | | | | | | |
| NM_004481.2 | UDP-N-acetyl-alpha-D- | \|1\|1q\|1q41\| | | | | | | | | | | 1.20 |
| | galactosamine: polypeptide N- | | | | | | | | | | | |
| | acetylgalactoasaminyltransfarase 2 | | | | | | | | | | | |
| | (GalNAc-T2) | | | | | | | | | | | |
| NM_016275.1 | selenoprotein T | \|3\|3q\|3q22\| | | | | 1.27 | | | | | 1.22 | 1.23 |
| NM_022748.1 | tumor endothelial marker 6 | \|7\|7p\|7p15\| | | | | | | 0.75 | | 0.58 | | |
| NM_002415.1 | macrophage migration inhibitory | \|22\|22q\|22q11\| | | | | | 0.82 | | | | | |
| | factor (glycosylation-inhibiting factor) | | | | | | | | | | | |
| NM_016289.1 | MO25 protein | \|2\|2q\|2q36\| | 1.28 | | | 1.25 | | 1.27 | | 1.36 | | 1.28 |
| NM_003849.1 | succinate-CoA ligase, GDP-forming, | \|2\|2p\|2p11\| | | | | | | | | 1.22 | | 1.24 |
| | alpha subunit | | | | | | | | | | | |
| NM_014315.1 | host cell factor homolog | \|14\|14q\|14q21\| | 1.24 | | | 1.32 | | | | 1.28 | | |
| NM_014041.1 | signal peptidase 12 kDa | \|3\|3p\|3p21\| | 1.20 | | | | | | | | | |
| NM_015907.1 | leucine aminopeptidase 3 | \|4\|4p\|4p15\| | 1.34 | | 1.30 | | 1.25 | | 1.31 | | | 1.34 |
| NM_018695.1 | erbb2 interacting protein | \|5\|5q\|5q12\| | | 1.44 | 1.58 | | 1.33 | | | | 1.23 | |
| NM_016146.1 | PTD009 protein | \|11\|11q\|11q23\| | | | | | | | 1.22 | | | 1.21 |
| NM_016146.1 | PTD009 protein | \|11\|11q\|11q23\| | | | | 1.21 | | | 1.22 | | | 1.28 |
| NM_019048.1 | hypothetical protein FLJ20752 | \|2\|2p\|2p24\| | 1.26 | | | 1.28 | | | | 1.34 | | |
| NM_015920.1 | ribosomal protein S27-like | \|15\|15q\|15q21\| | | 1.24 | 1.26 | | | | 1.23 | | | |
| NM_014078.1 | mitochondrial ribosomal protein L13 | \|8\|8q\|8q22\| | | | | | | | | | | 1.20 |
| NM_015961.1 | hypothetical protein HSPC177 | \|9\|9p\| | 1.31 | | | | | | 1.23 | | 1.25 | |
| NM_018478.1 | chromosome 20 open reading frame | \|20\|20q\|20q13\| | | | | 1.36 | | | | | 1.20 | 1.63 |
| | 35 | | | | | | | | | | | |
| NM_004549.1 | NADH dehydrogenase (ubiquinone) | \|11\|11q\|11q13\| | 1.21 | | | | | 1.20 | | | | |
| | 1, subcomplex unknown, 2, 14.5 kDa | | | | | | | | | | | |
| NM_018047.1 | hypothetical protein FLJ10290 | \|5\|5q\|5q33\| | | | 1.22 | | | | | | | |
| NM_018368.1 | hypothetical protein FLJ11240 | \|6\|6q\|6q14\| | | | | 1.29 | | | | | | 1.24 |
| NM_015523.1 | small fragment nuclease | 11\|11q\|11q23\| | | | | | | | 1.22 | | | 1.24 |
| NM_014206.1 | chromosome 11 open reading frame | \|11\|11q\|11q12\| | | | 1.27 | | | | 1.21 | | 1.21 | 1.33 |
| | 10 | | | | | | | | | | | |
| NM_004547.2 | NADH dehydrogenase (ubiquinone) 1 | \|3\|3q\|3q13\| | | | | | | | | | | |
| | beta subcomplex, 4, 15 kDa | | | | | | | | | | | |
| NM_015991.1 | complement component 1, q | \|1\|1p\|1p36\| | | | | | 1.25 | 1.24 | | | | |
| | subcomponent, alpha polypeptide | | | | | | | | | | | |
| NM_016618.1 | hypothetical protein LOC51315 | \|2\|2p\|2p11\| | | | | | | | | | | |
| NM_021730.1 | hypothetical protein PP1044 | \|17\|17p\|17p13\| | | | | | | | | | 1.54 | |
| NM_022496.1 | hypothetical protein FLJ13433 | \|12\|12q\|12q21\| | 1.40 | | | | | 1.20 | | 1.33 | | 1.24 |
| NM_015987.1 | heme binding protein 1 | \|12\|12p\|12p13\| | | | 1.33 | | | | 1.24 | | 1.23 | 1.26 |
| NM_013372.1 | cysteine knot superfamily 1, BMP | \|15\|15q\|15q13\| | | | | | | | | | | 1.27 |
| | antagonist 1 | | | | | | | | | | | |
| NM_017945.1 | hypothetical protein FLJ20730 | \|3\|3q\|3q13\| | 1.39 | | | 1.34 | | | | | | 1.47 |
| NM_019000.1 | hypothetical protein FLJ20152 | \|5\|5p\|5p15\| | | | | 1.41 | | | | 1.29 | | 1.30 |
| NM_005461.1 | v-maf musculoaponeurotic | \|20\|20q\|20q11\| | 1.29 | | | 1.56 | 1.55 | 1.31 | | 1.62 | | |
| | fibrosarcoma oncogene homolog B | | | | | | | | | | | |
| | (avian) | | | | | | | | | | | |
| NM_015980.1 | HMP 19 protein | \|5\|5q\|5q35\| | | | | | | | | | | 0.72 |
| NM_018263.1 | KIAA1685 protein | \|2\|2p\|2p24\| | | | | 1.26 | | | | | | |
| NM_018103.1 | leucine-rich repeat-containing 5 | \|1\|1p\|1p22\| | | | | | | | | | | |
| AW575493 | hypothetical protein FLJ21313 | \|5\|5q\|5q23\| | 1.39 | | 1.57 | 1.47 | | | | | 1.30 | |
| NM_016205.1 | platelet derived growth factor C | \|4\|4q\| | 1.21 | | | | | | | | | |
| NM_014059.1 | RGC32 protein | \|13\|13q\|13q13\| | | 1.72 | 1.57 | 1.30 | | | 1.20 | 0.77 | | 1.32 |
| NM_017610.1 | likely ortholog of mouse Arkadia | \|15\|15q\| | | | | | | | | 1.24 | | |
| NM_020445.1 | actin-related protein 3-beta | \|7\|7q\|7q32\| | | | | | | 1.24 | | | | |
| NM_017899.1 | hypothetical protein FLJ20607 | \|12\|12q\|12q24\| | | | | 0.78 | | | | | | |
| NM_016140.1 | brain specific protein | \|16\|16q\|16q23\| | | | 1.40 | | | | 1.22 | | 1.60 | |
| NM_020353.1 | phospholipid scramblase 4 | \|3\|3q\| | 1.84 | 1.41 | 1.72 | 1.53 | 1.35 | | | | 1.72 | |
| NM_018374.1 | hypothetical protein FLJ11273 | \|7\|7p\|7p22\| | | | | 1.29 | | | | | | |
| NM_014322.1 | opsin 3 (ecephalopsin, panopsin) | \|1\|1q\| | | | | | | | | | 0.06 | |
| NM_007246.1 | kelch-like 2, Mayven (Drosophila) | \|4\|4q\|4q21\| | | | | 1.25 | | 1.32 | | 1.22 | | |
| NM_022367.1 | hypothetical protein FLJ12287 similar | \|1\|1q\| | | | | | | | | 0.77 | | |
| | to semaphorins | | | | | | | | | | | |
| NM_016410.1 | hypothetical protein HSPC177 | \|9\|9p\| | 1.21 | | | | | | | | 1.20 | |
| NM_024306.1 | fatty acid hydroxylase | \|16\|16q\| | | | | | | | | | | 1.65 |
| NM_018644.1 | Homo sapiens mRNA; cDNA | | | | | | | | | | | 1.34 |
| | DKFZp547E046 (from clone | | | | | | | | | | | |
| | DKFZp547E046 | | | | | | | | | | | |
| NM_006054.1 | reticulon 3 | \|11\|11q\| | 1.20 | | | | | | | 1.28 | | |
| NM_018658.1 | potassium inwardly-rectifying | \|17\|17q\|17q23\| | | 1.38 | | | 0.81 | 0.77 | | 0.65 | 0.74 | |
| | channel, subfamily J, member 16 | | | | | | | | | | | |
| NM_016533.1 | ninjurin 2 | \|12\|12p\| | | | | | | | | | | 1.50 |
| NM_005713.1 | collagen, type IV, alpha 3 | \|5\|5q\|5q13\| | | | | | | 1.30 | | 1.24 | | |
| | (Goodpasture antigen) binding | | | | | | | | | | | |
| | protein | | | | | | | | | | | |
| AL136591.1 | hippocalcin like 4 | \|1\|1p\|1p34\| | | | | | | | | | | 0.75 |
| NM_006790.1 | titin immunoglobulin domain protein | \|5\|5q\| | | | 1.23 | | 1.25 | | | | 1.22 | 1.26 |
| | (myotilin) | | | | | | | | | | | |
| NM_012259.1 | hairy/enhancer-of-split related with | \|6\|6q\|6q22\| | | | | | | 0.79 | | | | |
| | YRPW motif 2 | | | | | | | | | | | |
| NM_018342.1 | hypothetical protein FLJ11155 | \|4\|4q\|4q32\| | 0.59 | | | | 0.67 | 0.46 | 0.77 | | 0.62 | |
| NM_015414.1 | ribosomal protein L36 | \|19\|19p\|19p13\| | | 1.20 | | | | | | | | |
| NM_012082.2 | Friend of GATA2 | \|8\|8q\| | | | | | | | | 1.29 | 0.77 | 0.67 |
| NM_014018.1 | mitochondrial ribosomal protein S28 | \|8\|8q\|8q21\| | | 1.20 | | 1.25 | | | 1.26 | | | 1.21 |
| NM_012450.1 | solute carrier family 13 | \|7\|7q\| | 0.80 | | | 0.48 | 0.52 | | | | | |
| | (sodium/sulfate symporters), member 4 | | | | | | | | | | | |
| NM_015722.2 | calcyon; D1 dopamine receptor- | \|10\|10q\|10q26\| | | | | | | 1.31 | | 1.29 | | |
| | interacting protein | | | | | | | | | | | |
| NM_013381.1 | thyrotropin-releasing hormone | \|12\|12q\|12q15\| | | | 0.81 | | 1.38 | | | | | 0.81 |
| | degrading ectoenzyme | | | | | | | | | | | |
| NM_013251.1 | tachykinin 3 (neuromedin K, | \|12\|12q\|12q13\| | | | | 0.83 | | | | 0.80 | | |
| | neurokinin beta) | | | | | | | | | | | |
| NM_013257.1 | serum/glucocorticoid regulated | \|8\|8q\|8q12\| | | | 1.46 | 1.31 | | | 1.26 | | | 1.41 |
| | kinase-like | | | | | | | | | | | |
| NM_012144.1 | dynein, axonemal, intermediate | \|9\|9p\|9p21\| | | | | | | 0.82 | | | | 0.83 |
| | poylpeptide 1 | | | | | | | | | | | |
| NM_006658.1 | G-substrate | \|7\|7p\| | | 0.83 | | | | | | | | |
| NM_018167.1 | function unknown protein 1 | \|14\|14q\|14q32\| | | | | | | | | | 0.83 | |
| NM_006668.1 | cytochrome P450, subfamily 46 | \|14\|14q\|14q32\| | | | | | 0.82 | | | | | |
| | (cholesterol 24-hydroxylase) | | | | | | | | | | | |
| NM_018945.1 | phosphodiesterase 7B | \|6\|6q\|6q23\| | 0.81 | | | | | | | | | |
| NM_007071.1 | HERV-H LTR-associating 3 | \|1\|1p\|1p31\| | | | | 1.20 | | | 1.25 | | | 1.23 |
| NM_017435.1 | solute carrier family 21 (organic anion | \|12\|12p\|12p13\| | | | 1.49 | | | | 1.33 | 0.75 | | |
| | transporter), member 14 | | | | | | | | | | | |
| NM_016348.1 | chromosome 5 open reading frame 4 | \|5\|5q\|5q31\| | | | | | | | | | | 1.23 |
| NM_022469.1 | hypothetical protein FLJ21195 similar | \|1\|1q\| | | | | | | | | | 0.82 | 0.81 |
| | to protein related to DAC and | | | | | | | | | | | |
| | cerberus | | | | | | | | | | | |
| NM_015965.1 | cell death-regulatory protein GRIM19 | \|19\|19p\|19p13\| | | | | | | | | | | 1.21 |
| NM_018513.1 | | | 0.80 | | | | | | | | | |
| NM_020178.1 | Carbonic anhydrase-related protein | \|17\|17q\| | | | | | | | | | | 0.78 |
| | 10 | | | | | | | | | | | |
| NM_021154.1 | phosphoserine aminotransferase | \|9\|9q\|9q21\| | 1.29 | | 1.43 | 1.52 | | | 1.31 | | 1.34 | |
| NM_031279.1 | alanine-glyoxylate aminotransferase | \|4\|4q\| | 1.63 | 1.30 | 2.08 | 1.29 | 1.24 | | 1.46 | | | |
| | 2-like 1 | | | | | | | | | | | |
| NM_030817.1 | hypothetical protein DKFZp434F0318 | \|12\|12p\|12p13\| | | 1.28 | | | 1.48 | | 1.36 | 1.49 | | 1.56 |
| NM_021615.1 | carbohydrate (N-acetylglucosamine | \|16\|16q\| | | | 1.22 | | | | 1.31 | | | 1.24 |
| | 6-O) sulfotransferase 6 | | | | | | | | | | | |
| NM_017650.1 | protein phosphatase 1, regulatory | \|7\|7q\|7q11\| | | | | | | 1.27 | | | | |
| | (inhibitor) subunit 9A | | | | | | | | | | | |
| NM_018723.1 | ataxin 2-binding protein 1 | \|16\|16p\|16p13\| | | | | | | 1.23 | | 1.23 | | |
| NM_004115.1 | fibroblast growth factor 14 | \|13\|13q\| | | | | | | 1.24 | | | | |
| NM_021191.1 | neurogenic differentiation 4 | \|12\|12q\| | 0.81 | | | | | | | | | |
| NM_005172.1 | atonal homolog 1 (Drosophila) | \|4\|4q\| | 0.80 | | | | | | | | | |
| AW173623 | tumor differentially expressed 1 | \|20\|20q\|20q13\| | 1.24 | | | | | | | | | |
| NM_004776.1 | UDP-Gal: betaGlcNAc beta 1,4- | \|20\|20q\|20q13\| | | | | | | 1.29 | | 1.30 | 1.22 | |
| | galactosyltransferase, polypeptide 5 | | | | | | | | | | | |
| AF034756.1 | karyopherin alpha 3 (importin alpha | \|13\|13q\|13q14\| | 1.23 | | | | | | | | | |
| | 4) | | | | | | | | | | | |
| AW612574 | lecuine-rich acidic protein-like protein | \|1\|1q\|1q21\| | | | | | | | | | | |
| AB044088.1 | basic helix-loop-helix domain | \|12\|12p\|2p11\| | 1.50 | | 1.52 | 1.36 | | | 1.40 | 1.52 | | 1.34 |
| | containing, class B, 3 | | | | | | | | | | | |
| AL442077.1 | chromosome 8 open reading frame 2 | \|8\|8p\|8p11\| | 1.34 | | 1.26 | | | | | | | |
| AU145941 | CDC14 cell division cycle 14 homolog | \|9\|9q\|19q22\| | | | 1.27 | | | | | | | |
| | B (S. cerevisiae) | | | | | | | | | | | |
| AF130089.1 | aldehyde dehydrogenase 6 family, | \|14\|14q\|14q24\| | | | 1.31 | | | | | | | |
| | member A1 | | | | | | | | | | | |
| AF130089.1 | aldehyde dehydrogenase 6 family, | \|14\|14q\|14q24\| | 1.40 | 1.35 | 1.61 | 1.34 | 1.28 | | 1.27 | | | |
| | member A1 | | | | | | | | | | | |
| AF246240.1 | uncharacterized hypothalamus | \|11\|11q\|11q14\| | | | | | | | | | | |
| | protien HT007 | | | | | | | | | | | |
| AF133207.1 | protien kinase H11 | \|12\|12q\|12q24\| | 1.34 | | 1.28 | 1.45 | | | 1.25 | 1.49 | 1.39 | |
| AB049652.1 | mitochondrial ribosomal protein L34 | \|19\|19p\|19p13\| | 1.21 | | | 1.24 | | 1.27 | 1.20 | 1.22 | | 1.31 |
| BF209507 | activated RNA polymerase II | \|5\|5p\|5p13\| | | | 0.77 | 0.78 | | | 0.80 | | | |
| | transcription cofactor 4 | | | | | | | | | | | |
| J02814.1 | chondroitin sulfate proteoglycan 2 | \|5\|5q\|5q14\| | | | 1.29 | 1.32 | | | | | | |
| | (versican) | | | | | | | | | | | |
| BG287153 | mannosidase, alpha, class 1A, | \|6\|6q\| | 1.36 | | | | 0.82 | | | | 1.26 | |
| | member 1 | | | | | | | | | | | |
| AF055030.1 | PHD zinc finger protein XAP135 | \|6\|6q\| | 1.33 | | | 1.39 | | | | | | 1.26 |
| AA707199 | neurotrophic tyrosine kinase, | \|9\|9q\|9q22\| | 1.29 | | 1.53 | | | | | 0.75 | | |
| | receptor, type 2 | | | | | | | | | | | |
| AA707199 | neurotrophic tyrosine kinase, | \|9\|9q\|9q22\| | 1.47 | | 1.67 | | | | | | | |
| | receptor, type 2 | | | | | | | | | | | |
| AU157109 | KIAA1598 protein | \|10\|10q\|10q26\| | | | | | | | | | | |
| NM_006158.1 | neurofilament, light polypeptide | \|8\|8p\| | | | 0.82 | | | 1.37 | | 1.35 | 0.59 | |
| | 68 kDa | | | | | | | | | | | |
| AI307759 | karyopherin (importin) beta 2 | \|5\|5q\|5q13\| | | | | | | | | | | |
| U70056 | seven in absentia homolog 1 | \|16\|16q\| | | | | 1.34 | | | | | 1.20 | |
| | (Drosophila) | | | | | | | | | | | |
| BF514079 | Kruppel-like factor 4 (gut) | \|9\|9q\| | | | 0.77 | 0.77 | 0.80 | 0.63 | 0.73 | 0.76 | 0.77 | |
| N34407 | KIAA0608 protein | \|10\|10q\|10q24\| | | | | | | | 1.26 | | | 1.50 |
| AF070641.1 | Homo sapiens clone 24421 mRNA | | | | | | | | | 0.81 | 0.56 | |
| | sequence | | | | | | | | | | | |
| AI719730 | guanylate cycase 1, soluble alpha 3 | \|4\|4q\|4q31\| | | | | | | 1.23 | | | | 0.67 |
| AW303136 | ribosomal protein L38 | \|17\|17q\|17q23\| | | | | | | | | | | |
| AW057781 | ribosomal protein L10 | \|X\|Xq\| | | | | | | | | | 1.22 | |
| AI984479 | Homo sapiens cDNA FLJ33067 fis, | | 1.32 | | | 1.39 | | 1.20 | 1.34 | | 1.31 | 1.27 |
| | clone TRACH2000148, weakly | | | | | | | | | | | |
| | similar to POLY(A) POLYMERASE | | | | | | | | | | | |
| | (EC 2.7.7.19) | | | | | | | | | | | |
| AI982754 | clusterin (complement lysis inhibitor, | \|8\|8p\|8p21\| | 1.33 | | 1.33 | | 1.24 | | 1.53 | 1.35 | | 1.28 |
| | SP-40,40, sulfated glycoprotein 2, | | | | | | | | | | | |
| | testosterone-repressed prostate | | | | | | | | | | | |
| | message 2, apolipoprotein J) | | | | | | | | | | | |
| AF015043.1 | SH3-domain binding protein 4 | \|2\|2q\|2q37\| | | | 1.33 | | | | 1.23 | | | 1.25 |
| X15306 | neurofilament, heavy polypeptide | \|22\|22q\|22q12\| | | | 0.73 | 0.77 | | 1.26 | 1.26 | | 0.55 | 1.65 |
| | 200 kDa | | | | | | | | | | | |
| U89281 | 3-hydroxysteroid epimerase | \|12\|12q\| | | | 1.21 | | | | | | | |
| L19185 | peroxiredoxin 2 | \|13\|13q\| | 1.21 | | | | | | | | | |
| R61374 | hairy/enhancer-of-split related with | \|8\|8q\| | | | | | | | | | | 1.24 |
| | YRPW motif 1 | | | | | | | | | | | |
| H93026 | elongation of very long chain fatty | \|1\|1p\|1p34\| | | | | 1.31 | | | 1.25 | | 1.25 | 1.65 |
| | acids (FEN1/Elo2, SUR4/Elo3, | | | | | | | | | | | |
| | yeast)-like 1 | | | | | | | | | | | |
| U84487 | chemokine (C—X3—C motif) ligand 1 | \|16\|16q\| | | | | | 0.82 | | | | | |

TABLE 2
ALL REGIONS - NEUROFILAMENT

| | | | | ACCESSION |
|---|---|---|---|---|
| UniGene | LocusID | Description | Symbol | (Probe ID) |
| Hs.198760 | 4744 | neurofilament, heavy | NEFH | NM_021076 |
| | | polypeptide 200 kDa | | (204412_s_at) |
| Hs.211584 | 4747 | neurofilament, light | NEFL | AL537457 |
| | | polypeptide 68 kDa | | (221805_at) |
| Hs.71346 | 4741 | neurofilament 3 | NEF3 | NM_005382 |
| | | (150 kDa medium) | | (205113_at) |

TABLE 3
ALL REGIONS - DEVELOPMENT

| UniGene | LocusID | Description | Symbol | ACCESSION (Probe ID) |
|---|---|---|---|---|
| Hs.103724 | 5376 | peripheral myelin protein 22 | PMP22 | L03203 (210139_s_at) |
| Hs.10526 | 1466 | cysteine and glycine-rich protein 2 | CSRP2 | NM_001321 |
| | | | | (207030_s_at) |
| Hs.111126 | 754 | pituitary tumor-transforming 1 | PTTG1IP | NM_004339 (200677_at) |
| | | interacting protein | | |
| Hs.111779 | 6678 | secreted protein, acidic, cysteine- | SPARC | AL575922 (212667_at) |
| | | rich (osteonectin) | | |
| Hs.117546 | 4826 | neuronatin | NNAT | NM_005386 |
| | | | | (204239_s_at) |
| Hs.141308 | 4340 | myelin oligodendrocyte glycoprotein | MOG | NM_002433 Table |
| | | | | 2: (205989_s_at) |
| Hs.153684 | 2487 | frizzled-related protein | FRZB | NM_001463 |
| | | | | (203698_s_at) |
| Hs.154654 | 1545 | cytochrome P450, family 1, | CYP1BI | NM_000104 |
| | | subfamily B, polypeptide 1 | | (202437_s_at) |
| Hs.158213 | 9576 | sperm associated antigen 6 | SPAG6 | AF079363 (210033_s_at) |
| Hs.158540 | 7368 | UDP glycosyltransferase 8 (UDP- | UGT8 | NM_003360 |
| | | galactose ceramide | | (208358_s_at) |
| | | galactosyltransferase) | | |
| Hs.169309 | 4336 | myelin-associated oligodendrocyte | MOBP | D28114 (210193_at) |
| | | basic protein | | |
| Hs.169401 | 348 | apolipoprotein E | APOE | N33009 (203381_s_at) |
| Hs.169487 | 9935 | v-maf musculoaponeurotic | MAFB | NM_005461 |
| | | fibrosarcoma oncogene homolog B | | (218559_s_at) |
| | | (avian) | | |
| Hs.173594 | 5176 | serine (or cysteine) proteinase | SERPINF1 | NM_002615 (202283_at) |
| | | inhibitor, clade F (alpha-2 | | |
| | | antiplasmin, pigment epithelium | | |
| | | derived factor), member 1 | | |
| Hs.194695 | 9077 | ras homolog gene family, member I | ARHI | AK021882 (215506_s_at) |
| Hs.198760 | 4744 | neurofilament, heavy polypeptide | NEFH | NM_021076 |
| | | 200 kDa | | (204412_s_at) |
| Hs.2316 | 6662 | SRY (sex determining region Y)- | SOX9 | AI382146 (202935_s_at) |
| | | box 9 (campomelic dysplasia, | | |
| | | autosomal sex-reversal) | | |
| Hs.2563 | 6863 | tachykinin, precursor 1 (substance | TAC1 | NM_003182 |
| | | K, substance P, neurokinin 1, | | (206552_s_at) |
| | | neurokinin 2, neuromedin L, | | |
| | | neurokinin alpha, neuropeptide K, | | |
| | | neuropeptide gamma) | | |
| Hs.25647 | 2353 | v-fos FBJ murine osteosarcoma | FOS | BC004490 (209189_at) |
| | | viral oncogene homolog | | |
| Hs.284122 | 11197 | WNT inhibitory factor 1 | WIF1 | NM_007191 (204712_at) |
| Hs.284244 | 2247 | fibroblast growth factor 2 (basic) | FGF2 | NM_002006 |
| | | | | (204422_s_at) |
| Hs.288650 | 361 | aquaporin 4 | AQP4 | D63412 |
| | | | | (210066_s_at,210067_at) |
| Hs.293267 | 825 | calpain 3, (p94) | CAPN3 | AF127764 (214475_x_at) |
| Hs.295449 | 5816 | parvalbumin | PVALB | NM_002854 (205336_at) |
| Hs.313 | 6696 | secreted phosphoprotein 1 | SPP1 | M83248 (209875_s_at) |
| | | (osteopontin, bone sialoprotein I, | | |
| | | early T-lymphocyte activation 1) | | |
| Hs.33829 | 79365 | basic helix-loop-helix domain | BHLHB3 | BE857425 (221530_s_at) |
| | | containing, class B, 3 | | |
| Hs.380674 | 302 | annexin A2 | ANXA2 | BC001388 (210427_x_at) |
| Hs.408061 | 2171 | fatty acid binding protein 5 | FABP5 | NM_001444 |
| | | (psoriasis-associated) | | (202345_s_at) |
| Hs.433399 | 6876 | transgelin | TAGLN | NM_003186 |
| | | | | (205547_s_at) |
| Hs.44 | 5764 | pleiotrophin (heparin binding growth | PTN | AL565812 (209465_x_at) |
| | | factor 8, neurite growth-promoting | | |
| | | factor 1) | | |
| Hs.69547 | 4155 | myelin basic protein | MBP | AW070431 (210136_at) |
| Hs.7306 | 6422 | secreted frizzled-related protein1 | SFRP1 | NM_003012 |
| | | | | (202037_s_at) |
| Hs.73793 | 7422 | vascular endothelial growth factor | VEGF | AF022375 (210512_s_at) |
| Hs.74471 | 2697 | gap junction protein, alpha 1, | GJA1 | NM_000165 (201667_at) |
| | | 43 kDa (connexin 43) | | |
| Hs.75106 | 1191 | clusterin (complement lysis | CLU | AI982754 (222043_at) |
| | | inhibitor, SP-40,40, sulfated | | |
| | | glycoprotein 2, testosterone- | | |
| | | repressed prostate message 2, | | |
| | | apolipoprotein J) | | |
| Hs.79368 | 2012 | epithelial membrane protein 1 | EMP1 | NM_001423 (201324_at) |
| Hs.80296 | 5121 | Purkinje cell protein 4 | PCP4 | NM_006198 (205549_at) |
| Hs.80395 | 4118 | mal, T-cell differentiation protein | MAL | NM_002371 |
| | | | | (204777_s_at) |
| Hs.82002 | 1910 | endothelin receptor type B | EDNRB | NM_000115 (204273_at) |
| Hs.83384 | 6285 | S100 calcium binding protein, beta | S100B | BC001766 (209686_at) |
| | | (neural) | | |
| Hs.85112 | 3479 | insulin-like growth factor 1 | IGF1 | AI972496 (209541_at) |
| Hs.89626 | 5744 | parathyroid hormone-like hormone | PTHLH | BC005961 (211756_at) |

TABLE 4
ALL REGIONS - EXTRACELLULAR

| UniGene | LocusID | Description | Symbol | ACCESSION (Probe ID) |
|---|---|---|---|---|
| Hs.111779 | 6678 | secreted protein, acidic, cysteine-rich | SPARC | AL575922 (212667_at) |
| | | (osteonectin) | | |
| Hs.12409 | 6750 | somatostatin | SST | NM_001048 (213921_at) |
| Hs.153684 | 2487 | frizzled-related protein | FRZB | NM_001463 |
| | | | | (203698_s_at) |
| Hs.154679 | 6857 | synaptotagmin I | SYT1 | AV723167 (203998_s_at) |
| Hs.169857 | 5445 | paraoxonase 2 | PON2 | AF001602 (210830_s_at) |
| Hs.1707 | 9607 | cocaine- and amphetamine- | CART | NM_004291 (206339_at) |
| | | regulated transcript | | |
| Hs.1832 | 4852 | neuropeptide Y | NPY | NM_000905 (206001_at) |
| Hs.20021 | 6843 | vesicle-associated membrane | VAMP1 | AU150319 (213326_at) |
| | | protein 1 (synaptobrevin 1) | | |
| Hs.2563 | 6863 | tachykinin, precursor 1 (substance K, | TAC1 | NM_003182 |
| | | substance P, neurokinin 1, | | (206552_s_at) |
| | | neurokinin 2, neuromedin L, | | |
| | | neurokinin alpha, neuropeptide K, | | |
| | | neuropeptide gamma) | | |
| Hs.284244 | 2247 | fibroblast growth factor 2 (basic) | FGF2 | NM_002006 |
| | | | | (204422_s_at) |
| Hs.313 | 6696 | secreted phosphoprotein 1 | SPP1 | M83248 (209875_s_at) |
| | | (osteopontin, bone sialoprotein I, | | |
| | | early T-lymphocyte activation 1) | | |
| Hs.336678 | 1837 | dystrobrevin, alpha | DTNA | NM_001392 |
| | | | | (205741_s_at) |
| Hs.386793 | 2878 | glutathione peroxidase 3 (plasma) | GPX3 | NM_002084 (201348_at) |
| Hs.396489 | 7018 | transferrin | TF | AI073407 (214063_s_at) |
| Hs.427202 | 7276 | transthyretin (prealbumin, | TTR | AF162690 (209660_at) |
| | | amyloidosis type I) | | |
| Hs.433721 | 721 | complement component 4B | C4B | NM_000592 |
| | | | | (208451_s_at) |
| Hs.44 | 5764 | pleiotrophin (heparin binding growth | PTN | AL565812 (209465_x_at) |
| | | factor 8, neurite growth-promoting | | |
| | | factor 1) | | |
| Hs.64016 | 5627 | protein S (alpha) | PROS1 | NM_000313 |
| | | | | (207808_s_at) |
| Hs.7306 | 6422 | secreted frizzled-related protein 1 | SFRP1 | NM_003012 |
| | | | | (202037_s_at) |
| Hs.73793 | 7422 | vascular endothelial growth factor | VEGF | AF022375 (210512_s_at) |
| Hs.75184 | 1116 | chitinase 3-like 1 (cartilage | CHI3L1 | M80927 |
| | | glycoprotein-39) | | (209395_at.209396_s_at) |
| Hs.75294 | 1392 | corticotropin releasing hormone | CRH | NM_000756 (205630_at) |
| Hs.76224 | 2202 | EGF-containing fibulin-like | EFEMP1 | AI826799 (201842_s_at) |
| | | extracellular matrix protein 1 | | |
| Hs.78224 | 6035 | ribonuclease, RNase A family, 1 | RNASE1 | NM_002933 (201785_at) |
| | | (pancreatic) | | |
| Hs.80247 | 885 | cholecystokinin | CCK | NM_000729 (205827_at) |
| Hs.8117 | 55914 | erbb2 interacting protein | ERBB2IP | NM_018695 |
| | | | | (217941_s_at) |
| Hs.83384 | 6285 | S100 calcium binding protein, beta | S100B | BC001766 (209686_at) |
| | | (neural) | | |
| Hs.89626 | 5744 | parathyroid hormone-like hormone | PTHLH | BC005961 (211756_at) |
| Hs.8986 | 713 | complement component 1, q | C1QB | NM_000491 (202953_at) |
| | | subcomponent, beta polypeptide | | |
| Hs.94592 | 9365 | klotho | KL | NM_004795 (205978_at) |

TABLE 5
ALL REGIONS - CELL TO CELL SIGNAL

| | | | | ACCESSION |
|---|---|---|---|---|
| UniGene | LocusID | Description | Symbol | (Probe ID) |
| Hs.103724 | 5376 | peripheral myelin protein 22 | PMP22 | L03203 |
| | | | | (210139_s_at) |
| Hs.12409 | 6750 | somatostatin | SST | NM_001048 |
| | | | | (213921_at) |
| Hs.141308 | 4340 | myelin oligodendrocyte | MOG | NM_002433 |
| | | glycoprotein | | (205989_s_at) |
| Hs.154679 | 6857 | synaptotagmin I | SYT1 | AV723167 |
| | | | | (203998_s_at) |
| Hs.170414 | 5046 | paired basic amino acid cleaving | PACE4 | NM_002570 |
| | | system 4 | | (207414_s_at) |
| Hs.1707 | 9607 | cocaine- and amphetamine- | CART | NM_004291 |
| | | regulated transcript | | (206339_at) |
| Hs.170808 | 2572 | glutamate decarboxylase 2 | GAD2 | NM_000818 |
| | | (pancreatic islets and brain, | | (206780_at) |
| | | 65 kDa) | | |
| Hs.1832 | 4852 | neuropeptide Y | NPY | NM_000905 |
| | | | | (206001_at) |
| Hs.19492 | 5100 | protocadherin 8 | PCDH8 | NM_002590 |
| | | | | (206935_at) |
| Hs.2563 | 6863 | tachykinin, precursor 1 (substance | TAC1 | NM_003182 |
| | | K, substance P, neurokinin 1, | | (206552_s_at) |
| | | neurokinin 2, neuromedin L, | | |
| | | neurokinin alpha, neuropeptide K, | | |
| | | neuropeptide gamma) | | |
| Hs.284122 | 11197 | WNT inhibitory factor 1 | WIF1 | NM_007191 |
| | | | | (204712_at) |
| Hs.284244 | 2247 | fibroblast growth factor 2 (basic) | FGF2 | NM_002006 |
| | | | | (204422_s_at) |
| Hs.324784 | 2571 | glutamate decarboxylase 1 (brain, | GAD1 | NM_000817 |
| | | 67 kDa) | | (205278_at) |
| Hs.3281 | 4885 | neuronal pentraxin II | NPTX2 | U26662 |
| | | | | (213479_at) |
| Hs.336678 | 1837 | dystrobrevin, alpha | DTNA | NM_001392 |
| | | | | (205741_s_at) |
| Hs.3697 | 183 | angiotensinogen (serine (or | AGT | NM_000029 |
| | | cysteine) proteinase inhibitor, | | (202834_at) |
| | | clade A (alpha-1 antiproteinase, | | |
| | | antitrypsin), member 8) | | |
| Hs.380 | 6506 | solute carrier family 1 (glial high | SLC1A2 | NM_004171 |
| | | affinity glutamate transporter), | | (208389_s_at) |
| | | member 2 | | |
| Hs.46362 | 3358 | 5-hydroxytryptamine (serotonin) | HTR2C | M81778 |
| | | receptor 2C | | (211479_s_at) |
| Hs.69547 | 4155 | myelin basic protein | MBP | AW070431 |
| | | | | (210136_at) |
| Hs.74471 | 2697 | gap junction protein, alpha 1, | GJA1 | NM_000165 |
| | | 43 kDa (connexin 43) | | (201667_at) |
| Hs.75294 | 1392 | corticotropin releasing hormone | CRH | NM_000756 |
| | | | | (205630_at) |
| Hs.75379 | 6507 | solute carrier family 1 (glial high | SLC1A3 | NM_004172 |
| | | affinity glutamate transporter), | | (202800_at) |
| | | member 3 | | |
| Hs.89626 | 5744 | parathyroid hormone-like hormone | PTHLH | BC005961 |
| | | | | (211756_at) |

TABLE 6
ALL REGIONS - SYNAPTIC TRANS.

| | | | | ACCESSION |
|---|---|---|---|---|
| UniGene | LocusID | Description | Symbol | (Probe ID) |
| Hs.103724 | 5376 | peripheral myelin protein 22 | PMP22 | L03203 |
| | | | | (210139_s_at) |
| Hs.12409 | 6750 | somatostatin | SST | NM_001048 |
| | | | | (213921_at) |
| Hs.141308 | 4340 | myelin oligodendrocyte glycoprotein | MOG | NM_002433 |
| | | | | (205989_s_at) |
| Hs.154679 | 6857 | synaptotagmin I | SYT1 | AV723167 |
| | | | | (203998_s_at) |
| Hs.1707 | 9607 | cocaine- and amphetamine-regulated transcript | CART | NM_004291 |
| | | | | (206339_at) |
| Hs.170808 | 2572 | glutamate decarboxylase 2 (pancreatic islets and | GAD2 | NM_000818 |
| | | brain, 65 kDa) | | (206780_at) |
| Hs.1832 | 4852 | neuropeptide Y | NPY | NM_000905 |
| | | | | (206001_at) |
| Hs.2563 | 6863 | tachykinin, precursor 1 (substance K, substance | TAC1 | NM_003182 |
| | | P, neurokinin 1, neurokinin 2, neuromedin L, | | (206552_s_at) |
| | | neurokinin alpha, neuropeptide K, neuropeptide | | |
| | | gamma) | | |
| Hs.324784 | 2571 | glutamate decarboxylase 1 (brain, 67 kDa) | GAD1 | NM_000817 |
| | | | | (205278_at) |
| Hs.3281 | 4885 | neuronal pentraxin II | NPTX2 | U26662 |
| | | | | (213479_at) |
| Hs.336678 | 1837 | dystrobrevin, alpha | DTNA | NM_001392 |
| | | | | (205741_s_at) |
| Hs.380 | 6506 | solute carrier family 1 (glial high affinity glutamate | SLC1A2 | NM_004171 |
| | | transporter), member 2 | | (208389_s_at) |
| Hs.46362 | 3358 | 5-hydroxytryptamine (serotonin) receptor 2C | HTR2C | M81778 |
| | | | | (211479_s_at) |
| Hs.69547 | 4155 | myelin basic protein | MBP | AW070431 |
| | | | | (210136_at) |
| Hs.75294 | 1392 | corticotropin releasing hormone | CRH | NM_000756 |
| | | | | (205630_at) |
| Hs.75379 | 6507 | solute carrier family 1 (glial high affinity glutamate | SLC1A3 | NM_004172 |
| | | transporter), member 3 | | (202800_at) |

TABLE 7
ALL REGIONS - ORGANOGENESIS

| UniGene | LocusID | Description | ACCESSION (Probe ID) |
|---|---|---|---|
| Hs.103724 | 5376 | peripheral myelin protein 22 | L03203 (210139_s_at) |
| Hs.10526 | 1466 | cysteine and glycine-rich protein 2 | NM_001321 |
| | | | (207030_s_at) |
| Hs.111779 | 6678 | secreted protein, acidic, cysteine-rich | AL575922 (212667_at) |
| | | (osteonectin) | |
| Hs.117546 | 4826 | neuronatin | NM_005386 |
| | | | (204239_s_at) |
| Hs.141308 | 4340 | myelin oligodendrocyte glycoprotein | NM_002433 |
| | | | (205989_s_at) |
| Hs.153684 | 2487 | frizzled-related protein | NM_001463 |
| | | | (203698_s_at) |
| Hs.154654 | 1545 | cytochrome P450, family 1, subfamily B, | NM_000104 |
| | | polypeptide 1 | (202437_s_at) |
| Hs.158540 | 7368 | UDP glycosyltransferase 8 (UDP-galactose | NM_003360 |
| | | ceramide galactosyltransferase) | (208358_s_at) |
| Hs.169309 | 4336 | myelin-associated oligodendrocyte basic protein | D28114 (210193_at) |
| Hs.169487 | 9935 | v-maf musculoaponeurotic fibrosarcoma | NM_005461 |
| | | oncogene homolog B (avian) | (218559_s_at) |
| Hs.173594 | 5176 | serine (or cysteine) proteinase inhibitor, clade F | NM_002615 (202283_at) |
| | | (alpha-2 antiplasmin, pigment epithelium | |
| | | derived factor), member 1 | |
| Hs.198760 | 4744 | neurofilament, heavy polypeptide 200 kDa | NM_021076 |
| | | | (204412_s_at) |
| Hs.2316 | 6662 | SRY (sex determining region Y)-box 9 | AI382146 (202935_s_at) |
| | | (campomelic dysplasia, autosomal sex- | |
| | | reversal) | |
| Hs.284244 | 2247 | fibroblast growth factor 2 (basic) | NM_002006 |
| | | | (204422_s_at) |
| Hs.288650 | 361 | aquaporin 4 | D63412 |
| | | | (210066_s_at, 210067_at) |
| Hs.293267 | 825 | calpain 3, (p94) | AF127764 (214475_x_at) |
| Hs.295449 | 5816 | parvalbumin | NM_002854 (205336_at) |
| Hs.313 | 6696 | secreted phosphoprotein 1 (osteopontin, bone | M83248 (209875_s_at) |
| | | sialoprotein I, early T-lymphocyte activation 1) | |
| Hs.33829 | 79365 | basic helix-loop-helix domain containing, class | BE857425 (221530_s_at) |
| | | B, 3 | |
| Hs.380674 | 302 | annexin A2 | BC001388 (210427_x_at) |
| Hs.408061 | 2171 | fatty acid binding protein 5 (psoriasis- | NM_001444 |
| | | associated) | (202345_s_at) |
| Hs.433399 | 6876 | transgelin | NM_003186 |
| | | | (205547_s_at) |
| Hs.44 | 5764 | pleiotrophin (heparin binding growth factor 8, | AL565812 (209465_x_at) |
| | | neurite growth-promoting factor 1) | |
| Hs.69547 | 4155 | myelin basic protein | AW070431 (210136_at) |
| Hs.73793 | 7422 | vascular endothelial growth factor | AF022375 (210512_s_at) |
| Hs.74471 | 2697 | gap junction protein, alpha 1, 43 kDa (connexin | NM_000165 (201667_at) |
| | | 43) | |
| Hs.79368 | 2012 | epithelial membrane protein 1 | NM_001423 (201324_at) |
| Hs.80296 | 5121 | Purkinje cell protein 4 | NM_006198 (205549_at) |
| Hs.80395 | 4118 | mal, T-cell differentiation protein | NM_002371 |
| | | | (204777_s_at) |
| Hs.82002 | 1910 | endothelin receptor type B | NM_000115 (204273_at) |
| Hs.83384 | 6285 | S100 calcium binding protein, beta (neural) | BC001766 (209686_at) |
| Hs.85112 | 3479 | insulin-like growth factor 1 (somatomedin C) | AI972496 (209541_at) |
| Hs.89626 | 5744 | parathyroid hormone-like hormone | BC005961 (211756_at) |

TABLE 8
VThal - CYTOPLASM

| UniGene | LocusID | Description | Symbol | ACCESSION (Probe ID) |
|---|---|---|---|---|
| Hs.104717 | 7461 | cytoplasmic linker 2 | CYLN2 | BC006259 (211031_s_at) |
| Hs.1050 | 9267 | pleckstrin homology, | PSCD1 | NM_004762 (202880_s_at) |
| | | Sec7 and coiled-coil | | |
| | | domains 1(cytohesin 1) | | |
| Hs.106529 | 51103 | CGI-65 protein | CIA30 | NM_016013 (204125_at) |
| Hs.107164 | 6711 | spectrin, beta, non- | SPTBN1 | AA131826 (215918_s_at) |
| | | erythrocytic 1 | | |
| Hs.111126 | 754 | pituitary tumor- | PTTG1IP | NM_004339 (200677_at) |
| | | transforming 1 interacting | | |
| | | protein | | |
| Hs.112667 | 27019 | dynein, axonemal, | DNAI1 | NM_012144 (220125_at) |
| | | intermediate polypeptide 1 | | |
| Hs.113503 | 3843 | karyopherin (importin) | KPNB3 | AU148466 (211953_s_at) |
| | | beta 3 | | |
| Hs.11465 | 9446 | glutathione-S-transferase | GSTTLp28 | NM_004832 (201470_at) |
| | | like; glutathione | | |
| | | transferase omega | | |
| Hs.11806 | 1717 | 7-dehydrocholesterol | DHCR7 | AW150953 (201790_s_at) |
| | | reductase | | |
| Hs.119251 | 7384 | ubiquinol-cytochrome c | UQCRC1 | NM_003365 (201903_at) |
| | | reductase core protein I | | |
| Hs.1197 | 3336 | heat shock 10 kDa protein | HSPE1 | NM_002157 (205133_s_at) |
| | | 1 (chaperonin 10) | | |
| Hs.12163 | 8894 | eukaryotic translation | EIF2S2 | BC000461 (208726_s_at) |
| | | initiation factor 2, subunit | | |
| | | 2 beta, 38 kDa | | |
| Hs.12956 | 30851 | Tax interaction protein 1 | TIP-1 | AF234997 (209154_at) |
| Hs.1342 | 1329 | cytochrome c oxidase | COX5B | AI557312 (213735_s_at) |
| | | subunit Vb | | |
| Hs.138617 | 9320 | thyroid hormone receptor | TRIP12 | NM_004238 (201546_at) |
| | | interactor 12 | | |
| Hs.144063 | 6301 | seryl-tRNA synthetase | SARS | NM_006513 (200802_at) |
| Hs.144672 | 8632 | dynein, axonemal, heavy | DNAH17 | AL122077 (214229_at) |
| | | polypeptide 17 | | |
| Hs.146388 | 9053 | microtubule-associated | MAP7 | AW242297 (202890_at) |
| | | protein 7 | | |
| Hs.146393 | 9709 | homocysteine-inducible, | HERPUD1 | AF217990 (217168_s_at) |
| | | endoplasmic reticulum | | |
| | | stress-inducible, ubiquitin- | | |
| | | like domain member 1 | | |
| Hs.148495 | 5710 | proteasome (prosome, | PSMD4 | AB033605 (210460_s_at) |
| | | macropain) 26S subunit, | | |
| | | non-ATPase, 4 | | |
| Hs.150101 | 3916 | lysosomal-associated | LAMP1 | J03263 (201551_s_at) |
| | | membrane protein 1 | | |
| Hs.150580 | 10209 | putative translation | SUI1 | AF083441 (202021_x_at) |
| | | initiation factor | | |
| Hs.154654 | 1545 | cytochrome P450, family | CYP1B1 | AU144855 (202436_s_at) |
| | | 1, subfamily B, | | |
| | | polypeptide 1 | | |
| Hs.154672 | 10797 | methylene | MTHFD2 | NM_006636 (201761_at) |
| | | tetrahydrofolate | | |
| | | dehydrogenase (NAD+ | | |
| | | dependent), | | |
| | | methenyltetrahydrofolate | | |
| | | cyclohydrolase | | |
| Hs.154679 | 6857 | synaptotagmin I | SYT1 | AV723167 (203998_s_at) |
| Hs.155097 | 760 | carbonic anhydrase II | CA2 | M36532 (209301_at) |
| Hs.157439 | 4166 | carbohydrate (N- | CHST6 | NM_021615 (221059_s_at) |
| | | acetylglucosamine 6-O) | | |
| | | sulfotransferase 6 | | |
| Hs.159604 | 833 | cysteinyl-tRNA | CARS | AI769685 (212971_at) |
| | | synthetase | | |
| Hs.16297 | 10063 | COX17 homolog, | COX17 | NM_005694 (203880_at) |
| | | cytochrome c oxidase | | |
| | | assembly protein (yeast) | | |
| Hs.1708 | 7203 | chaperonin containing | CCT3 | NM_005998 (200910_at) |
| | | TCP1, subunit 3 (gamma) | | |
| Hs.173125 | 10105 | peptidylprolyl isomerase F | PPIF | BC005020 (201489_at) |
| | | (cyclophilin F) | | |
| Hs.173987 | 8669 | eukaryotic translation | EIF3S1 | BC002719 (208985_s_at) |
| | | initiation factor 3, subunit | | |
| | | 1 alpha, 35 kDa | | |
| Hs.177556 | 1495 | catenin (cadherin- | CTNNA1 | D14705 (210844_x_at) |
| | | associated protein), alpha | | |
| | | 1, 102 kDa | | |
| Hs.178551 | 6132 | ribosomal protein L8 | RPL8 | NM_000973 (200936_at) |
| Hs.180062 | 5696 | proteasome (prosome, | PSMB8 | U17496 (209040_s_at) |
| | | macropain) subunit, beta | | |
| | | type, 8 (large | | |
| | | multifunctional protease | | |
| | | 7) | | |
| Hs.180383 | 1848 | dual specificity | DUSP6 | BC003143 (208891_at) |
| | | phosphatase 6 | | |
| Hs.180920 | 6203 | ribosomal protein S9 | RPS9 | NM_001013 (217747_s_at) |
| Hs.180946 | 6125 | ribosomal protein L5 | RPL5 | AK027146 (216044_x_at) |
| Hs.182579 | 51056 | leucine aminopeptidase 3 | LAP3 | NM_015907 (217933_s_at) |
| Hs.183583 | 1992 | serine (or cysteine) | SERPINB1 | AI554300 (213572_s_at) |
| | | proteinase inhibitor, clade | | |
| | | B (ovalbumin), member 1 | | |
| Hs.193163 | 274 | bridging integrator 1 | BIN1 | AF001383 (210201_x_at) |
| Hs.20021 | 6843 | vesicle-associated | VAMP1 | AU150319 (213326_at) |
| | | membrane protein 1 | | |
| | | (synaptobrevin 1) | | |
| Hs.20478 | 1200 | ceroid-lipofuscinosis, | CLN2 | BG231932 (200742_s_at) |
| | | neuronal 2, late infantile | | |
| | | (Jansky-Bielschowsky | | |
| | | disease) | | |
| Hs.211914 | 4727 | NADH dehydrogenase | NDUFS7 | BC005954 (211752_s_at) |
| | | (ubiquinone) Fe-S protein | | |
| | | 7, 20 kDa (NADH- | | |
| | | coenzyme Q reductase) | | |
| Hs.220689 | 10146 | Ras-GTPase-activating | G3BP | BG500067 (201503_at) |
| | | protein SH3-domain- | | |
| | | binding protein | | |
| Hs.23259 | 64431 | actin-related protein 6 | ACTR6 | NM_022496 (218395_at) |
| Hs.23488 | 9861 | KIAA0107 gene product | P44S10 | NM_014814 (202753_at) |
| Hs.239926 | 6307 | sterol-C4-methyl oxidase- | SC4MOL | AV704962 (209146_at) |
| | | like | | |
| Hs.24587 | 10278 | embryonal Fyn- | EFS | NM_005864 (204400_at) |
| | | associated substrate | | |
| Hs.251531 | 5685 | proteasome (prosome, | PSMA4 | NM_002789 (203396_at) |
| | | macropain) subunit, alpha | | |
| | | type, 4 | | |
| Hs.2554 | 6480 | sialyltransferase 1 (beta- | SIAT1 | AI743792 (201998_at) |
| | | galactoside alpha-2,6- | | |
| | | sialyltransferase) | | |
| Hs.25597 | 64834 | elongation of very long | ELOVL1 | H93026 (57163_at) |
| | | chain fatty acids | | |
| | | (FEN1/Elo2, SUR4/Elo3, | | |
| | | yeast)-like 1 | | |
| Hs.25691 | 10268 | receptor (calcitonin) | RAMP3 | NM_005856 (205326_at) |
| | | activity modifying protein 3 | | |
| Hs.26350 | 8459 | tyrosylprotein | TPST2 | NM_003595 (204079_at) |
| | | sulfotransferase 2 | | |
| Hs.266940 | 6993 | t-complex-associated- | TCTEL1 | NM_006519 (201999_s_at) |
| | | testis-expressed 1-like 1 | | |
| Hs.273 | 2581 | galactosylceramidase | GALC | NM_000153 (204417_at) |
| | | (Krabbe disease) | | |
| Hs.274402 | 3304 | heat shock 70 kDa protein | HSPA1B | NM_005346 (202581_at) |
| | | 1B | | |
| Hs.279554 | 5719 | proteasome (prosome, | PSMD13 | NM_002817 (201232_s_at) |
| | | macropain) 26S subunit, | | |
| | | non-ATPase, 13 | | |
| Hs.279574 | 51079 | cell death-regulatory | GRIM19 | NM_015965 (220864_s_at) |
| | | protein GRIM19 | | |
| Hs.279901 | 51399 | synbindin | CGI-104 | NM_016146 |
| | | | | (217958 at, 217959_s_at) |
| Hs.288654 | 3208 | hippocalcin | HPCA | BC001777 (205454_at) |
| Hs.290070 | 2934 | gelsolin (amyloidosis, | GSN | NM_000177 (200696_s_at) |
| | | Finnish type) | | |
| Hs.291904 | 10134 | accessory protein BAP31 | BCAP31 | NM_005745 (200837_at) |
| Hs.293885 | 2617 | glycyl-tRNA synthetase | GARS | D30658 (208693_s_at) |
| Hs.30212 | 9318 | thyroid receptor | TRIP15 | NM_004236 (202467_s_at) |
| | | interacting protein 15 | | |
| Hs.3069 | 3313 | heat shock 70 kDa protein | HSPA9B | BC000478 (200691_s_at) |
| | | 9B (mortalin-2) | | |
| Hs.323567 | 950 | scavenger receptor class | SCARB2 | NM_005506 (201647_s_at) |
| | | B, member 2 | | |
| Hs.333823 | 28998 | mitochondrial ribosomal | MRPL13 | NM_014078 (218049_s_at) |
| | | protein L13 | | |
| Hs.334534 | 2799 | glucosamine (N-acetyl)-6- | GNS | AW167793 (212335_at) |
| | | sulfatase (Sanfilippo | | |
| | | disease IIID) | | |
| Hs.334842 | 10376 | tubulin, alpha, ubiquitous | K-ALPHA-1 | AL581768 (212639_x_at) |
| Hs.336678 | 1837 | dystrobrevin, alpha | DTNA | NM_001392 (205741_s_at) |
| Hs.337766 | 6142 | ribosomal protein L18a | RPL18A | NM_000980 (200869_at) |
| Hs.346918 | 5684 | proteasome (prosome, | PSMA3 | NM_002788 (201532_at) |
| | | macropain) subunit, alpha | | |
| | | type, 3 | | |
| Hs.351872 | 6272 | sortilin 1 | SORT1 | BF447105 (212807_s_at) |
| Hs.355957 | 6231 | ribosomal protein S26 | RPS26 | NM_001029 (217753_s_at) |
| Hs.356386 | 7879 | RAB7, member RAS | RAB7 | AK000826 (211961_s_at) |
| | | oncogene family | | |
| Hs.356463 | 2339 | farnesyltransferase, | FNTA | BG168896 (200090_at) |
| | | CAAX box, alpha | | |
| Hs.377915 | 4124 | mannosidase, alpha, | MAN2A1 | NM_002372 (205105_at) |
| | | class 2A, member 1 | | |
| Hs.380439 | 23208 | synaptotagmin XI | SYT11 | BC004291 (209198_s_at) |
| Hs.381255 | 8665 | eukaryotic translation | EIF3S5 | NM_003754 (200023_s_at) |
| | | initiation factor 3, subunit | | |
| | | 5 epsilon, 47 kDa | | |
| Hs.394389 | 5479 | peptidylprolyl isomerase | PPIB | NM_000942 (200968_s_at) |
| | | B (cyclophilin B) | | |
| Hs.395771 | 847 | catalase | CAT | AY028632 (211922_s_at) |
| Hs.397609 | 6217 | ribosomal protein S16 | RPS16 | NM_001020 (201258_at) |
| Hs.39871 | 4642 | myosin ID | MYO1D | AA621962 (212338_at) |
| Hs.40500 | 11079 | similar to S. cerevisiae | RER1 | NM_007033 (202296_s_at) |
| | | RER1 | | |
| Hs.406341 | 6187 | ribosomal protein S2 | RPS2 | AA630314 (212433_x_at) |
| Hs.406532 | 6185 | ribophorin II | RPN2 | AI560720 (213399_x_at) |
| Hs.407981 | 5689 | proteasome (prosome, | PSMB1 | NM_002793 (200876_s_at) |
| | | macropain) subunit, beta | | |
| | | type, 1 | | |
| Hs.408061 | 2171 | fatty acid binding protein | FABP5 | NM_001444 (202345_s_at) |
| | | 5 (psoriasis-associated) | | |
| Hs.408767 | 1410 | crystallin, alpha B | CRYAB | AF007162 (209283_at) |
| Hs.408943 | 5216 | profilin 1 | PFN1 | NM_005022 (200634_at) |
| Hs.410276 | 5687 | proteasome (prosome, | PSMA6 | BC002979 (208805_at) |
| | | macropain) subunit, alpha | | |
| | | type, 6 | | |
| Hs.410578 | 5034 | procollagen-proline, 2- | P4HB | J02783 (200654_at) |
| | | oxoglutarate 4- | | |
| | | dioxygenase (proline 4- | | |
| | | hydroxylase), beta | | |
| | | polypeptide (protein | | |
| | | disulfide isomerase; | | |
| | | thyroid hormone binding | | |
| | | protein p55) | | |
| Hs.411773 | 5683 | proteasome (prosome, | PSMA2 | NM_002787 (201317_s_at) |
| | | macropain) subunit, alpha | | |
| | | type, 2 | | |
| Hs.4147 | 23471 | translocation associated | TRAM1 | BC000687 (201398_s_at) |
| | | membrane protein 1 | | |
| Hs.421194 | 8460 | tyrosylprotein | TPST1 | NM_003596 (204140_at) |
| | | sulfotransferase 1 | | |
| Hs.424961 | 64981 | mitochondrial ribosomal | MRPL34 | AB049652 (221692_s_at) |
| | | protein L34 | | |
| Hs.426142 | 5281 | phosphatidylinositol | PIGF | NM_002643 (205077_s_at) |
| | | glycan, class F | | |
| Hs.426930 | 60 | actin, beta | ACTB | X00351 (AFFX- |
| | | | | HSAC07/X00351_5_at, AFFX- |
| | | | | HSAC07/X00351_M_at) |
| Hs.429621 | 11091 | WD repeat domain 5 | WDR5 | AF092131 (208714_at) |
| Hs.433394 | 7846 | tubulin, alpha 3 | TUBA3 | AF141347 (209118_s_at) |
| Hs.433506 | 10095 | actin related protein 2/3 | ARPC1B | NM_005720 (201954_at) |
| | | complex, subunit 1B, | | |
| | | 41 kDa | | |
| Hs.433702 | 1983 | eukaryotic translation | EIF5 | AL080102 (208708_x_at) |
| | | initiation factor 5 | | |
| Hs.448357 | 4500 | metallothionein 1L | MT1L | NM_002450 (204326_x_at) |
| Hs.4835 | 8663 | eukaryotic translation | EIF3S8 | AA679705 (215230_x_at) |
| | | initiation factor 3, subunit | | |
| | | 8, 110 kDa | | |
| Hs.48876 | 2222 | farnesyl-diphosphate | FDFT1 | AA872727 (208647_at) |
| | | farnesyltransferase 1 | | |
| Hs.49007 | 10914 | poly(A) polymerase alpha | PAPOLA | AI984479 (222035_s_at) |
| Hs.49346 | 6729 | signal recognition particle | SRP54 | NM_003136 (203605_at) |
| | | 54 kDa | | |
| Hs.49767 | 4726 | NADH dehydrogenase | NDUFS6 | NM_004553 (203606_at) |
| | | (ubiquinone) Fe-S protein | | |
| | | 6, 13 kDa (NADH- | | |
| | | coenzyme, Q reductase) | | |
| Hs.55097 | 28957 | mitochondrial ribosomal | MRPS28 | NM_014018 (219819_s_at) |
| | | protein S28 | | |
| Hs.55099 | 23637 | rab6 GTPase activating | GAPCENA | AI922519 (213313_at) |
| | | protein (GAP and | | |
| | | centrosome-associated) | | |
| Hs.55682 | 8664 | eukaryotic translation | EIF3S7 | NM_003753 (200005_at) |
| | | initiation factor 3, subunit | | |
| | | 7 zeta, 66/67 kDa | | |
| Hs.61490 | 29970 | schwannomin interacting | SCHIP1 | NM_014575 (204030_s_at) |
| | | protein 1 | | |
| Hs.66708 | 9341 | vesicle-associated | VAMP3 | BC003570 (201336_at) |
| | | membrane protein 3 | | |
| | | (cellubrevin) | | |
| Hs.66881 | 1781 | dynein, cytoplasmic, | DNCI2 | AF250307 (211684_s_at) |
| | | intermediate polypeptide 2 | | |
| Hs.7043 | 8802 | succinate-CoA ligase, | SUCLG1 | NM_003849 (217874_at) |
| | | GDP-forming, alpha | | |
| | | subunit | | |
| Hs.70669 | 51617 | HMP19 protein | HMP19 | NM_015980 (218623_at) |
| Hs.74137 | 10972 | transmembrane trafficking | TMP21 | BE780075 (212352_s_at) |
| | | protein | | |
| Hs.74619 | 5708 | proteasome (prosome, | PSMD2 | NM_002808 (200830_at) |
| | | macropain) 26S subunit, | | |
| | | non-ATPase, 2 | | |
| Hs.75232 | 6397 | SEC14-like 1 (S. cerevisiae) | SEC14L1 | AI017770 (202083_s_at) |
| Hs.7527 | 25996 | small fragment nuclease | DKFZP566E144 | NM_015523 (218194_at) |
| UniGene | LocusID | Description | Symbol | ACCESSION (Probe ID) |
| Hs.75283 | 6642 | sorting nexin 1 | SNX1 | AF065484 (214531_s_at) |
| Hs.75318 | 7277 | tubulin, alpha 1 (testis | TUBA1 | AL565074 (212242_at) |
| | | specific) | | |
| Hs.75410 | 3309 | heat shock 70 kDa protein | HSPA5 | AF216292 (211936_at) |
| | | 5 (glucose-regulated | | |
| | | protein, 78 kDa) | | |
| Hs.75428 | 6647 | superoxide dismutase 1, | SOD1 | NM_000454 (200642_at) |
| | | soluble (amyotrophic | | |
| | | lateral sclerosis 1 (adult)) | | |
| Hs.75607 | 4082 | myristoylated alanine-rich | MARCKS | AA770596 (213002_at) |
| | | protein kinase C substrate | | |
| Hs.75914 | 10959 | coated vesicle membrane | RNP24 | AK024976 (200087_s_at) |
| | | protein | | |
| Hs.76067 | 3315 | heat shock 27 kDa protein 1 | HSPB1 | NM_001540 (201841_s_at) |
| Hs.76293 | 9168 | thymosin, beta 10 | TMSB10 | NM_021103 (217733_s_at) |
| Hs.76640 | 28984 | RGC32 protein | RGC32 | NM_014059 (218723_s_at) |
| Hs.76918 | 4864 | Niemann-Pick disease, | NPC1 | NM_000271 (202679_at) |
| | | type C1 | | |
| Hs.77290 | 6888 | transaldolase 1 | TALDO1 | NM_006755 (201463_s_at) |
| Hs.77356 | 7037 | transferrin receptor (p90, | TFRC | BC001188 (208691_at) |
| | | CD71) | | |
| Hs.77432 | 1956 | epidermal growth factor | EGFR | AW 157070 (201983_s_at) |
| | | receptor (erythroblastic | | |
| | | leukemia viral (v-erb-b) | | |
| | | oncogene homolog, | | |
| | | avian) | | |
| Hs.77501 | 6443 | sarcoglycan, beta (43 kDa | SGCB | U29586 (205120_s_at) |
| | | dystrophin-associated | | |
| | | glycoprotein) | | |
| Hs.77805 | 529 | ATPase, H+ transporting, | ATP6V1E1 | BC004443 (208678_at) |
| | | lysosomal 31 kDa, V1 | | |
| | | subunit E isoform 1 | | |
| Hs.77890 | 2983 | guanylate cyclase 1, | GUCY1B3 | AF020340 (211555_s_at) |
| | | soluble, beta 3 | | |
| Hs.77899 | 7168 | tropomyosin 1 (alpha) | TPM1 | Z24727 (210986_s_at) |
| Hs.78305 | 5862 | RAB2, member RAS | RAB2 | AA535244 (208730_x_at) |
| | | oncogene family | | |
| Hs.78996 | 5111 | proliferating cell nuclear | PCNA | NM_002592 (201202_at) |
| | | antigen | | |
| Hs.79150 | 10575 | chaperonin containing | CCT4 | NM_006430 (200877_at) |
| | | TCP1, subunit 4 (delta) | | |
| Hs.79226 | 9638 | fasciculation and | FEZ1 | NM_005103 (203562_at) |
| | | elongation protein zeta 1 | | |
| | | (zygin I) | | |
| Hs.79357 | 5706 | proteasome (prosome, | PSMC6 | NM_002806 (201699_at) |
| | | macropain) 26S subunit, | | |
| | | ATPase, 6 | | |
| Hs.79378 | 8900 | cyclin A1 | CCNA1 | NM_003914 (205899_at) |
| Hs.79381 | 25801 | grancalcin, EF-hand | GCA | NM_012198 (203765_at) |
| | | calcium binding protein | | |
| Hs.8021 | 23348 | zizimin1 | zizimin1 | AL576253 (212538_at) |
| Hs.80395 | 4118 | mal, T-cell differentiation | MAL | NM_002371 (204777_s_at) |
| | | protein | | |
| Hs.80680 | 9961 | major vault protein | MVP | NM_017458 (202180_s_at) |
| Hs.80712 | 23176 | likely ortholog of mouse | 8-Sep | BC001329 (209000_s_at) |
| | | septin 8 | | |
| Hs.80919 | 6856 | synaptophysin-like protein | SYPL | AI768845 (201259_s_at) |
| Hs.82030 | 7453 | tryptophanyl-tRNA | WARS | NM_004184 (200629_at) |
| | | synthetase | | |
| Hs.82222 | 7869 | sema domain, | SEMA3B | NM_004636 (203071_at) |
| | | immunoglobulin domain | | |
| | | (Ig), short basic domain, | | |
| | | secreted, (semaphorin) | | |
| | | 3B | | |
| Hs.82425 | 10092 | actin related protein 2/3 | ARPC5 | AL516350 (211963_s_at) |
| | | complex, subunit 5, | | |
| | | 16 kDa | | |
| Hs.82568 | 1593 | cytochrome P450, family | CYP27A1 | NM_000784 (203979_at) |
| | | 27, subfamily A, | | |
| | | polypeptide 1 | | |
| Hs.8262 | 3920 | lysosomal-associated | LAMP2 | J04183 (203041_s_at) |
| | | membrane protein 2 | | |
| Hs.82890 | 1603 | defender against cell | DAD1 | NM_001344 (200046_at) |
| | | death 1 | | |
| Hs.84665 | 9499 | titin immunoglobulin | TTID | NM_006790 (219728_at) |
| | | domain protein (myotilin) | | |
| Hs.85226 | 3988 | lipase A, lysosomal acid, | LIPA | NM_000235 (201847_at) |
| | | cholesterol esterase | | |
| | | (Wolman disease) | | |
| Hs.88778 | 873 | carbonyl reductase 1 | CBR1 | BC002511 (209213_at) |
| Hs.89399 | 517 | ATP synthase, H+ | ATP5G2 | D13119 (208764_s_at) |
| | | transporting, | | |
| | | mitochondrial F0 | | |
| | | complex, subunit c | | |
| | | (subunit 9), isoform 2 | | |
| Hs.89545 | 5692 | proteasome (prosome, | PSMB4 | NM_002796 (202243_s_at) |
| | | macropain) subunit, beta | | |
| | | type, 4 | | |
| Hs.89866 | 1371 | coproporphyrinogen | CPO | NM_000097 (204172_at) |
| | | oxidase (coproporphyria, | | |
| | | harderoporphyria) | | |
| Hs.9006 | 9218 | VAMP (vesicle-associated | VAPA | AF154847 (208780_x_at) |
| | | membrane protein)- | | |
| | | associated protein A, | | |
| | | 33 kDa | | |
| Hs.90073 | 1434 | CSE1 chromosome | CSE1L | AF053640 (210766_s_at) |
| | | segregation 1-like (yeast) | | |
| Hs.90998 | 23157 | septin 6 | 6-Sep | AV721177 (215236_s_at) |
| Hs.96427 | 23150 | GRP1-binding protein | GRSP1 | AU145019 (213056_at) |
| | | GRSP1 | | |
| Hs.9964 | 6183 | mitochondrial ribosomal | MRPS12 | NM_021107 (204331_s_at) |
| | | protein S12 | | |
| Hs.99858 | 6130 | ribosomal protein L7a | RPL7A | NM_000972 (217740_x_at) |

TABLE 9
VThal - SYNAPTIC TRANSMISSION

| | | | | ACCESSION |
|---|---|---|---|---|
| UniGene | LocusID | Description | Symbol | (Probe ID) |
| Hs.103724 | 5376 | peripheral myelin | PMP22 | L03203 |
| | | protein 22 | | |
| Hs.141308 | 4340 | myelin | MOG | NM_002433 |
| | | oligodendrocyte | | |
| | | glycoprotein | | |
| Hs.154679 | 6857 | synaptotagmin I | SYT1 | AV723167 |
| Hs.170808 | 2572 | glutamate | GAD2 | NM_000818 |
| | | decarboxylase 2 | | |
| | | (pancreatic islets | | |
| | | and brain, 65 kDa) | | |
| Hs.324784 | 2571 | glutamate | GAD1 | NM_000817 |
| | | decarboxylase 1 | | |
| | | (brain, 67 kDa) | | |
| Hs.3281 | 4885 | neuronal pentraxin II | NPTX2 | U26662 |
| Hs.336678 | 1837 | dystrobrevin, alpha | DTNA | NM_001392 |
| Hs.69547 | 4155 | myelin basic protein | MBP | AW070431 |

TABLE 10
VThal-26S PROTEOSOME

| | | | | ACCESSION |
|---|---|---|---|---|
| UniGene | LocusID | Description | Symbol | (Probe ID) |
| Hs.148495 | 5710 | proteasome (prosome, macropain) 26S subunit, | PSMD4 | AB033605 |
| | | non-ATPase, 4 | | (210460_s_at) |
| Hs.180062 | 5696 | proteasome (prosome, macropain) subunit, beta | PSMB8 | U17496 |
| | | type, 8 (large multifunctional protease 7) | | (209040_s_at) |
| Hs.23488 | 9861 | KIAA0107 gene product | P44S10 | NM_014814 |
| | | | | (202753_at) |
| Hs.251531 | 5685 | proteasome (prosome, macropain) subunit, alpha | PSMA4 | NM_002789 |
| | | type, 4 | | (203396_at) |
| Hs.346918 | 5684 | proteasome (prosome, macropain) subunit, alpha | PSMA3 | NM_002788 |
| | | type, 3 | | (201532_at) |
| Hs.407981 | 5689 | proteasome (prosome, macropain) subunit, beta | PSMB1 | NM_002793 |
| | | type, 1 | | (200876_s_at) |
| Hs.411773 | 5683 | proteasome (prosome, macropain) subunit, alpha | PSMA2 | NM_002787 |
| | | type, 2 | | (201317_s_at) |
| Hs.74619 | 5708 | proteasome (prosome, macropain) 26S subunit, | PSMD2 | NM_002808 |
| | | non-ATPase, 2 | | (200830_at) |
| Hs.79357 | 5706 | proteasome (prosome, macropain) 26S subunit, | PSMC6 | NM_002806 |
| | | ATPase, 6 | | (201699_at) |
| Hs.89545 | 5692 | proteasome (prosome, macropain) subunit, beta | PSMB4 | NM_002796 |
| | | type, 4 | | (202243_s_at) |

TABLE 11
VThal - MACROMOLECULE BIOSYNTH

| UniGene | LocusID | Description | Symbol | ACCESSION (Probe ID) |
|---|---|---|---|---|
| Hs.111039 | 4836 | N-myristoyltransferase 1 | NMT1 | AF020500 (201157_s_at) |
| Hs.12163 | 8894 | eukaryotic translation initiation | EIF2S2 | BC000461 (208726_s_at) |
| | | factor 2, subunit 2 beta, 38 kDa | | |
| Hs.130181 | 2590 | UDP-N-acetyl-alpha-D- | GALNT2 | NM_004481 |
| | | galactosamine:polypeptide N- | | (217788_s_at) |
| | | acetylgalactosaminyltransferase 2 | | |
| | | (GalNAc-T2) | | |
| Hs.144063 | 6301 | seryl-tRNA synthetase | SARS | NM_006513 (200802_at) |
| Hs.150580 | 10209 | putative translation initiation factor | SUI1 | AF083441 (202021_x_at) |
| Hs.155103 | 9086 | eukaryotic translation initiation factor | EIF1AY | BC005248 (204409_s_at) |
| | | factor 1A, Y chromosome | | |
| Hs.159604 | 833 | cysteinyl-tRNA synthetase | CARS | AI769685 (212971_at) |
| Hs.170204 | 23043 | KIAA0551 protein | KIAA0551 | AF172268 (211828_s_at) |
| Hs.173987 | 8669 | eukaryotic translation initiation | EIF3S1 | BC002719 (208985_s_at) |
| | | factor 3, subunit 1 alpha, 35 kDa | | |
| Hs.178551 | 6132 | ribosomal protein L8 | RPL8 | NM_000973 (200936_at) |
| Hs.180920 | 6203 | ribosomal protein S9 | RPS9 | NM_001013 |
| | | | | (217747_s_at) |
| Hs.180946 | 6125 | ribosomal protein L5 | RPL5 | AK027146 (216044_x_at) |
| Hs.213289 | 3949 | low density lipoprotein receptor | LDLR | NM_000527 |
| | | (familial hypercholesterolemia) | | (202068_s_at) |
| Hs.2554 | 6480 | sialyltransferase 1 (beta-galactoside | SIAT1 | AI743792 (201998_at) |
| | | alpha-2,6-sialyltransferase) | | |
| Hs.279901 | 51399 | synbindin | CGI-104 | NM_016146 |
| | | | | (217958_at, 217959_s_at) |
| Hs.293885 | 2617 | glycyl-tRNA synthetase | GARS | D30658 (208693_s_at) |
| Hs.333513 | 9255 | small inducible cytokine subfamily | SCYE1 | NM_004757 |
| | | E, member 1 (endothelial monocyte- | | (202542_s_at) |
| | | activating) | | |
| Hs.333823 | 28998 | mitochondrial ribosomal protein L13 | MRPL13 | NM_014078 |
| | | | | (218049_s_at) |
| Hs.337766 | 6142 | ribosomal protein L18a | RPL18A | NM_000980 (200869_at) |
| Hs.355957 | 6231 | ribosomal protein S26 | RPS26 | NM_001029 |
| | | | | (217753_s_at) |
| Hs.377915 | 4124 | mannosidase, alpha, class 2A, | MAN2A1 | NM_002372 (205105_at) |
| | | member 1 | | |
| Hs.381050 | 27087 | beta-1,3-glucuronyltransferase 1 | B3GAT1 | NM_018644 (219521_at) |
| | | (glucuronosyltransferase P) | | |
| Hs.381255 | 8665 | eukaryotic translation initiation | EIF3S5 | NM_003754 |
| | | factor 3, subunit 5 epsilon, 47 kDa | | (200023_s_at) |
| Hs.397609 | 6217 | ribosomal protein S16 | RPS16 | NM_001020 (201258_at) |
| Hs.406341 | 6187 | ribosomal protein S2 | RPS2 | AA630314 (212433_x_at) |
| Hs.424961 | 64981 | mitochondrial ribosomal protein L34 | MRPL34 | AB049652 (221692_s_at) |
| Hs.426142 | 5281 | phosphatidylinositol glycan, class F | PIGF | NM_002643 |
| | | | | (205077_s_at) |
| Hs.433702 | 1983 | eukaryotic translation initiation | EIF5 | AL080102 (208708_x_at) |
| | | factor 5 | | |
| Hs.4835 | 8663 | eukaryotic translation initiation | EIF3S8 | AA679705 (215230_x_at) |
| | | factor 3, subunit 8, 110 kDa | | |
| Hs.55682 | 8664 | eukaryotic translation initiation | EIF3S7 | NM_003753 (200005_at) |
| | | factor 3, subunit 7 zeta, 66/67 kDa | | |
| Hs.76067 | 3315 | heat shock 27 kDa protein 1 | HSPB1 | NM_001540 |
| | | | | (201841_s_at) |
| Hs.8148 | 51714 | selenoprotein T | SELT | NM_016275 (217811_at) |
| Hs.82030 | 7453 | tryptophanyl-tRNA synthetase | WARS | NM_004184 (200629_at) |
| Hs.82890 | 1603 | defender against cell death 1 | DAD1 | NM_001344 (200046_at) |
| Hs.85226 | 3988 | lipase A, lysosomal acid, cholesterol | LIPA | NM_000235 (201847_at) |
| | | esterase (Wolman disease) | | |
| Hs.9964 | 6183 | mitochondrial ribosomal protein S12 | MRPS12 | NM_021107 |
| | | | | (204331_s_at) |
| Hs.99858 | 6130 | ribosomal protein L7a | RPL7A | NM_000972 |
| | | | | (217740_x_at) |

TABLE 12
Mthal - NEUROFILAMENT

| | | | Sym- | ACCESSION |
|---|---|---|---|---|
| UniGene | LocusID | Description | bol | (Probe ID) |
| Hs.198760 | 4744 | neurofilament, | NEFH | NM_021076 |
| | | heavy polypeptide | | (204412_s_at) |
| | | 200 kDa | | |
| Hs.211584 | 4747 | neurofilament, | NEFL | AL537457 |
| | | light polypeptide | | (221805_at) |
| | | 68 kDa | | |
| Hs.71346 | 4741 | neurofilament 3 | NEF3 | NM_005382 |
| | | (150 kDa medium) | | (205113_at) |

TABLE 13
HC - EXTRACELLULAR

| | | | | ACCESSION |
|---|---|---|---|---|
| UniGene | LocusID | Description | Symbol | (Probe ID) |
| Hs.12409 | 6750 | somatostatin | SST | NM_001048 |
| | | | | (213921_at) |
| Hs.2563 | 6863 | tachykinin, precursor 1 | TAC1 | NM_003182 |
| | | (substance K, substance P, | | (206552_s_at) |
| | | neurokinin 1, neurokinin 2, | | |
| | | neuromedin L, neurokinin alpha, | | |
| | | neuropeptide K, neuropeptide gamma) | | |
| Hs.336678 | 1837 | dystrobrevin, alpha | DTNA | NM_001392 |
| | | | | (205741_s_at) |
| Hs.386793 | 2878 | glutathione peroxidase 3 | GPX3 | NM_002084 |
| | | (plasma) | | (201348_at) |
| Hs.427202 | 7276 | transthyretin (prealbumin, | TTR | AF162690 |
| | | amyloidosis type 1) | | (209660_at) |
| Hs.64016 | 5627 | protein S (alpha) | PROS1 | NM_000313 |
| | | | | (207808_s_at) |
| Hs.7306 | 6422 | secreted frizzled-related | SFRP1 | NM_003012 |
| | | protein 1 | | (202037_s_at) |
| Hs.75184 | 1116 | chitinase 3-like 1 | CHI3L1 | M80927 |
| | | (cartilage glycoprotein-39) | | (209395_at, |
| | | | | 209396_s_at) |
| Hs.76224 | 2202 | EGF-containing fibulin-like | EFEMP1 | AI826799 |
| | | extracellular matrix protein 1 | | (201842_s_at) |
| Hs.94592 | 9365 | klotho | KL | NM_004795 |
| | | | | (205978_at) |

TABLE 14
AnCg - PROTEOSOME COMPLEX

| | | | | ACCESSION |
|---|---|---|---|---|
| UniGene | LocusID | Description | Symbol | (Probe ID) |
| Hs.251531 | 5685 | proteasome (prosome, macropain) | PSMA4 | NM_002789 |
| | | subunit, alpha type, 4 | | (203396_at) |
| Hs.346918 | 5684 | proteasome (prosome, macropain) | PSMA3 | NM_002788 |
| | | subunit, alpha type, 3 | | (201532_at) |
| Hs.3887 | 5707 | proteasome (prosome, macropain) | PSMD1 | AI860431 |
| | | 26S subunit, non-ATPase, 1 | | (201198_s_at) |
| Hs.407981 | 5689 | proteasome (prosome, macropain) | PSMB1 | W86293 |
| | | subunit, beta type, 1 | | (214288_s_at) |
| Hs.410276 | 5687 | proteasome (prosome, macropain) | PSMA6 | BC002979 |
| | | subunit, alpha type, 6 | | (208805_at) |
| Hs.78466 | 5714 | proteasome (prosome, macropain) | PSMD8 | NM_002812 |
| | | 26S subunit, non-ATPase, 8 | | (200820_at) |
| Hs.82159 | 5682 | proteasome (prosome, macropain) | PSMA1 | BC005932 |
| | | subunit, alpha type, 1 | | (211746_x_at) |
| Hs.89545 | 5692 | proteasome (prosome, macropain) | PSMB4 | NM_002796 |
| | | subunit, beta type, 4 | | (202243_s_at) |

TABLE 15
PC - STEROL BIOSYNTHESIS

| | | | | ACCESSION |
|---|---|---|---|---|
| UniGene | LocusID | Description | Symbol | (Probe ID) |
| Hs.11806 | 1717 | 7-dehydrocholesterol | DHCR7 | NM_001360 |
| | | reductase | | (201791_s_at) |
| Hs.226213 | 1595 | cytochrome P450, family 51, | CYP51A1 | NM_000786 |
| | | subfamily A, polypeptide 1 | | (202314_at) |
| Hs.239926 | 6307 | sterol-C4-methyl | SC4MOL | AV704962 |
| | | oxidase-like | | (209146_at) |
| Hs.48876 | 2222 | farnesyl-diphosphate | FDFT1 | AA872727 |
| | | farnesyltransferase 1 | | (208647_at) |
| Hs.71465 | 6713 | squalene epoxidase | SQLE | AF098865 |
| | | | | (209218_at) |
| Hs.75616 | 1718 | 24-dehydrocholesterol | DHCR24 | NM_014762 |
| | | reductase | | (200862_at) |
| Hs.76038 | 3422 | isopentenyl-diphosphate | IDI1 | BC005247 |
| | | delta isomerase | | (208881_x_at) |

TABLE 16
nAcc-26S proteosome

| | | | | ACCESSION |
|---|---|---|---|---|
| UniGene | LocusID | Description | Symbol | (Probe ID) |
| Hs.148495 | 5710 | proteasome (prosome, macropain) | PSMD4 | AB033605 |
| | | 26S subunit, non-ATPase, 4 | | (210460_s_at) |
| Hs.251531 | 5685 | proteasome (prosome, macropain) | PSMA4 | NM_002789 |
| | | subunit, alpha type, 4 | | (203396_at) |
| Hs.346918 | 5684 | proteasome (prosome, macropain) | PSMA3 | NM_002788 |
| | | subunit, alpha type, 3 | | (201532_at) |
| Hs.3887 | 5707 | proteasome (prosome, macropain) | PSMD1 | AI860431 |
| | | 26S subunit, non-ATPase, 1 | | (201198_s_at) |
| Hs.79357 | 5706 | proteasome (prosome, macropain) | PSMC6 | NM_002806 |
| | | 26S subunit, ATPase, 6 | | (201699_at) |
| Hs.82159 | 5682 | proteasome (prosome, macropain) | PSMA1 | BC005932 |
| | | subunit, alpha type, 1 | | (211746_x_at) |
| Hs.82793 | 5691 | proteasome (prosome, macropain) | PSMB3 | NM_002795 |
| | | subunit, beta type, 3 | | (201400_at) |
| Hs.89545 | 5692 | proteasome (prosome, macropain) | PSMB4 | NM_002796 |
| | | subunit, beta type, 4 | | (202243_s_at) |

TABLE 17
nAcc - CYTOPLASM

| | | | | ACCESSION |
|---|---|---|---|---|
| UniGene | LocusID | Description | Symbol | (Probe ID) |
| Hs.100527 | 22866 | connector enhancer | CNK2 | NM_014927 |
| | | of KSR2 | | (206731_at) |
| Hs.107476 | 10632 | ATP synthase, H+ transporting, | ATP5L | AF070655 |
| | | mitochondrial F0 complex, subunit g | | (208746_x_at) |
| Hs.107526 | 9334 | UDP-Gal: betaGlcNAc beta 1,4- | B4GALT5 | BF691447 |
| | | galactosyltransferase, polypeptide 5 | | (221484_at) |
| Hs.108802 | 4905 | N-ethylmaleimide-sensitive factor | NSF | NM_006178 |
| | | | | (202395_at) |
| Hs.108809 | 10574 | chaperonin containing TCP1, | CCT7 | NM_006429 |
| | | subunit 7 (eta) | | (200812_at) |
| Hs.112667 | 27019 | dynein, axonemal, intermediate | DNAI1 | NM_012144 |
| | | polypeptide 1 | | (220125_at) |
| Hs.11465 | 9446 | glutathione-S-transferase like; | GSTTLp28 | NM_004832 |
| | | glutathione transferase omega | | (201470_at) |
| Hs.11899 | 3156 | 3-hydroxy-3-methylglutaryl-Coenzyme | HMGCR | AL518627 |
| | | A reductase | | (202539_s_at) |
| Hs.119000 | 87 | actinin, alpha 1 | ACTN1 | BC003576 |
| | | | | (208637_x_at) |
| Hs.12163 | 8894 | eukaryotic translation initiation | EIF2S2 | BC000461 |
| | | factor 2, subunit 2 beta, 38 kDa | | (208726_s_at) |
| Hs.122967 | 11275 | kelch-like 2, Mayven (Drosophila) | KLHL2 | NM_007246 |
| | | | | (219157_at) |
| Hs.12376 | 27445 | piccolo (presynaptic cytomatrix | PCLO | AB011131 |
| | | protein) | | (213558_at) |
| Hs.12451 | 2009 | echinoderm microtubule associated | EML1 | NM_004434 |
| | | protein like 1 | | (204797_s_at) |
| Hs.12887 | 57180 | actin-related protein 3-beta | ARP3BETA | NM_020445 |
| | | | | (218868_at) |
| Hs.12956 | 30851 | Tax interaction protein 1 | TIP-1 | AF234997 |
| | | | | (209154_at) |
| Hs.14376 | 71 | actin, gamma 1 | ACTG1 | AL567820 |
| | | | | (211995_x_at) |
| Hs.146580 | 2026 | enolase 2, (gamma, neuronal) | ENO2 | NM_001975 |
| | | | | (201313_at) |
| Hs.148495 | 5710 | proteasome (prosome, macropain) | PSMD4 | AB033605 |
| | | 26S subunit, non-ATPase, 4 | | (210460_s_at) |
| Hs.15071 | 10694 | chaperonin containing TCP1, subunit | CCT8 | NM_006585 |
| | | 8 (theta) | | (200873_s_at) |
| Hs.152936 | 1173 | adaptor-related protein complex 2, | AP2M1 | NM_004068 |
| | | mu 1 subunit | | (200613_at) |
| Hs.154654 | 1545 | cytochrome P450, family 1, | CYP1B1 | AU144855 |
| | | subfamily B, polypeptide 1 | | (202436_s_at) |
| Hs.154679 | 6857 | synaptotagmin I | SYT1 | AV723167 |
| | | | | (203998_s_at) |
| Hs.159154 | 10381 | tubulin, beta, 4 | TUBB4 | AL565749 |
| | | | | (213476_x_at) |
| Hs.167791 | 5954 | reticulocalbin 1, EF-hand calcium | RCN1 | NM_002901 |
| | | binding domain | | (201063_at) |
| Hs.168075 | 3842 | karyopherin (importin) beta 2 | KPNB2 | U72069 |
| | | | | (209226_s_at) |
| Hs.169476 | 2597 | glyceraldehyde-3-phosphate | GAPD | M33197 (AFFX- |
| | | dehydrogenase | | HUMGAPDH/ |
| | | | | M33197_5_at) |
| Hs.1708 | 7203 | chaperonin containing | CCT3 | NM_005998 |
| | | TCP1, subunit 3 (gamma) | | (200910_at) |
| Hs.172471 | 7881 | potassium voltage-gated channel, | KCNAB1 | NM_003471 |
| | | shaker-related subfamily, beta member 1 | | (208213_s_at) |
| Hs.173554 | 7385 | ubiquinol-cytochrome c reductase core | UQCRC2 | NM_003366 |
| | | protein II | | (200883_at) |
| Hs.179661 | 203068 | beta 5-tubulin | OK/SW- | AF141349 |
| | | | cl.56 | (209026_x_at) |
| Hs.180920 | 6203 | ribosomal protein S9 | RPS9 | NM_001013 |
| | | | | (217747_s_at) |
| Hs.182217 | 8803 | succinate-CoA ligase, ADP-forming, | SUCLA2 | NM_003850 |
| | | beta subunit | | (202930_s_at) |
| Hs.193163 | 274 | bridging integrator 1 | BIN1 | U87558 |
| | | | | (210202_s_at) |
| Hs.2043 | 291 | solute carrier family 25 (mitochondrial | SLC25A4 | NM_001151 |
| | | carrier; adenine nucleotide translocator), | | (202825_at) |
| | | member 4 | | |
| Hs.211584 | 4747 | neurofilament, light polypeptide | NEFL | AL537457 |
| | | 68 kDa | | (221805_at) |
| Hs.21276 | 10087 | collagen, type IV, alpha 3 (Goodpasture | COL4A3BP | NM_005713 |
| | | antigen) binding protein | | (219625_s_at) |
| Hs.23259 | 64431 | actin-related protein 6 | ACTR6 | NM_022496 |
| | | | | (218395_at) |
| Hs.239356 | 6812 | syntaxin binding protein 1 | STXBP1 | NM_003165 |
| | | | | (202260_s_at) |
| Hs.239926 | 6307 | sterol-C4-methyl oxidase-like | SC4MOL | AV704962 |
| | | | | (209146_at) |
| Hs.251531 | 5685 | proteasome (prosome, macropain) | PSMA4 | NM_002789 |
| | | subunit, alpha type, 4 | | (203396_at) |
| Hs.2554 | 6480 | sialyltransferase 1 (beta-galactoside | SIAT1 | AI743792 |
| | | alpha-2,6-sialyltransferase) | | (201998_at) |
| Hs.256697 | 3094 | histidine triad nucleotide binding | HINT1 | N32864 |
| | | protein 1 | | (200093_s_at) |
| Hs.2642 | 1917 | eukaryotic translation elongation | EEF1A2 | NM_001958 |
| | | factor 1 alpha 2 | | (204540_at) |
| Hs.272927 | 10484 | Sec23 homolog A (S. cerevisiae) | SEC23A | AI753659 |
| | | | | (212887_at) |
| Hs.2795 | 3939 | lactate dehydrogenase A | LDHA | NM_005566 |
| | | | | (200650_s_at) |
| Hs.279554 | 5719 | proteasome (prosome, macropain) | PSMD13 | NM_002817 |
| | | 26S subunit, non-ATPase, 13 | | (201232_s_at) |
| Hs.281866 | 523 | ATPase, H+ transporting, lysosomal | ATP6V1A | NM_001690 |
| | | 70 kDa, V1 subunit A | | (201971_s_at) |
| Hs.297939 | 1508 | cathepsin B | CTSB | NM_001908 |
| | | | | (200838_at, |
| | | | | 200839_s_at) |
| Hs.30212 | 9318 | thyroid receptor interacting | TRIP15 | NM_004236 |
| | | protein 15 | | (202467_s_at) |
| Hs.334842 | 10376 | tubulin, alpha, ubiquitous | K-ALPHA-1 | AL581768 |
| | | | | (212639_x_at) |
| Hs.336678 | 1837 | dystrobrevin, alpha | DTNA | NM_001392 |
| | | | | (205741_s_at) |
| Hs.337766 | 6142 | ribosomal protein L18a | RPL18A | NM_000980 |
| | | | | (200869_at) |
| Hs.346918 | 5684 | proteasome (prosome, macropain) | PSMA3 | NM_002788 |
| | | subunit, alpha type, 3 | | (201532_at) |
| Hs.347939 | 3040 | hemoglobin, alpha 2 | HBA2 | BC005931 |
| | | | | (211745_x_at) |
| Hs.353170 | 811 | calreticulin | CALR | AA910371 |
| | | | | (212952_at) |
| Hs.355957 | 6231 | ribosomal protein S26 | RPS26 | NM_001029 |
| | | | | (217753_s_at) |
| Hs.36587 | 5510 | protein phosphatase 1, regulatory | PPP1R7 | BF718769 |
| | | subunit 7 | | (21 3465_s_at) |
| Hs.36927 | 10808 | heat shock 105 kDa/110 kDa | HSPH1 | NM_006644 |
| | | protein 1 | | (206976_s_at) |
| Hs.386017 | 3831 | kinesin 2 60/70 kDa | KNS2 | AA284075 |
| | | | | (212877_at, |
| | | | | 212878_s_at) |
| Hs.3887 | 5707 | proteasome (prosome, macropain) | PSMD1 | AI860431 |
| | | 26S subunit, non-ATPase, 1 | | (201198_s_at) |
| Hs.408767 | 1410 | crystallin, alpha B | CRYAB | AF007162 |
| | | | | (209283_at) |
| Hs.409829 | 4725 | NADH dehydrogenase (ubiquinone) | NDUFS5 | NM_004552 |
| | | Fe—S protein 5, 15 kDa | | (201757_at) |
| | | (NADH-coenzyme Q reductase) | | |
| Hs.424220 | 3039 | hemoglobin, alpha 1 | HBA1 | AF349571 |
| | | | | (211699_x_at) |
| Hs.424961 | 64981 | mitochondrial ribosomal protein L34 | MRPL34 | AB049652 |
| | | | | (221692_s_at) |
| Hs.425293 | 4736 | ribosomal protein L10a | RPL10A | NM_007104 |
| | | | | (200036_s_at) |
| Hs.426142 | 5281 | phosphatidylinositol glycan, | PIGF | NM_002643 |
| | | class F | | (205078_at) |
| Hs.426930 | 60 | actin, beta | ACTS | X00351 (AFFX- |
| | | | | HSAC07/X00351_5_at) |
| Hs.429621 | 11091 | WD repeat domain 5 | WDR5 | AF092131 |
| | | | | 208714_at) |
| Hs.430207 | 6159 | ribosomal protein L29 | RPL29 | NM_000992 |
| | | | | (200823_x_at) |
| Hs.432605 | 7357 | UDP-glucose ceramide | UGCG | NM_003358 |
| | | glucosyltransferase | | (204881_s_at) |
| Hs.433496 | 9908 | Ras-GTPase activating protein SH3 | G3BP2 | AB014560 |
| | | domain-binding protein 2 | | (208841_s_at) |
| Hs.43670 | 11127 | kinesin family member 3A | KIF3A | NM_007054 |
| | | | | (213623_at) |
| Hs.4835 | 8663 | eukaryotic translation initiation | EIF3S8 | AA679705 |
| | | factor 3, subunit 8, 110 kDa | | (215230_x_at) |
| Hs.48876 | 2222 | farnesyl-diphosphate | FDFT1 | AA872727 |
| | | farnesyltransferase 1 | | (208647_at) |
| Hs.49767 | 4726 | NADH dehydrogenase (ubiquinone) | NDUFS6 | NM_004553 |
| | | Fe—S protein 6, 13 kDa | | (203606_at) |
| | | (NADH-coenzyme Q reductase) | | |
| Hs.5556 | 4706 | NADH dehydrogenase (ubiquinone) 1, | NDUFAB1 | NM_005003 |
| | | alpha/beta subcomplex, 1, 8 kDa | | (202077_at) |
| Hs.57937 | 54715 | ataxin 2-binding protein 1 | A2BP1 | NM_018723 |
| | | | | (221217_s_at) |
| Hs.65248 | 1780 | dynein, cytoplasmic, intermediate | DNCI1 | NM_004411 |
| | | polypeptide 1 | | (205348_s_at) |
| Hs.71346 | 4741 | neurofilament 3 (150 kDa medium) | NEF3 | NM_005382 |
| | | | | (205113_at) |
| Hs.74571 | 375 | ADP-ribosylation factor 1 | ARF1 | AA580004 |
| | | | | (208750_s_at) |
| Hs.74617 | 1639 | dynactin 1 (p150, glued homolog, | DCTN1 | NM_004082 |
| | | Drosophila) | | (201082_s_at) |
| Hs.75149 | 6456 | SH3-domain GRB2-like 2 | SH3GL2 | NM_003026 |
| | | | | (205751_at) |
| Hs.75187 | 9804 | translocase of outermitochondrial | TOMM20- | BG165094 |
| | | membrane 20 (yeast) homolog | PENDING | (212773_s_at) |
| Hs.75318 | 7277 | tubulin, alpha 1 (testis specific) | TUBA1 | AL565074 |
| | | | | (212242_at) |
| Hs.75616 | 1718 | 24-dehydrocholesterol reductase | DHCR24 | NM_014762 |
| | | | | (200862_at) |
| Hs.75893 | 288 | ankyrin 3, node of Ranvier | ANK3 | NM_020987 |
| | | (ankyrin G) | | (206385_s_at) |
| Hs.76067 | 3315 | heat shock 27 kDa protein 1 | HSPB1 | NM_001540 |
| | | | | (201841_s_at) |
| Hs.76293 | 9168 | thymosin, beta 10 | TMSB10 | NM_021103 |
| | | | | (217733_s_at) |
| Hs.76930 | 6622 | synuclein, alpha (non A4 component | SNCA | BG260394 |
| | | of amyloid precursor) | | (204466_s_at) |
| Hs.77385 | 4637 | myosin, light polypeptide 6, alkali, | MYL6 | BE734356 |
| | | smooth muscle and non-muscle | | (212082_s_at) |
| Hs.77917 | 7347 | ubiquitin carboxyl-terminal esterase | UCHL3 | NM_006002 |
| | | L3 (ubiquitin thiolesterase) | | (204616_at) |
| Hs.78979 | 2734 | golgi apparatus protein 1 | GLG1 | AK025457 |
| | | | | (214730_s_at) |
| Hs.79356 | 7805 | Lysosomal-associated multispanning | LAPTM5 | AI589086 |
| | | membrane protein-5 | | (201720_s_at) |
| Hs.79357 | 5706 | proteasome (prosome, macropain) | PSMC6 | NM_002806 |
| | | 26S subunit, ATPase, 6 | | (201699_at) |
| Hs.7936 | 10458 | BAI1-associated protein 2 | BAIAP2 | AB017120 |
| | | | | (205293_x_at) |
| Hs.79381 | 25801 | grancalcin, EF-hand calcium binding | GCA | NM_012198 |
| | | protein | | (203765_at) |
| Hs.79404 | 27065 | DNA segment on chromosome 4 | D4S234E | BC001745 |
| | | (unique) 234 expressed sequence | | (209570_s_at) |
| Hs.80680 | 9961 | major vault protein | MVP | NM_017458 |
| | | | | (202180_s_at) |
| Hs.82030 | 7453 | tryptophanyl-tRNA synthetase | WARS | NM_004184 |
| | | | | (200629_at) |
| Hs.82159 | 5682 | proteasome (prosome, macropain) | PSMA1 | BC005932 |
| | | subunit, alpha type, 1 | | (211746_x_at) |
| Hs.82314 | 3251 | hypoxanthine phosphoribosyltransferase | HPRT1 | NM_000194 |
| | | 1 (Lesch-Nyhan syndrome) | | (202854_at) |
| Hs.8262 | 3920 | lysosomal-associated membrane protein 2 | LAMP2 | J04183 |
| | | | | (203041_s_at) |
| Hs.82793 | 5691 | proteasome (prosome, macropain) | PSMB3 | NM_002795 |
| | | subunit, beta type, 3 | | (201400_at) |
| Hs.85226 | 3988 | lipase A, lysosomal acid, cholesterol | LIPA | NM_000235 |
| | | esterase (Wolman disease) | | (201847_at) |
| Hs.8526 | 11041 | UDP-GlcNAc: betaGal beta-1,3-N- | B3GNT6 | NM_006876 |
| | | acetylglucosaminyltransferase 6 | | (203188_at) |
| Hs.8679 | 11332 | brain acyl-CoA hydrolase | BACH | NM_007274 |
| | | | | (208002_s_at) |
| Hs.89399 | 517 | ATP synthase, H+ transporting, | ATP5G2 | D13119 |
| | | mitochondrial FO complex, subunit c | | (208764_s_at) |
| | | (subunit 9), isoform 2 | | |
| Hs.89545 | 5692 | proteasome (prosome, macropain) | PSMB4 | NM_002796 |
| | | subunit, beta type, 4 | | (202243_s_at) |
| Hs.90005 | 11075 | stathmin-like 2 | STMN2 | NM_007029 |
| | | | | (203001_s_at) |
| Hs.90073 | 1434 | CSE1 chromosome segregation 1-like | CSE1L | AF053641 |
| | | (yeast) | | (201111_at) |
| Hs.9950 | 23480 | Sec61 gamma | SEC61G | NM_014302 |
| | | | | (203484_at) |
| Hs.99947 | 6252 | reticulon 1 | RTN1 | BC000314 |
| | | | | (210222_s_at) |

TABLE 18
HC - RESPONSE TO BIOTIC STIM

| | | | | ACCESSION |
|---|---|---|---|---|
| UniGene | LocusID | Description | Symbol | (Probe ID) |
| Hs.155024 | 604 | B-cell CLL/lymphoma 6 (zinc finger | BCL6 | NM_001706 |
| | | protein 51) | | (203140_at) |
| Hs.159590 | 4062 | lymphocyte antigen 6 complex, | LY6H | NM_002347 |
| | | locus H | | (206773_at) |
| Hs.174142 | 1436 | colony stimulating factor 1 receptor, | CSF1R | NM_005211 |
| | | formerly McDonough feline sarcoma | | (203104_at) |
| | | viral (v-fms) oncogene homolog | | |
| Hs.174195 | 10581 | interferon induced transmembrane | IFITM2 | NM_006435 |
| | | protein 2 (1-8D) | | (201315_x_at) |
| Hs.181301 | 1520 | cathepsin S | CTSS | BC002642 |
| | | | | (202901_x_at) |
| Hs.184018 | 9450 | lymphocyte antigen 86 | LY86 | NM_004271 |
| | | | | (205859_at) |
| Hs.234726 | 12 | serine (or cysteine) proteinase | SERPINA3 | NM_001085 |
| | | inhibitor, clade A (alpha-1 | | (202376_at) |
| | | antiproteinase, antitrypsin), member 3 | | |
| Hs.25647 | 2353 | v-fos FBJ murine osteosarcoma viral | FOS | BC004490 |
| | | oncogene homolog | | (209189_at) |
| Hs.277477 | 3107 | major histocompatibility complex, | HLA-C | BC004489 |
| | | class I, C | | (208812_x_at) |
| Hs.278613 | 3429 | interferon, alpha-inducible | IFI27 | NM_005532 |
| | | protein 27 | | (202411_at) |
| Hs.297681 | 5265 | serine (or cysteine) proteinase | SERPINA1 | NM_000295 |
| | | inhibitor, clade A (alpha-1 | | (202833_s_at) |
| | | antiproteinase, antitrypsin), member 1 | | |
| Hs.303649 | 6347 | chemokine (C—C motif) ligand 2 | CCL2 | S69738 |
| | | | | (216598_s_at) |
| Hs.372679 | 2215 | Fc fragment of IgG, low affinity | FCGR3B | NM_000570 |
| | | IIIb, receptor for (CD16) | | (204006_s_at) |
| Hs.375108 | 934 | CD24 antigen (small cell lung | CD24 | BG327863 |
| | | carcinoma cluster 4 antigen) | | (208650_s_at) |
| Hs.386793 | 2878 | glutathione peroxidase 3 (plasma) | GPX3 | NM_002084 |
| | | | | (201348_at) |
| Hs.407995 | 4282 | macrophage migration inhibitory | MIF | NM_002415 |
| | | factor (glycosylation-inhibiting factor) | | (217871_s_at) |
| Hs.433300 | 2207 | Fc fragment of IgE, high affinity I, | FCER1G | NM_004106 |
| | | receptor for; gamma polypeptide | | (204232_at) |
| Hs.433414 | 10410 | interferon induced transmembrane | IFITM3 | BF338947 |
| | | protein 3 (1-8U) | | (212203_x_at) |
| Hs.458286 | 8519 | interferon induced transmembrane | IFITM1 | AA749101 |
| | | protein 1 (9-27) | | (214022_s_at) |
| Hs.62192 | 2152 | coagulation factor III | F3 | NM_001993 |
| | | (thromboplastin, tissue factor) | | (204363_at) |
| Hs.6510 | 29953 | thyrotropin-releasing hormone | TRHDE | NM_013381 |
| | | degrading ectoenzyme | | (219937_at) |
| Hs.69328 | 23643 | lymphocyte antigen 96 | LY96 | NM_015364 |
| | | | | (206584_at) |
| Hs.69547 | 4155 | myelin basic protein | MBP | AW070431 |
| | | | | (210136_at) |
| Hs.72050 | 8382 | non-metastatic cells 5, protein | NME5 | NM_003551 |
| | | expressed in (nucleoside-diphosphate | | (206197_at) |
| | | kinase) | | |
| Hs.73817 | 6348 | chemokine (C—C motif) ligand 3 | CCL3 | NM_002983 |
| | | | | (205114_s_at) |
| Hs.75106 | 1191 | clusterin (complement lysis inhibitor, | CLU | AI982754 |
| | | SP-40, 40, sulfated glycoprotein 2, | | (222043_at) |
| | | testosterone-repressed prostate | | |
| | | message 2, apolipoprotein J) | | |
| Hs.753 | 2357 | formyl peptide receptor 1 | FPR1 | NM_002029 |
| | | | | (205119_s_at) |
| Hs.77424 | 2209 | Fc fragment of IgG, high affinity Ia, | FCGR1A | L03419 |
| | | receptor for (CD64) | | (214511_x_at) |
| Hs.77961 | 3106 | major histocompatibility complex, | HLA-B | D83043 |
| | | class I, B | | (208729_x_at) |
| Hs.79022 | 2669 | GTP binding protein overexpressed in | GEM | NM_005261 |
| | | skeletal muscle | | (204472_at) |
| Hs.80420 | 6376 | chemokine (C-X3-C motif) ligand 1 | CX3CL1 | U84487 (823_at) |
| Hs.82112 | 3554 | interleukin 1 receptor, type I | IL1R1 | NM_000877 |
| | | | | (202948_at) |
| Hs.82212 | 963 | CD53 antigen | CD53 | NM_000560 |
| | | | | (203416_at) |
| Hs.83384 | 6285 | S100 calcium binding protein, | S100B | BC001766 |
| | | beta (neural) | | (209686_at) |
| Hs.83656 | 397 | Rho GDP dissociation inhibitor | ARHGDIB | NM_001175 |
| | | (GDI) beta | | (201288_at) |
| Hs.89499 | 240 | arachidonate 5-lipoxygenase | ALOX5 | NM_000698 |
| | | | | (204446_s_at) |
| Hs.8986 | 713 | complement component 1, q subcomponent, | C1QB | NM_000491 |
| | | beta polypeptide | | (202953_at) |
| Hs.9098 | 26266 | solute carrier family 13 (sodium/sulfate | SLC13A4 | NM_012450 |
| | | symporters), member 4 | | (219824_at) |
| Hs.9641 | 712 | complement component 1, q subcomponent, | C1QA | NM_015991 |
| | | alpha polypeptide | | (218232_at) |
| Hs.9963 | 7305 | TYRO protein tyrosine kinase | TYROBP | NM_003332 |
| | | binding protein | | (204122_at) |

TABLE 19
DLPFC - RIBOSOME

| | | | | ACCESSION |
|---|---|---|---|---|
| UniGene | LocusID | Description | Symbol | (Probe ID) |
| Hs.108957 | 51065 | ribosomal | RPS27L | NM_015920 |
| | | protein | | (218007_s_at) |
| | | S27-like | | |
| Hs.274417 | 28973 | mitochondrial | MRPS18B | AL050361 |
| | | ribosomal | | (217408_at) |
| | | protein S18B | | |
| Hs.275865 | 6222 | ribosomal | RPS18 | NM_022551 |
| | | protein S18 | | (201049_s_at) |
| Hs.298262 | 6223 | ribosomal | RPS19 | BE259729 |
| | | protein S19 | | (213414_s_at) |
| Hs.302588 | 6181 | ribosomal | RPLP2 | NM_001004 |
| | | protein, | | (200909_s_at) |
| | | large P2 | | |
| Hs.334807 | 6156 | ribosomal | RPL30 | L05095 |
| | | protein L30 | | (200062_s_at) |
| Hs.337766 | 6142 | ribosomal | RPL18A | NM_000980 |
| | | protein L18a | | (200869_at) |
| Hs.354176 | 6188 | ribosomal | RPS3 | U14990 |
| | | protein S3 | | (208692_at) |
| Hs.355957 | 6231 | ribosomal | RPS26 | NM_001029 |
| | | protein S26 | | (217753_s_at) |
| Hs.397609 | 6217 | ribosomal | RPS16 | AI200589 |
| | | protein S16 | | (213890_x_at) |
| Hs.399720 | 6202 | ribosomal | RPS8 | NM_001012 |
| | | protein S8 | | (200858_s_at) |
| Hs.406341 | 6187 | ribosomal | RPS2 | AA630314 |
| | | protein S2 | | (212433_x_at) |
| Hs.406478 | 6170 | ribosomal | RPL39 | BC001019 |
| | | protein L39 | | (208695_s_at) |
| Hs.424299 | 6176 | ribosomal protein, | RPLP1 | NM_001003 |
| | | large, P1 | | (200763_s_at) |
| Hs.430207 | 6159 | ribosomal | RPL29 | NM_000992 |
| | | protein L29 | | (200823_x_at) |
| Hs.431392 | 6137 | ribosomal | RPL13 | BC004954 |
| | | protein L13 | | (208929_x_at) |
| Hs.433406 | 6210 | ribosomal | RPS15A | NM_001019 |
| | | protein S15a | | (200781_s_at) |
| Hs.434029 | 6206 | ribosomal | RPS12 | AI799007 |
| | | protein S12 | | (213377_x_at) |
| Hs.458148 | 6134 | ribosomal | RPL10 | NM_006013 |
| | | protein L10 | | (200725_x_at) |
| Hs.76064 | 6157 | ribosomal | RPL27A | NM_000990 |
| | | protein L27a | | (203034_s_at) |
| Hs.76698 | 27230 | stress-asso- | SERP1 | BG107676 |
| | | ciated endo- | | (200969_at) |
| | | plasmic reticulum | | |
| | | protein 1 | | |

TABLE 20
ANCg - PROTEIN TARGETING

| | | | | ACCESSION |
|---|---|---|---|---|
| UniGene | LocusID | Description | Symbol | (Probe ID) |
| Hs.10842 | 5901 | RAN, member RAS | RAN | AF054183 |
| | | oncogene family | | (200750_s_at) |
| Hs.111126 | 754 | pituitary tumor-transforming 1 | PTTG1IP | NM_004339 |
| | | interacting protein | | (200677_at) |
| Hs.113503 | 3843 | karyopherin | KPNB3 | AU148466 |
| | | (importin) beta 3 | | (211953_s_at) |
| Hs.159557 | 3838 | karyopherin alpha 2 (RAG cohort | KPNA2 | NM_002266 |
| | | 1, importin alpha 1) | | (201088_at) |
| Hs.199179 | 5903 | RAN binding protein 2 | RANBP2 | D42063 |
| | | | | (201713_s_at) |
| Hs.220689 | 10146 | Ras-GTPase-activating protein | G3BP | BG500067 |
| | | SH3-domain-binding protein | | (201503_at) |
| Hs.3886 | 3839 | karyopherin alpha 3 | KPNA3 | AF034756 |
| | | (importin alpha 4) | | (221503_s_at) |
| Hs.433496 | 9908 | Ras-GTPase activating protein SH3 | G3BP2 | AB014560 |
| | | domain-binding protein 2 | | (208841_s_at) |
| Hs.49346 | 6729 | signal recognition | SRP54 | NM_003136 |
| | | particle 54 kDa | | (203605_at) |
| Hs.72916 | 2737 | GLI-Kruppel family member GLI3 | GLI3 | NM_000168 |
| | | (Greig cephalopolysyndactyly | | (205201_at) |
| | | syndrome) | | |
| Hs.75187 | 9804 | translocase of outer mitochondrial | TOMM20- | NM_014765 |
| | | membrane 20 (yeast) homolog | PENDING | (200662_s_at) |
| Hs.79037 | 3329 | heat shock 60 kDa protein 1 | HSPD1 | BE256479 |
| | | (chaperonin) | | (200806_s_at) |
| Hs.79090 | 7514 | exportin 1 (CRM1 homolog, yeast) | XPO1 | D89729 |
| | | | | (208775_at) |
| Hs.8180 | 6386 | syndecan binding protein | SDCBP | NM_005625 |
| | | (syntenin) | | (200958_s_at) |
| Hs.90073 | 1434 | CSE1 chromosome segregation 1- | CSE1L | AF053641 |
| | | like (yeast) | | (201111_at) |
| Hs.9950 | 23480 | Sec61 gamma | SEC61G | NM_014302 |
| | | | | (203484_at) |

TABLE 21
AnCg-ER

| | | | | ACCESSION |
|---|---|---|---|---|
| UniGene | LocusID | Description | Symbol | (Probe ID) |
| Hs.11125 | 28972 | signal peptidase 12 kDa | SPC12 | NM_014041 |
| | | | | (217927_at) |
| Hs.11899 | 3156 | 3-hydroxy-3-methylglutaryl- | HMGCR | AL518627 |
| | | Coenzyme A reductase | | (202539_s_at) |
| Hs.154654 | 1545 | cytochrome P450, family 1, | CYP1B1 | NM_000104 |
| | | subfamily B, polypeptide 1 | | (202437_s_at) |
| Hs.185973 | 8560 | degenerative spermatocyte homolog, | DEGS | BC000961 |
| | | lipid desaturase (Drosophila) | | (209250_at) |
| Hs.227327 | 5861 | RAB1A, member RAS oncogene family | RAB1A | BC000905 |
| | | | | (208724_s_at) |
| Hs.237825 | 6731 | signal recognition particle 72 kDa | SRP72 | AI493872 |
| | | | | (208802_at) |
| Hs.239926 | 6307 | sterol-C4-methyl oxidase-like | SC4MOL | AV704962 |
| | | | | (209146_at) |
| Hs.24752 | 10006 | spectrin SH3 domain binding | SSH3BP1 | AF006516 |
| | | protein 1 | | (209028_s_at) |
| Hs.251531 | 5685 | proteasome (prosome, macropain) | PSMA4 | NM_002789 |
| | | subunit, alpha type, 4 | | (203396_at) |
| Hs.25253 | 4121 | mannosidase, alpha, class 1A, | MAN1A1 | BG287153 |
| | | member 1 | | (221760_at) |
| Hs.252831 | 10313 | reticulon 3 | RTN3 | NM_006054 |
| | | | | (219549_s_at) |
| Hs.272927 | 10484 | Sec23 homolog A (S. cerevisiae) | SEC23A | AI753659 |
| | | | | (212887_at) |
| Hs.283664 | 444 | aspartate beta-hydroxylase | ASPH | AF289489 |
| | | | | (209135_at) |
| Hs.296244 | 10652 | SNARE protein Ykt6 | YKT6 | NM_006555 |
| | | | | (217785_s_at) |
| Hs.346918 | 5684 | proteasome (prosome, macropain) | PSMA3 | NM_002788 |
| | | subunit, alpha type, 3 | | (201532_at) |
| Hs.353170 | 811 | calreticulin | CALR | AI348935 |
| | | | | (214315_x_at) |
| Hs.3887 | 5707 | proteasome (prosome, macropain) | PSMD1 | AI860431 |
| | | 26S subunit, non-ATPase, 1 | | (201198_s_at) |
| Hs.406532 | 6185 | ribophorin II | RPN2 | AI560720 |
| | | | | (213399_x_at) |
| Hs.407981 | 5689 | proteasome (prosome, macropain) | PSMB1 | W86293 |
| | | subunit, beta type, 1 | | (214288_s_at) |
| Hs.410276 | 5687 | proteasome (prosome, macropain) | PSMA6 | BC002979 |
| | | subunit, alpha type, 6 | | (208805_at) |
| Hs.41270 | 5352 | procollagen-lysine, 2-oxoglutarate | PLOD2 | NM_000935 |
| | | 5-dioxygenase (lysine hydroxylase) 2 | | (202620_s_at) |
| Hs.426142 | 5281 | phosphatidylinositol glycan, | PIGF | NM_002643 |
| | | class F | | (205078_at) |
| Hs.432605 | 7357 | UDP-glucose ceramide | UGCG | NM_003358 |
| | | glucosyltransferase | | (204881_s_at) |
| Hs.48876 | 2222 | farnesyl-diphosphate | FDFT1 | AA872727 |
| | | farnesyltransferase 1 | | (208647_at) |
| Hs.49346 | 6729 | signal recognition particle 54 kDa | SRP54 | NM_003136 |
| | | | | (203605_at) |
| Hs.5085 | 8813 | dolichyl-phosphate mannosyltransferase | DPMI | NM_003859 |
| | | polypeptide 1, catalytic subunit | | (202673_at) |
| Hs.585 | 338 | apolipoprotein B (including Ag(x) | APOB | NM_000384 |
| | | antigen) | | (205108_s_at) |
| Hs.7239 | 10427 | SEC24 related gene family, member B | SEC24B | NM_006323 |
| | | (S. cerevisiae) | | (202798_at) |
| Hs.76698 | 27230 | stress-associated endoplasmic | SERP1 | AL136807 |
| | | reticulum protein 1 | | (200970_s_at) |
| Hs.78305 | 5862 | RAB2, member RAS oncogene family | RAB2 | AU158062 |
| | | | | (208731_at) |
| Hs.78466 | 5714 | proteasome (prosome, macropain) | PSMD8 | NM_002812 |
| | | 26S subunit, non-ATPase, 8 | | (200820_at) |
| Hs.79137 | 5110 | protein-L-isoaspartate (D-aspartate) | PCMT1 | D25547 |
| | | O-methyltransferase | | (210156_s_at) |
| Hs.8180 | 6386 | syndecan binding protein (syntenin) | SDCBP | NM_005625 |
| | | | | (200958_s_at) |
| Hs.82159 | 5682 | proteasome (prosome, macropain) | PSMA1 | BC005932 |
| | | subunit, alpha type, 1 | | (211746_x_at) |
| Hs.89545 | 5692 | proteasome (prosome, macropain) | PSMB4 | NM_002796 |
| | | subunit, beta type, 4 | | (202243_s_at) |
| Hs.9950 | 23480 | Sec61 gamma | SEC61G | NM_014302 |
| | | | | (203484_at) |

TABLE 22
ALL REGIONS - 1.2 FOLD

| | | Gene | |
|---|---|---|---|
| LOCUSLINK | Gene Name | Symbol | GO Category |
| 6347 | chemokine (C—C motif) ligand 2 | CCL2 | cation homeostasis |
| 54997 | hypothetical protein FLJ20607 | TSC | cation homeostasis |
| 11147 | HERV-H LTR-associating 3 | HHLA3 | cation homeostasis |
| 9900 | synaptic vesicle glycoprotein 2A | SV2A | cation homeostasis |
| 475 | ATX1 antioxidant protein 1 homolog (yeast) | ATOX1 | cation homeostasis |
| 9843 | hephaestin | HEPH | cation homeostasis |
| 10479 | solute carrier family 9 (sodium/hydrogen | SLC9A6 | cation homeostasis |
| | exchanger), isoform 6 | | |
| 4495 | metallothionein 1G | MT1G | cation homeostasis |
| 1356 | ceruloplasmin (ferroxidase) | CP | cation homeostasis |
| 6348 | chemokine (C—C motif) ligand 3 | CCL3 | cation homeostasis |
| 4306 | nuclear receptor subfamily 3, group C, member 2 | NR3C2 | cation homeostasis |
| 6358 | chemokine (C—C motif) ligand 14 | CCL14 | cation homeostasis |
| 794 | calbindin 2, 29 kDa (calretinin) | CALB2 | cation homeostasis |
| 793 | calbindin 1, 28 kDa | CALB1 | cation homeostasis |
| 4504 | metallothionein 3 (growth inhibitory factor | MT3 | cation homeostasis |
| | (neurotrophic)) | | |
| 4496 | metallothionein 1H | MT1H | cation homeostasis |
| 4501 | metallothionein 1X | MT1X | cation homeostasis |
| 7037 | transferrin receptor (p90, CD71) | TFRC | cation homeostasis |
| 6717 | sorcin | SRI | cation homeostasis |
| 6387 | chemokine (C—X—C motif) ligand 12 (stromal cell- | CXCL12 | cation homeostasis |
| | derived factor 1) | | |
| 27032 | ATPase, Ca⁺⁺ transporting, type 2C, member 1 | ATP2C1 | cation homeostasis |
| 6356 | chemokine (C—C motif) ligand 11 | CCL11 | cation homeostasis |
| 8671 | solute carrier family 4, sodium bicarbonate | SLC4A4 | cation homeostasis |
| | cotransporter, member 4 | | |
| 846 | calcium-sensing receptor (hypocalciuric | CASR | cation homeostasis |
| | hypercalcemia 1, severe neonatal | | |
| | hyperparathyroidism) | | |
| 23333 | KIAA0877 protein | KIAA0877 | cation homeostasis |
| 2512 | ferritin, light polypeptide | FTL | cation homeostasis |
| 7439 | ferritin, heavy polypeptide 1 | FTH1 | cation homeostasis |
| 351 | amyloid beta (A4) precursor protein (protease | APP | cation homeostasis |
| | nexin-II, Alzheimer disease) | | |
| 4502 | metallothionein 2A | MT2A | Copper ion homeostasis |
| 3337 | DnaJ (Hsp40) homolog, subfmaily B, member 1 | DNAJB1 | Heat shock protein |
| 3313 | heat shock 70 kDa protein 9B (mortalin-2) | HSPA9B | Heat shock protein |
| 3329 | heat shock 60 kDa protein 1 (chaperonin) | HSPD1 | Heat shock protein |
| 3301 | DnaJ (Hsp40) homolog, subfamily A, member 1 | DNAJA1 | Heat shock protein |
| 3300 | DnaJ (Hsp40) homolog, subfamily B, member 2 | DNAJB2 | Heat shock protein |
| 11080 | DnaJ (Hsp40) homolog, subfamily B, member 4 | DNAJB4 | Heat shock protein |
| 3336 | heat shock 10 kDa protein 1 (chaperonin 10) | HSPE1 | Heat shock protein |
| 10808 | heat shock 105 kDa/110 kDa protein 1 | HSPH1 | Heat shock protein |
| 3298 | heat shock transcription factor 2 | HSF2 | Heat shock protein |
| 3320 | heat shock 90 kDa protein 1, alpha | HSPCA | Heat shock protein |
| 3312 | heat shock 70 kDa protein 8 | HSPA8 | Heat shock protein |
| 3308 | heat shock 70 kDa protein 4 | HSPA4 | Heat shock protein |
| 25822 | DnaJ (Hsp40) homolog, subfamily B, member 5 | DNAJB5 | Heat shock protein |
| 3326 | heat shock 90 kDa protein 1, beta | HSPCB | Heat shock protein |
| 26353 | protein kinase H11 | H11 | Heat shock protein |
| 56034 | platelet derived growth factor C | PDGFC | Neurogenesis |
| 474 | atonal homolog 1 (Drosophila) | ATOH1 | Neurogenesis |
| 6647 | superoxide dismutase 1, soluble (amyotrophic | SOD1 | Neurogenesis |
| | lateral sclerosis 1 (adult)) | | |
| 65108 | MARCKS-like protein | MLP | Neurogenesis |
| 1639 | dynactin 1 (p150, glued homolog, Drosophila) | DCTN1 | Neurogenesis |
| 8507 | ectodermal-neural cortex (with BTB-like domain) | ENC1 | Neurogenesis |
| 26227 | phosphoglycerate dehydrogenase | PHGDH | Neurogenesis |
| 1809 | dihydropyrimidinase-like 3 | DPYSL3 | Neurogenesis |
| 10523 | calcium homeostasis endoplasmic reticulum protein | CHERP | Neurogenesis |
| 648 | B lymphoma Mo-MLV insertion region (mouse) | BMI1 | Neurogenesis |
| 6695 | sparc/osteonectin, cwcv and kazal-like domains | SPOCK | Neurogenesis |
| | proteoglycan (testican) | | |
| 1400 | collapsin response mediator protein 1 | CRMP1 | Neurogenesis |
| 51232 | cysteine-rich motor neuron 1 | CRIM1 | Neurogenesis |
| 7466 | Wolfram syndrome 1 (wolframin) | WFS1 | Neurogenesis |
| 7869 | sema domain, immunoglobulin domain (Ig), short | SEMA3B | Neurogenesis |
| | basic domain, secreted, (semaphorin) 3B | | |
| 3720 | jumonji homolog (mouse) | JMJ | Neurogenesis |
| 3280 | hairy and enhancer of split 1, (Drosophila) | HES1 | Neurogenesis |
| 5634 | phosphoribosyl pyrophosphate synthetase 2 | PRPS2 | Neurogenesis |
| 10231 | Down syndrome critical region gene 1-like 1 | DSCR1L1 | Neurogenesis |
| 10507 | sema domain, immunoglobulin domain (Ig), | SEMA4D | Neurogenesis |
| | transmembrane domain (TM) and short cytoplasmic | | |
| | domain, (semaphorin) 4D | | |
| 9638 | fasciculation and elongation protein zeta 1 (zygin I) | FEZ1 | Neurogenesis |
| 9839 | zinc finger homeobox 1b | ZFHX1B | Neurogenesis |
| 10512 | sema domain, immunoglobulin domain (Ig), short | SEMA3C | Neurogenesis |
| | basic domain, secreted, (semaphorin) 3C | | |
| 104 | adenosine deaminase, RNA-specific, B1 (RED1 | ADARB1 | Neurogenesis |
| | homolog rat) | | |
| 4900 | neurogranin (protein kinase C substrate, RC3) | NRGN | Neurogenesis |
| 27333 | golgi phosphoprotein 4 | GOLPH4 | Neurogenesis |
| 2247 | fibroblast growth factor 2 (basic) | FGF2 | Neurogenesis |
| 5803 | protein tyrosine phosphatase, receptor-type, Z | PTPRZ1 | Neurogenesis |
| | polypeptide 1 | | |
| 4929 | nuclear receptor subfamily 4, group A, member 2 | NR4A2 | Neurogenesis |
| 2775 | guanine nucleotide binding protein (G protein), | GNAO1 | Neurogenesis |
| | alpha activating activity polypeptide O | | |
| 1641 | doublecortex; lissencephaly, X-linked (doublecortin) | DCX | Neurogenesis |
| 2705 | gap junction protein, beta 1, 32 kDa (connexin 32, | GJB1 | Neurogenesis |
| | Charcot-Marie-Tooth neuropathy, X-linked) | | |
| 2173 | fatty acid binding protein 7, brain | FABP7 | Neurogenesis |
| 3730 | Kallmann syndrome 1 sequence | KAL1 | Neurogenesis |
| 10458 | BAI1-associated protein 2 | BAIAP2 | Neurogenesis |
| 5274 | serine (or cysteine) proteinase inhibitor, clade I | SERPINI1 | Neurogenesis |
| | (neuroserpin), member 1 | | |
| 9201 | doublecortin and CaM kinase-like 1 | DCAMKL1 | Neurogenesis |
| 11341 | scrapie responsive protein 1 | SCRG1 | Neurogenesis |
| 10178 | odz, odd Oz/ten-m homolog 1(Drosophila) | ODZ1 | Neurogenesis |
| 6456 | SH3-domain GRB2-like 2 | SH3GL2 | Neurogenesis |
| 4693 | Norrie disease (pseudoglioma) | NDP | Neurogenesis |
| 4745 | NEL-like 1 (chicken) | NELL1 | Neurogenesis |
| 4081 | mab-21-like 1 (C. elegans) | MAB21L1 | Neurogenesis |
| 4760 | neurogenic differentiation 1 | NEUROD1 | Neurogenesis |
| 9211 | leucine-rich, glioma inactivated 1 | LGI1 | Neurogenesis |
| 7545 | Zic family member 1 (odd-paired homolog, | ZIC1 | Neurogenesis |
| | Drosophila) | | |
| 6496 | sine oculis homeobox homolog 3 (Drosophila) | SIX3 | Neurogenesis |
| 320 | amyloid beta (A4) precursor protein-binding, family | APBA1 | Neurogenesis |
| | A, member 1 (X11) | | |
| 1910 | endothelin receptor type B | EDNRB | Neurogenesis |
| 22865 | KIAA0848 protein | KIAA0848 | Neurogenesis |
| 4062 | lymphocyte antigen 6 complex, locus H | LY6H | Neurogenesis |
| 1415 | crystallin, beta B2 | CRYBB2 | Neurogenesis |
| 4821 | NK2 transcription factor related, locus 2 | NKX2-2 | Neurogenesis |
| | (Drosophila) | | |
| 27255 | contactin 6 | CNTN6 | Neurogenesis |
| 7101 | nuclear receptor subfamily 2, group E, member 1 | NR2E1 | Neurogenesis |
| 1821 | dystrophin related protein 2 | DRP2 | Neurogenesis |
| 4336 | myelin-associated oligodendrocyte basic protein | MOBP | Neurogenesis |
| 10787 | NCK-associated protein 1 | NCKAP1 | Neurogenesis |
| 6622 | synuclein, alpha (non A4 component of amyloid | SNCA | Neurogenesis |
| | precursor) | | |
| 2821 | glucose phosphate isomerase | GPI | Neurogenesis |
| 4762 | neurogenin 1 | NEUROG1 | Neurogenesis |
| 1859 | dual-specificity tyrosine-(Y)-phosphorylation | DYRK1A | Neurogenesis |
| | regulated kinase 1A | | |
| 2824 | glycoprotein M6B | GPM6B | Neurogenesis |
| 4208 | MADS box transcription enhancer factor 2, | MEF2C | Neurogenesis |
| | polypeptide C (myocyte enhancer factor 2C) | | |
| 333 | amyloid beta (A4) precursor-like protein 1 | APLP1 | Neurogenesis |
| 5764 | pleiotrophin (heparin binding growth factor 8, | PTN | Neurogenesis |
| | neurite growth-promoting factor 1) | | |
| 2823 | glycoprotein M6A | GPM6A | Neurogenesis |
| 2048 | EphB2 | EPHB2 | Neurogenesis |
| 9353 | slit homolog 2 (Drosophila) | SLIT2 | Neurogenesis |
| 9048 | artemin | ARTN | Neurogenesis |
| 3785 | potassium voltage-gated channel, KQT-like | KCNQ2 | Neurogenesis |
| | subfamily, member 2 | | |
| 1742 | discs, large (Drosophila) homolog 4 | DLG4 | Neurogenesis |
| 195 | AHNAK nucleoprotein (desmoyokin) | AHNAK | Neurogenesis |
| 7155 | topoisomerase (DNA) II beta 180 kDa | TOP2B | Neurogenesis |
| 8829 | neuropilin 1 | NRP1 | Neurogenesis |
| 3241 | hippocalcin-like 1 | HPCAL1 | Neurogenesis |
| 9444 | quaking homolog, KH domain RNA binding (mouse) | QKI | Neurogenesis |
| 10376 | tubulin, alpha 3 | TUBA3 | Neurogenesis |
| 23180 | KIAA0084 protein | KIAA0084 | Neurogenesis |
| 10439 | olfactomedin 1 | OLFM1 | Neurogenesis |
| 429 | achaete-scute complex-like 1 (Drosophila) | ASCL1 | Neurogenesis |
| 1200 | ceroid-lipofuscinosis, neuronal 2, late infantile | CLN2 | Neurogenesis |
| | (Jansky-Bielschowsky disease) | | |
| 2257 | fibroblast growth factor 12 | FGF12 | Neurogenesis |
| 4915 | neurotrophic tyrosine kinase, receptor, type 2 | NTRK2 | Neurogenesis |
| 5361 | plexin A1 | PLXNA1 | Neurogenesis |
| 26050 | KIAA0918 protein | KIAA0918 | Neurogenesis |
| 773 | calcium channel, voltage-dependent, P/Q type, | CACNA1A | Neurogenesis |
| | alpha 1A subunit | | |
| 2752 | glutamate-ammonia ligase (glutamine synthase) | GLUL | Neurogenesis |
| 51399 | PTD009 protein | PTD009 | Neurogenesis |
| 26585 | cysteine knot superfamily 1, BMP antagonist 1 | CKTSF1B1 | Neurogenesis |
| 64218 | hypothetical protein FLJ12287 similar to | FLJ12287 | Neurogenesis |
| | semaphorins | | |
| 10313 | reticulon 3 | RTN3 | Neurogenesis |
| 4815 | ninjurin 2 | NINJ2 | Neurogenesis |
| 51440 | hippocalcin like 4 | HPCAL4 | Neurogenesis |
| 10842 | chromosome 7 open reading frame 16 | C7orf16 | Neurogenesis |
| 55727 | function unknown protein 1 | FLJ10648 | Neurogenesis |
| 64388 | hypothetical protein FLJ21195 similar to protein | FLJ21195 | Neurogenesis |
| | related to DAC and cerberus | | |
| 56934 | carbonic anhydrase X | CA10 | Neurogenesis |
| 55607 | protein phosphatase 1, regulatory (inhibitor) subunit | PPP1R9A | Neurogenesis |
| | 9A | | |
| 2259 | fibroblast growth factor 14 | FGF14 | Neurogenesis |
| 58158 | neurogenic differentiation 4 | NEUROD4 | Neurogenesis |
| 23462 | hairy/enhancer-of-split related with YRPW motif 1 | HEY1 | Neurogenesis |
| 23493 | hairy/enhancer-of-split related with YRPW motif 2 | HEY2 | Neurogenesis |
| 6477 | seven in absentia homolog 1 (Drosophila) | SIAH1 | Neurogenesis |
| 9637 | fasciculation and elongation protein zeta 2 (zygin II) | FEZ2 | Neurogenesis |
| 11189 | trinucleotide repeat containing 4 | TNRC4 | Neurogenesis |
| 2596 | growth associated protein 43 | GAP43 | Neurogenesis |
| 10858 | cytochrome P450, family 46, subfamily A, | CYP46A1 | Neurogenesis |
| | polypeptide 1 | | |
| 6134 | ribosomal protein L10 | RPL10 | Ribosome |
| 6158 | ribosomal protein L28 | RPL28 | Ribosome |
| 4736 | ribosomal protein L10a | RPL10A | Ribosome |
| 9045 | ribosomal protein L14 | RPL14 | Ribosome |
| 6176 | ribosomal protein, large, P1 | RPLP1 | Ribosome |
| 6210 | ribosomal protein S15a | RPS15A | Ribosome |
| 6159 | ribosomal protein L29 | RPL29 | Ribosome |
| 6202 | ribosomal protein S8 | RPS8 | Ribosome |
| 6142 | ribosomal protein L18a | RPL18A | Ribosome |
| 6181 | ribosomal protein, large P2 | RPLP2 | Ribosome |
| 6132 | ribosomal protein L8 | RPL8 | Ribosome |
| 6160 | ribosomal protein L31 | RPL31 | Ribosome |
| 6169 | ribosomal protein L38 | RPL38 | Ribosome |
| 6157 | ribosomal protein L27a | RPL27A | Ribosome |
| 6188 | ribosomal protein S3 | RPS3 | Ribosome |
| 6170 | ribosomal protein L39 | RPL39 | Ribosome |
| 6137 | ribosomal protein L13 | RPL13 | Ribosome |
| 6187 | ribosomal protein S2 | RPS2 | Ribosome |
| 6218 | ribosomal protein S17 | RPS17 | Ribosome |
| | ribosomal protein L35a | RPL35A | Ribosome |
| 716 | ribosomal protein S12 | RPS12 | Ribosome |
| 6223 | ribosomal protein S19 | RPS19 | Ribosome |
| 6217 | ribosomal protein S16 | RPS16 | Ribosome |
| 6168 | ribosomal protein L37a | RPL37A | Ribosome |
| 6175 | ribosomal protein, large, P0 | RPLP0 | Ribosome |
| 6130 | ribosomal protein L7a | RPL7A | Ribosome |
| 6203 | ribosomal protein S9 | RPS9 | Ribosome |
| 6125 | ribosomal protein L5 | RPL5 | Ribosome |
| 6231 | ribosomal protein S26 | RPS26 | Ribosome |
| 27115 | phosphodiesterase 7B | PDE7B | Synaptic transmission |
| 6866 | tachykinin 3 (neuromedin K, neurokinin beta) | TAC3 | Synaptic transmission |
| 2558 | gamma-aminobutyric acid (GABA) A receptor, | GABRA5 | Synaptic transmission |
| | alpha 5 | | |
| 4684 | neural cell adhesion molecule 1 | NCAM1 | Synaptic transmission |
| 8938 | BAI1-associated protein 3 | BAIAP3 | Synaptic transmission |
| 6386 | syndecan binding protein (syntenin) | SDCBP | Synaptic transmission |
| 6856 | synaptophysin-like protein | SYPL | Synaptic transmission |
| 6535 | solute carrier family 6 (neurotransmitter transporter, | SLC6A8 | Synaptic transmission |
| | creatine), member 8 | | |
| 22839 | KIAA0964 protein | KIAA0964 | Synaptic transmission |
| 5864 | RAB3A, member RAS oncogene family | RAB3A | Synaptic transmission |
| 6529 | solute carrier family 6 (neurotransmitter transporter, | SLC6A1 | Synaptic transmission |
| | GABA), member 1 | | |
| 6712 | spectrin, beta, non-erythrocytic 2 | SPTBN2 | Synaptic transmission |
| 273 | amphiphysin (Stiff-Man syndrome with breast | AMPH | Synaptic transmission |
| | cancer 128 kDa autoantigen) | | |
| 2743 | glycine receptor, beta | GLRB | Synaptic transmission |
| 43 | acetylcholinesterase (YT blood group) | ACHE | Synaptic transmission |
| 590 | butyrylcholinesterase | BCHE | Synaptic transmission |
| 6324 | sodium channel, voltage-gated, type I, beta | SCN1B | Synaptic transmission |
| 1392 | corticotropin releasing hormone | CRH | Synaptic transmission |
| 869 | cerebellin 1 precursor | CBLN1 | Synaptic transmission |
| 2913 | glutamate receptor, metabotropic 3 | GRM3 | Synaptic transmission |
| 6861 | synaptotagmin V | SYT5 | Synaptic transmission |
| 9229 | discs, large (Drosophila) homolog-associated | DLGAP1 | Synaptic transmission |
| | protein 1 | | |
| 1134 | cholinergic receptor, nicotinic, alpha polypeptide 1 | CHRNA1 | Synaptic transmission |
| | (muscle) | | |
| 2571 | glutamate decarboxylase 1 (brain, 67 kDa) | GAD1 | Synaptic transmission |
| 2554 | gamma-aminobutyric acid (GABA) A receptor, | GABRA1 | Synaptic transmission |
| | alpha 1 | | |
| 5173 | prodynorphin | PDYN | Synaptic transmission |
| 2566 | gamma-aminobutyric acid (GABA) A receptor, | GABRG2 | Synaptic transmission |
| | gamma 2 | | |
| 6511 | solute carrier family 1 (high affinity | SLC1A6 | Synaptic transmission |
| | aspartate/glutamate transporter), member 6 | | |
| 6014 | Ras-like without CAAX 2 | RIT2 | Synaptic transmission |
| 2560 | gamma-aminobutyric acid (GABA) A receptor, beta 1 | GABRB1 | Synaptic transmission |
| 2555 | gamma-aminobutyric acid (GABA) A receptor, | GABRA2 | Synaptic transmission |
| | alpha 2 | | |
| 5297 | phosphatidylinositol 4-kinase, catalytic, alpha | PIK4CA | Synaptic transmission |
| | polypeptide | | |
| 3356 | 5-hydroxytryptamine (serotonin) receptor 2A | HTR2A | Synaptic transmission |
| 2559 | gamma-aminobutyric acid (GABA) A receptor, | GABRA6 | Synaptic transmission |
| | alpha 6 | | |
| 2911 | glutamate receptor, metabotropic 1 | GRM1 | Synaptic transmission |
| 3352 | 5-hydroxytryptamine (serotonin) receptor 1D | HTR1D | Synaptic transmission |
| 1175 | adaptor-related protein complex 2, sigma 1 subunit | AP2S1 | Synaptic transmission |
| 2563 | gamma-aminobutyric acid (GABA) A receptor, delta | GABRD | Synaptic transmission |
| 1312 | catechol-O-methyltransferase | COMT | Synaptic transmission |
| 1267 | 2′,3′-cyclic nucleotide 3′ phosphodiesterase | CNP | Synaptic transmission |
| 2890 | glutamate receptor, ionotropic, AMPA 1 | GRIA1 | Synaptic transmission |
| 9568 | G protein-coupled receptor 51 | GPR51 | Synaptic transmission |
| 1325 | cortistatin | CORT | Synaptic transmission |
| 1136 | cholinergic receptor, nicotinic, alpha polypeptide 3 | CHRNA3 | Synaptic transmission |
| 6854 | synapsin II | SYN2 | Synaptic transmission |
| 9362 | copine VI (neuronal) | CPNE6 | Synaptic transmission |
| 1728 | NAD(P)H dehydrogenase, quinone 1 | NQO1 | Synaptic transmission |
| 3351 | 5-hydroxytryptamine (serotonin) receptor 1B | HTR1B | Synaptic transmission |
| 2902 | glutamate receptor, ionotropic, N-methyl D- | GRIN1 | Synaptic transmission |
| | aspartate 1 | | |
| 51552 | RAB14, member RAS oncogene family | RAB14 | Synaptic transmission |
| 5594 | mitogen-activated protein kinase 1 | MAPK1 | Synaptic transmission |
| 4128 | monoamine oxidase A | MAOA | Synaptic transmission |
| 8867 | synaptojanin 1 | SYNJ1 | Synaptic transmission |
| 23467 | neuronal pentraxin receptor | NPTXR | Synaptic transmission |
| 27065 | DNA segment on chromosome 4 (unique) 234 | D4S234E | Synaptic transmission |
| | expressed sequence | | |
| 27445 | piccolo (presynaptic cytomatrix protein) | PCLO | Synaptic transmission |
| 6505 | solute carrier family 1 (neuronal/epithelial high | SLC1A1 | Synaptic transmission |
| | affinity glutamate transporter, system Xag), member 1 | | |
| 9456 | homer homolog 1 (Drosophila) | HOMER1 | Synaptic transmission |
| 274 | bridging integrator 1 | BIN1 | Synaptic transmission |
| 1759 | dynamin 1 | DNM1 | Synaptic transmission |
| 10142 | A kinase (PRKA) anchor protein (yotiao) 9 | AKAP9 | Synaptic transmission |
| 25873 | ribosomal protein L36 | RPL36 | Synaptic transmission |
| 50632 | calcyon; D1 dopamine receptor-interacting protein | CALCYON | Synaptic transmission |

TABLE 22

| | Repre- | | | | | | | | DLPFC | | AnCg | | Amy | |
|---|---|---|---|---|---|---|---|---|---|---|---|---|---|---|
| UniGene | sentative | | Chromosomal | | Probe set ID | | Gene | Brain region | p | Fold | p | Fold | p | Fold |
| ID | Public ID | LocusLink | Location | OMIM | UG-1 | Gene Title | Symbol | affected | value | change | value | change | value | change |
| Hs.259768 | AL120173 | 107 | chr7p13-p12 | 103072 | Hs.259768_at | adenylate cyclase 1 (brain) | ADCY1 | DLPFC, | 0.044 | 1.213 | 0.005 | 1.238 | 0.005 | 1.520 |
| | | | | | | | | AnCg, Amy | | | | | | |
| Hs.272891 | AL136591 | 51440 | chr1p34.2 | — | Hs.272891_at | hippocalcin like 4 | HPCAL4 | DLPFC, | 0.046 | 1.243 | 0.010 | 1.326 | 0.007 | 1.458 |
| | | | | | | | | AnCg, Amy | | | | | | |
| Hs.283110 | NM_020178 | 56934 | chr17q21 | 604642 | Hs.283110_at | carbonic anhydrase X | CA10 | DLPFC, | 0.019 | 1.309 | 0.025 | 1.236 | 0.010 | 1.344 |
| | | | | | | | | AnCg, Amy | | | | | | |
| Hs.284394 | NM_000064 | 718 | chr19p13.3-p13.2 | 120700 | Hs.284394_at | complement component 3 | C3 | DLPFC, | 0.039 | 0.613 | 0.026 | 0.637 | 0.006 | 0.419 |
| | | | | | | | | AnCg, Amy | | | | | | |
| Hs.356523 | NM_012324 | 23542 | chr22q13.33 | 607755 | Hs.356523_at | mitogen-activated protein | MAPK8IP2 | DLPFC, | 0.018 | 1.270 | 0.002 | 1.239 | 0.032 | 1.328 |
| | | | | | | kinase 8 interacting protein 2 | | AnCg, Amy | | | | | | |
| Hs.379386 | AA928255 | 115286 | chr3p14.1 | — | Hs.379386_at | solute carrier family 25 | SLC25A26 | DLPFC, | 0.030 | 1.243 | 0.002 | 1.595 | 0.019 | 1.675 |
| | | | | | | (mitochondrial carrier, | | AnCg, Amy | | | | | | |
| | | | | | | phosphate carrier), member 26 | | | | | | | | |
| Hs.429761 | NM_022159 | 64123 | chr1p33-p32 | — | Hs.429761_at | EGF, latrophilin and seven | ELTD1 | DLPFC, | 0.016 | 0.822 | 0.016 | 0.733 | 0.045 | 0.668 |
| | | | | | | transmembrane domain | | AnCg, Amy | | | | | | |
| | | | | | | containing 1 | | | | | | | | |
| Hs.74561 | BF056828 | 2 | chr12p13.3-p12.3 | 103950 | Hs.74561_at | alpha-2-macroglobulin | A2M | DLPFC, | 0.033 | 0.645 | 0.009 | 0.728 | 0.000 | 0.570 |
| | | | | | | | | AnCg, Amy | | | | | | |
| Hs.151032 | BC001072 | 79033 | chr1p32 | — | Hs.151032_at | prion protein interacting protein | PRNPIP | DLPFC, | 0.022 | 1.339 | 0.035 | 1.280 | 0.091 | 1.473 |
| | | | | | | | | AnCg | | | | | | |
| Hs.201920 | X95425 | 2044 | chr4q13.1 | 600004 | Hs.201920_at | EphA5 | EPHA5 | DLPFC, | 0.023 | 1.377 | 0.027 | 1.411 | 0.059 | 1.543 |
| | | | | | | | | AnCg | | | | | | |
| Hs.274404 | NM_000930 | 5327 | chr8p12 | 173370 | Hs.274404_at | plasminogen activator, tissue | PLAT | DLPFC, | 0.016 | 0.639 | 0.019 | 0.697 | 0.164 | 0.763 |
| | | | | | | | | AnCg | | | | | | |
| Hs.288654 | BC001777 | 3208 | chr1p35-p34.2 | 142622 | Hs.288654_at | hippocalcin | HPCA | DLPFC, | 0.025 | 1.300 | 0.049 | 1.233 | 0.056 | 1.366 |
| | | | | | | | | AnCg | | | | | | |
| Hs.418083 | NM_006744 | 5950 | chr10q23-q24 | 180250 | Hs.418083_at | retinol binding protein 4, plasma | RBP4 | DLPFC, | 0.041 | 1.362 | 0.046 | 1.312 | 0.699 | 1.095 |
| | | | | | | | | AnCg | | | | | | |
| Hs.4221 | AL359592 | 55530 | chr12q24.11 | — | Hs.4221_at | hypothetical protein | DKFZp761H039 | DLPFC, | 0.016 | 1.288 | 0.029 | 1.257 | 0.118 | 1.302 |
| | | | | | | DKFZp761H039 | | AnCg | | | | | | |
| Hs.437224 | NM_002894 | 5932 | chr18q11.2 | 604124 | Hs.437224_at | retinoblastoma binding protein 8 | RBBP8 | DLPFC, | 0.020 | 0.830 | 0.005 | 0.774 | 0.666 | 0.921 |
| | | | | | | | | AnCg | | | | | | |
| Hs.527093 | NM_000740 | 1131 | chr1q41-q44 | 118494 | Hs.527093_at | cholinergic receptor, muscarinic 3 | CHRM3 | DLPFC, | 0.024 | 1.369 | 0.031 | 1.312 | 0.240 | 1.094 |
| | | | | | | | | AnCg | | | | | | |
| Hs.155090 | BC011671 | 10681 | chr15q21.2 | 604447 | Hs.155090_at | guanine nucleotide binding | GNB5 | DLPFC, Amy | 0.026 | 1.584 | 0.160 | 1.239 | 0.041 | 1.467 |
| | | | | | | protein (G protein), beta 5 | | | | | | | | |
| Hs.343586 | NM_003407 | 7538 | chr19q13.1 | 190700 | Hs.343586_at | zinc finger protein 36, C3H type, | ZFP36 | DLPFC, Amy | 0.031 | 0.711 | 0.289 | 0.704 | 0.045 | 0.575 |
| | | | | | | homolog (mouse) | | | | | | | | |
| Hs.369441 | NM_014030 | 28964 | chr17p11.2 | 608434 | Hs.369441_at | G protein-coupled receptor | GIT1 | DLPFC, Amy | 0.040 | 1.323 | 0.155 | 1.206 | 0.027 | 1.307 |
| | | | | | | kinase-interactor 1 | | | | | | | | |
| Hs.105352 | Y11339 | 55808 | chr17q25.1 | — | Hs.105352_at | sialyltransferase 7 ((alpha-N- | SIAT7A | AnCg, Amy | 0.258 | 1.076 | 0.033 | 0.783 | 0.036 | 0.702 |
| | | | | | | acetylneuraminyl-2,3-beta- | | | | | | | | |
| | | | | | | galactosyl-1,3)-N-acetyl | | | | | | | | |
| | | | | | | galactosaminide alpha-2,6- | | | | | | | | |
| | | | | | | sialyltransferase) A | | | | | | | | |
| Hs.105468 | NM_024711 | 79765 | chr7q36.1 | — | Hs.105468_at | human Immune associated | hIAN2 | AnCg, Amy | 0.064 | 0.912 | 0.048 | 0.810 | 0.001 | 0.631 |
| | | | | | | nucleotide 2 | | | | | | | | |
| Hs.106674 | AB002534 | 8314 | chr3p21.31-p21.2 | 603089 | Hs.106674_at | BRCA1 associated protein-1 | BAP1 | AnCg, Amy | 0.484 | 1.150 | 0.003 | 1.308 | 0.008 | 1.478 |
| | | | | | | (ubiquitin carboxy-terminal | | | | | | | | |
| | | | | | | hydrolase) | | | | | | | | |
| Hs.108222 | NM_020248 | 56998 | chr1p36.22 | 607758 | Hs.108222_at | catenin, beta interacting protein 1 | CTNNBIP1 | AnCg, Amy | 0.366 | 1.130 | 0.000 | 1.217 | 0.004 | 1.239 |
| Hs.118554 | NM_016027 | 51110 | chr8p22-q22.3 | — | Hs.118554_at | lactamase, beta 2 | CGI-83 | AnCg, Amy | 0.862 | 1.015 | 0.039 | 0.739 | 0.012 | 0.684 |
| Hs.120228 | AA731713 | 23109 | chr12q13.12 | — | Hs.120228_at | dendrin | DDN | AnCg, Amy | 0.508 | 1.085 | 0.024 | 1.276 | 0.014 | 1.348 |
| Hs.124675 | AA858297 | 168537 | chr7q36.1 | — | Hs.124675_at | immune associated nucleotide | hIAN7 | AnCg, Amy | 0.493 | 0.902 | 0.029 | 0.737 | 0.013 | 0.528 |
| Hs.15099 | AB018283 | 9886 | chr10q21.2 | 607351 | Hs.15099_at | Rho-related BTB domain | RHOBTB1 | AnCg, Amy | 0.747 | 1.049 | 0.033 | 0.814 | 0.001 | 0.607 |
| | | | | | | containing 1 | | | | | | | | |
| Hs.169182 | NM_017596 | 23046 | chr1pter-q31.3 | 608322 | Hs.169182_at | kinesin family member 21B | KIF21B | AnCg, Amy | 0.427 | 1.094 | 0.010 | 1.246 | 0.009 | 1.327 |
| Hs.17109 | AL021786 | 9452 | chrxq13.3-xq21.2 | 300222 | Hs.17109_at | integral membrane protein 2A | ITM2A | AnCg, Amy | 0.116 | 0.772 | 0.008 | 0.743 | 0.000 | 0.503 |
| Hs.182536 | AB006622 | 283638 | chr14q32.33 | — | Hs.182536_at | KIAA0284 | KIAA0284 | AnCg, Amy | 0.063 | 1.156 | 0.025 | 1.236 | 0.008 | 1.322 |
| Hs.194720 | AF098951 | 9429 | chr4q22 | 603756 | Hs.194720_at | ATP-binding cassette, sub- | ABCG2 | AnCg, Amy | 0.063 | 0.640 | 0.019 | 0.604 | 0.001 | 0.429 |
| | | | | | | family G (WHITE), member 2 | | | | | | | | |
| Hs.21330 | AF016535 | 5243 | chr7q21.1 | 171050 | Hs.21330_at | ATP-binding cassette, sub- | ABCB1 | AnCg, Amy | 0.052 | 0.810 | 0.013 | 0.745 | 0.001 | 0.528 |
| | | | | | | family B (MDR/TAP), member 1 | | | | | | | | |
| Hs.21611 | AF035621 | 3797 | chr2p23 | 602845 | Hs.21611_at | kinesin family member 3C | KIF3C | AnCg, Amy | 0.176 | 1.279 | 0.042 | 1.248 | 0.034 | 1.448 |
| Hs.22026 | AI721164 | 116441 | chr3q25.1 | — | Hs.22026_at | hypothetical protein BC014339 | LOC116441 | AnCg, Amy | 0.074 | 0.921 | 0.045 | 0.771 | 0.005 | 0.637 |
| Hs.235750 | AF152354 | 26519 | chr11q12.1-q12.3 | 602251 | Hs.235750_at | translocase of inner | TIMM10 | AnCg, Amy | 0.192 | 1.139 | 0.008 | 1.233 | 0.025 | 1.298 |
| | | | | | | mitochondrial membrane 10 | | | | | | | | |
| | | | | | | homolog (yeast) | | | | | | | | |
| Hs.274256 | AW138767 | 79993 | chr5q12.1 | — | Hs.274256_at | hypothetical protein FLJ23563 | FLJ23563 | AnCg, Amy | 0.449 | 0.886 | 0.049 | 0.594 | 0.021 | 0.451 |
| Hs.279909 | AW166283 | 5522 | chr4p16.1 | 605997 | Hs.279909_at | protein phosphatase 2 (formerly | PPP2R2C | AnCg, Amy | 0.019 | 1.188 | 0.012 | 1.214 | 0.008 | 1.373 |
| | | | | | | 2A), regulatory subunit B (PR | | | | | | | | |
| | | | | | | 52), gamma isoform | | | | | | | | |
| Hs.29169 | NM_024610 | 79663 | chr3q21.1 | 608263 | Hs.29169_at | HSPB (heat shock 27 kDa) | HSPBAP1 | AnCg, Amy | 0.048 | 0.863 | 0.007 | 0.660 | 0.011 | 0.737 |
| | | | | | | associated protein 1 | | | | | | | | |
| Hs.303172 | AL120332 | 220164 | chr18q22.2 | — | Hs.303172_at | hypothetical protein MGC20785 | MGC20785 | AnCg, Amy | 0.101 | 1.279 | 0.023 | 1.330 | 0.046 | 1.415 |
| Hs.30822 | NM_018326 | 55303 | chr7q36.1 | 608087 | Hs.30822_at | immunity associated protein 4 | HIMAP4 | AnCg, Amy | 0.078 | 0.857 | 0.010 | 0.817 | 0.003 | 0.484 |
| Hs.311553 | NM_001270 | 1105 | chr5q15-q21 | 602118 | Hs.311553_at | chromodomain helicase DNA | CHD1 | AnCg, Amy | 0.066 | 0.907 | 0.001 | 0.798 | 0.004 | 0.754 |
| | | | | | | binding protein 1 | | | | | | | | |
| Hs.346203 | AK090879 | 55084 | chr6q21 | — | Hs.346203_at | hypothetical protein FLJ10159 | FLJ10159 | AnCg, Amy | 0.022 | 1.135 | 0.016 | 1.206 | 0.002 | 1.324 |
| Hs.348478 | NM_021105 | 5359 | chr3q23 | 604170 | Hs.348478_at | phospholipid scramblase 1 | PLSCR1 | AnCg, Amy | 0.044 | 0.841 | 0.011 | 0.781 | 0.001 | 0.625 |
| Hs.374638 | NM_000801 | 2280 | chr20p13 | 186945 | Hs.374638_at | FK506 binding protein 1A, | FKBP1A | AnCg, Amy | 0.482 | 1.042 | 0.030 | 1.301 | 0.032 | 1.218 |
| | | | | | | 12 kDa | | | | | | | | |
| Hs.37706 | NM_017612 | 55596 | chr12q24.31 | — | Hs.37706_at | hypothetical protein | DKFZp434E2220 | AnCg, Amy | 0.453 | 0.965 | 0.003 | 0.788 | 0.001 | 0.701 |
| | | | | | | DKFZp434E2220 | | | | | | | | |
| Hs.381008 | NM_005516 | 3133 | chr6p21.3 | 143010 | Hs.381008_at | major histocompatibility | HLA-E | AnCg, Amy | 0.219 | 0.843 | 0.005 | 0.806 | 0.000 | 0.725 |
| | | | | | | complex, class I, E | | | | | | | | |
| Hs.381214 | AL136871 | 84221 | chr21q22.3 | — | Hs.381214_at | chromosome 21 open reading | C21orf56 | AnCg, Amy | 0.217 | 1.383 | 0.048 | 1.440 | 0.011 | 1.382 |
| | | | | | | frame 56 | | | | | | | | |
| Hs.388014 | NM_013352 | 29940 | chr6q22 | 605942 | Hs.388014_at | squamous cell carcinoma | SART2 | AnCg, Amy | 0.511 | 0.961 | 0.031 | 0.811 | 0.011 | 0.787 |
| | | | | | | antigen recognized by T cells 2 | | | | | | | | |
| Hs.408302 | AL522296 | 92703 | chr1q32.1 | — | Hs.408302_at | chromosome 1 open reading | C1orf37 | AnCg, Amy | 0.461 | 1.122 | 0.036 | 1.410 | 0.035 | 1.495 |
| | | | | | | frame 37 | | | | | | | | |
| Hs.428112 | BC038383 | 10522 | chr11p15.5 | 602635 | Hs.428112_at | deformed epidermal | DEAF1 | AnCg, Amy | 0.623 | 1.161 | 0.001 | 1.317 | 0.005 | 1.357 |
| | | | | | | autoregulatory factor 1 | | | | | | | | |
| | | | | | | (Drosophila) | | | | | | | | |
| Hs.428446 | AB018195 | 770 | chr19q13.3 | 604644 | Hs.428446_at | carbonic anhydrase XI | CA11 | AnCg, Amy | 0.823 | 1.094 | 0.002 | 1.290 | 0.019 | 1.428 |
| Hs.433303 | NM_000873 | 3384 | chr17q23-q25 | 146630 | Hs.433303_at | Intercellular adhesion molecule 2 | ICAM2 | AnCg, Amy | 0.259 | 0.958 | 0.030 | 0.812 | 0.024 | 0.816 |
| Hs.436200 | AI589086 | 7805 | chr1p34 | 601476 | Hs.436200_at | Lysosomal-associated | LAPTM5 | AnCg, Amy | 0.146 | 0.787 | 0.031 | 0.656 | 0.006 | 0.379 |
| | | | | | | multispanning membrane | | | | | | | | |
| | | | | | | protein-5 | | | | | | | | |
| Hs.445552 | AI539370 | 221424 | chr6p21.1 | — | Hs.445552_at | chromosome 6 open reading | C6orf154 | AnCg, Amy | 0.294 | 1.291 | 0.030 | 1.377 | 0.033 | 1.621 |
| | | | | | | frame 154 | | | | | | | | |
| Hs.6434 | NM_020215 | 56967 | chr14q32.2 | — | Hs.6434_at | chromosome 14 open reading | C14orf132 | AnCg, Amy | 0.935 | 1.022 | 0.015 | 1.219 | 0.003 | 1.370 |
| | | | | | | frame 132 | | | | | | | | |
| Hs.71791 | NM_013313 | 29799 | chr22q11.2 | 608082 | Hs.71791_at | yippee-like 1 (Drosophila) | YPEL1 | AnCg, Amy | 0.020 | 1.139 | 0.006 | 1.266 | 0.017 | 1.468 |
| Hs.74624 | NM_002847 | 5799 | chr7q36 | 601698 | Hs.74624_at | protein tyrosine phosphatase, | PTPRN2 | AnCg, Amy | 0.164 | 1.184 | 0.011 | 1.269 | 0.036 | 1.262 |
| | | | | | | receptor type, N polypeptide 2 | | | | | | | | |
| Hs.75262 | AV729484 | 1519 | chr4q31-q32 | 600550 | Hs.75262_at | cathepsin O | CTSO | AnCg, Amy | 0.069 | 0.865 | 0.034 | 0.696 | 0.027 | 0.687 |
| Hs.80658 | U82819 | 7351 | chr11q13 | 601693 | Hs.80658_at | uncoupling protein 2 | UCP2 | AnCg, Amy | 0.356 | 0.915 | 0.000 | 0.745 | 0.003 | 0.786 |
| | | | | | | (mitochondrial, proton carrier) | | | | | | | | |
| Hs.94953 | AI184968 | 714 | chr1p36.11 | 120575 | Hs.94953_at | complement component 1, q | C1QG | AnCg, Amy | 0.199 | 0.869 | 0.026 | 0.558 | 0.003 | 0.339 |
| | | | | | | subcomponent, gamma | | | | | | | | |
| | | | | | | polypeptide | | | | | | | | |
| Hs.9963 | NM_003332 | 7305 | chr19q13.1 | 604142 | Hs.9963_at | TYRO protein tyrosine kinase | TYROBP | AnCg, Amy | 0.784 | 1.027 | 0.033 | 0.796 | 0.031 | 0.563 |
| | | | | | | binding protein | | | | | | | | |
| Hs.100914 | NM_018069 | 55125 | chr18p11.21 | — | Hs.100914_at | hypothetical protein FLJ10352 | FLJ10352 | DLPFC | 0.034 | 1.284 | 0.702 | 1.057 | 0.542 | 0.911 |
| Hs.10119 | AB051536 | 84952 | chr15q21.3 | 607856 | Hs.10119_at | hypothetical protein FLJ14957 | FLJ14957 | DLPFC | 0.030 | 0.671 | 0.744 | 0.955 | 0.480 | 0.876 |
| Hs.106552 | AC005378 | 26047 | chr7q35-q36 | 604569 | Hs.106552_at | contactin associated protein-like 2 | CNTNAP2 | DLPFC | 0.014 | 1.247 | 0.303 | 1.121 | 0.083 | 1.273 |
| Hs.109760 | NM_002491 | 4709 | chr2q31.3 | 603839 | Hs.109760_at | NADH dehydrogenase | NDUFB3 | DLPFC | 0.032 | 1.210 | 0.327 | 1.111 | 0.283 | 1.165 |
| | | | | | | (ubiquinone) 1 beta | | | | | | | | |
| | | | | | | subcomplex, 3, 12 kDa | | | | | | | | |
| Hs.11614 | BG054922 | 29070 | chr16q21 | — | Hs.11614_at | HSPC065 protein | HSPC065 | DLPFC | 0.036 | 1.216 | 0.061 | 1.158 | 0.594 | 1.078 |
| Hs.117865 | NM_012434 | 26503 | chr6q14-q15 | 604322 | Hs.117865_at | solute carrier family 17 | SLC17A5 | DLPFC | 0.007 | 0.714 | 0.937 | 1.014 | 0.955 | 0.995 |
| | | | | | | (anion/sugar transporter), | | | | | | | | |
| | | | | | | member 5 | | | | | | | | |
| Hs.118684 | NM_006923 | 6388 | chr17q11.2 | 602934 | Hs.118684_at | stromal cell-derived factor 2 | SDF2 | DLPFC | 0.042 | 1.239 | 0.198 | 1.550 | 0.472 | 1.177 |
| Hs.12313 | BE856822 | 84892 | chr3p22.1 | — | Hs.12313_at | hypothetical protein FLJ14566 | FLJ14566 | DLPFC | 0.045 | 1.214 | 0.165 | 1.140 | 0.149 | 1.156 |
| Hs.12887 | BC015207 | 57180 | chr7q32-q36 | — | Hs.12887_at | actin-related protein 3-beta | ARP3BETA | DLPFC | 0.046 | 1.272 | 0.069 | 1.272 | 0.158 | 1.330 |
| Hs.13423 | AI634532 | 138046 | chr8q21.2 | — | Hs.13423_at | hypothetical protein LOC138046 | LOC138046 | DLPFC | 0.032 | 1.291 | 0.249 | 1.104 | 0.058 | 1.372 |
| Hs.134640 | AI082251 | 3888 | chr12q13 | 601078 | Hs.134640_at | keratin, hair, basic, 2 | KRTHB2 | DLPFC | 0.023 | 0.738 | 0.098 | 0.790 | 0.119 | 0.829 |
| Hs.13885 | BC003353 | 84246 | chr5p15.31 | — | Hs.13885_at | hypothetical protein MGC5309 | MGC5309 | DLPFC | 0.017 | 1.259 | 0.162 | 1.198 | 0.782 | 1.045 |
| Hs.139226 | M87338 | 5982 | chr7q11.23 | 600404 | Hs.139226_at | replication factor C (activator 1) | RFC2 | DLPFC | 0.026 | 1.273 | 0.150 | 1.198 | 0.499 | 1.098 |
| | | | | | | 2, 40 kDa | | | | | | | | |
| Hs.151408 | AL535113 | 5332 | chr20p12 | 600810 | Hs.151408_at | phospholipase C, beta 4 | PLCB4 | DLPFC | 0.039 | 1.351 | 0.367 | 1.188 | 0.933 | 1.013 |
| Hs.157236 | BC001913 | 10493 | chr17q21 | 604631 | Hs.157236_at | vesicle amine transport protein | VAT1 | DLPFC | 0.033 | 0.823 | 0.379 | 0.924 | 0.224 | 0.857 |
| | | | | | | 1 homolog (T. californica) | | | | | | | | |
| Hs.158748 | BF968270 | 148641 | chr1q42.2 | — | Hs.158748_at | solute carrier family 35, member | SLC35F3 | DLPFC | 0.032 | 1.303 | 0.779 | 1.036 | 0.130 | 1.386 |
| | | | | | | F3 | | | | | | | | |
| Hs.159161 | D13989 | 396 | chr17q25.3 | 601925 | Hs.159161_at | Rho GDP dissociation inhibitor | ARHGDIA | DLPFC | 0.003 | 1.243 | 0.069 | 1.132 | 0.424 | 1.092 |
| | | | | | | (GDI) alpha | | | | | | | | |
| Hs.166351 | NM_014906 | 22843 | chr17q23.2 | — | Hs.166351_at | protein phosphatase 1E (PP2C | PPM1E | DLPFC | 0.016 | 1.273 | 0.396 | 1.118 | 0.549 | 1.118 |
| | | | | | | domain containing) | | | | | | | | |
| Hs.170673 | AI440266 | 195814 | chr8q12.1 | — | Hs.170673_at | retinal short chain | RDH-E2 | DLPFC | 0.025 | 1.291 | 0.183 | 1.249 | 0.582 | 1.126 |
| | | | | | | dehydrogenase reductase | | | | | | | | |
| Hs.171835 | NM_018180 | 55760 | chr10q26.2 | 607960 | Hs.171835_at | DEAD/H (Asp-Glu-Ala-Asp/His) | DDX32 | DLPFC | 0.000 | 0.752 | 0.157 | 0.929 | 0.669 | 1.036 |
| | | | | | | box polypeptide 32 | | | | | | | | |
| Hs.172589 | BE796924 | 11137 | chr12q23.3 | — | Hs.172589_at | nuclear phosphoprotein similar | PWP1 | DLPFC | 0.013 | 1.275 | 0.562 | 1.058 | 0.343 | 1.089 |
| | | | | | | to S. cerevisiae PWP1 | | | | | | | | |
| Hs.182626 | NM_012264 | 25829 | chr22q12 | — | Hs.182626_at | chromosome 22 open reading | C22orf5 | DLPFC | 0.029 | 1.212 | 0.940 | 1.007 | 0.257 | 1.121 |
| | | | | | | frame 5 | | | | | | | | |
| Hs.198288 | NM_002849 | 5801 | chr12q15 | 602853 | Hs.198288_at | protein tyrosine phosphatase, | PTPRR | DLPFC | 0.043 | 1.396 | 0.395 | 1.179 | 0.382 | 1.280 |
| | | | | | | receptor type, R | | | | | | | | |
| Hs.200666 | AL548363 | 9270 | chr2p25.2 | 607153 | Hs.200666_at | integrin beta 1 binding protein 1 | ITGB1BP1 | DLPFC | 0.028 | 1.410 | 0.094 | 1.333 | 0.276 | 1.281 |
| Hs.209983 | NM_005563 | 3925 | chr1p36.1-p35 | 151442 | Hs.209983_at | stathmin 1/oncoprotein 18 | STMN1 | DLPFC | 0.010 | 1.271 | 0.651 | 1.038 | 0.344 | 1.200 |
| Hs.21577 | NM_005701 | 10073 | chr15q23 | 607902 | Hs.21577_at | RNA, U transporter 1 | RNUT1 | DLPFC | 0.023 | 1.205 | 0.120 | 1.213 | 0.185 | 1.232 |
| Hs.21943 | NM_021824 | 60491 | chr2q33 | 605778 | Hs.21943_at | NIF3 NGG1 interacting factor 3- | NIF3L1 | DLPFC | 0.011 | 1.564 | 0.876 | 1.014 | 0.853 | 1.024 |
| | | | | | | like 1 (S. pombe) | | | | | | | | |
| Hs.223296 | NM_017861 | 54965 | chr3q29 | — | Hs.223296_at | hypothetical protein FLJ20522 | FLJ20522 | DLPFC | 0.003 | 1.231 | 0.375 | 1.085 | 0.261 | 1.154 |
| Hs.22791 | AB017269 | 23671 | chr2q32.3 | 605734 | Hs.22791_at | transmembrane protein with | TMEFF2 | DLPFC | 0.032 | 1.394 | 0.894 | 1.017 | 0.874 | 0.967 |
| | | | | | | EGF-like and two follistatin-like | | | | | | | | |
| | | | | | | domains 2 | | | | | | | | |
| Hs.2281 | NM_001819 | 1114 | chr20pter-p12 | 118920 | Hs.2281_at | chromogranin B (secretogranin | CHGB | DLPFC | 0.046 | 1.389 | 0.350 | 1.172 | 0.520 | 1.195 |
| | | | | | | 1) | | | | | | | | |
| Hs.237517 | AV703769 | 25791 | chr2q37 | 605991 | Hs.237517_at | neuronal guanine nucleotide | NGEF | DLPFC | 0.007 | 1.361 | 0.107 | 1.086 | 0.115 | 1.224 |
| | | | | | | exchange factor | | | | | | | | |
| Hs.239758 | NM_023928 | 65985 | chr12q24.31 | — | Hs.239758_at | acetoacetyl-CoA synthetase | AACS | DLPFC | 0.034 | 1.207 | 0.511 | 1.064 | 0.146 | 1.295 |
| Hs.241523 | NM_018008 | 55079 | chr3p14.2 | 607414 | Hs.241523_at | likely ortholog of mouse and | FEZL | DLPFC | 0.004 | 1.238 | 0.680 | 1.049 | 0.164 | 1.291 |
| | | | | | | zebrafish forebrain embryonic | | | | | | | | |
| | | | | | | zinc finger-like | | | | | | | | |
| Hs.24970 | AV724323 | 116442 | chrxq28 | — | Hs.24970_at | RAB39B, member RAS | RAB39B | DLPFC | 0.026 | 1.287 | 0.239 | 1.140 | 0.381 | 1.144 |
| | | | | | | oncogene family | | | | | | | | |
| Hs.24979 | NM_018423 | 55359 | chr12p13.2 | — | Hs.24979_at | hypothetical protein | DKFZp761P1010 | DLPFC | 0.028 | 1.284 | 0.184 | 1.313 | 0.194 | 1.322 |
| | | | | | | DKFZp761P1010 | | | | | | | | |
| Hs.255149 | NM_005907 | 4121 | chr6q22 | 604344 | Hs.255149_at | mannosidase, alpha, class 1A, | MAN1A1 | DLPFC | 0.032 | 1.203 | 0.434 | 1.099 | 0.813 | 1.042 |
| | | | | | | member 1 | | | | | | | | |
| Hs.27160 | R75637 | 113444 | chr1p34.3 | — | Hs.27160_at | hypothetical protein BC011880 | LOC113444 | DLPFC | 0.020 | 1.261 | 0.384 | 1.105 | 0.399 | 1.140 |
| Hs.27524 | BF673779 | 132332 | chr4q27 | — | Hs.27524−_at | hypothetical protein FLJ30834 | FLJ30834 | DLPFC | 0.040 | 1.274 | 0.935 | 1.011 | 0.555 | 1.314 |
| Hs.279939 | AF189289 | 23787 | chr6pter-p24.1 | — | Hs.279939_at | mitochondrial carrier homolog 1 | MTCH1 | DLPFC | 0.037 | 1.303 | 0.054 | 1.137 | 0.269 | 1.158 |
| | | | | | | (C. elegans) | | | | | | | | |
| Hs.283393 | AL575735 | 1290 | chr2q14-q32 | 120190 | Hs.283393_at | collagen, type V, alpha 2 | COL5A2 | DLPFC | 0.018 | 1.255 | 0.677 | 1.039 | 0.814 | 1.014 |
| Hs.285280 | AA534210 | 122830 | chr14q22.3 | — | Hs.285280+_at | chromosome 14 open reading | C14orf35 | DLPFC | 0.021 | 1.239 | 0.258 | 1.136 | 0.076 | 1.272 |
| | | | | | | frame 35 | | | | | | | | |
| Hs.286173 | AB046815 | 57696 | chr12q24.31 | — | Hs.286173_at | DEAD (Asp-Glu-Ala-Asp) box | DDX55 | DLPFC | 0.005 | 1.258 | 0.360 | 1.057 | 0.888 | 0.988 |
| | | | | | | polypeptide 55 | | | | | | | | |
| Hs.291070 | NM_138687 | 8396 | chr17q12 | 603261 | Hs.291070_at | phosphatidylinositol-4- | PIP5K2B | DLPFC | 0.013 | 1.254 | 0.118 | 1.207 | 0.092 | 1.169 |
| | | | | | | phosphate 5-kinase, type II, | | | | | | | | |
| | | | | | | beta | | | | | | | | |
| Hs.29222 | NM_003427 | 7629 | chr6p21.3-p21.2 | 194549 | Hs.29222_at | zinc finger protein 76 | ZNF76 | DLPFC | 0.034 | 1.216 | 0.507 | 1.080 | 0.967 | 1.003 |
| | | | | | | (expressed in testis) | | | | | | | | |
| Hs.29344 | AF070530 | 148022 | chr19p13.3 | 607601 | Hs.29344_at | TIR domain containing adaptor | TRIF | DLPFC | 0.023 | 1.297 | 0.324 | 1.132 | 0.394 | 1.095 |
| | | | | | | inducing interferon-beta | | | | | | | | |
| Hs.300670 | AB033030 | 57514 | chr3q13.32-q13.33 | — | Hs.300670_at | KIAA1204 protein | CDGAP | DLPFC | 0.042 | 0.808 | 0.556 | 0.952 | 0.653 | 0.945 |
| Hs.302460 | NM_021930 | 60561 | chr7q22.3 | — | Hs.302460_at | Rad50-interacting protein 1 | FLJ11785 | DLPFC | 0.017 | 1.303 | 0.881 | 0.988 | 0.676 | 0.937 |
| Hs.30991 | BE677131 | 22881 | chr6q14.2-q16.1 | — | Hs.30991_at | ankyrin repeat domain 6 | ANKRD6 | DLPFC | 0.041 | 1.257 | 0.058 | 1.328 | 0.282 | 1.174 |
| Hs.317335 | NM_006012 | 8192 | chr19p13.3 | 601119 | Hs.317335_at | ClpP caseinolytic protease, | CLPP | DLPFC | 0.047 | 1.294 | 0.106 | 1.144 | 0.462 | 1.101 |
| | | | | | | ATP-dependent, proteolytic | | | | | | | | |
| | | | | | | subunit homolog (E. coli) | | | | | | | | |
| Hs.321501 | NM_025238 | 53339 | chr15q24 | 608530 | Hs.321501_at | BTB (POZ) domain containing 1 | BTBD1 | DLPFC | 0.018 | 1.331 | 0.179 | 1.130 | 0.436 | 1.101 |
| Hs.323537 | AK023015 | 84058 | chr2p13.1 | — | Hs.323537_at | hypothetical protein FLJ12953 | FLJ12953 | DLPFC | 0.046 | 1.220 | 0.588 | 1.070 | 0.592 | 1.119 |
| | | | | | | similar to Mus musculus | | | | | | | | |
| | | | | | | D3Mm3e | | | | | | | | |
| Hs.32539 | AB040812 | 57144 | chr20p12 | 608038 | Hs.32539_at | p21(CDKN1A)-activated kinase 7 | PAK7 | DLPFC | 0.009 | 1.223 | 0.679 | 1.039 | 0.069 | 1.348 |
| Hs.333118 | AL136879 | 84222 | chr22q11.21 | — | Hs.333118_at | hypothetical protein | DKFZp434N035 | DLPFC | 0.038 | 1.698 | 0.502 | 1.214 | 0.254 | 1.331 |
| | | | | | | DKFZp434N035 | | | | | | | | |
| Hs.335433 | AF397394 | 118427 | chr1p22 | 607567 | Hs.335433_at | olfactomedin 3 | OLFM3 | DLPFC | 0.022 | 1.340 | 0.760 | 1.031 | 0.108 | 1.369 |
| Hs.348446 | W37431 | 5601 | chr5q35 | 602896 | Hs.348446_at | mitogen-activated protein | MAPK9 | DLPFC | 0.047 | 1.280 | 0.196 | 1.108 | 0.320 | 1.222 |
| | | | | | | kinase 9 | | | | | | | | |
| Hs.349111 | AL022237 | 27349 | chr22q13.31 | — | Hs.349111_at | malonyl-CoA: acyl carrier protein | MT | DLPFC | 0.041 | 1.375 | 0.309 | 1.205 | 0.157 | 1.303 |
| | | | | | | transacylase | | | | | | | | |
| | | | | | | (malonyltransferase) | | | | | | | | |
| Hs.349695 | | | | | Hs.349695_at | tubulin, alpha 2 | TUBA2 | DLPFC | 0.009 | 1.346 | 0.086 | 1.136 | 0.245 | 1.146 |
| Hs.350065 | | | | | Hs.350065_at | hypothetical protein FLJ30634 | FLJ30634 | DLPFC | 0.031 | 0.801 | 0.516 | 0.933 | 0.229 | 1.180 |
| Hs.355141 | NM_006058 | 10318 | chr5q32-q33.1 | 607714 | Hs.355141_at | TNFAIP3 interacting protein 1 | TNIP1 | DLPFC | 0.043 | 1.358 | 0.832 | 1.012 | 0.281 | 1.055 |
| Hs.355281 | NM_003586 | 8448 | chr16p11.2 | 604567 | Hs.355281_at | double C2-like domains, alpha | DOC2A | DLPFC | 0.015 | 1.400 | 0.493 | 1.102 | 0.090 | 1.515 |
| Hs.356358 | BC014311 | 115548 | chr5q13.2 | — | Hs.356358_at | hypothetical protein BC014311 | LOC115548 | DLPFC | 0.007 | 0.833 | 0.995 | 1.000 | 0.076 | 0.832 |
| Hs.362806 | BF941499 | 221395 | chr6p12.3 | — | Hs.362806_at | G protein-coupled receptor 116 | GPR116 | DLPFC | 0.039 | 0.792 | 0.061 | 0.883 | 0.308 | 0.887 |
| Hs.36761 | NM_020386 | 57110 | chr3q29 | 606487 | Hs.36761_at | HRAS-like suppressor | HRASLS | DLPFC | 0.031 | 1.241 | 0.227 | 1.185 | 0.268 | 1.169 |
| Hs.380887 | NM_018141 | 55173 | chr6p21.1-p12.1 | — | Hs.380887_at | mitochondrial ribosomal protein | MRPS10 | DLPFC | 0.033 | 1.243 | 0.545 | 1.081 | 0.278 | 1.133 |
| | | | | | | S10 | | | | | | | | |
| Hs.386392 | BC000009 | 55651 | chr5q35.3 | 606470 | Hs.386392_at | nucleolar protein family A, | NOLA2 | DLPFC | 0.014 | 1.291 | 0.134 | 1.220 | 0.218 | 1.230 |
| | | | | | | member 2 (H/ACA small | | | | | | | | |
| | | | | | | nucleolar RNPs) | | | | | | | | |
| Hs.40510 | NM_004277 | 9481 | chr6p11.2-q12 | — | Hs.40510_at | solute carrier family 25, member | SLC25A27 | DLPFC | 0.005 | 1.408 | 0.148 | 1.191 | 0.192 | 1.269 |
| | | | | | | 27 | | | | | | | | |
| Hs.4082 | AI659005 | 3964 | chr1q42-q43 | 606099 | Hs.4082+_at | lectin, galactoside-binding, | LGALS8 | DLPFC | 0.032 | 1.201 | 0.990 | 0.999 | 0.776 | 1.038 |
| | | | | | | soluble, 8 (galectin 8) | | | | | | | | |
| Hs.410618 | BC034626 | 9675 | chr20q12 | — | Hs.410618_at | KIAA0406 gene product | KIAA0406 | DLPFC | 0.030 | 1.221 | 0.551 | 1.055 | 0.618 | 1.058 |
| Hs.410745 | AB014486 | 8935 | chr7p21-p15 | 605215 | Hs.410745_at | src family associated | SCAP2 | DLPFC | 0.036 | 1.288 | 0.343 | 1.100 | 0.723 | 1.060 |
| | | | | | | phosphoprotein 2 | | | | | | | | |
| Hs.410748 | NM_022003 | 53826 | chr11q23.3 | 606683 | Hs.410748_at | FXYD domain containing ion | FXYD6 | DLPFC | 0.016 | 1.349 | 0.119 | 1.188 | 0.387 | 1.157 |
| | | | | | | transport regulator 6 | | | | | | | | |
| Hs.416061 | BQ894022 | 5136 | chr2q32.1 | 171890 | Hs.416061_at | phosphodiesterase 1A, | PDE1A | DLPFC | 0.038 | 1.598 | 0.274 | 1.253 | 0.324 | 1.405 |
| | | | | | | calmodulin-dependent | | | | | | | | |
| Hs.416216 | NM_007240 | 11266 | chr1q21-q22 | 604835 | Hs.416216_at | dual specificity phosphatase 12 | DUSP12 | DLPFC | 0.025 | 1.284 | 0.303 | 1.138 | 0.843 | 0.966 |
| Hs.419151 | NM_017917 | 55012 | chr14q13.2 | — | Hs.419151_at | chromosome 14 open reading | C14orf10 | DLPFC | 0.046 | 1.338 | 0.098 | 1.401 | 0.347 | 1.180 |
| | | | | | | frame 10 | | | | | | | | |
| Hs.419240 | AI631159 | 6515 | chr12p13.3 | 138170 | Hs.419240_at | solute carrier family 2 (facilitated | SLC2A3 | DLPFC | 0.009 | 1.352 | 0.505 | 1.066 | 0.820 | 1.048 |
| | | | | | | glucose transporter), member 3 | | | | | | | | |
| Hs.421202 | AF327657 | 20 | chr9q34 | 600047 | Hs.421202_at | ATP-binding cassette, sub- | ABCA2 | DLPFC | 0.048 | 1.229 | 0.720 | 1.065 | 0.215 | 0.771 |
| | | | | | | family A (ABC1), member 2 | | | | | | | | |
| Hs.422662 | NM_003384 | 7443 | chr14q32 | 602168 | Hs.422662_at | vaccinia related kinase 1 | VRK1 | DLPFC | 0.019 | 1.466 | 0.867 | 1.013 | 0.446 | 1.153 |
| Hs.422688 | AI733027 | 116362 | chr1p36.22 | 608604 | Hs.422688_at | retinoid binding protein 7 | CRBPIV | DLPFC | 0.045 | 1.241 | 0.916 | 1.036 | 0.122 | 0.639 |
| Hs.423348 | NM_000244 | 4221 | chr11q13 | 131100 | Hs.423348_at | multiple endocrine neoplasia I | MEN1 | DLPFC | 0.008 | 0.795 | 0.925 | 1.006 | 0.545 | 1.072 |
| Hs.424551 | BC000027 | 23423 | chr15q24-q25 | — | Hs.424551_at | integral type I protein | P24B | DLPFC | 0.050 | 1.262 | 0.203 | 1.146 | 0.404 | 1.109 |
| Hs.426324 | AU158251 | 7991 | chr8p22 | 601385 | Hs.426324_at | Putative prostate cancer tumor | N33 | DLPFC | 0.008 | 1.320 | 0.162 | 1.169 | 0.530 | 1.084 |
| | | | | | | suppressor | | | | | | | | |
| Hs.43112 | NM_173517 | 154807 | chr7q11.21 | 608838 | Hs.43112_at | hypothetical protein LOC154807 | LOC154807 | DLPFC | 0.044 | 1.254 | 0.020 | 1.118 | 0.145 | 1.124 |
| Hs.433326 | NM_000597 | 3485 | chr2q33-q34 | 146731 | Hs.433326_at | insulin-like growth factor binding | IGFBP2 | DLPFC | 0.044 | 1.451 | 0.867 | 0.968 | 0.359 | 1.147 |
| | | | | | | protein 2, 36 kDa | | | | | | | | |
| Hs.435342 | NM_006425 | 10569 | chr5q33.3 | 605974 | Hs.435342_at | step II splicing factor SLU7 | SLU7 | DLPFC | 0.034 | 1.223 | 0.294 | 1.097 | 0.316 | 1.113 |
| Hs.440950 | AK021433 | 25844 | chr6p21.1 | — | Hs.440950_at | chromosome 6 open reading | C6orf109 | DLPFC | 0.006 | 1.235 | 0.046 | 1.124 | 0.417 | 1.107 |
| | | | | | | frame 109 | | | | | | | | |
| Hs.443683 | NM_003970 | 9172 | chr8p23.3 | 603509 | Hs.443683_at | myomesin (M-protein) 2, | MYOM2 | DLPFC | 0.020 | 0.742 | 0.487 | 0.914 | 0.539 | 1.078 |
| | | | | | | 165 kDa | | | | | | | | |
| Hs.444846 | AU147317 | 55831 | chr3p25.3 | — | Hs.444846_at | 30 kDa protein | LOC55831 | DLPFC | 0.021 | 1.284 | 0.233 | 1.114 | 0.242 | 1.140 |
| Hs.445132 | NM_145257 | 126731 | chr1q42.13 | — | Hs.445132_at | LOC126731 | LOC126731 | DLPFC | 0.007 | 1.229 | 0.028 | 1.099 | 0.185 | 1.065 |
| Hs.458268 | | | | | Hs.458268_at | potassium inwardly-rectifying | KCNJ6 | DLPFC | 0.043 | 1.241 | 0.105 | 1.190 | 0.098 | 1.217 |
| | | | | | | channel, subfamily J, member 6 | | | | | | | | |
| Hs.4742 | NM_003801 | 8733 | chr8q24.3 | 603048 | Hs.4742_at | GPAA1P anchor attachment | GPAA1 | DLPFC | 0.009 | 1.454 | 0.107 | 1.222 | 0.262 | 1.222 |
| | | | | | | protein 1 homolog (yeast) | | | | | | | | |
| Hs.4817 | NM_002545 | 4978 | chr11q25 | 600632 | Hs.4817_at | oploid binding protein/cell | OPCML | DLPFC | 0.013 | 1.293 | 0.493 | 1.052 | 0.155 | 1.253 |
| | | | | | | adhesion molecule-like | | | | | | | | |
| Hs.500495 | W68731 | 374819 | chr17q24.2 | — | Hs.500495_at | FLJ34306 protein | FLJ34306 | DLPFC | 0.038 | 1.610 | 0.234 | 1.149 | 0.614 | 1.121 |
| Hs.5019 | BC004344 | 91782 | chr8p21.3 | — | Hs.5019_at | hypothetical protein MGC29816 | MGC29816 | DLPFC | 0.045 | 1.271 | 0.299 | 1.053 | 0.819 | 1.015 |
| Hs.511768 | NM_007234 | 11258 | chr9p13 | 607387 | Hs.511768_at | dynactin 3 (p22) | DCTN3 | DLPFC | 0.034 | 1.277 | 0.180 | 1.158 | 0.332 | 1.175 |
| Hs.511974 | CA411757 | 255403 | chr4p16.3 | — | Hs.511974_at | hypothetical protein FLJ90036 | FLJ90036 | DLPFC | 0.035 | 0.789 | 0.382 | 0.927 | 0.186 | 0.882 |
| Hs.512644 | AF249278 | 56479 | chr6q14 | 607357 | Hs.512644_at | potassium voltage-gated | KCNQ5 | DLPFC | 0.041 | 1.560 | 0.300 | 1.266 | 0.219 | 1.042 |
| | | | | | | channel, KQT-like subfamily, | | | | | | | | |
| | | | | | | member 5 | | | | | | | | |
| Hs.54886 | AL117515 | 23228 | chr3p24.3 | — | Hs.54886_at | phospholipase C-like 2 | PLCL2 | DLPFC | 0.019 | 1.228 | 0.103 | 1.119 | 0.245 | 1.122 |
| Hs.7117 | AL567302 | 2890 | chr5q31.1 | 138248 | Hs.7117_at | glutamate receptor, ionotropic, | GRIA1 | DLPFC | 0.043 | 1.328 | 0.266 | 1.112 | 0.673 | 1.062 |
| | | | | | | AMPA 1 | | | | | | | | |
| Hs.76206 | NM_001795 | 1003 | chr16q22.1 | 601120 | Hs.76206_at | cadherin 5, type 2, VE-cadherin | CDH5 | DLPFC | 0.042 | 0.779 | 0.229 | 0.874 | 0.054 | 0.777 |
| | | | | | | (vascular epithelium) | | | | | | | | |
| Hs.77917 | NM_006002 | 7347 | chr13q22.2 | 603090 | Hs.77917_at | ubiquitin carboxyl-terminal | UCHL3 | DLPFC | 0.003 | 1.408 | 0.634 | 1.047 | 0.472 | 0.887 |
| | | | | | | esterase L3 (ubiquitin | | | | | | | | |
| | | | | | | thiolesterase) | | | | | | | | |
| Hs.80296 | NM_006198 | 5121 | chr21q22.2 | 601629 | Hs.80296_at | Purkinje cell protein 4 | PCP4 | DLPFC | 0.028 | 1.397 | 0.129 | 1.196 | 0.909 | 0.970 |
| Hs.8040 | AF105378 | 9951 | chr16p11.2 | 604059 | Hs.8040_at | heparan sulfate (glucosamine) | HS3ST4 | DLPFC | 0.048 | 1.269 | 0.072 | 1.221 | 0.200 | 1.512 |
| | | | | | | 3-O-sulfotransferase 4 | | | | | | | | |
| Hs.83753 | J04564 | 6628 | chr20p13 | 182282 | Hs.83753_at | small nuclear ribonucleoprotein | SNRPB | DLPFC | 0.043 | 1.258 | 0.173 | 1.192 | 0.251 | 1.255 |
| | | | | | | polypeptides B and B1 | | | | | | | | |
| Hs.84120 | BC006123 | 84303 | chr3q21.2 | — | Hs.84120_at | hypothetical protein MGC13016 | MGC13016 | DLPFC | 0.010 | 1.234 | 0.135 | 1.239 | 0.092 | 1.441 |
| Hs.85539 | NM_007100 | 521 | chr4p16.3 | 601519 | Hs.85539_at | ATP synthase, H+ transporting, | ATP5I | DLPFC | 0.004 | 1.209 | 0.184 | 1.132 | 0.869 | 0.984 |
| | | | | | | mitochondrial F0 complex, | | | | | | | | |
| | | | | | | subunit e | | | | | | | | |
| Hs.9003 | NM_022744 | 64755 | chr16p11.2 | — | Hs.9003_at | hypothetical protein FLJ13868 | FLJ13868 | DLPFC | 0.020 | 1.218 | 0.211 | 1.185 | 0.607 | 1.025 |
| Hs.91390 | NM_003631 | 8505 | chr10q11.23 | 603501 | Hs.91390_at | poly (ADP-ribose) | PARG | DLPFC | 0.040 | 1.257 | 0.325 | 1.127 | 0.887 | 1.022 |
| | | | | | | glycohydrolase | | | | | | | | |
| Hs.93872 | NM_019061 | 54545 | chr5p13.3 | 606501 | Hs.93872_at | phosphatidylinositol-3- | PIP3AP | DLPFC | 0.012 | 1.240 | 0.017 | 1.131 | 0.286 | 1.085 |
| | | | | | | phosphate associated protein | | | | | | | | |
| Hs.98493 | NM_006297 | 7515 | chr19q13.2 | 194360 | Hs.98493_at | X-ray repair complementing | XRCC1 | DLPFC | 0.033 | 1.355 | 0.393 | 1.117 | 0.903 | 0.989 |
| | | | | | | defective repair in Chinese | | | | | | | | |
| | | | | | | hamster cells 1 | | | | | | | | |
| Hs.102456 | NM_003616 | 8487 | chr14q13 | 602595 | Hs.102456_at | survival of motor neuron protein | SIP1 | AnCg | 0.042 | 1.193 | 0.003 | 1.246 | 0.998 | 1.000 |
| | | | | | | interacting protein 1 | | | | | | | | |
| Hs.104576 | NM_003654 | 8534 | chr11p11.2-p11.1 | 603797 | Hs.104576_at | carbohydrate (keratan sulfate | CHST1 | AnCg | 0.313 | 1.345 | 0.011 | 1.259 | 0.257 | 1.215 |
| | | | | | | Gal-6) sulfotransferase 1 | | | | | | | | |
| Hs.110488 | NM_014918 | 22856 | chr15q26.3 | 608183 | Hs.110488_at | carbohydrate (chondroitin) | CHSY1 | AnCg | 0.848 | 0.975 | 0.017 | 0.723 | 0.224 | 0.839 |
| | | | | | | synthase 1 | | | | | | | | |
| Hs.115721 | NM_013247 | 27429 | chr2p12 | 606441 | Hs.115721_at | protease, serine, 25 | PRSS25 | AnCg | 0.785 | 1.014 | 0.013 | 1.206 | 0.284 | 1.097 |
| Hs.1176 | NM_005070 | 6508 | chr2q36 | 106195 | Hs.1176_at | solute carrier family 4, anion | SLC4A3 | AnCg | 0.949 | 1.008 | 0.022 | 1.227 | 0.282 | 1.168 |
| | | | | | | exchanger, member 3 | | | | | | | | |
| Hs.119302 | AF329838 | 114900 | chr11q11 | — | Hs.119302_at | C1q and tumor necrosis factor | C1QTNF4 | AnCg | 0.425 | 1.158 | 0.042 | 1.313 | 0.092 | 1.258 |
| | | | | | | related protein 4 | | | | | | | | |
| Hs.119598 | NM_000967 | 6122 | chr22q13 | 604163 | Hs.119598_at | ribosomal protein L3 | RPL3 | AnCg | 0.626 | 1.321 | 0.008 | 1.261 | 0.863 | 1.015 |
| Hs.12751 | R46128 | 2849 | chr10q26.2 | 604847 | Hs.12751_at | G protein-coupled receptor 26 | GPR26 | AnCg | 0.900 | 1.020 | 0.025 | 1.286 | 0.191 | 1.155 |
| Hs.130979 | NM_173528 | 161502 | chr15q25.1 | — | Hs.130979_at | hypothetical protein FLJ38615 | FLJ38615 | AnCg | 0.174 | 0.864 | 0.043 | 1.201 | 0.188 | 0.915 |
| Hs.13308 | AI744123 | 134548 | chr5q23.3 | — | Hs.13308_at | hypothetical protein LOC134548 | LOC134548 | AnCg | 0.169 | 1.158 | 0.016 | 1.410 | 0.058 | 1.255 |
| Hs.140720 | AB045118 | 23401 | chr10q24.1 | 605006 | Hs.140720_at | frequently rearranged in | FRAT2 | AnCg | 0.174 | 1.077 | 0.027 | 1.282 | 0.864 | 1.020 |
| | | | | | | advanced T-cell lymphomas 2 | | | | | | | | |
| Hs.14511 | AF183424 | 6341 | chr17p12-p13 | 603644 | Hs.14511_at | SCO cytochrome oxidase | SCO1 | AnCg | 0.324 | 1.019 | 0.033 | 1.411 | 0.453 | 1.110 |
| | | | | | | deficient homolog 1 (yeast) | | | | | | | | |
| Hs.145156 | NM_024046 | 79012 | chr3p21.31 | — | Hs.145156_at | hypothetical protein MGC8407 | MGC8407 | AnCg | 0.100 | 1.219 | 0.029 | 1.306 | 0.090 | 1.322 |
| Hs.158798 | H17349 | 254778 | chr8q13.1 | — | Hs.158798_at | hypothetical protein MGC33510 | MGC33510 | AnCg | 0.054 | 1.588 | 0.036 | 1.378 | 0.524 | 1.138 |
| Hs.1608 | BC005264 | 6119 | chr7p22 | 179837 | Hs.1608_at | replication protein A3, 14 kDa | RPA3 | AnCg | 0.067 | 1.248 | 0.025 | 1.311 | 0.564 | 1.153 |
| Hs.173902 | NM_014225 | 5518 | chr19q13.41 | 605983 | Hs.173902_at | protein phosphatase 2 (formerly | PPP2R1A | AnCg | 0.680 | 1.168 | 0.036 | 1.230 | 0.131 | 1.214 |
| | | | | | | 2A), regulatory subunit A (PR | | | | | | | | |
| | | | | | | 65), alpha isoform | | | | | | | | |
| Hs.179770 | NM_002842 | 5794 | chr19q13.4 | 602510 | Hs.179770_at | protein tyrosine phosphatase, | PTPRH | AnCg | 0.560 | 1.056 | 0.041 | 1.229 | 0.836 | 0.984 |
| | | | | | | receptor type, H | | | | | | | | |
| Hs.183646 | NM_016011 | 51102 | chr1pter-p22.3 | 608205 | Hs.183646_at | nuclear receptor binding factor 1 | CGI-63 | AnCg | 0.638 | 1.087 | 0.033 | 1.208 | 0.121 | 1.459 |
| Hs.185202 | BC035082 | 286032 | chr8p22 | — | Hs.185202_at | hypothetical protein FLJ36980 | FLJ36980 | AnCg | 0.881 | 1.002 | 0.011 | 1.224 | 0.617 | 1.010 |
| Hs.190559 | AI828026 | 255426 | chr5q35.3 | — | Hs.190559_at | hypothetical protein LOC255426 | LOC255426 | AnCg | 0.525 | 0.981 | 0.000 | 1.282 | 0.005 | 1.124 |
| Hs.192822 | N32557 | 81706 | chr6q24.3-q25.3 | — | Hs.192822_at | protein phosphatase 1, | PPP1R14C | AnCg | 0.159 | 1.105 | 0.035 | 1.357 | 0.219 | 1.366 |
| | | | | | | regulatory (inhibitor) subunit | | | | | | | | |
| | | | | | | 14C | | | | | | | | |
| Hs.194673 | BC002426 | 8682 | chr1q21.1 | 603434 | Hs.194673_at | phosphoprotein enriched in | PEA15 | AnCg | 0.607 | 1.150 | 0.006 | 1.228 | 0.044 | 1.173 |
| | | | | | | astrocytes 15 | | | | | | | | |
| Hs.197922 | NM_018584 | 55450 | chr1p36.12 | — | Hs.197922_at | calcium/calmodulin-dependent | CaMKIINalpha | AnCg | 0.430 | 1.278 | 0.013 | 1.356 | 0.071 | 1.372 |
| | | | | | | protein kinase II | | | | | | | | |
| Hs.198998 | AF080157 | 1147 | chr10q24-q25 | 600664 | Hs.198998_at | conserved helix-loop-helix | CHUK | AnCg | 0.998 | 0.999 | 0.020 | 0.798 | 0.022 | 0.852 |
| | | | | | | ubiquitous kinase | | | | | | | | |
| Hs.201058 | NM_030795 | 81551 | chr8p21.2 | — | Hs.201058_at | stathmin-like 4 | STMN4 | AnCg | 0.102 | 1.105 | 0.037 | 1.230 | 0.635 | 1.107 |
| Hs.20191 | U76248 | 6478 | chr3q25 | 602213 | Hs.20191_at | seven in absentia homolog 2 | SIAH2 | AnCg | 0.658 | 1.064 | 0.030 | 1.482 | 0.307 | 1.140 |
| | | | | | | (Drosophila) | | | | | | | | |
| Hs.21415 | | | | | Hs.21415_at | hypothetical protein MGC39820 | MGC39820 | AnCg | 0.076 | 1.282 | 0.034 | 1.226 | 0.099 | 1.336 |
| Hs.22181 | AL031658 | 81572 | chr20q11.21 | — | Hs.22181_at | chromosome 20 open reading | C20orf126 | AnCg | 0.603 | 0.967 | 0.007 | 1.256 | 0.322 | 1.160 |
| | | | | | | frame 126 | | | | | | | | |
| Hs.235195 | NM_018019 | 55090 | chr17p11.2 | — | Hs.235195_at | hypothetical protein FLJ10193 | FLJ10193 | AnCg | 0.716 | 0.974 | 0.030 | 1.262 | 0.345 | 1.130 |
| Hs.23735 | AF029780 | 3754 | chr2p25 | 603787 | Hs.23735_at | potassium voltage-gated | KCNF1 | AnCg | 0.081 | 1.170 | 0.024 | 1.222 | 0.025 | 1.187 |
| | | | | | | channel, subfamily F, member 1 | | | | | | | | |
| Hs.241564 | AB014559 | 747 | chr11q12.2 | — | Hs.241564_at | chromosome 11 open reading | C11orf11 | AnCg | 0.844 | 1.004 | 0.015 | 1.263 | 0.071 | 1.170 |
| | | | | | | frame 11 | | | | | | | | |
| Hs.24969 | NM_000810 | 2558 | chr15q11.2-q12 | 137142 | Hs.24969_at | gamma-aminobutyric acid | GABRA5 | AnCg | 0.381 | 1.686 | 0.027 | 1.475 | 0.306 | 1.524 |
| | | | | | | (GABA) A receptor, alpha 5 | | | | | | | | |
| Hs.250692 | AI810712 | 3131 | chr17q22 | 142385 | Hs.250692_at | hepatic leukemia factor | HLF | AnCg | 0.070 | 1.334 | 0.049 | 1.221 | 0.086 | 1.366 |
| Hs.254406 | NM_013375 | 29777 | chr6p22.1 | — | Hs.254406_at | activator of basal transcription 1 | ABT1 | AnCg | 0.669 | 0.990 | 0.014 | 1.440 | 0.591 | 1.053 |
| Hs.254414 | NM_080743 | 135295 | chr6q15 | — | Hs.254414_at | serine-arginine repressor | SRrp35 | AnCg | 0.660 | 0.988 | 0.050 | 1.211 | 0.329 | 1.065 |
| | | | | | | protein (35 kDa) | | | | | | | | |
| Hs.2624 | X58987 | 1812 | chr5q35.1 | 126449 | Hs.2624_at | dopamine receptor D1 | DRD1 | AnCg | 0.163 | 1.093 | 0.044 | 1.227 | 0.538 | 1.060 |
| Hs.271272 | BF510581 | 121551 | chr12q23.3 | — | Hs.271272_at | BTB (POZ) domain containing | BTBD11 | AnCg | 0.798 | 0.984 | 0.020 | 1.241 | 0.118 | 1.283 |
| | | | | | | 11 | | | | | | | | |
| Hs.273307 | BG398597 | 6730 | chr17q25.1 | 604858 | Hs.273307_at | signal recognition particle | SRP68 | AnCg | 0.877 | 1.032 | 0.041 | 1.204 | 0.168 | 1.153 |
| | | | | | | 68 kDa | | | | | | | | |
| Hs.277517 | NM_013265 | 738 | chr11q13 | — | Hs.277517_at | chromosome 11 open reading | C11orf2 | AnCg | 0.229 | 1.181 | 0.040 | 1.217 | 0.124 | 1.232 |
| | | | | | | frame2 | | | | | | | | |
| Hs.288520 | AK024467 | 90011 | chr19q13.42 | — | Hs.288520_at | hypothetical gene FLJ00060 | FLJ00060 | AnCg | 0.665 | 0.894 | 0.017 | 0.547 | 0.444 | 0.898 |
| Hs.288932 | NM_025146 | 80218 | chr3q13.2 | — | Hs.288932_at | likely ortholog of mouse Mak3p | MAK3P | AnCg | 0.244 | 1.251 | 0.024 | 1.210 | 0.548 | 1.074 |
| | | | | | | homolog (S. cerevisiae) | | | | | | | | |
| Hs.2890 | NM_002739 | 5582 | chr19q13.4 | 176980 | Hs.2890_at | protein kinase C, gamma | PRKCG | AnCg | 0.168 | 1.179 | 0.043 | 1.214 | 0.102 | 1.292 |
| Hs.28980 | AI674647 | 84888 | chr15q21.2 | 608238 | Hs.28980_at | putative intramembrane | SPPL2A | AnCg | 0.695 | 1.048 | 0.018 | 0.774 | 0.128 | 0.726 |
| | | | | | | cleaving protease | | | | | | | | |
| Hs.293336 | NM_018264 | 55253 | chr7q11.21 | — | Hs.293336_at | hypothetical protein FLJ10900 | FLJ10900 | AnCg | 0.206 | 0.834 | 0.026 | 0.749 | 0.813 | 0.977 |
| Hs.293660 | AW249467 | 91107 | chr17q24-q25 | — | Hs.293660_at | tripartite motif-containing 47 | TRIM47 | AnCg | 0.196 | 0.917 | 0.037 | 0.828 | 0.064 | 0.809 |
| Hs.295137 | NM_001144 | 267 | chr16q21 | 603243 | Hs.295137_at | autocrine motility factor receptor | AMFR | AnCg | 0.806 | 0.958 | 0.023 | 0.482 | 0.095 | 0.562 |
| Hs.298351 | NM_024083 | 79058 | chr17q25 | 606236 | Hs.298351_at | alveolar soft part sarcoma | ASPSCR1 | AnCg | 0.215 | 0.982 | 0.014 | 1.323 | 0.470 | 0.955 |
| | | | | | | chromosome region, candidate 1 | | | | | | | | |
| Hs.300906 | BC010136 | 55197 | chr18q12.2 | — | Hs.300906_at | hypothetical protein FLJ10656 | P15RS | AnCg | 0.399 | 1.242 | 0.015 | 1.245 | 0.306 | 1.148 |
| Hs.312129 | NM_015925 | 51599 | chr19q13.12 | — | Hs.312129_at | liver-specific bHLH-Zip | LISCH7 | AnCg | 0.765 | 1.018 | 0.027 | 0.800 | 0.397 | 0.893 |
| | | | | | | transcription factor | | | | | | | | |
| Hs.325321 | BC001648 | 57418 | chr19p13.3 | — | Hs.325321_at | WD repeat domain 18 | WDR18 | AnCg | 0.717 | 0.996 | 0.016 | 1.278 | 0.685 | 1.037 |
| Hs.348380 | NM_005748 | 10138 | chr12q12 | 607534 | Hs.348380_at | YY1 associated factor 2 | YAF2 | AnCg | 0.035 | 1.093 | 0.029 | 1.256 | 0.226 | 1.158 |
| Hs.350194 | AA205643 | 153527 | chr5q31.3 | — | Hs.350194_at | hypothetical protein FLJ31121 | FLJ31121 | AnCg | 0.172 | 1.258 | 0.044 | 1.346 | 0.111 | 1.349 |
| Hs.365690 | NM_021161 | 54207 | chr14q31 | 605873 | Hs.365690_at | potassium channel, subfamily K, | KCNK10 | AnCg | 0.786 | 1.005 | 0.044 | 0.733 | 0.005 | 0.852 |
| | | | | | | member 10 | | | | | | | | |
| Hs.367669 | BG285881 | 166336 | chr3p14.1 | 608501 | Hs.367669_at | prickle-like 2 (Drosophila) | PRICKLE2 | AnCg | 0.095 | 1.192 | 0.023 | 1.259 | 0.075 | 1.297 |
| Hs.368866 | BC017771 | 60492 | chr11q14.1 | — | Hs.368866_at | hypothetical protein MDS025 | MDS025 | AnCg | 0.324 | 0.926 | 0.014 | 0.808 | 0.064 | 0.803 |
| Hs.369579 | BC015963 | 830 | chr7q31.2-q31.3 | 601571 | Hs.369579_at | capping protein (actin filament) | CAPZA2 | AnCg | 0.059 | 1.237 | 0.029 | 1.227 | 0.943 | 1.010 |
| | | | | | | muscle Z-line, alpha 2 | | | | | | | | |
| Hs.374285 | AA307731 | 339344 | chr19q13.32 | — | Hs.374285_at | hypothetical protein LOC339344 | LOC339344 | AnCg | 0.190 | 0.951 | 0.011 | 1.222 | 0.901 | 1.013 |
| Hs.375641 | NM_019042 | 54517 | chr7q22.3 | — | Hs.375641_at | hypothetical protein FLJ20485 | FLJ20485 | AnCg | 0.525 | 1.142 | 0.036 | 0.779 | 0.925 | 0.973 |
| Hs.379010 | AK092432 | 8000 | chr8q24.2 | 602470 | Hs.379010+_at | prostate stem cell antigen | PSCA | AnCg | 0.864 | 1.021 | 0.018 | 1.314 | 0.874 | 0.981 |
| Hs.381050 | NM_018644 | 27087 | chr11q25 | 606375 | Hs.381050_at | beta-1,3-glucuronyltransferase | B3GAT1 | AnCg | 0.355 | 1.209 | 0.031 | 1.243 | 0.053 | 1.200 |
| | | | | | | 1 (glucuronosyltransferase P) | | | | | | | | |
| Hs.381264 | U37028 | 3681 | chr16p11.2 | 602453 | Hs.381264_at | integrin, alpha D | ITGAD | AnCg | 0.648 | 0.968 | 0.009 | 0.829 | 0.903 | 0.992 |
| Hs.388645 | BF203664 | 84987 | chr12q13.12 | — | Hs.388645_at | hypothetical protein MGC14288 | MGC14288 | AnCg | 0.270 | 1.126 | 0.045 | 1.224 | 0.203 | 1.226 |
| Hs.38961 | AL121756 | 149954 | chr20q11.21 | — | Hs.38961_at | chromosome 20 open reading | C20orf186 | AnCg | 0.206 | 0.923 | 0.024 | 0.762 | 0.902 | 1.011 |
| | | | | | | frame 186 | | | | | | | | |
| Hs.410953 | AK094809 | 5924 | chr5q13 | 606614 | Hs.410953_at | Ras protein-specific guanine | RASGRF2 | AnCg | 0.085 | 1.199 | 0.026 | 1.377 | 0.114 | 1.372 |
| | | | | | | nucleotide-releasing factor 2 | | | | | | | | |
| Hs.411358 | NM_012286 | 9643 | chrxq22 | 300409 | Hs.411358_at | mortality factor 4 like 2 | MORF4L2 | AnCg | 0.727 | 1.033 | 0.008 | 0.733 | 0.091 | 0.745 |
| Hs.412587 | NM_002876 | 5889 | chr17q22-q23 | 602774 | Hs.412587_at | RAD51 homolog C (S. cerevisiae) | RAD51C | AnCg | 0.124 | 1.158 | 0.037 | 1.252 | 0.150 | 1.226 |
| Hs.418367 | NM_006681 | 10874 | chr4q12 | 605103 | Hs.418367_at | neuromedin U | NMU | AnCg | 0.143 | 1.086 | 0.024 | 1.213 | 0.134 | 1.225 |
| Hs.422986 | BC000182 | 307 | chr2p13 | 106491 | Hs.422986_at | annexin A4 | ANXA4 | AnCg | 0.953 | 1.005 | 0.024 | 0.791 | 0.480 | 0.887 |
| Hs.426142 | NM_002643 | 5281 | chr2p21-p16 | 600153 | Hs.426142_at | phosphatidylinositol glycan, | PIGF | AnCg | 0.321 | 1.182 | 0.029 | 1.353 | 0.910 | 0.985 |
| | | | | | | class F | | | | | | | | |
| Hs.429904 | BC000975 | 283755 | chr15q11.2 | — | Hs.429904_at | hypothetical protein LOC283755 | LOC283755 | AnCg | 0.062 | 0.709 | 0.049 | 0.792 | 0.114 | 0.730 |
| Hs.434124 | AK057562 | 149086 | chr1p35.2 | — | Hs.434124+_at | hypothetical protein LOC149086 | LOC149086 | AnCg | 0.195 | 0.795 | 0.042 | 0.640 | 0.562 | 1.079 |
| Hs.435051 | U20498 | 1032 | chr19p13 | 600927 | Hs.435051_at | cyclin-dependent kinase | CDKN2D | AnCg | 0.147 | 1.261 | 0.027 | 1.234 | 0.173 | 1.206 |
| | | | | | | inhibitor 2D (p19, inhibits CDK4) | | | | | | | | |
| Hs.437186 | NM_016310 | 51728 | chr16p13.3 | 606007 | Hs.437186_at | polymerase (RNA) III (DNA | POLR3K | AnCg | 0.187 | 1.305 | 0.043 | 1.241 | 0.397 | 1.135 |
| | | | | | | directed) polypeptide K, 12.3 kDa | | | | | | | | |
| Hs.438830 | NM_013238 | 29103 | chr13q14.1 | — | Hs.438830_at | DnaJ (Hsp40) homolog, | DNAJD1 | AnCg | 0.598 | 1.087 | 0.028 | 1.375 | 0.154 | 1.285 |
| | | | | | | subfamily D, member 1 | | | | | | | | |
| Hs.439909 | NM_020689 | 57419 | chr20p13 | — | Hs.439909_at | solute carrier family 24 | SLC24A3 | AnCg | 0.260 | 1.086 | 0.031 | 1.214 | 0.881 | 0.974 |
| | | | | | | (sodium/potassium/calcium | | | | | | | | |
| | | | | | | exchanger), member 3 | | | | | | | | |
| Hs.440310 | AF151034 | 51239 | chr2q11.2 | — | Hs.440310_at | hypothetical protein MGC41816 | MGC41816 | AnCg | 0.067 | 1.116 | 0.009 | 1.288 | 0.788 | 1.045 |
| Hs.444749 | NM_001001 | 6166 | chr14q21 | 180469 | Hs.444749_at | ribosomal protein L36a-like | RPL36AL | AnCg | 0.230 | 1.274 | 0.040 | 1.222 | 0.748 | 0.959 |
| Hs.447902 | AF094508 | 1834 | chr4q21.3 | 125485 | Hs.447902_at | dentin sialophosphoprotein | DSPP | AnCg | 0.779 | 0.968 | 0.021 | 1.234 | 0.344 | 0.900 |
| Hs.448353 | R24798 | 58512 | chr1p35.3-p34.1 | — | Hs.448353_at | SAP90/PSD-95-associated | SAPAP3 | AnCg | 0.112 | 1.045 | 0.003 | 1.318 | 0.062 | 1.407 |
| | | | | | | protein 3 | | | | | | | | |
| Hs.451604 | NM_003803 | 8736 | chr18p11.32-p11.31 | 603508 | Hs.451604_at | myomesin 1 (skelemin) 185 kDa | MYOM1 | AnCg | 0.488 | 0.912 | 0.014 | 0.762 | 0.346 | 0.927 |
| Hs.458308 | | | | | Hs.458308_at | chromosome 21 open reading | C21orf55 | AnCg | 0.440 | 1.064 | 0.043 | 0.794 | 0.888 | 0.986 |
| | | | | | | frame 55 | | | | | | | | |
| Hs.467587 | | | | | Hs.467587_at | hypothetical protein AE2 | AE2 | AnCg | 0.994 | 0.999 | 0.007 | 1.433 | 0.664 | 1.036 |
| Hs.46794 | AA872588 | 148979 | chr1p32.3 | — | Hs.46794_at | likely ortholog of mouse Gli- | FLJ36155 | AnCg | 0.244 | 0.930 | 0.019 | 0.787 | 0.260 | 1.105 |
| | | | | | | similar 1 Kruppel-like zinc finger | | | | | | | | |
| | | | | | | (Glis1) | | | | | | | | |
| Hs.473788 | AL523776 | 55611 | chr11q13.1 | 608337 | Hs.473788_at | OTU domain, ubiquitin aldehyde | OTUB1 | AnCg | 0.073 | 1.255 | 0.043 | 1.207 | 0.111 | 1.291 |
| | | | | | | binding 1 | | | | | | | | |
| Hs.49230 | R38624 | 140767 | chr6p22.2 | — | Hs.49230_at | vesicular membrane protein p24 | VMP | AnCg | 0.460 | 1.192 | 0.018 | 1.234 | 0.051 | 1.479 |
| Hs.4992 | NM_003310 | 7260 | chr2p25.2 | — | Hs.4992_at | tumor suppressing | TSSC1 | AnCg | 0.307 | 1.073 | 0.005 | 1.303 | 0.388 | 1.189 |
| | | | | | | subtransferable candidate 1 | | | | | | | | |
| Hs.500165 | AL528911 | 84313 | chr17q21.2 | — | Hs.500165_at | hypothetical protein MGC10540 | MGC10540 | AnCg | 0.351 | 1.063 | 0.041 | 1.214 | 0.468 | 1.088 |
| Hs.50282 | NM_016656 | 10325 | chrxp11.22 | — | Hs.50282_at | Ras-related GTP binding B | RRAGB | AnCg | 0.664 | 1.004 | 0.042 | 1.217 | 0.330 | 1.120 |
| Hs.511807 | | | | | Hs.511807_at | hypothetical protein FLJ90005 | FLJ90005 | AnCg | 0.624 | 1.017 | 0.036 | 1.293 | 0.233 | 1.136 |
| Hs.511950 | AF083108 | 23410 | chr11p15.5 | 604481 | Hs.511950_at | sirtuin (silent mating type | SIRT3 | AnCg | 0.786 | 1.021 | 0.028 | 1.224 | 0.079 | 1.185 |
| | | | | | | information regulation 2 | | | | | | | | |
| | | | | | | homolog) 3 (S. cerevisiae) | | | | | | | | |
| Hs.512579 | | | | | Hs.512579_at | keratin, hair, acidic, 3A | KRTHA3A | AnCg | 0.036 | 1.108 | 0.010 | 1.291 | 0.483 | 1.032 |
| Hs.512607 | NM_016641 | 51573 | chr16p12-p11.2 | 605943 | Hs.512607_at | membrane interacting protein of | MIR16 | AnCg | 0.464 | 1.197 | 0.028 | 1.241 | 0.537 | 1.074 |
| | | | | | | RGS16 | | | | | | | | |
| Hs.512743 | NM_024920 | 79982 | chr4q23 | — | Hs.512743_at | hypothetical protein FLJ14281 | FLJ14281 | AnCg | 0.613 | 1.147 | 0.032 | 0.822 | 0.791 | 1.022 |
| Hs.515246 | | | | | Hs.515246_at | UDP-GlcNAc:betaGal beta-1,3- | B3GNT3 | AnCg | 0.463 | 0.910 | 0.023 | 0.744 | 0.229 | 1.154 |
| | | | | | | N- | | | | | | | | |
| | | | | | | acetylglucosaminyltransferase 3 | | | | | | | | |
| Hs.518164 | AK025047 | 80193 | chr3p21.1-q13.13 | — | Hs.518164_at | hypothetical protein FLJ21394 | FLJ21394 | AnCg | 0.461 | 0.893 | 0.023 | 0.826 | 0.490 | 1.060 |
| Hs.58488 | NM_003798 | 8727 | chr9q31.2 | 604785 | Hs.58488_at | catenin (cadherin-associated | CTNNAL1 | AnCg | 0.299 | 0.877 | 0.025 | 0.775 | 0.243 | 0.825 |
| | | | | | | protein), alpha-like 1 | | | | | | | | |
| Hs.59729 | NM_020163 | 56920 | chr3p21.1 | — | Hs.59729_at | semaphorin sem2 | LOC56920 | AnCg | 0.105 | 0.834 | 0.015 | 0.770 | 0.062 | 0.759 |
| Hs.6140 | AL566367 | 84966 | chr1p36.13 | — | Hs.6140_at | hypothetical protein MGC15730 | MGC15730 | AnCg | 0.893 | 1.010 | 0.004 | 1.400 | 0.574 | 1.100 |
| Hs.6877 | NM_018108 | 55148 | chr14q32.13 | — | Hs.6877_at | chromosome 14 open reading | C14orf130 | AnCg | 0.270 | 1.174 | 0.032 | 1.205 | 0.407 | 1.094 |
| | | | | | | frame 130 | | | | | | | | |
| Hs.75137 | NM_014766 | 9805 | chr7p14.3-p14.1 | — | Hs.75137_at | secemin 1 | SES1 | AnCg | 0.633 | 1.196 | 0.020 | 1.204 | 0.049 | 1.200 |
| Hs.79058 | BC002802 | 6827 | chr17q21-q23 | 603555 | Hs.79058_at | suppressor of Ty 4 homolog 1 | SUPT4H1 | AnCg | 0.784 | 1.029 | 0.034 | 1.201 | 0.271 | 1.211 |
| | | | | | | (S. cerevisiae) | | | | | | | | |
| Hs.7935 | NM_014962 | 22903 | chr20p12.2 | — | Hs.7935_at | BTB (POZ) domain containing 3 | BTBD3 | AnCg | 0.165 | 1.365 | 0.019 | 1.203 | 0.420 | 1.153 |
| Hs.7991 | AA206763 | 128434 | chr20q11.23 | — | Hs.7991_at | chromosome 20 open reading | C20orf102 | AnCg | 0.216 | 1.190 | 0.030 | 1.203 | 0.060 | 1.425 |
| | | | | | | frame 102 | | | | | | | | |
| Hs.82023 | BC000850 | 54461 | chr9q34.3 | — | Hs.82023_at | F-box and WD-40 domain | FBXW5 | AnCg | 0.389 | 1.156 | 0.006 | 1.206 | 0.094 | 1.216 |
| | | | | | | protein 5 | | | | | | | | |
| Hs.82120 | AI935096 | 4929 | chr2q22-q23 | 601828 | Hs.82120_at | nuclear receptor subfamily 4, | NR4A2 | AnCg | 0.351 | 0.875 | 0.016 | 0.655 | 0.765 | 0.847 |
| | | | | | | group A, member 2 | | | | | | | | |
| Hs.88367 | NM_017714 | 55617 | chr20p12.1 | 608270 | Hs.88367_at | chromosome 20 open reading | C20orf13 | AnCg | 0.584 | 1.090 | 0.028 | 1.273 | 0.427 | 1.096 |
| | | | | | | frame 13 | | | | | | | | |
| Hs.90063 | AF251061 | 83988 | chr8q22-q23 | 606722 | Hs.90063_at | neurocalcin delta | NCALD | AnCg | 0.406 | 1.314 | 0.017 | 1.236 | 0.173 | 1.566 |
| Hs.9629 | BC004913 | 5546 | chr1q21.1 | 179755 | Hs.9629_at | papillary renal cell carcinoma | PRCC | AnCg | 0.237 | 1.278 | 0.003 | 1.202 | 0.868 | 1.020 |
| | | | | | | (translocation-associated) | | | | | | | | |
| Hs.100194 | NM_001629 | 241 | chr13q12 | 603700 | Hs.100194_at | arachidonate 5-lipoxygenase- | ALOX5AP | Amy | 0.847 | 1.015 | 0.445 | 0.920 | 0.025 | 0.340 |
| | | | | | | activating protein | | | | | | | | |
| Hs.100343 | NM_152704 | 219287 | chr13q12.13 | — | Hs.100343_at | hypothetical protein FLJ25477 | FLJ25477 | Amy | 0.862 | 1.033 | 0.412 | 0.868 | 0.015 | 0.714 |
| Hs.10043 | NM_022062 | 63876 | chr11q24 | — | Hs.10043_at | PBX/knotted 1 homeobox 2 | PKNOX2 | Amy | 0.525 | 1.038 | 0.933 | 0.996 | 0.001 | 1.521 |
| Hs.101672 | AW157571 | 152789 | chr4p16.1 | — | Hs.101672_at | hypothetical protein FLJ31564 | FLJ31564 | Amy | 0.169 | 1.098 | 0.071 | 1.137 | 0.023 | 1.264 |
| Hs.103291 | NM_016588 | 51299 | chr6p25.1 | 607409 | Hs.103291_at | neuritin 1 | NRN1 | Amy | 0.432 | 1.214 | 0.040 | 1.140 | 0.019 | 1.360 |
| Hs.103378 | AL136885 | 83641 | chr10p13 | — | Hs.103378_at | chromosome 10 open reading | C10orf45 | Amy | 0.612 | 1.213 | 0.764 | 0.881 | 0.025 | 0.613 |
| | | | | | | frame 45 | | | | | | | | |
| Hs.103395 | NM_024709 | 79762 | chr1q41 | — | Hs.103395_at | hypothetical protein FLJ14146 | FLJ14146 | Amy | 0.286 | 1.105 | 0.106 | 1.116 | 0.006 | 1.438 |
| Hs.10526 | NM_001321 | 1466 | chr12q1.1 | 601871 | Hs.10526_at | cysteine and glycine-rich protein 2 | CSRP2 | Amy | 0.580 | 0.921 | 0.313 | 0.863 | 0.047 | 0.698 |
| Hs.106688 | NM_004709 | 9142 | chrxq27.3 | — | Hs.106688_at | chromosome X open reading | CXorf1 | Amy | 0.575 | 1.062 | 0.282 | 1.245 | 0.011 | 1.463 |
| | | | | | | frame 1 | | | | | | | | |
| Hs.107056 | AF200715 | 51454 | chr2q32.3-q33 | 608165 | Hs.107056_at | GULP, engulfment adaptor PTB | GULP1 | Amy | 0.570 | 1.075 | 0.267 | 1.069 | 0.044 | 1.310 |
| | | | | | | domain containing 1 | | | | | | | | |
| Hs.107393 | BC013610 | 56650 | chr3p11-q11 | — | Hs.107393_at | chromosome 3 open reading | C3orf4 | Amy | 0.611 | 1.160 | 0.791 | 0.945 | 0.024 | 0.666 |
| | | | | | | frame 4 | | | | | | | | |
| Hs.108689 | BE513151 | 6721 | chr22q13 | 600481 | Hs.108689_at | sterol regulatory element | SREBF2 | Amy | 0.532 | 1.073 | 0.239 | 1.057 | 0.043 | 1.208 |
| | | | | | | binding transcription factor 2 | | | | | | | | |
| Hs.109299 | BE501428 | 8541 | chr19q13.33 | 603144 | Hs.109299_at | protein tyrosine phosphatase, | PPFIA3 | Amy | 0.690 | 0.944 | 0.382 | 1.071 | 0.001 | 1.515 |
| | | | | | | receptor type, f polypeptide | | | | | | | | |
| | | | | | | (PTPRF), interacting protein | | | | | | | | |
| | | | | | | (liprin), alpha 3 | | | | | | | | |
| Hs.109309 | NM_017631 | 55601 | chr4q32.3 | — | Hs.109309_at | hypothetical protein FLJ20035 | FLJ20035 | Amy | 0.033 | 0.868 | 0.188 | 0.911 | 0.019 | 0.759 |
| Hs.109358 | AW006935 | 23120 | chr5q34 | — | Hs.109358_at | ATPase, Class V, type 10B | ATP10B | Amy | 0.998 | 1.000 | 0.658 | 0.975 | 0.021 | 0.773 |
| Hs.109438 | AA551075 | 115207 | chr13q22.3 | — | Hs.109438_at | potassium channel | KCTD12 | Amy | 0.449 | 0.935 | 0.153 | 0.880 | 0.009 | 0.746 |
| | | | | | | tetramerisation domain | | | | | | | | |
| | | | | | | containing 12 | | | | | | | | |
| Hs.11065 | BF515889 | 85313 | chr6q24-q25 | 607609 | Hs.11065_at | peptidylprolyl isomerase | PPIL4 | Amy | 0.916 | 1.016 | 0.381 | 0.880 | 0.014 | 0.709 |
| | | | | | | (cyclophilin)-like 4 | | | | | | | | |
| Hs.110736 | NM_001046 | 6558 | chr5q23.3 | 600840 | Hs.110736_at | solute carrier family 12 | SLC12A2 | Amy | 0.883 | 0.979 | 0.129 | 0.779 | 0.016 | 0.637 |
| | | | | | | (sodium/potassium/chloride | | | | | | | | |
| | | | | | | transporters), member 2 | | | | | | | | |
| Hs.111676 | AF133207 | 26353 | chr12q24.23 | 608014 | Hs.111676_at | protein kinase H11 | H11 | Amy | 0.790 | 0.910 | 0.827 | 1.017 | 0.005 | 0.732 |
| Hs.111779 | NM_003118 | 6678 | chr5q31.3-q32 | 182120 | Hs.111779_at | secreted protein, acidic, | SPARC | Amy | 0.917 | 0.987 | 0.783 | 1.038 | 0.047 | 0.658 |
| | | | | | | cysteine-rich (osteonectin) | | | | | | | | |
| Hs.112356 | NM_015929 | 51601 | chr2q11.2 | — | Hs.112356_at | lipoyltransferase | LIPT1 | Amy | 0.446 | 0.958 | 0.302 | 0.861 | 0.024 | 0.677 |
| Hs.112499 | NM_004758 | 9256 | chr17q22-q23 | — | Hs.112499_at | benzodiazapine receptor | BZRAP1 | Amy | 0.820 | 0.977 | 0.620 | 1.034 | 0.007 | 1.445 |
| | | | | | | (peripheral) associated protein 1 | | | | | | | | |
| Hs.112928 | AF493870 | 54331 | chr14q21 | 606981 | Hs.112928_at | guanine nucleotide binding | GNG2 | Amy | 0.071 | 1.104 | 0.080 | 1.156 | 0.020 | 1.277 |
| | | | | | | protein (G protein), gamma 2 | | | | | | | | |
| Hs.112933 | NM_016948 | 50855 | chr16q22.1 | 607484 | Hs.112933_at | par-6 partitioning defective 6 | PARD6A | Amy | 0.953 | 0.995 | 0.135 | 1.191 | 0.001 | 1.407 |
| | | | | | | homolog alpha (C. elegans) | | | | | | | | |
| Hs.1162 | NM_002118 | 3109 | chr6p21.3 | 142856 | Hs.1162_at | major histocompatibility | HLA-DMB | Amy | 0.339 | 0.911 | 0.106 | 0.914 | 0.027 | 0.750 |
| | | | | | | complex, class II, DM beta | | | | | | | | |
| Hs.117060 | AI473096 | 1842 | chr9q22.3 | 603479 | Hs.117060_at | extracellular matrix protein 2, | ECM2 | Amy | 0.511 | 0.987 | 0.694 | 0.959 | 0.046 | 0.760 |
| | | | | | | female organ and adipocyte | | | | | | | | |
| | | | | | | specific | | | | | | | | |
| Hs.117339 | AF285447 | 10870 | chr19q13.1 | 604089 | Hs.117339_at | hematopoietic cell signal | HCST | Amy | 0.756 | 0.972 | 0.449 | 0.947 | 0.033 | 0.738 |
| | | | | | | transducer | | | | | | | | |
| Hs.117780 | NM_002251 | 3787 | chr20q12 | 602905 | Hs.117780_at | potassium voltage-gated | KCNS1 | Amy | 0.773 | 0.986 | 0.460 | 0.940 | 0.015 | 1.329 |
| | | | | | | channel, delayed-rectifier, | | | | | | | | |
| | | | | | | subfamily S, member 1 | | | | | | | | |
| Hs.117956 | NM_006914 | 6096 | chr9q22 | 601972 | Hs.117956_at | RAR-related orphan receptor B | RORB | Amy | 0.108 | 1.156 | 0.577 | 0.922 | 0.009 | 1.507 |
| Hs.118110 | NM_004335 | 684 | chr19p13.2 | 600534 | Hs.118110_at | bone marrow stromal cell | BST2 | Amy | 0.108 | 0.938 | 0.439 | 0.923 | 0.001 | 0.741 |
| | | | | | | antigen 2 | | | | | | | | |
| Hs.118281 | AA868898 | 10781 | chr19p13.2 | 604751 | Hs.118281_at | zinc finger protein 266 | ZNF266 | Amy | 0.584 | 1.105 | 0.336 | 0.909 | 0.044 | 0.776 |
| Hs.118483 | AA877789 | 4646 | chr6q13 | 600970 | Hs.118483_at | myosin VI | MYO6 | Amy | 0.858 | 0.978 | 0.447 | 0.898 | 0.044 | 0.798 |
| Hs.118843 | AB049127 | 57787 | chr19q13.3 | 606495 | Hs.118843_at | MAP/microtubule affinity- | MARK4 | Amy | 0.562 | 1.084 | 0.320 | 1.053 | 0.006 | 1.280 |
| | | | | | | regulating kinase 4 | | | | | | | | |
| Hs.119062 | AW612149 | 285800 | chr6q27 | — | Hs.119062_at | hypothetical protein MGC35308 | MGC35308 | Amy | 0.721 | 1.104 | 0.534 | 0.778 | 0.028 | 0.550 |
| Hs.120963 | BC016343 | 55421 | chr17p13.3 | — | Hs.120963_at | ELG protein | HSA277841 | Amy | 0.073 | 0.975 | 0.933 | 1.006 | 0.034 | 0.802 |
| Hs.122440 | AL050376 | 130872 | chr2p16.1-p15 | — | Hs.122440_at | AHA1, activator of heat shock | AHSA2 | Amy | 0.679 | 1.019 | 0.500 | 0.953 | 0.004 | 0.726 |
| | | | | | | 90 kDa protein ATPase homolog | | | | | | | | |
| | | | | | | 2 (yeast) | | | | | | | | |
| Hs.12264 | AA001423 | 57463 | chr1p13.3 | — | Hs.12264_at | amphoterin-induced gene | KIAA1163 | Amy | 0.271 | 1.046 | 0.727 | 1.016 | 0.044 | 1.308 |
| Hs.123119 | | | | | Hs.123119_at | MAD, mothers against | MADH9 | Amy | 0.751 | 1.075 | 0.472 | 0.905 | 0.018 | 0.647 |
| | | | | | | decapentaplegic homolog 9 | | | | | | | | |
| | | | | | | (Drosophila) | | | | | | | | |
| Hs.12332 | AA758861 | 144100 | chr11p15.1 | — | Hs.12332_at | hypothetical protein LOC144100 | LOC144100 | Amy | 0.844 | 1.008 | 0.308 | 0.953 | 0.006 | 0.809 |
| Hs.123464 | BC039373 | 10161 | chr13q14 | — | Hs.123464_at | purinergic receptor P2Y, G- | P2RY5 | Amy | 0.378 | 0.882 | 0.194 | 0.795 | 0.003 | 0.374 |
| | | | | | | protein coupled, 5 | | | | | | | | |
| Hs.12381 | AI041543 | 144423 | chr12q24.32 | — | Hs.12381_at | hypothetical protein FLJ31978 | FLJ31978 | Amy | 0.468 | 1.096 | 0.358 | 1.065 | 0.025 | 1.302 |
| Hs.12409 | NM_001048 | 6750 | chr3q28 | 182450 | Hs.12409_at | somatostatin | SST | Amy | 0.585 | 1.129 | 0.430 | 1.137 | 0.045 | 0.547 |
| Hs.124177 | NM_016090 | 10179 | chr11q23.1-q23.2 | — | Hs.124177_at | RNA binding motif protein 7 | RBM7 | Amy | 0.556 | 0.886 | 0.901 | 1.021 | 0.010 | 0.698 |
| Hs.12449 | N63401 | 93377 | chr10q23-q24 | — | Hs.12449_at | transmembrane protein 10 | TMEM10 | Amy | 0.298 | 1.472 | 0.774 | 0.907 | 0.020 | 0.567 |
| Hs.124951 | AL039862 | 157638 | chr8q24.21 | — | Hs.124951_at | breast cancer membrane | NSE2 | Amy | 0.785 | 1.014 | 0.761 | 0.980 | 0.001 | 0.781 |
| | | | | | | protein 101 | | | | | | | | |
| Hs.125221 | NM_030755 | 81542 | chr14q22.1 | — | Hs.125221_at | thioredoxin domain containing | TXNDC | Amy | 0.646 | 1.050 | 0.340 | 0.910 | 0.006 | 0.748 |
| Hs.125293 | BF446578 | 221002 | chr10q11.21 | — | Hs.125293_at | CG4853 gene product | LOC221002 | Amy | 0.840 | 1.009 | 0.224 | 1.081 | 0.004 | 1.235 |
| Hs.126357 | NM_000276 | 4952 | chrxq25-q26.1 | 309000 | Hs.126357_at | oculocerebrorenal syndrome of | OCRL | Amy | 0.775 | 1.060 | 0.019 | 1.178 | 0.035 | 1.410 |
| | | | | | | Lowe | | | | | | | | |
| Hs.126372 | NM_016056 | 51643 | chr12q14.1-q15 | — | Hs.126372_at | CGI-119 protein | CGI-119 | Amy | 0.631 | 1.072 | 0.468 | 0.938 | 0.028 | 0.789 |
| Hs.126825 | BE672097 | 348487 | chr1p36.13 | — | Hs.126825_at | hypothetical protein FLJ36766 | FLJ36766 | Amy | 0.774 | 0.974 | 0.485 | 1.167 | 0.047 | 1.482 |
| Hs.12696 | AF131790 | 22941 | chr11q13.3-q13.4 | 603290 | Hs.12696_at | SH3 and multiple ankyrin repeat | SHANK2 | Amy | 0.571 | 1.027 | 0.290 | 1.070 | 0.001 | 1.341 |
| | | | | | | domains 2 | | | | | | | | |
| Hs.127196 | AI970061 | 151556 | chr2q31.1 | — | Hs.127196_at | G protein-coupled receptor 155 | GPR155 | Amy | 0.054 | 1.195 | 0.381 | 1.082 | 0.036 | 1.245 |
| Hs.12813 | BC033324 | 25976 | chr3q25.31 | — | Hs.12813_at | TCDD-inducible poly(ADP- | TIPARP | Amy | 0.316 | 0.933 | 0.121 | 0.796 | 0.036 | 0.709 |
| | | | | | | ribose) polymerase | | | | | | | | |
| Hs.128690 | AW172584 | 255783 | chr19q13.33 | — | Hs.128690_at | hypothetical protein LOC255783 | LOC255783 | Amy | 0.783 | 1.018 | 0.417 | 1.056 | 0.046 | 1.258 |
| Hs.129051 | BE550452 | 9456 | chr5q14.2 | 604798 | Hs.129051_at | homer homolog 1 (Drosophila) | HOMER1 | Amy | 0.137 | 1.191 | 0.544 | 1.067 | 0.047 | 1.314 |
| Hs.12953 | AI692180 | 8495 | chr11p15.4 | 603142 | Hs.12953_at | PTPRF interacting protein, | PPFIBP2 | Amy | 0.770 | 1.013 | 0.179 | 0.839 | 0.003 | 0.589 |
| | | | | | | binding protein 2 (liprin beta 2) | | | | | | | | |
| Hs.129783 | AF107028 | 6327 | chr11q23 | 601327 | Hs.129783_at | sodium channel, voltage-gated, | SCN2B | Amy | 0.374 | 1.184 | 0.139 | 1.196 | 0.037 | 1.275 |
| | | | | | | type II, beta | | | | | | | | |
| Hs.130065 | NM_020397 | 57118 | chr10p13 | 607957 | Hs.130065_at | calcium/calmodulin-dependent | CAMK1D | Amy | 0.929 | 1.009 | 0.647 | 0.976 | 0.022 | 1.269 |
| | | | | | | protein kinase ID | | | | | | | | |
| Hs.13014 | BC005122 | 26286 | chr22q13.2-q13.3 | — | Hs.13014_at | ADP-ribosylation factor GTPase | ARFGAP3 | Amy | 0.717 | 1.017 | 0.849 | 1.016 | 0.041 | 0.802 |
| | | | | | | activating protein 3 | | | | | | | | |
| Hs.13040 | NM_023914 | 53829 | chr3q24 | 606380 | Hs.13040_at | G protein-coupled receptor 86 | GPR86 | Amy | 0.472 | 0.990 | 0.460 | 0.981 | 0.030 | 0.573 |
| Hs.131055 | AI016313 | 10361 | chr8p21.3 | 608073 | Hs.131055_at | nucleophosmin/nucleoplasmin, 2 | NPM2 | Amy | 0.171 | 1.128 | 0.216 | 1.079 | 0.011 | 1.328 |
| Hs.131315 | AF307338 | 83666 | chr3q13-q21 | — | Hs.131315_at | B aggressive lymphoma gene | BAL | Amy | 0.220 | 0.975 | 0.079 | 0.926 | 0.000 | 0.717 |
| Hs.132275 | AI458003 | 116159 | chr21q21.2 | — | Hs.132275_at | cysteine and tyrosine-rich 1 | CYYR1 | Amy | 0.244 | 0.948 | 0.124 | 0.931 | 0.013 | 0.806 |
| Hs.132380 | NM_024306 | 79152 | chr16q23 | — | Hs.132380_at | fatty acid 2-hydroxylase | FA2H | Amy | 0.608 | 1.085 | 0.643 | 0.886 | 0.023 | 0.594 |
| Hs.132554 | R15072 | 151473 | chr2q36.3 | — | Hs.132554_at | hypothetical protein FLJ30794 | FLJ30794 | Amy | 0.082 | 1.251 | 0.140 | 1.142 | 0.025 | 1.359 |
| Hs.13340 | NM_003642 | 8520 | chr2q31.2-q33.1 | 603053 | Hs.13340_at | histone acetyltransferase 1 | HAT1 | Amy | 0.362 | 0.881 | 0.730 | 1.039 | 0.020 | 0.786 |
| Hs.134065 | NM_138339 | 171582 | chr10q26.13 | — | Hs.134065_at | hypothetical protein | DKFZp761H2121 | Amy | 0.078 | 1.331 | 0.102 | 1.278 | 0.043 | 1.260 |
| | | | | | | DKFZp761H2121 | | | | | | | | |
| Hs.134292 | BC006362 | 84814 | chr9q34.13 | — | Hs.134292_at | chromosome 9 open reading | C9orf67 | Amy | 0.657 | 1.048 | 0.342 | 1.105 | 0.027 | 1.399 |
| | | | | | | frame 67 | | | | | | | | |
| Hs.135056 | AL121758 | 140809 | chr20p13 | — | Hs.135056_at | chromosome 20 open reading | C20orf139 | Amy | 0.862 | 1.019 | 0.271 | 1.067 | 0.002 | 1.277 |
| | | | | | | frame 139 | | | | | | | | |
| Hs.135183 | NM_006869 | 11033 | chr7p22.3 | 608114 | Hs.135183_at | centaurin, alpha 1 | CENTA1 | Amy | 0.976 | 1.004 | 0.492 | 1.050 | 0.038 | 1.212 |
| Hs.141308 | U18840 | 4340 | chr6p22-p21.3 | 159465 | Hs.141308_at | myelin oligodendrocyte | MOG | Amy | 0.501 | 1.355 | 0.801 | 0.894 | 0.017 | 0.455 |
| | | | | | | glycoprotein | | | | | | | | |
| Hs.144287 | NM_012259 | 23493 | chr6q22.2-q22.33 | 604674 | Hs.144287_at | hairy/enhancer-of-split related | HEY2 | Amy | 0.087 | 0.914 | 0.514 | 0.935 | 0.015 | 0.754 |
| | | | | | | with YRPW motif 2 | | | | | | | | |
| Hs.144502 | NM_024779 | 79837 | chr12q13.3 | — | Hs.144502_at | phosphatidylinositol-4- | PIP5K2C | Amy | 0.007 | 1.171 | 0.110 | 1.137 | 0.036 | 1.225 |
| | | | | | | phosphate 5-kinase, type II, | | | | | | | | |
| | | | | | | gamma | | | | | | | | |
| Hs.14453 | | | | | Hs.14453_at | interferon consensus sequence | ICSBP1 | Amy | 0.287 | 0.871 | 0.186 | 0.862 | 0.027 | 0.760 |
| | | | | | | binding protein 1 | | | | | | | | |
| Hs.145741 | NM_001154 | 308 | chr4q28-q32 | 131230 | Hs.145741_at | annexin A5 | ANXA5 | Amy | 0.631 | 0.832 | 0.354 | 0.878 | 0.005 | 0.651 |
| Hs.14601 | NM_005335 | 3059 | chr3q13 | 601306 | Hs.14601_at | hematopoletic cell-specific Lyn | HCLS1 | Amy | 0.117 | 0.936 | 0.062 | 0.830 | 0.006 | 0.543 |
| | | | | | | substrate 1 | | | | | | | | |
| Hs.146393 | AF217990 | 9709 | chr16q12.2-q13 | 608070 | Hs.146393_at | homocysteine-inducible, | HERPUD1 | Amy | 0.729 | 1.034 | 0.418 | 0.971 | 0.010 | 0.796 |
| | | | | | | endoplasmic reticulum stress- | | | | | | | | |
| | | | | | | inducible, ubiquitin-like domain | | | | | | | | |
| | | | | | | member 1 | | | | | | | | |
| Hs.14845 | N25732 | 2309 | chr6q21 | 602681 | Hs.14845_at | forkhead box O3A | FOXO3A | Amy | 0.432 | 1.130 | 0.075 | 1.091 | 0.027 | 1.249 |
| Hs.149156 | NM_000170 | 2731 | chr9p22 | 238300 | Hs.149156_at | glycine dehydrogenase | GLDC | Amy | 0.542 | 1.022 | 0.120 | 1.134 | 0.026 | 1.280 |
| | | | | | | (decarboxylating; glycine | | | | | | | | |
| | | | | | | decarboxylase, glycine | | | | | | | | |
| | | | | | | cleavage system protein P) | | | | | | | | |
| Hs.150101 | J03263 | 3916 | chr13q34 | 153330 | Hs.150101_at | lysosomal-associated | LAMP1 | Amy | 0.207 | 1.077 | 0.866 | 1.015 | 0.045 | 0.807 |
| | | | | | | membrane protein 1 | | | | | | | | |
| Hs.150956 | NM_004455 | 2134 | chr1p36.1 | 601738 | Hs.150956_at | exostoses (multiple)-like 1 | EXTL1 | Amy | 0.843 | 1.011 | 0.130 | 1.133 | 0.003 | 1.322 |
| Hs.151414 | AW409611 | 91404 | chr2q31.2 | — | Hs.151414_at | hypothetical protein | DKFZp434O | Amy | 0.917 | 1.024 | 0.567 | 0.949 | 0.017 | 0.781 |
| | | | | | | DKFZp434O0515 | 0515 | | | | | | | |
| Hs.15159 | NM_016951 | 51192 | chr16q22.1 | — | Hs.15159_at | chemokine-like factor | CKLF | Amy | 0.468 | 0.970 | 0.841 | 1.008 | 0.019 | 0.803 |
| Hs.152149 | AL137589 | 54620 | chr16p11.2 | — | Hs.152149_at | hypothetical protein | DKFZP434K0410 | Amy | 0.849 | 1.020 | 0.876 | 1.012 | 0.031 | 1.288 |
| | | | | | | DKFZp434K0410 | | | | | | | | |
| Hs.153355 | AF142421 | 9444 | chr6q26-27 | — | Hs.153355_at | quaking homolog, KH domain | QKI | Amy | 0.784 | 0.969 | 0.585 | 0.926 | 0.020 | 0.789 |
| | | | | | | RNA binding (mouse) | | | | | | | | |
| Hs.153563 | NM_002349 | 4065 | chr2q24 | 604524 | Hs.153563_at | lymphocyte antigen 75 | LY75 | Amy | 0.689 | 0.976 | 0.082 | 0.789 | 0.037 | 0.669 |
| Hs.153647 | NM_002380 | 4147 | chr8q22 | 602108 | Hs.153647_at | matrilin 2 | MATN2 | Amy | 0.813 | 0.984 | 0.262 | 0.835 | 0.042 | 0.635 |
| Hs.153716 | AI377497 | 259230 | chr10q11.2 | — | Hs.153716_at | mob protein | MOB | Amy | 0.940 | 1.034 | 0.317 | 0.844 | 0.050 | 0.781 |
| Hs.153792 | N29717 | 4552 | chr5p15.3-p15.2 | 602568 | Hs.153792_at | 5-methyltetrahydrofolate- | MTRR | Amy | 0.351 | 0.957 | 0.143 | 0.863 | 0.007 | 0.765 |
| | | | | | | homocysteine | | | | | | | | |
| | | | | | | methyltransferase reductase | | | | | | | | |
| Hs.153837 | NM_002432 | 4332 | chr1q22 | 159553 | Hs.153837_at | myeloid cell nuclear | MNDA | Amy | 0.216 | 0.889 | 0.805 | 0.964 | 0.018 | 0.597 |
| | | | | | | differentiation antigen | | | | | | | | |
| Hs.154437 | NM_002599 | 5138 | chr11q13.4 | 602658 | Hs.154437_at | phosphodiesterase 2A, cGMP- | PDE2A | Amy | 0.057 | 1.191 | 0.072 | 1.152 | 0.014 | 1.343 |
| | | | | | | stimulated | | | | | | | | |
| Hs.154658 | NM_002779 | 5662 | chr10q24 | 602327 | Hs.154658_at | pleckstrin and Sec7 domain | PSD | Amy | 0.791 | 0.982 | 0.123 | 1.182 | 0.044 | 1.248 |
| | | | | | | protein | | | | | | | | |
| Hs.154729 | NM_002613 | 5170 | chr16p13.3 | 605213 | Hs.154729_at | 3-phosphoinositide dependent | PDPK1 | Amy | 0.999 | 1.000 | 0.725 | 1.013 | 0.000 | 1.213 |
| | | | | | | protein kinase-1 | | | | | | | | |
| Hs.155718 | NM_018434 | 55819 | chr5q35.3 | — | Hs.155718_at | ring finger protein 130 | RNF130 | Amy | 0.426 | 1.116 | 0.867 | 0.975 | 0.007 | 0.706 |
| Hs.155935 | U62027 | 719 | chr12p13.31 | 605246 | Hs.155935_at | complemet component 3a | C3AR1 | Amy | 0.261 | 0.935 | 0.011 | 0.856 | 0.017 | 0.751 |
| | | | | | | receptor 1 | | | | | | | | |
| Hs.15711 | NM_015254 | 23303 | chr8p12 | 607350 | Hs.15711_at | kinesin family member 13B | KIF13B | Amy | 0.214 | 1.108 | 0.903 | 0.982 | 0.013 | 0.677 |
| Hs.157427 | | | | | Hs.157427_at | ret finger protein-like 2 | RFPL2 | Amy | 0.512 | 0.863 | 0.779 | 0.954 | 0.040 | 1.355 |
| Hs.15783 | AW664953 | 85362 | chr20q13.33 | — | Hs.15783_at | chromosome 20 open reading | C20orf158 | Amy | 0.271 | 1.093 | 0.146 | 0.916 | 0.018 | 0.815 |
| | | | | | | frame 158 | | | | | | | | |
| Hs.158867 | AU145019 | 23150 | chr3p14.2 | — | Hs.158867_at | GRP1-binding protein GRSP1 | GRSP1 | Amy | 0.629 | 0.966 | 0.862 | 0.977 | 0.012 | 0.679 |
| Hs.1588 | NM_000663 | 18 | chr16p13.2 | 137150 | Hs.1588_at | 4-aminobutyrate | ABAT | Amy | 0.595 | 1.119 | 0.041 | 1.080 | 0.007 | 1.222 |
| | | | | | | aminotransferase | | | | | | | | |
| Hs.159425 | AB056866 | 50859 | chr4q32.3 | 607989 | Hs.159425_at | sparc/osteonectin, cwcv and | SPOCK3 | Amy | 0.164 | 1.148 | 0.690 | 1.040 | 0.009 | 0.655 |
| | | | | | | kazal-like domains proteoglycan | | | | | | | | |
| | | | | | | (testican) 3 | | | | | | | | |
| Hs.161999 | NM_003244 | 7050 | chr18p11.3 | 602630 | Hs.161999_at | TGFB-induced factor (TALE | TGIF | Amy | 0.447 | 0.938 | 0.297 | 1.079 | 0.017 | 0.691 |
| | | | | | | family homeobox) | | | | | | | | |
| Hs.1619 | BE797438 | 429 | chr12q22-q23 | 100790 | Hs.1619_at | achaete-scute complex-like 1 | ASCL1 | Amy | 0.066 | 0.777 | 0.140 | 0.788 | 0.012 | 0.779 |
| | | | | | | (Drosophila) | | | | | | | | |
| Hs.163113 | NM_024510 | 79415 | chr17q25.3 | — | Hs.163113_at | hypothetical protein MGC4368 | MGC4368 | Amy | 0.957 | 0.995 | 0.437 | 0.966 | 0.048 | 0.813 |
| Hs.16512 | NM_024576 | 79627 | chr6q13 | — | Hs.16512_at | oploid growth factor receptor- | OGFRL1 | Amy | 0.128 | 0.959 | 0.078 | 0.866 | 0.012 | 0.824 |
| | | | | | | like 1 | | | | | | | | |
| Hs.165563 | AL118506 | 80331 | chr20q13.33 | — | Hs.165563_at | DnaJ (Hsp40) homolog, | DNAJC5 | Amy | 0.063 | 1.135 | 0.073 | 1.126 | 0.030 | 1.266 |
| | | | | | | subfamily C, member 5 | | | | | | | | |
| Hs.166017 | BC012503 | 4286 | chr3p14.2-p14.1 | 156845 | Hs.166017_at | microphthalmia-associated | MITF | Amy | 0.880 | 1.011 | 0.679 | 0.960 | 0.033 | 0.781 |
| | | | | | | transcription factor | | | | | | | | |
| Hs.166244 | NM_023933 | 65990 | chr16p13.3 | — | Hs.166244_at | hypothetical protein MGC2494 | MGC2494 | Amy | 0.964 | 0.993 | 0.421 | 1.082 | 0.018 | 1.201 |
| Hs.166684 | NM_006281 | 6788 | chr8q22.2 | 605030 | Hs.166684_at | serine/threonine kinase 3 | STK3 | Amy | 0.718 | 0.958 | 0.176 | 0.820 | 0.026 | 0.677 |
| | | | | | | (STE20 homolog, yeast) | | | | | | | | |
| Hs.166705 | AF062006 | 8549 | chr12q22-q23 | 606667 | Hs.166705_at | G protein-coupled receptor 49 | GPR49 | Amy | 0.265 | 1.041 | 0.649 | 1.049 | 0.039 | 0.829 |
| Hs.167746 | NM_013314 | 29760 | chr10q23.2-q23.33 | 604515 | Hs.167746_at | B-cell linker | BLNK | Amy | 0.284 | 0.961 | 0.348 | 0.858 | 0.003 | 0.467 |
| Hs.167 | U89330 | 4133 | chr2q34-q35 | 157130 | Hs.167_at | microtubule-associated protein 2 | MAP2 | Amy | 0.239 | 1.254 | 0.043 | 1.090 | 0.030 | 1.272 |
| Hs.169600 | BC021803 | 23045 | chr4p12 | — | Hs.169600_at | KIAA0826 protein | KIAA0826 | Amy | 0.641 | 1.023 | 0.908 | 0.990 | 0.032 | 0.796 |
| Hs.170087 | NM_001621 | 196 | chr7p15 | 600253 | Hs.170087_at | aryl hydrocarbon receptor | AHR | Amy | 0.757 | 1.055 | 0.273 | 0.875 | 0.032 | 0.591 |
| Hs.170198 | BF214329 | 9650 | chr8q13.1 | — | Hs.170198_at | likely ortholog of chicken | CHPPR | Amy | 0.222 | 0.972 | 0.991 | 1.000 | 0.017 | 0.805 |
| | | | | | | chondrocyte protein with a polyproline | | | | | | | | |
| | | | | | | region | | | | | | | | |
| Hs.170328 | NM_002444 | 4478 | chrxq11.2-q12 | 309845 | Hs.170328_at | moesin | MSN | Amy | 0.125 | 0.764 | 0.208 | 0.867 | 0.029 | 0.800 |
| Hs.1706 | | | | | Hs.1706_at | interferon-stimulated | ISGF3G | Amy | 0.121 | 0.780 | 0.168 | 0.866 | 0.001 | 0.764 |
| | | | | | | transcription factor 3, gamma | | | | | | | | |
| | | | | | | 48 kDa | | | | | | | | |
| Hs.171834 | NM_006201 | 5127 | chrxp11.3-p11.23 | 311550 | Hs.171834_at | PCTAIRE protein kinase 1 | PCTK1 | Amy | 0.474 | 1.038 | 0.005 | 1.162 | 0.027 | 1.264 |
| | | | p11.23 | | | | | | | | | | | |
| Hs.172089 | BG538627 | 114908 | chr11q22.1 | 606356 | Hs.172089_at | pro-oncosis receptor inducing | PORIMIN | Amy | 0.630 | 0.828 | 0.137 | 0.735 | 0.006 | 0.637 |
| | | | | | | membrane injury gene | | | | | | | | |
| Hs.172550 | NM_002819 | 5725 | chr19p13.3 | 600693 | Hs.172550_at | polypyrimidine tract binding | PTBP1 | Amy | 0.101 | 0.921 | 0.147 | 0.877 | 0.033 | 0.789 |
| | | | | | | protein 1 | | | | | | | | |
| Hs.172631 | NM_000632 | 3684 | chr16p11.2 | 120980 | Hs.172631_at | integrin, alpha M (complement | ITGAM | Amy | 0.180 | 0.952 | 0.217 | 0.843 | 0.010 | 0.534 |
| | | | | | | component receptor 3, alpha; | | | | | | | | |
| | | | | | | also known as CD11b (p170), | | | | | | | | |
| | | | | | | macrophage antigen alpha | | | | | | | | |
| | | | | | | polypeptide) | | | | | | | | |
| Hs.173392 | BG177562 | 57458 | chr12q22 | — | Hs.173392_at | KIAA1145 protein | KIAA1145 | Amy | 0.588 | 1.068 | 0.527 | 0.873 | 0.011 | 0.595 |
| Hs.173438 | NM_018147 | 55179 | chr3q22.3 | — | Hs.173438_at | Fas apoptotic inhibitory | FAIM | Amy | 0.403 | 1.135 | 0.848 | 0.978 | 0.049 | 0.833 |
| | | | | | | molecule | | | | | | | | |
| Hs.173560 | AB032953 | 57451 | chr5q34 | — | Hs.173560_at | odd Oz/ten-m homolog 2 | ODZ2 | Amy | 0.293 | 1.216 | 0.147 | 1.142 | 0.009 | 1.338 |
| Hs.173864 | AB011133 | 23031 | chr19p13.12-p13.11 | — | Hs.173864_at | KIAA0561 protein | KIAA0561 | Amy | 0.605 | 1.170 | 0.094 | 1.203 | 0.027 | 1.346 |
| Hs.174142 | NM_005211 | 1436 | chr5q33-q35 | 164770 | Hs.174142_at | colony stimulating factor 1 | CSF1R | Amy | 0.538 | 0.907 | 0.054 | 0.628 | 0.004 | 0.472 |
| | | | | | | receptor, formerly McDonough | | | | | | | | |
| | | | | | | feline sarcoma viral (v-fms) | | | | | | | | |
| | | | | | | oncogene homolog | | | | | | | | |
| Hs.174195 | NM_006435 | 10581 | chr11p15.5 | 605578 | Hs.174195_at | interferon induced | IFITM2 | Amy | 0.794 | 1.106 | 0.370 | 0.892 | 0.021 | 0.700 |
| | | | | | | transmembrane protein 2 (1-8D) | | | | | | | | |
| Hs.1742 | NM_003870 | 8826 | chr15q26.1 | 603379 | Hs.1742_at | IQ motif containing GTPase | IQGAP1 | Amy | 0.321 | 0.964 | 0.072 | 0.924 | 0.014 | 0.820 |
| | | | | | | activating protein 1 | | | | | | | | |
| Hs.174312 | NM_138557 | 7099 | chr9q32-q33 | 603030 | Hs.174312_at | toll-like receptor 4 | TLR4 | Amy | 0.049 | 0.945 | 0.958 | 0.997 | 0.043 | 0.803 |
| Hs.17466 | NM_004585 | 5920 | chr11q23 | 605092 | Hs.17466_at | retinoic acid receptor responder | RARRES3 | Amy | 0.599 | 0.968 | 0.936 | 1.012 | 0.030 | 0.728 |
| | | | | | | (tazarotene induced) 3 | | | | | | | | |
| Hs.176227 | BC035811 | 55314 | chr4q32.1 | — | Hs.176227_at | hypothetical protein FLJ11155 | FLJ11155 | Amy | 0.710 | 1.064 | 0.638 | 0.871 | 0.012 | 0.541 |
| Hs.177534 | AK022513 | 11221 | chr1q41 | 608867 | Hs.177534_at | dual specificity phosphatase 10 | DUSP10 | Amy | 0.812 | 0.989 | 0.530 | 1.041 | 0.026 | 0.831 |
| Hs.1780 | X98405 | 4099 | chr19q13.1 | 159460 | Hs.1780_at | myelin associated glycoprotein | MAG | Amy | 0.256 | 1.331 | 0.850 | 0.937 | 0.028 | 0.533 |
| Hs.178137 | AA675892 | 10140 | chr17q21 | 605523 | Hs.178137_at | transducer of ERBB2, 1 | TOB1 | Amy | 0.541 | 0.836 | 0.064 | 0.788 | 0.027 | 0.662 |
| Hs.1787 | BC002665 | 5354 | chrxq22 | 300401 | Hs.1787_at | proteolipid protein 1 (Pelizaeus- | PLP1 | Amy | 0.625 | 1.246 | 0.739 | 0.927 | 0.009 | 0.741 |
| | | | | | | Merzbacher disease, spastic | | | | | | | | |
| | | | | | | paraplegia 2, uncomplicated) | | | | | | | | |
| Hs.180141 | AF134802 | 1073 | chr14q12 | 601443 | Hs.180141_at | cofilin2 (muscle) | CFL2 | Amy | 0.693 | 1.093 | 0.893 | 0.982 | 0.020 | 0.719 |
| Hs.180403 | NM_016271 | 51444 | chr18q12.1 | — | Hs.180403_at | ring finger protein 138 | RNF138 | Amy | 0.415 | 1.127 | 0.244 | 0.875 | 0.037 | 0.800 |
| Hs.180545 | AJ245600 | 91614 | chr11p13 | — | Hs.180545_at | novel 58.3 KDA protein | LOC91614 | Amy | 0.774 | 0.989 | 0.737 | 0.967 | 0.014 | 0.646 |
| Hs.180866 | NM_000416 | 3459 | chr6q23-q24 | 107470 | Hs.180866_at | interferon gamma receptor 1 | IFNGR1 | Amy | 0.703 | 0.927 | 0.185 | 0.854 | 0.009 | 0.687 |
| Hs.180877 | BC001124 | 3021 | chr17q25 | 601058 | Hs.180877_at | H3 histone, family 3B (H3.3B) | H3F3B | Amy | 1.000 | 1.000 | 0.816 | 1.021 | 0.017 | 0.794 |
| Hs.181046 | AL048503 | 1845 | chr17q21 | 600183 | Hs.181046_at | dual specificity phosphatase 3 | DUSP3 | Amy | 0.077 | 1.252 | 0.296 | 1.095 | 0.020 | 1.245 |
| | | | | | | (vaccinia virus phosphatase | | | | | | | | |
| | | | | | | VH1-related) | | | | | | | | |
| Hs.181244 | AA573862 | 3105 | chr6p21.3 | 142800 | Hs.181244_at | major histocompatibility | HLA-A | Amy | 0.464 | 0.921 | 0.208 | 0.916 | 0.030 | 0.769 |
| | | | | | | complex, class I, A | | | | | | | | |
| Hs.181301 | BC002642 | 1520 | chr1q21 | 116845 | Hs.181301_at | cathepsin S | CTSS | Amy | 0.167 | 0.943 | 0.370 | 0.956 | 0.004 | 0.770 |
| Hs.182579 | NM_015907 | 51056 | chr4p15.32 | 170250 | Hs.182579_at | leucine aminopeptidase 3 | LAP3 | Amy | 0.263 | 1.098 | 0.319 | 1.079 | 0.002 | 0.789 |
| Hs.1832 | NM_000905 | 4852 | chr7p15.1 | 162640 | Hs.1832_at | neuropeptide Y | NPY | Amy | 0.403 | 1.128 | 0.751 | 0.942 | 0.002 | 0.491 |
| Hs.183506 | BC008922 | 79899 | chr11p13-p12 | — | Hs.183506_at | hypothetical protein FLJ14213 | FLJ14213 | Amy | 0.807 | 1.010 | 0.932 | 1.004 | 0.011 | 0.739 |
| Hs.183702 | AK021814 | 92344 | chr1q24.2 | 607983 | Hs.183702_at | hypothetical protein FLJ11752 | FLJ11752 | Amy | 0.260 | 1.077 | 0.765 | 0.964 | 0.024 | 0.811 |
| Hs.184018 | NM_004271 | 9450 | chr6p25.1 | 605241 | Hs.184018_at | lymphocyte antigen 86 | LY86 | Amy | 0.938 | 0.993 | 0.052 | 0.810 | 0.043 | 0.603 |
| Hs.187377 | NM_024847 | 79905 | chr16p12.3 | — | Hs.187377_at | transmembrane channel-like 7 | TMC7 | Amy | 0.393 | 1.051 | 0.147 | 0.867 | 0.016 | 0.795 |
| Hs.188011 | AI301935 | 58475 | chr11q12 | 606502 | Hs.188011_at | membrane-spanning 4- | MS4A7 | Amy | 0.040 | 0.943 | 0.052 | 0.884 | 0.047 | 0.621 |
| | | | | | | domains, subfamily A, member 7 | | | | | | | | |
| Hs.188 | NM_002600 | 5142 | chr1p31 | 600127 | Hs.188_at | phosphodiesterase 4B, cAMP- | PDE4B | Amy | 0.483 | 1.076 | 0.922 | 0.994 | 0.024 | 0.792 |
| | | | | | | specific (phosphodiesterase E4 | | | | | | | | |
| | | | | | | dunce homolog, Drosophila) | | | | | | | | |
| Hs.190043 | AW469184 | 158747 | chrxp22.2 | — | Hs.190043_at | hypothetical protein MGC26706 | MGC26706 | Amy | 0.818 | 1.033 | 0.618 | 0.918 | 0.003 | 0.605 |
| Hs.1908 | BC022313 | 5552 | chr10q22.1 | 177040 | Hs.1908_at | proteoglycan 1, secretory | PRG1 | Amy | 0.091 | 0.952 | 0.258 | 0.810 | 0.001 | 0.524 |
| | | | | | | granule | | | | | | | | |
| Hs.1915 | NM_004476 | 2346 | chr11p11.2 | 600934 | Hs.1915_at | folate hydrolase (prostate- | FOLH1 | Amy | 0.465 | 1.109 | 0.620 | 0.799 | 0.011 | 0.402 |
| | | | | | | specific membrane antigen) 1 | | | | | | | | |
| Hs.19210 | BF690150 | 84975 | chr12q13.13 | — | Hs.19210_at | hypothetical protein MGC11308 | MGC11308 | Amy | 0.305 | 1.045 | 0.102 | 1.104 | 0.006 | 1.252 |
| Hs.192491 | NM_012113 | 23632 | chr1q21 | 604832 | Hs.192491_at | carbonic anhydrase XIV | CA14 | Amy | 0.544 | 0.971 | 0.541 | 0.943 | 0.029 | 0.786 |
| Hs.193115 | T52285 | 85444 | chr8q21.2 | — | Hs.193115_at | KIAA1764 protein | KIAA1764 | Amy | 0.024 | 0.842 | 0.246 | 0.807 | 0.022 | 0.577 |
| Hs.194684 | NM_003458 | 8927 | chr3p21.31 | 604020 | Hs.194684_at | bassoon (presynaptic cytomatrix | BSN | Amy | 0.955 | 0.987 | 0.999 | 1.000 | 0.025 | 1.529 |
| | | | | | | protein) | | | | | | | | |
| Hs.195471 | NM_004566 | 5209 | chr10p14-p15 | 605319 | Hs.195471_at | 6-phosphofructo-2- | PFKFB3 | Amy | 0.922 | 0.988 | 0.060 | 0.865 | 0.013 | 0.762 |
| | | | | | | kinase/fructose-2,6- | | | | | | | | |
| | | | | | | biphosphatase 3 | | | | | | | | |
| Hs.196083 | AL137653 | 1487 | chr4p16 | 602618 | Hs.196083_at | C-terminal binding protein 1 | CTBP1 | Amy | 0.230 | 1.215 | 0.109 | 1.166 | 0.035 | 1.247 |
| Hs.197045 | BF435286 | 25938 | chr14q12 | — | Hs.197045_at | chromosome 14 open reading | C14orf125 | Amy | 0.196 | 0.841 | 0.314 | 0.817 | 0.027 | 0.739 |
| | | | | | | frame 125 | | | | | | | | |
| Hs.197298 | AF205218 | 10625 | chr1q25.1-q31.1 | — | Hs.197298_at | influenza virus NS1A binding | IVNS1ABP | Amy | 0.337 | 1.150 | 0.907 | 0.986 | 0.049 | 0.794 |
| | | | | | | protein | | | | | | | | |
| Hs.197335 | NM_006102 | 10404 | chr8q22.2 | — | Hs.197335_at | plasma glutamate | PGCP | Amy | 0.127 | 0.955 | 0.507 | 0.927 | 0.023 | 0.807 |
| | | | | | | carboxypeptidase | | | | | | | | |
| Hs.199068 | AW016039 | 57172 | chr1q32-q41 | — | Hs.199068_at | calcium/calmodulin-dependent | CAMK1G | Amy | 0.208 | 1.179 | 0.087 | 1.164 | 0.037 | 1.410 |
| | | | | | | protein kinase IG | | | | | | | | |
| Hs.199364 | NM_018103 | 55144 | chr1p22.2 | — | Hs.199364_at | leucine rich repeat containing 5 | LRRC5 | Amy | 0.130 | 1.129 | 0.669 | 0.963 | 0.039 | 0.750 |
| Hs.200481 | NM_018340 | 55313 | chr16p13.12 | — | Hs.200481_at | hypothetical protein FLJ11151 | FLJ11151 | Amy | 0.155 | 1.035 | 0.964 | 1.001 | 0.013 | 0.825 |
| Hs.200586 | NM_001703 | 576 | chr1p35 | 602683 | Hs.200586_at | brain-specific angiogenesis | BAI2 | Amy | 0.714 | 1.150 | 0.191 | 1.095 | 0.046 | 1.267 |
| | | | | | | inhibitor 2 | | | | | | | | |
| Hs.201369 | NM_015559 | 26040 | chr18q21.1 | — | Hs.201369_at | SET binding protein 1 | SETBP1 | Amy | 0.996 | 1.000 | 0.103 | 1.093 | 0.047 | 1.236 |
| Hs.20137 | AW504018 | 55589 | chr4q21.23 | — | Hs.20137_at | BMP2 inducible kinase | BMP2K | Amy | 0.945 | 0.994 | 0.827 | 0.980 | 0.002 | 0.781 |
| Hs.201702 | AI868167 | 286097 | chr8p22 | — | Hs.201702_at | hypothetical protein LOC286097 | LOC286097 | Amy | 0.316 | 1.218 | 0.763 | 1.031 | 0.027 | 1.267 |
| Hs.202308 | BC003686 | 8773 | chr15q15.1 | 602534 | Hs.202308_at | synaptosomal-associated | SNAP23 | Amy | 0.376 | 0.969 | 0.563 | 0.963 | 0.000 | 0.781 |
| | | | | | | protein, 23 kDa | | | | | | | | |
| Hs.20252 | AL096776 | 58480 | chr1q42.11-q42.3 | 606366 | Hs.20252_at | ras homolog gene family, | ARHU | Amy | 0.841 | 1.023 | 0.678 | 0.914 | 0.006 | 0.604 |
| | | | | | | member U | | | | | | | | |
| Hs.202687 | NM_012281 | 3751 | chr7q31 | 605410 | Hs.202687_at | potassium voltage-gated | KCND2 | Amy | 0.384 | 1.332 | 0.149 | 1.178 | 0.010 | 1.346 |
| | | | | | | channel, Shal-related subfamily, | | | | | | | | |
| | | | | | | member 2 | | | | | | | | |
| Hs.203213 | NM_000461 | 7068 | chr3p24.3 | 190160 | Hs.203213_at | thyroid hormone receptor, beta | THRB | Amy | 0.352 | 1.080 | 0.367 | 1.053 | 0.012 | 1.473 |
| | | | | | | (erythroblastic leukemia viral (verb- | | | | | | | | |
| | | | | | | a) oncogene homolog 2, | | | | | | | | |
| | | | | | | avian) | | | | | | | | |
| Hs.20369 | BE466825 | 147929 | chr19q13.12 | — | Hs.20369_at | hypothetical protein FLJ36991 | FLJ36991 | Amy | 0.102 | 0.838 | 0.132 | 0.870 | 0.027 | 0.740 |
| Hs.204539 | BC002461 | 663 | chr15q22.2 | 603292 | Hs.204539_at | BCL2/adenovirus E1B 19 kDa | BNIP2 | Amy | 0.483 | 0.979 | 0.638 | 0.960 | 0.011 | 0.812 |
| | | | | | | interacting protein 2 | | | | | | | | |
| Hs.205088 | AK002207 | 23111 | chr13q13.3 | 607111 | Hs.205088_at | spastic paraplegia 20, spartin | SPG20 | Amy | 0.816 | 0.975 | 0.759 | 0.972 | 0.012 | 0.781 |
| | | | | | | (Troyer syndrome) | | | | | | | | |
| Hs.205865 | AW043782 | 143458 | chr11p13 | — | Hs.205865_at | hypothetical protein LOC143458 | LOC143458 | Amy | 0.025 | 0.849 | 0.062 | 0.753 | 0.037 | 0.698 |
| Hs.206770 | AL523144 | 9278 | chr6p21.3 | — | Hs.206770_at | zinc finger protein 297 | ZNF297 | Amy | 0.271 | 1.080 | 0.208 | 1.113 | 0.029 | 1.247 |
| Hs.207428 | BF213279 | 10160 | chr13q32.2 | 602654 | Hs.207428_at | FERM, RhoGEF (ARHGEF) and | FARP1 | Amy | 0.402 | 1.069 | 0.294 | 1.102 | 0.004 | 1.305 |
| | | | | | | pleckstrin domain protein 1 | | | | | | | | |
| | | | | | | (chondrocyte-derived) | | | | | | | | |
| Hs.207776 | | | | | Hs.207776_at | aspartylglucosaminidase | AGA | Amy | 0.824 | 1.015 | 0.326 | 0.861 | 0.008 | 0.648 |
| Hs.21104 | NM_173602 | 57609 | chr12q13.13 | — | Hs.21104_at | KIAA1463 protein | KIAA1463 | Amy | 0.667 | 1.024 | 0.490 | 0.945 | 0.031 | 0.772 |
| Hs.21126 | AB014594 | 55619 | chr2q36.3 | — | Hs.21126_at | dedicator of cytokinesis 10 | DOCK10 | Amy | 0.579 | 1.025 | 0.399 | 1.090 | 0.023 | 0.744 |
| Hs.211569 | AI338653 | 2869 | chr10q24-qter | 600870 | Hs.211569_at | G protein-coupled receptor | GPRK5 | Amy | 0.046 | 1.050 | 0.561 | 1.041 | 0.045 | 1.237 |
| | | | | | | kinase 5 | | | | | | | | |
| Hs.211751 | NM_020836 | 57596 | chr14q32.2 | — | Hs.211751_at | brain-enriched guanylate | KIAA1446 | Amy | 0.421 | 0.969 | 0.330 | 1.078 | 0.010 | 1.232 |
| | | | | | | kinase-associated protein | | | | | | | | |
| Hs.212296 | NM_000210 | 3655 | chr2q31.1 | 147556 | Hs.212296_at | integrin, alpha 6 | ITGA6 | Amy | 0.386 | 0.901 | 0.042 | 0.875 | 0.017 | 0.671 |
| Hs.212957 | BC034803 | 286827 | chr3q26.1 | — | Hs.212957_at | tumor suppressor TSBF1 | TSBF1 | Amy | 0.963 | 1.003 | 0.550 | 0.873 | 0.030 | 0.551 |
| Hs.21420 | BC035596 | 56924 | chr15q14 | 608110 | Hs.21420_at | p21(CDKN1A)-activated kinase 6 | PAK6 | Amy | 0.988 | 1.001 | 0.035 | 1.198 | 0.041 | 1.336 |
| Hs.217112 | BC004271 | 84735 | chr18q22.3 | — | Hs.217112_at | camosinase 1 | CN1 | Amy | 0.416 | 0.768 | 0.207 | 0.545 | 0.001 | 0.340 |
| Hs.217409 | AA908763 | 118812 | chr10q24.2 | — | Hs.217409_at | 44050 protein | LOC118812 | Amy | 0.820 | 1.007 | 0.004 | 1.164 | 0.001 | 1.225 |
| Hs.21938 | NM_024586 | 114883 | chr1p32.3 | 606737 | Hs.21938_at | oxysterol binding protein-like 9 | OSBPL9 | Amy | 0.749 | 0.914 | 0.656 | 0.976 | 0.025 | 0.820 |
| Hs.22140 | NM_016564 | 51286 | chr11p15.5 | 608213 | Hs.22140_at | BM88 antigen | BM88 | Amy | 0.394 | 1.109 | 0.185 | 1.092 | 0.029 | 1.211 |
| Hs.22270 | AI879661 | 120196 | chr11p13 | — | Hs.22270_at | hypothetical protein MGC34830 | MGC34830 | Amy | 0.309 | 1.222 | 0.141 | 1.145 | 0.010 | 1.700 |
| Hs.2236 | Z25434 | 4752 | chr13q14.13 | 604044 | Hs.2236_at | NIMA (never in mitosis gene a)- | NEK3 | Amy | 0.843 | 0.995 | 0.921 | 1.012 | 0.020 | 0.685 |
| | | | | | | related kinase 3 | | | | | | | | |
| Hs.224505 | AV710838 | 83875 | chr11q22.3-q23.1 | — | Hs.224505_at | beta-carotene dioxygenase 2 | BCDO2 | Amy | 0.397 | 1.056 | 0.158 | 0.890 | 0.004 | 0.802 |
| Hs.227059 | NM_012128 | 22802 | chr1p31-p22 | — | Hs.227059_at | chloride channel, calcium | CLCA4 | Amy | 0.725 | 0.962 | 0.879 | 0.954 | 0.003 | 0.328 |
| | | | | | | activated, family member 4 | | | | | | | | |
| Hs.23106 | AI023317 | 9862 | chr17q21.1 | 607000 | Hs.23106_at | thyraid hormone receptor- | TRAP100 | Amy | 0.337 | 1.109 | 0.168 | 1.102 | 0.011 | 1.527 |
| | | | | | | associated protein (100 kDa) | | | | | | | | |
| Hs.23158 | AA906578 | 150726 | chr2p13.2 | — | Hs.23158_at | KIAA1940 protein | KIAA1940 | Amy | 0.419 | 1.107 | 0.047 | 1.123 | 0.012 | 1.297 |
| Hs.232004 | NM_006176 | 4900 | chr11q24 | 602350 | Hs.232004_at | neurogranin (protein kinase C | NRGN | Amy | 0.482 | 1.165 | 0.048 | 1.136 | 0.003 | 1.641 |
| | | | | | | substrate, RC3) | | | | | | | | |
| Hs.232400 | NM_002137 | 3181 | chr7p15 | 600124 | Hs.232400_at | heterogeneous nuclear | HNRPA2B1 | Amy | 0.832 | 1.027 | 0.969 | 1.002 | 0.039 | 0.828 |
| | | | | | | ribonucleoprotein A2/B1 | | | | | | | | |
| Hs.232432 | NM_013995 | 3920 | chrxq24 | 309060 | Hs.232432_at | lysosomal-associated | LAMP2 | Amy | 0.626 | 1.102 | 0.410 | 0.853 | 0.008 | 0.595 |
| | | | | | | membrane protein 2 | | | | | | | | |
| Hs.23567 | NM_003787 | 8715 | chr18q12 | 603577 | Hs.23567_at | nucleolar protein 4 | NOL4 | Amy | 0.499 | 1.056 | 0.195 | 1.093 | 0.002 | 1.289 |
| Hs.237536 | AL526783 | 115024 | chr17q21.2 | — | Hs.237536_at | hypothetical protein MGC20781 | MGC20781 | Amy | 0.964 | 1.005 | 0.064 | 1.118 | 0.017 | 1.229 |
| Hs.23765 | AL359575 | 127281 | chr1p36.32 | — | Hs.23765_at | hypothetical protein MGC26818 | MGC26818 | Amy | 0.827 | 1.016 | 0.374 | 1.054 | 0.029 | 1.261 |
| Hs.2391 | NM_001649 | 357 | chrxp22.3 | 300103 | Hs.2391_at | apical protein-like (Xenopus | APXL | Amy | 0.456 | 1.128 | 0.242 | 1.163 | 0.005 | 1.522 |
| | | | | | | laevis) | | | | | | | | |
| Hs.239500 | BC007207 | 84326 | chr16p13.3 | — | Hs.239500_at | hypothetical protein MGC13114 | MGC13114 | Amy | 0.718 | 1.059 | 0.052 | 1.244 | 0.029 | 1.294 |
| Hs.23954 | AA654142 | 51148 | chr9q34.11 | — | Hs.23954_at | cerebral endothelial cell | CEECAM1 | Amy | 0.940 | 1.018 | 0.450 | 0.809 | 0.013 | 0.527 |
| | | | | | | adhesion molecule 1 | | | | | | | | |
| Hs.24030 | NM_001860 | 1318 | chr9q31-q32 | 603088 | Hs.24030_at | solute carrier family 31 (copper | SLC31A2 | Amy | 0.922 | 0.968 | 0.446 | 0.762 | 0.009 | 0.392 |
| | | | | | | transporters), member 2 | | | | | | | | |
| Hs.241471 | NM_016337 | 51466 | chr14q32.2 | — | Hs.241471_at | Enah/Vasp-like | EVL | Amy | 0.355 | 1.093 | 0.043 | 1.097 | 0.031 | 1.209 |
| Hs.242947 | NM_004717 | 9162 | chr7q32.3-q33 | 604072 | Hs.242947_at | diacylglycerol kinase, lota | DGKI | Amy | 0.082 | 1.357 | 0.491 | 1.122 | 0.027 | 1.355 |
| Hs.24341 | AA081084 | 25937 | chr3q23-q24 | 607392 | Hs.24341_at | transcriptional co-activator with | TAZ | Amy | 0.413 | 0.957 | 0.183 | 0.900 | 0.002 | 0.771 |
| | | | | | | PDZ-binding motif (TAZ) | | | | | | | | |
| Hs.245537 | AI474054 | 128338 | chr1p13.3 | — | Hs.245537_at | hypothetical protein MGC54289 | MGC54289 | Amy | 0.467 | 0.968 | 0.580 | 0.942 | 0.006 | 0.708 |
| Hs.24587 | NM_005864 | 10278 | chr14q11.2-q12 | — | Hs.24587_at | embryonal Fyn-associated | EFS | Amy | 0.755 | 0.979 | 0.506 | 0.880 | 0.007 | 0.720 |
| | | | | | | substrate | | | | | | | | |
| Hs.246381 | NM_001251 | 968 | chr17p13 | 153634 | Hs.246381_at | CD68 antigen | CD68 | Amy | 0.697 | 0.943 | 0.681 | 0.972 | 0.001 | 0.595 |
| Hs.246970 | AW298170 | 11183 | chr14q11.2-q21 | 604923 | Hs.246970_at | mitogen-activated protein | MAP4K5 | Amy | 0.449 | 1.198 | 0.377 | 0.903 | 0.030 | 0.694 |
| | | | | | | kinase kinase kinase kinase 5 | | | | | | | | |
| Hs.24725 | AI251283 | 246330 | chr11q13.2 | — | Hs.24725_at | pellino 3 alpha | MGC35521 | Amy | 0.487 | 1.074 | 0.974 | 0.997 | 0.007 | 1.281 |
| Hs.248112 | NM_000809 | 2557 | chr4p12 | 137141 | Hs.248112_at | gamma-aminobutyric acid | GABRA4 | Amy | 0.656 | 0.951 | 0.757 | 1.029 | 0.002 | 1.278 |
| | | | | | | (GABA) A receptor, alpha 4 | | | | | | | | |
| Hs.24879 | AF047760 | 8612 | chr19p13 | 607126 | Hs.24879_at | phosphatidic acid phosphatase | PPAP2C | Amy | 0.416 | 1.158 | 0.623 | 0.872 | 0.011 | 0.615 |
| | | | | | | type 2C | | | | | | | | |
| Hs.25035 | AF109196 | 25932 | chr1p36.11 | 606536 | Hs.25035_at | chloride intracellular channel 4 | CLIC4 | Amy | 0.944 | 1.014 | 0.162 | 0.809 | 0.005 | 0.534 |
| Hs.250712 | U07139 | 784 | chr12q13 | 601958 | Hs.250712_at | calcium channel, voltage- | CACNB3 | Amy | 0.197 | 1.136 | 0.243 | 1.124 | 0.014 | 1.417 |
| | | | | | | dependent, beta 3 subunit | | | | | | | | |
| Hs.25155 | AW263232 | 10276 | chr10p15 | 606450 | Hs.25155_at | neuroepithelial cell transforming | NET1 | Amy | 0.960 | 1.014 | 0.467 | 0.907 | 0.024 | 0.721 |
| | | | | | | gene 1 | | | | | | | | |
| Hs.254122 | BC035848 | 55580 | — | — | Hs.254122_at | hypothetical protein LOC55580 | LOC55580 | Amy | 0.967 | 1.007 | 0.104 | 0.723 | 0.037 | 0.560 |
| Hs.2551 | | | | | Hs.2551_at | adrenergic, beta-2-, receptor, | ADRB2 | Amy | 0.286 | 1.034 | 0.676 | 1.027 | 0.010 | 0.745 |
| | | | | | | surface | | | | | | | | |
| Hs.255230 | NM_000181 | 2990 | chr7q21.11 | 253220 | Hs.255230_at | glucuronidase, beta | GUSB | Amy | 0.689 | 0.979 | 0.297 | 0.937 | 0.026 | 0.807 |
| Hs.25597 | NM_016031 | 64834 | chr1p34.2 | — | Hs.25597_at | elongation of very long chain | ELOVL1 | Amy | 0.343 | 1.245 | 0.912 | 0.967 | 0.041 | 0.578 |
| | | | | | | fatty acids (FEN1/Elo2, | | | | | | | | |
| | | | | | | SUR4/Elo3, yeast)-like 1 | | | | | | | | |
| Hs.25601 | BE379542 | 1107 | chr17p13.1 | 602120 | Hs.25601_at | chromodomain helicase DNA | CHD3 | Amy | 0.684 | 1.033 | 0.251 | 1.108 | 0.014 | 1.330 |
| | | | | | | binding protein 3 | | | | | | | | |
| Hs.25647 | BC004490 | 2353 | chr14q24.3 | 164810 | Hs.25647_at | v-fos FBJ murine osteosarcoma | FOS | Amy | 0.142 | 0.491 | 0.174 | 0.357 | 0.015 | 0.164 |
| | | | | | | viral oncogene homolog | | | | | | | | |
| Hs.25748 | AV722990 | 56121 | chr5q31 | 606341 | Hs.25748_at | protocadherin beta 15 | PCDHB15 | Amy | 0.129 | 0.959 | 0.568 | 1.018 | 0.043 | 0.733 |
| Hs.258326 | NM_016524 | 51760 | chr16p12.3 | — | Hs.258326_at | B/K protein | LOC51760 | Amy | 0.313 | 1.189 | 0.476 | 1.073 | 0.029 | 1.315 |
| Hs.25999 | NM_024294 | 64771 | chr6p21.31 | — | Hs.25999_at | chromosome 6 open reading | C6orf106 | Amy | 0.694 | 1.034 | 0.270 | 1.070 | 0.043 | 1.209 |
| | | | | | | frame 106 | | | | | | | | |
| Hs.263671 | NM_002906 | 5962 | chr11q23 | 179410 | Hs.263671_at | radixin | RDX | Amy | 0.527 | 1.149 | 0.457 | 0.907 | 0.007 | 0.688 |
| Hs.263928 | AI817976 | 126259 | chr19p13.3 | — | Hs.263928_at | hypothetical protein MGC23244 | MGC23244 | Amy | 0.281 | 0.915 | 0.411 | 0.937 | 0.032 | 0.828 |
| Hs.26458 | BC040270 | 4482 | chr8p23.1 | 601250 | Hs.26458_at | methionine sulfoxide reductase A | MSRA | Amy | 0.049 | 1.198 | 0.160 | 1.110 | 0.040 | 1.276 |
| Hs.266175 | AI860212 | 55824 | chr8q21.13 | 605767 | Hs.266175_at | phosphoprotein associated with | PAG | Amy | 0.456 | 0.957 | 0.768 | 0.974 | 0.037 | 0.827 |
| | | | | | | glycosphingolipid-enriched | | | | | | | | |
| | | | | | | microdomains | | | | | | | | |
| Hs.26663 | NM_016323 | 51191 | chr4q22.1-q23 | 608242 | Hs.26663_at | cyclin-E binding protein 1 | CEB1 | Amy | 0.008 | 0.952 | 0.280 | 0.949 | 0.001 | 0.713 |
| Hs.26704 | BC005097 | 23191 | chr15q11 | 606322 | Hs.26704_at | cytoplasmic FMR1 interacting | CYFIP1 | Amy | 0.087 | 0.889 | 0.067 | 0.873 | 0.020 | 0.725 |
| | | | | | | protein 1 | | | | | | | | |
| Hs.26812 | BC004870 | 84886 | chr1q42.13-q43 | — | Hs.26812_at | hypothetical protein FLJ14525 | FLJ14525 | Amy | 0.523 | 0.911 | 0.519 | 0.911 | 0.019 | 0.802 |
| Hs.268545 | NM_006078 | 10369 | chr22q13.1 | 602911 | Hs.268545_at | calcium channel, voltage- | CACNG2 | Amy | 0.154 | 1.051 | 0.117 | 1.148 | 0.002 | 1.365 |
| | | | | | | dependent, gamma subunit 2 | | | | | | | | |
| Hs.272254 | AB037738 | 57528 | chr5q31.3 | — | Hs.272254_at | KIAA1317 protein | KIAA1317 | Amy | 0.130 | 1.181 | 0.209 | 1.143 | 0.010 | 1.328 |
| Hs.2722 | NM_002220 | 3706 | chr15q14-q21 | 147521 | Hs.2722_at | inositol 1,4,5-trisphosphate 3- | ITPKA | Amy | 0.352 | 1.281 | 0.207 | 1.190 | 0.039 | 1.778 |
| | | | | | | kinase A | | | | | | | | |
| Hs.272458 | NM_000944 | 5530 | chr4q21-q24 | 114105 | Hs.272458_at | protein phosphatase 3 (formerly | PPP3CA | Amy | 0.472 | 1.144 | 0.428 | 1.037 | 0.014 | 1.361 |
| | | | | | | 2B), catalytic subunit, alpha | | | | | | | | |
| | | | | | | isoform (calcineurin A alpha) | | | | | | | | |
| Hs.274122 | NM_001978 | 2039 | chr8p21.1 | 125305 | Hs.274122_at | erythrocyte membrane protein | EPB49 | Amy | 0.450 | 1.168 | 0.082 | 1.149 | 0.028 | 1.396 |
| | | | | | | band 4.9 (dematin) | | | | | | | | |
| Hs.274293 | NM_003360 | 7368 | chr4q26 | 601291 | Hs.274293_at | UDP glycosyltransferase 8 | UGT8 | Amy | 0.624 | 1.230 | 0.479 | 0.705 | 0.005 | 0.385 |
| | | | | | | (UDP-galactose ceramide | | | | | | | | |
| | | | | | | galactosyltransferase) | | | | | | | | |
| Hs.274317 | NM_016222 | 51428 | chr5q35.3 | 608170 | Hs.274317_at | DEAD (Asp-Glu-Ala-Asp) box | DDX41 | Amy | 0.089 | 1.240 | 0.054 | 1.149 | 0.026 | 1.281 |
| | | | | | | polypeptide 41 | | | | | | | | |
| Hs.274351 | BC003128 | 51114 | chr9 | — | Hs.274351_at | zinc finger, DHHC domain | ZDHHC9 | Amy | 0.339 | 1.084 | 0.518 | 0.898 | 0.032 | 0.683 |
| | | | | | | containing 9 | | | | | | | | |
| Hs.274363 | NM_021257 | 58157 | chr14q24 | 605304 | Hs.274363_at | neuroglobin | NGB | Amy | 0.706 | 1.041 | 0.727 | 1.021 | 0.006 | 1.230 |
| Hs.275675 | NM_005886 | 10300 | chr16q13 | 602703 | Hs.275675_at | katanin p80 (WD repeat | KATNB1 | Amy | 0.306 | 1.047 | 0.051 | 1.121 | 0.011 | 1.251 |
| | | | | | | containing) subunit B 1 | | | | | | | | |
| Hs.275775 | NM_005410 | 6414 | chrsq31 | 601484 | Hs.275775_at | selenoprotein P, plasma, 1 | SEPP1 | Amy | 0.972 | 1.004 | 0.288 | 0.863 | 0.008 | 0.727 |
| Hs.276210 | BC037315 | 286002 | chr7q22.3 | — | Hs.276210_at | hypothetical protein LOC286002 | LOC286002 | Amy | 0.493 | 1.085 | 0.899 | 1.013 | 0.048 | 1.617 |
| Hs.278613 | NM_005532 | 3429 | chr14q32 | 600009 | Hs.278613_at | interferon, alpha-inducible | IFI27 | Amy | 0.882 | 0.969 | 0.203 | 0.861 | 0.002 | 0.576 |
| | | | | | | protein 27 | | | | | | | | |
| Hs.278954 | NM_033136 | 2246 | chr5q31 | 131220 | Hs.278954_at | fibroblast growth factor 1 | FGF1 | Amy | 0.974 | 1.010 | 0.951 | 1.012 | 0.049 | 0.746 |
| | | | | | | (acidic) | | | | | | | | |
| Hs.279008 | NM_017696 | 54844 | chr6q22.31 | — | Hs.279008_at | chromosome 6 open reading | C6orf61 | Amy | 0.391 | 0.970 | 0.398 | 0.933 | 0.036 | 0.790 |
| | | | | | | frame 61 | | | | | | | | |
| Hs.27935 | BC004233 | 94015 | chr17q24 | 608855 | Hs.27935_at | tweety homolog 2 (Drosophila) | TTYH2 | Amy | 0.431 | 1.142 | 0.492 | 0.853 | 0.014 | 0.597 |
| Hs.279669 | NM_016437 | 27175 | chr17q21 | 605785 | Hs.279669_at | tubulin, gamma 2 | TUBG2 | Amy | 0.309 | 1.101 | 0.445 | 1.059 | 0.013 | 1.295 |
| Hs.279912 | BC030223 | 9738 | chr16p12.3 | — | Hs.279912_at | CP110 protein | CP110 | Amy | 0.471 | 1.038 | 0.717 | 0.967 | 0.028 | 0.769 |
| Hs.280311 | AK026977 | 4628 | chr17p13 | 160776 | Hs.280311_at | myosin, heavy polypeptide 10, | MYH10 | Amy | 0.105 | 1.149 | 0.583 | 1.036 | 0.046 | 1.240 |
| | | | | | | non-muscle | | | | | | | | |
| Hs.280354 | NM_172193 | 122773 | chr14q21.3 | — | Hs.280354_at | kelch domain containing 1 | KLHDC1 | Amy | 0.465 | 0.943 | 0.766 | 0.983 | 0.012 | 0.771 |
| Hs.282177 | AB011161 | 23396 | chr19p13.3 | 606102 | Hs.282177_at | phosphatidylinositol-4- | PIP5K1C | Amy | 0.349 | 1.082 | 0.260 | 1.087 | 0.018 | 1.300 |
| | | | | | | phosphate 5-kinase, type I, | | | | | | | | |
| | | | | | | gamma | | | | | | | | |
| Hs.282233 | BC007859 | 4302 | chr17q21 | 600328 | Hs.282233_at | myeloid/lymphoid or mixed- | MLLT6 | Amy | 0.820 | 0.992 | 0.441 | 1.037 | 0.013 | 1.251 |
| | | | | | | lineage leukemia (trithorax | | | | | | | | |
| | | | | | | homolog, Drosophila); | | | | | | | | |
| | | | | | | translocated to, 6 | | | | | | | | |
| Hs.283063 | NM_005574 | 4005 | chr11p13 | 180385 | Hs.283063_at | LIM domain only 2 (rhombotin- | LMO2 | Amy | 0.183 | 0.869 | 0.100 | 0.817 | 0.005 | 0.611 |
| | | | | | | like 1) | | | | | | | | |
| Hs.283091 | NM_020415 | 56729 | chr19p13.2 | 605565 | Hs.283091_at | resistin | RETN | Amy | 0.627 | 1.105 | 0.361 | 1.089 | 0.032 | 1.238 |
| Hs.283661 | NM_018938 | 56131 | chr5q31 | 606330 | Hs.283661_at | protocadherin beta 4 | PCDHB4 | Amy | 0.719 | 1.010 | 0.371 | 0.932 | 0.018 | 0.781 |
| Hs.283902 | AC007743 | 114800 | chr2p16.1 | — | Hs.283902_at | KIAA1912 protein | KIAA1912 | Amy | 0.083 | 1.086 | 0.241 | 1.039 | 0.042 | 1.201 |
| Hs.28423 | NM_014517 | 7342 | chr3p23 | — | Hs.28423_at | upstream binding protein 1 | UBP1 | Amy | 0.024 | 1.191 | 0.048 | 1.173 | 0.042 | 1.220 |
| | | | | | | (LBP-1a) | | | | | | | | |
| Hs.285976 | AK001105 | 29956 | chr1q21.2 | 606920 | Hs.285976_at | LAG1 longevity assurance | LASS2 | Amy | 0.991 | 0.999 | 0.548 | 0.894 | 0.012 | 0.688 |
| | | | | | | homolog 2 (S. cerevisiae) | | | | | | | | |
| Hs.28607 | BE963444 | 57149 | chr16p11.2 | — | Hs.28607_at | hypothetical protein A-211C6.1 | LOC57149 | Amy | 0.375 | 1.082 | 0.110 | 1.119 | 0.031 | 1.230 |
| Hs.286849 | NM_021822 | 60489 | chr22q13.1-q13.2 | 607113 | Hs.286849_at | apolipoprotein B mRNA editing | APOBEC3G | Amy | 0.656 | 0.967 | 0.760 | 0.979 | 0.008 | 0.716 |
| | | | | | | enzyme, catalytic polypeptide- | | | | | | | | |
| | | | | | | like 3G | | | | | | | | |
| Hs.2868 | NM_002677 | 5375 | chr8q21.3-q22.1 | 170715 | Hs.2868_at | peripheral myelin protein 2 | PMP2 | Amy | 0.395 | 0.800 | 0.102 | 0.736 | 0.024 | 0.754 |
| Hs.287521 | NM_024988 | 80054 | chr19q13.11 | — | Hs.287521_at | hypothetical protein FLJ12355 | FLJ12355 | Amy | 0.832 | 0.984 | 0.842 | 1.011 | 0.000 | 1.241 |
| Hs.287921 | AF029674 | 10488 | chr9pter-p22.1 | 606443 | Hs.287921_at | cAMP responsive element | CREB3 | Amy | 0.824 | 1.058 | 0.130 | 1.102 | 0.031 | 1.235 |
| | | | | | | binding protein 3 | | | | | | | | |
| Hs.288010 | AW662189 | 128346 | chr1p13.2 | — | Hs.288010_at | hypothetical protein MGC24133 | MGC24133 | Amy | 0.118 | 0.935 | 0.136 | 0.908 | 0.019 | 0.802 |
| Hs.288284 | NM_025078 | 80148 | chr18q23 | — | Hs.288284_at | PQ loop repeat containing 1 | PQLC1 | Amy | 0.808 | 0.986 | 0.143 | 1.083 | 0.001 | 1.283 |
| Hs.290270 | NM_004746 | 9229 | chr18p11.3 | 605445 | Hs.290270_at | discs, large (Drosophila) | DLGAP1 | Amy | 0.202 | 1.114 | 0.293 | 1.067 | 0.036 | 1.283 |
| | | | | | | homolog-associated protein 1 | | | | | | | | |
| Hs.29052 | NM_017704 | 54851 | chr11q21 | — | Hs.29052_at | fetal globin-inducing factor | FGIF | Amy | 0.062 | 0.851 | 0.180 | 0.886 | 0.020 | 0.757 |
| Hs.29190 | AI732488 | 149563 | chr1p36.13 | — | Hs.29190_at | hypothetical protein MGC24047 | MGC24047 | Amy | 0.119 | 0.877 | 0.535 | 0.935 | 0.048 | 0.826 |
| Hs.29202 | AF039686 | 2857 | chrxp11.4-p11.3 | 300241 | Hs.29202_at | G protein-coupled receptor 34 | GPR34 | Amy | 0.217 | 0.952 | 0.076 | 0.774 | 0.014 | 0.324 |
| Hs.292738 | AF498927 | 397 | chr12p12.3 | 602843 | Hs.292738_at | Rho GDP dissociation inhibitor | ARHGDIB | Amy | 0.042 | 0.836 | 0.020 | 0.849 | 0.002 | 0.548 |
| | | | | | | (GDI) beta | | | | | | | | |
| Hs.293907 | NM_022068 | 63895 | chr18p11.22 | — | Hs.293907_at | hypothetical protein FLJ23403 | FLJ23403 | Amy | 0.584 | 1.036 | 0.912 | 0.990 | 0.019 | 0.764 |
| Hs.294027 | AA057445 | 283225 | chr11q14.1 | — | Hs.294027_at | hypothetical protein FLJ37266 | FLJ37266 | Amy | 0.259 | 1.172 | 0.058 | 1.157 | 0.001 | 1.315 |
| Hs.294145 | AL577758 | 133957 | chr5p15.33 | — | Hs.294145_at | similar to RIKEN cDNA | LOC133957 | Amy | 0.109 | 1.145 | 0.105 | 1.118 | 0.013 | 1.252 |
| | | | | | | 0610011N22 | | | | | | | | |
| Hs.297343 | NM_006549 | 10645 | chr12q24.2 | — | Hs.297343_at | calcium/calmodulin-dependent | CAMKK2 | Amy | 0.382 | 1.162 | 0.060 | 1.245 | 0.008 | 1.460 |
| | | | | | | protein kinase kinase 2, beta | | | | | | | | |
| Hs.298275 | NM_018573 | 54407 | chr12q | 605180 | Hs.298275_at | solute carrier family 38, member 2 | SLC38A2 | Amy | 0.971 | 1.010 | 0.677 | 0.960 | 0.009 | 0.706 |
| Hs.2998 | NM_005076 | 6900 | chr1q32.1 | 190197 | Hs.2998_at | contactin 2 (axonal) | CNTN2 | Amy | 0.132 | 1.303 | 0.848 | 0.935 | 0.050 | 0.553 |
| Hs.300642 | N32526 | 10000 | chr1q43-q44 | — | Hs.300642_at | v-akt murine thymoma viral | AKT3 | Amy | 0.062 | 1.169 | 0.150 | 1.081 | 0.005 | 1.244 |
| | | | | | | oncogene homolog 3 (protein | | | | | | | | |
| | | | | | | kinase B, gamma) | | | | | | | | |
| Hs.301206 | NM_004798 | 9371 | chr20q11.21 | 603754 | Hs.301206_at | kinesin family member 3B | KIF3B | Amy | 0.177 | 1.195 | 0.022 | 1.197 | 0.009 | 1.243 |
| Hs.301394 | AK022644 | 79007 | chr16q24.3 | — | Hs.301394_at | hypothetical protein MGC3101 | MGC3101 | Amy | 0.139 | 1.120 | 0.017 | 1.174 | 0.038 | 1.345 |
| Hs.301760 | NM_014286 | 23413 | chr9q34 | 603315 | Hs.301760_at | frequenin homolog (Drosophila) | FREQ | Amy | 0.783 | 1.031 | 0.663 | 1.026 | 0.024 | 1.285 |
| Hs.301920 | AI799702 | 80863 | chr6p21.32 | — | Hs.301920_at | chromosome 6 open reading | C6orf31 | Amy | 0.069 | 1.062 | 0.008 | 1.166 | 0.034 | 1.203 |
| | | | | | | frame 31 | | | | | | | | |
| Hs.30213 | AI911687 | 1203 | chr13q21.1-q32 | 608102 | Hs.30213_at | ceroid-lipofuscinosis, neuronal 5 | CLN5 | Amy | 0.801 | 1.008 | 0.665 | 0.970 | 0.010 | 0.779 |
| Hs.303649 | S69738 | 6347 | chr17q11.2-q21.1 | 158105 | Hs.303649_at | chemokine (C—C motif) ligand 2 | CCL2 | Amy | 0.252 | 0.883 | 0.345 | 0.820 | 0.006 | 0.695 |
| Hs.306221 | AI057121 | 284695 | chr1p22.2 | — | Hs.306221_at | hypothetical protein FLJ20403 | FLJ20403 | Amy | 0.830 | 1.006 | 0.345 | 0.953 | 0.008 | 0.813 |
| Hs.30818 | AK023743 | 91351 | chr4q32.3 | — | Hs.30818_at | hypothetical protein FLJ31033 | FLJ31033 | Amy | 0.104 | 0.822 | 0.068 | 0.836 | 0.001 | 0.708 |
| Hs.311559 | NM_017617 | 4851 | chr9q34.3 | 190198 | Hs.311559_at | Notch homolog 1, translocation- | NOTCH1 | Amy | 0.103 | 0.953 | 0.853 | 0.990 | 0.026 | 0.833 |
| | | | | | | associated (Drosophila) | | | | | | | | |
| Hs.312503 | NM_018315 | 55294 | chr4q31.3 | 606278 | Hs.312503_at | F-box and WD-40 domain | FBXW7 | Amy | 0.449 | 1.143 | 0.852 | 1.012 | 0.021 | 1.333 |
| | | | | | | protein 7 (archipelago homolog, | | | | | | | | |
| | | | | | | Drosophila) | | | | | | | | |
| Hs.313247 | BC002331 | 54949 | chr11q12.2 | — | Hs.313247_at | hypothetical protein FLJ20487 | FLJ20487 | Amy | 0.922 | 1.001 | 0.098 | 1.084 | 0.012 | 1.311 |
| Hs.313 | AB019562 | 6696 | chr4q21-q25 | 166490 | Hs.313_at | secreted phosphoprotein 1 | SPP1 | Amy | 0.740 | 1.121 | 0.201 | 0.630 | 0.002 | 0.360 |
| | | | | | | (osteopontin, bone sialoprotein | | | | | | | | |
| | | | | | | I, early T-lymphocyte activation | | | | | | | | |
| | | | | | | 1) | | | | | | | | |
| Hs.315379 | N24643 | 26118 | chr17q11.1 | — | Hs.315379_at | SOCS box-containing WD | WSB1 | Amy | 0.834 | 1.040 | 0.066 | 0.873 | 0.017 | 0.701 |
| | | | | | | protein SWIP-1 | | | | | | | | |
| Hs.31595 | NM_005602 | 5010 | chr3q26.2-q26.3 | 601326 | Hs.31595_at | claudin 11 (oligodendrocyte | CLDN11 | Amy | 0.419 | 1.168 | 0.532 | 0.860 | 0.026 | 0.568 |
| | | | | | | transmembrane protein) | | | | | | | | |
| Hs.317614 | BQ022804 | 143903 | chr11q23.2 | — | Hs.317614+_at | layilin | L0C143903 | Amy | 0.482 | 1.059 | 0.292 | 0.747 | 0.026 | 0.496 |
| Hs.318501 | AA083478 | 10346 | chr11p15 | 606559 | Hs.318501_at | tripartite motif-containing 22 | TRIM22 | Amy | 0.276 | 0.855 | 0.151 | 0.774 | 0.002 | 0.500 |
| Hs.318529 | BG258131 | 339983 | chr4p16.3 | — | Hs.318529_at | hypothetical protein FLJ37478 | FLJ37478 | Amy | 0.762 | 1.044 | 0.526 | 1.042 | 0.011 | 1.257 |
| Hs.32017 | NM_020645 | 56675 | chr11p15.3 | — | Hs.32017_at | nuclear receptor interacting | NRIP3 | Amy | 0.085 | 1.169 | 0.271 | 1.098 | 0.004 | 1.242 |
| | | | | | | protein 3 | | | | | | | | |
| Hs.320834 | AL136903 | 84937 | chr16q22.3 | — | Hs.320834_at | zinc and ring finger protein 1 | ZNRF1 | Amy | 0.258 | 1.022 | 0.087 | 1.099 | 0.021 | 1.229 |
| Hs.321164 | NM_002518 | 4862 | chr2q11.2 | 603347 | Hs.321164_at | neuronal PAS domain protein 2 | NPAS2 | Amy | 0.405 | 1.030 | 0.013 | 1.114 | 0.000 | 1.330 |
| Hs.321653 | AK022832 | 84134 | chr1q23.3 | — | Hs.321653_at | hypothetical protein FLJ12770 | FLJ12770 | Amy | 0.695 | 1.022 | 0.127 | 1.166 | 0.016 | 1.361 |
| Hs.323562 | AL136636 | 84061 | chrxq21.1 | — | Hs.323562_at | implantation-associated protein | DKFZp564K142 | Amy | 0.731 | 0.927 | 0.308 | 0.778 | 0.046 | 0.704 |
| Hs.323949 | | | | | Hs.323949_at | kangai 1 (suppression of | KAI1 | Amy | 0.327 | 1.017 | 0.798 | 0.956 | 0.043 | 0.812 |
| | | | | | | tumorigenicity 6, prostate; CD82 | | | | | | | | |
| | | | | | | antigen (R2 leukocyte antigen, | | | | | | | | |
| | | | | | | antigen detected by monoclonal | | | | | | | | |
| | | | | | | and antibody IA4)) | | | | | | | | |
| Hs.324051 | NM_006663 | 10848 | chr19q13.32 | 607463 | Hs.324051_at | ReIA-associated inhibitor | RAI | Amy | 0.425 | 0.872 | 0.206 | 0.852 | 0.024 | 0.733 |
| Hs.333303 | NM_000166 | 2705 | chrxq13.1 | 304040 | Hs.333303_at | gap junction protein, beta 1, | GJB1 | Amy | 0.782 | 0.964 | 0.872 | 1.031 | 0.045 | 0.800 |
| | | | | | | 32 kDa (connexin 32, Charcot- | | | | | | | | |
| | | | | | | Marie-Tooth neuropathy, X- | | | | | | | | |
| | | | | | | linked) | | | | | | | | |
| Hs.33461 | | | | | Hs.33461_at | formin-like 2 | FMNL2 | Amy | 0.791 | 1.036 | 0.193 | 0.816 | 0.021 | 0.763 |
| Hs.334688 | NM_014759 | 9796 | chr8p21.3 | 608511 | Hs.334688_at | phytanoyl-CoA hydroxylase | PHYHIP | Amy | 0.645 | 1.140 | 0.237 | 1.097 | 0.028 | 1.328 |
| | | | | | | interacting protein | | | | | | | | |
| Hs.334873 | NM_001874 | 1368 | chr12q14.3 | 114860 | Hs.334873_at | carboxypeptidase M | CPM | Amy | 0.099 | 0.917 | 0.530 | 0.957 | 0.001 | 0.831 |
| Hs.33922 | AL035369 | 92342 | chr1q24.2 | — | Hs.33922_at | hypothetical protein MGC9084 | MGC9084 | Amy | 0.817 | 0.988 | 0.963 | 1.008 | 0.030 | 0.774 |
| Hs.342307 | NM_018061 | 55119 | chr1p13.3 | — | Hs.342307_at | hypothetical protein FLJ10330 | FLJ10330 | Amy | 0.542 | 0.944 | 0.097 | 0.851 | 0.013 | 0.748 |
| Hs.3459 | NM_019116 | 56061 | chr16p12 | — | Hs.3459_at | similar to ubiquitin binding | UBPH | Amy | 0.502 | 1.098 | 0.799 | 1.018 | 0.029 | 1.213 |
| | | | | | | protein | | | | | | | | |
| Hs.347534 | BE044440 | 57476 | chr11q24.1 | — | Hs.347534_at | KIAA1201 protein | KIAA1201 | Amy | 0.667 | 1.057 | 0.192 | 1.099 | 0.026 | 1.266 |
| Hs.34780 | NM_000555 | 1641 | chrxq22.3-q23 | 300121 | Hs.34780_at | doublecortex; lissencephaly, X- | DCX | Amy | 0.427 | 1.058 | 0.280 | 1.116 | 0.022 | 1.286 |
| | | | | | | linked (doublecortin) | | | | | | | | |
| Hs.348037 | AA156998 | 94274 | chr19q13.1 | 608153 | Hs.348037_at | protein phosphatase 1, | PPP1R14A | Amy | 0.423 | 1.157 | 0.623 | 0.876 | 0.043 | 0.618 |
| | | | | | | regulatory (inhibitor) subunit | | | | | | | | |
| | | | | | | 14A | | | | | | | | |
| Hs.348260 | NM_014770 | 116986 | chr12q14.1 | 605476 | Hs.348260_at | centaurin, gamma 1 | CENTG1 | Amy | 0.554 | 0.928 | 0.114 | 1.379 | 0.006 | 1.675 |
| Hs.348415 | NM_030786 | 81493 | chr1p34.3-p33 | — | Hs.348415_at | intermediate filament protein | SYNCOILIN | Amy | 0.908 | 1.047 | 0.278 | 1.134 | 0.021 | 0.787 |
| | | | | | | syncoilin | | | | | | | | |
| Hs.349227 | NM_012219 | 22808 | chr3q22.3 | 608435 | Hs.349227_at | muscle RAS oncogene homolog | MRAS | Amy | 0.596 | 0.915 | 0.250 | 1.072 | 0.014 | 1.247 |
| Hs.349262 | BF515750 | 128061 | chr1q42.2 | — | Hs.349262_at | hypothetical protein | DKFZp547B1713 | Amy | 0.349 | 0.975 | 0.864 | 0.981 | 0.013 | 0.774 |
| | | | | | | DKFZp547B1713 | | | | | | | | |
| Hs.349955 | AV730849 | 122060 | chr13q22.3 | — | Hs.349955_at | hypothetical protein FLJ30046 | FLJ30046 | Amy | 0.657 | 1.141 | 0.500 | 0.844 | 0.020 | 0.601 |
| Hs.35086 | AW499935 | 7398 | chr1p32.1-p31.3 | 603478 | Hs.35086_at | ubiquitin specific protease 1 | USP1 | Amy | 0.424 | 1.173 | 0.858 | 1.017 | 0.048 | 0.813 |
| Hs.351279 | X76775 | 3108 | chr6p21.3 | 142855 | Hs.351279_at | major histocompatibility | HLA-DMA | Amy | 0.779 | 1.024 | 0.087 | 0.683 | 0.020 | 0.519 |
| | | | | | | complex, class II, DM alpha | | | | | | | | |
| Hs.351623 | AK022955 | 55536 | chr7p15.3 | — | Hs.351623_at | transcription factor RAM2 | RAM2 | Amy | 0.536 | 1.036 | 0.672 | 1.074 | 0.007 | 0.640 |
| Hs.352153 | NM_138818 | 158471 | chr9q21.2 | — | Hs.352153_at | chromosome 9 open reading | C9orf65 | Amy | 0.581 | 1.076 | 0.167 | 0.662 | 0.004 | 0.395 |
| | | | | | | frame 65 | | | | | | | | |
| Hs.352388 | AI218954 | 157310 | chr8p21.3 | — | Hs.352388_at | hypothetical protein MGC22776 | MGC22776 | Amy | 0.367 | 1.025 | 0.306 | 0.927 | 0.047 | 0.821 |
| Hs.353087 | N74056 | 57465 | chr16p13.3 | — | Hs.353087_at | KIAA1171 protein | KIAA1171 | Amy | 0.194 | 1.089 | 0.787 | 0.978 | 0.044 | 1.292 |
| Hs.35380 | BU689085 | 56987 | chr3q13.1 | — | Hs.35380_at | bobby sox homolog | BBX | Amy | 0.654 | 0.978 | 0.540 | 0.917 | 0.029 | 0.819 |
| | | | | | | (Drosophila) | | | | | | | | |
| Hs.355933 | AI679968 | 84327 | chr5q13.3 | — | Hs.355933_at | zinc finger, BED domain | ZBED3 | Amy | 0.498 | 1.028 | 0.265 | 0.878 | 0.011 | 0.745 |
| | | | | | | containing 3 | | | | | | | | |
| Hs.356130 | | | | | Hs.356130_at | proline rich membrane anchor 1 | PRIMA1 | Amy | 0.816 | 0.962 | 0.264 | 0.743 | 0.012 | 0.520 |
| Hs.356359 | BF338045 | 126282 | chr19p13.3 | — | Hs.356359_at | hypothetical protein MGC17791 | MGC17791 | Amy | 0.957 | 0.995 | 0.464 | 1.081 | 0.018 | 1.413 |
| Hs.356416 | AV648364 | 23492 | chr22q13.1 | 608457 | Hs.356416_at | chromobox homolog 7 | CBX7 | Amy | 0.366 | 1.130 | 0.073 | 1.137 | 0.034 | 1.295 |
| Hs.368109 | AF285109 | 55964 | chr22q13.2 | 608314 | Hs.368109_at | septin 3 | SEPT3 | Amy | 0.579 | 1.124 | 0.065 | 1.115 | 0.008 | 1.235 |
| Hs.368861 | NM_005163 | 207 | chr14q32.32 | 164730 | Hs.368861_at | v-akt murine thymoma viral | AKT1 | Amy | 0.893 | 0.995 | 0.523 | 0.963 | 0.023 | 1.231 |
| | | | | | | oncogene homolog 1 | | | | | | | | |
| Hs.369026 | NM_004339 | 754 | chr21q22.3 | 603784 | Hs.369026_at | pituitary tumor-transforming 1 | PTTG1IP | Amy | 0.187 | 0.799 | 0.353 | 0.880 | 0.037 | 0.778 |
| | | | | | | interacting protein | | | | | | | | |
| Hs.369288 | NM_017797 | 55643 | chr19p13.3 | 608531 | Hs.369288_at | BTB (POZ) domain containing 2 | BTBD2 | Amy | 0.723 | 1.079 | 0.005 | 1.176 | 0.044 | 1.366 |
| Hs.369994 | NM_004775 | 9331 | chr18q11 | 604017 | Hs.369994_at | UDP-Gal: betaGIcNAc beta 1,4- | B4GALT6 | Amy | 0.147 | 1.329 | 0.318 | 1.120 | 0.012 | 1.313 |
| | | | | | | galactosyltransferase, | | | | | | | | |
| | | | | | | polypeptide 6 | | | | | | | | |
| Hs.37054 | AW189015 | 1944 | chr1q21-q22 | 601381 | Hs.37054_at | ephrin-A3 | EFNA3 | Amy | 0.694 | 1.032 | 0.814 | 1.014 | 0.032 | 1.272 |
| Hs.370873 | NM_005531 | 3428 | chr1q22 | 147586 | Hs.370873_at | interferon, gamma-inducible | IFI16 | Amy | 0.173 | 0.950 | 0.091 | 0.830 | 0.000 | 0.611 |
| | | | | | | protein 16 | | | | | | | | |
| Hs.371416 | AA551784 | 10498 | chr19p13.2 | 603934 | Hs.371416_at | coactivator-associated arginine | CARM1 | Amy | 0.381 | 1.112 | 0.347 | 1.056 | 0.003 | 1.307 |
| | | | | | | methyltransferase 1 | | | | | | | | |
| Hs.371468 | BC000076 | 595 | chr11q13 | 168461 | Hs.371468_at | cyclin D1 (PRAD1: parathyroid | CCND1 | Amy | 0.203 | 0.926 | 0.286 | 0.918 | 0.037 | 0.773 |
| | | | | | | adenomatosis 1) | | | | | | | | |
| Hs.371612 | NM_022349 | 64231 | chr11q12.1 | 606548 | Hs.371612_at | membrane-spanning 4- | MS4A6A | Amy | 0.302 | 0.974 | 0.286 | 0.981 | 0.049 | 0.830 |
| | | | | | | domains, subfamily A, member | | | | | | | | |
| | | | | | | 6A | | | | | | | | |
| Hs.372031 | L03203 | 5376 | chr17p12-p11.2 | 601097 | Hs.372031_at | peripheral myelin protein 22 | PMP22 | Amy | 0.825 | 1.060 | 0.659 | 0.882 | 0.017 | 0.622 |
| Hs.374350 | NM_016575 | 51559 | chr12q22-q23.1 | — | Hs.374350_at | TU12B1-TY protein | TU12B1-TY | Amy | 0.978 | 1.003 | 0.169 | 1.136 | 0.013 | 1.279 |
| Hs.374649 | AB037854 | 55917 | chr1p13.2 | — | Hs.374649_at | hypothetical protein | DKFZp547A023 | Amy | 0.107 | 0.963 | 0.043 | 0.929 | 0.005 | 0.816 |
| | | | | | | DKFZp547A023 | | | | | | | | |
| Hs.376984 | AI367319 | 6663 | chr22q13.1 | 602229 | Hs.376984_at | SRY (sex determining region | SOX10 | Amy | 0.736 | 1.030 | 0.668 | 0.919 | 0.045 | 0.747 |
| | | | | | | Y)-box 10 | | | | | | | | |
| Hs.377593 | AA196034 | 79041 | chr19p13.11 | — | Hs.377593_at | hypothetical protein MGC3169 | MGC3169 | Amy | 0.281 | 1.092 | 0.349 | 1.158 | 0.021 | 1.257 |
| Hs.378949 | NM_000328 | 6103 | chrxp11.4 | 312610 | Hs.378949_at | retinitis pigmentosa GTPase | RPGR | Amy | 0.428 | 0.951 | 0.435 | 0.927 | 0.037 | 0.747 |
| | | | | | | regulator | | | | | | | | |
| Hs.380089 | | | | | Hs.380089_at | EphB6 | EPHB6 | Amy | 0.809 | 1.072 | 0.530 | 1.044 | 0.041 | 1.335 |
| Hs.380976 | NM_016533 | 4815 | chr12p13 | 607297 | Hs.380976_at | ninjurin 2 | NINJ2 | Amy | 0.959 | 1.009 | 0.769 | 0.885 | 0.028 | 0.466 |
| Hs.381099 | J02923 | 3936 | chr13q14.3 | 153430 | Hs.381099_at | lymphocyte cytosolic protein 1 | LCP1 | Amy | 0.306 | 0.958 | 0.104 | 0.869 | 0.011 | 0.694 |
| | | | | | | (L-plastin) | | | | | | | | |
| Hs.381256 | NM_016433 | 51228 | chr12q24.11 | — | Hs.381256_at | glycolipid transfer protein | GLTP | Amy | 0.709 | 0.973 | 0.156 | 0.796 | 0.012 | 0.674 |
| Hs.382202 | M80927 | 1116 | chr1q32.1 | 601525 | Hs.382202_at | chitinase 3-like 1 (cartilage | CHI3L1 | Amy | 0.066 | 0.797 | 0.065 | 0.601 | 0.021 | 0.555 |
| | | | | | | glycoprotein-39) | | | | | | | | |
| Hs.38516 | AI130705 | 375061 | chr1q42.2 | — | Hs.38516_at | hypothetical gene supported by | MGC15887 | Amy | 0.228 | 0.911 | 0.215 | 0.880 | 0.018 | 0.792 |
| | | | | | | BC009447 | | | | | | | | |
| Hs.387400 | BG289001 | 253782 | chr2q24.3 | — | Hs.387400_at | hypothetical protein LOC253782 | LOC253782 | Amy | 0.061 | 1.272 | 0.190 | 1.120 | 0.026 | 1.255 |
| Hs.387579 | NM_001769 | 928 | chr12p13.3 | 143030 | Hs.387579_at | CD9 antigen (p24) | CD9 | Amy | 0.533 | 1.251 | 0.871 | 0.955 | 0.008 | 0.525 |
| Hs.387871 | U57059 | 8743 | chr3q26 | 603598 | Hs.387871_at | tumor necrosis factor (ligand) | TNFSF10 | Amy | 0.141 | 0.896 | 0.160 | 0.747 | 0.010 | 0.542 |
| | | | | | | superfamily, member 10 | | | | | | | | |
| Hs.388126 | AL035406 | 26038 | chr1p36.31 | — | Hs.388126_at | chromodomain helicase DNA | CHD5 | Amy | 0.104 | 1.068 | 0.323 | 1.065 | 0.003 | 1.337 |
| | | | | | | binding protein 5 | | | | | | | | |
| Hs.389057 | NM_004647 | 8193 | chr19q13.13-q13.2 | 601670 | Hs.389057_at | D4, zinc and double PHD | DPF1 | Amy | 0.099 | 1.098 | 0.170 | 1.097 | 0.021 | 1.251 |
| | | | | | | fingers family 1 | | | | | | | | |
| Hs.389724 | NM_006820 | 10964 | chr1p31.1 | — | Hs.389724_at | chromosome 1 open reading | C1orf29 | Amy | 0.148 | 0.642 | 0.084 | 0.602 | 0.001 | 0.437 |
| | | | | | | frame 29 | | | | | | | | |
| Hs.391858 | BC015944 | 7072 | chr2p13 | 603518 | Hs.391858_at | TIA1 cytotoxic granule- | TIA1 | Amy | 0.688 | 1.027 | 0.713 | 0.984 | 0.034 | 0.814 |
| | | | | | | associated RNA binding protein | | | | | | | | |
| Hs.392004 | AI967971 | 87178 | chr2p15 | — | Hs.392004_at | polyribonucleotide | PNPT1 | Amy | 0.948 | 1.009 | 0.953 | 0.993 | 0.003 | 0.677 |
| | | | | | | nucleotidyltransferase 1 | | | | | | | | |
| Hs.39252 | NM_007166 | 8301 | chr11q14 | 603025 | Hs.39252_at | phosphatidylinositol binding | PICALM | Amy | 0.530 | 1.108 | 0.230 | 0.926 | 0.032 | 0.776 |
| | | | | | | clathrin assembly protein | | | | | | | | |
| Hs.394609 | BE622952 | 6272 | chr1p21.3-p13.1 | 602458 | Hs.394609_at | sortilin 1 | SORT1 | Amy | 0.435 | 1.139 | 0.631 | 1.034 | 0.037 | 0.780 |
| Hs.400383 | BE963437 | 145567 | chr14q32.12 | — | Hs.400383_at | tetratricopeptide repeat domain | TTC7L1 | Amy | 0.427 | 1.061 | 0.020 | 1.172 | 0.008 | 1.245 |
| | | | | | | 7 like 1 | | | | | | | | |
| Hs.400556 | NM_003657 | 8537 | chr20q13.2-q13.3 | 602968 | Hs.400556_at | breast carcinoma amplified | BCAS1 | Amy | 0.943 | 0.993 | 0.810 | 0.970 | 0.017 | 0.774 |
| | | | | | | sequence 1 | | | | | | | | |
| Hs.40098 | AF154054 | 26585 | chr15q13-q15 | 603054 | Hs.40098_at | cysteine knot superfamily 1, | CKTSF1B1 | Amy | 0.392 | 1.303 | 0.734 | 0.899 | 0.018 | 0.329 |
| | | | | | | BMP antagonist 1 | | | | | | | | |
| Hs.404930 | NM_000271 | 4864 | chr18q11-q12 | 607623 | Hs.404930_at | Niemann-Pick disease, type C1 | NPC1 | Amy | 0.368 | 1.140 | 0.446 | 0.878 | 0.004 | 0.602 |
| Hs.406094 | U87460 | 2861 | chr7q31 | 602583 | Hs.406094_at | G protein-coupled receptor 37 | GPR37 | Amy | 0.654 | 1.185 | 0.475 | 0.724 | 0.007 | 0.381 |
| | | | | | | (endothelin receptor type B-like) | | | | | | | | |
| Hs.406266 | AI761561 | 3099 | chr2p13 | 601125 | Hs.406266_at | hexokinase 2 | HK2 | Amy | 0.812 | 1.009 | 0.877 | 0.987 | 0.024 | 0.664 |
| Hs.40637 | NM_000950 | 5638 | chrxp21.1 | 604428 | Hs.40637_at | proline-rich Gla (G- | PRRG1 | Amy | 0.517 | 1.112 | 0.641 | 0.871 | 0.030 | 0.588 |
| | | | | | | carboxyglutamic acid) | | | | | | | | |
| | | | | | | polypeptide 1 | | | | | | | | |
| Hs.406397 | NM_002055 | 2670 | chr17q21 | 137780 | Hs.406397_at | glial fibrillary acidic protein | GFAP | Amy | 0.582 | 0.805 | 0.308 | 0.891 | 0.001 | 0695 |
| Hs.406612 | AL021707 | 25777 | chr22q13.1 | — | Hs.406612_at | unc-84 homolog B (C. elegans) | UNC84B | Amy | 0.534 | 1.106 | 0.916 | 1.015 | 0.039 | 0.812 |
| Hs.407474 | BC035157 | 26033 | chr10q26 | — | Hs.407474_at | KIAA0534 protein | KIAA0534 | Amy | 0.117 | 1.219 | 0.250 | 1.144 | 0.023 | 1.518 |
| Hs.408658 | NM_004702 | 9134 | chr8q22.1 | 603775 | Hs.408658_at | cyclin E2 | CCNE2 | Amy | 0.865 | 1.009 | 0.859 | 1.032 | 0.046 | 0.668 |
| Hs.408767 | AF007162 | 1410 | chr11q22.3-q23.1 | 123590 | Hs.408767_at | crystallin, alpha B | CRYAB | Amy | 0.688 | 1.176 | 0.896 | 0.968 | 0.010 | 0.673 |
| Hs.40919 | BE967331 | 85365 | chr9q22.33 | 607905 | Hs.40919_at | asparagine-linked glycosylation | ALG2 | Amy | 0.195 | 1.099 | 0.679 | 0.957 | 0.043 | 0.821 |
| | | | | | | 2 homolog (yeast, alpha-1,3- | | | | | | | | |
| | | | | | | mannosyltransferase) | | | | | | | | |
| Hs.409826 | BC006230 | 11343 | chr3q21.3 | — | Hs.409826_at | monoglyceride lipase | MGLL | Amy | 0.844 | 0.963 | 0.219 | 1.117 | 0.036 | 1.369 |
| Hs.410629 | NM_144633 | 131096 | chr3p24.3 | — | Hs.410629_at | potassium voltage-gated | KCNH8 | Amy | 0.451 | 1.152 | 0.809 | 0.925 | 0.028 | 0.377 |
| | | | | | | channel, subfamily H (eag- | | | | | | | | |
| | | | | | | related), member 8 | | | | | | | | |
| Hs.411308 | BF055343 | 117248 | chr3p25.1 | — | Hs.411308_at | UDP-N-acetyl-alpha-D- | GALNT7 | Amy | 0.473 | 0.933 | 0.528 | 0.912 | 0.000 | 0.592 |
| | | | | | | galactosamine: polypeptide N- | | | | | | | | |
| | | | | | | acetylgalactosaminyltransferase 7 | | | | | | | | |
| Hs.41135 | NM_016242 | 51705 | chr4q24 | 608350 | Hs.41135_at | endomucin | EMCN | Amy | 0.191 | 0.918 | 0.352 | 0.923 | 0.003 | 0.806 |
| Hs.41154 | U79264 | 7545 | chr3q24 | 600470 | Hs.41154_at | Zic family member 1 (odd- | ZIC1 | Amy | 0.625 | 0.905 | 0.345 | 0.831 | 0.038 | 0.648 |
| | | | | | | paired homolog, Drosophila) | | | | | | | | |
| Hs.411865 | NM_024658 | 79711 | chr14q11.2 | — | Hs.411865_at | importin 4 | IPO4 | Amy | 0.974 | 0.998 | 0.405 | 0.950 | 0.015 | 0.814 |
| Hs.411958 | NM_018950 | 3134 | chr6p21.3 | 143110 | Hs.411958_at | major histocompatibility | HLA-F | Amy | 0.679 | 1.015 | 0.345 | 0.953 | 0.027 | 0.795 |
| | | | | | | complex, class I, F | | | | | | | | |
| Hs.412286 | BG036668 | 84909 | chr9q22.32 | — | Hs.412286_at | hypothetical protein FLJ14675 | FLJ14675 | Amy | 0.822 | 0.969 | 0.944 | 1.003 | 0.022 | 0.805 |
| Hs.412468 | BC001793 | 116138 | chr6p21.1 | — | Hs.412468_at | kelch domain containing 3 | KLHDC3 | Amy | 0.173 | 1.297 | 0.078 | 1.130 | 0.022 | 1.306 |
| Hs.412836 | BC036122 | 84230 | chr1p22.2 | — | Hs.412836_at | hypothetical protein AD158 | AD158 | Amy | 0.045 | 0.872 | 0.526 | 0.960 | 0.045 | 0.824 |
| Hs.41296 | NM_013281 | 23767 | chr20p11 | 604808 | Hs.41296_at | fibronectin leucine rich | FLRT3 | Amy | 0.025 | 1.104 | 0.777 | 0.976 | 0.005 | 1.267 |
| | | | | | | transmembrane protein 3 | | | | | | | | |
| Hs.414151 | BQ024796 | 23500 | chr6p21.1 | 606627 | Hs.414151_at | dishevelled associated activator | DAAM2 | Amy | 0.776 | 1.143 | 0.997 | 0.999 | 0.024 | 0.753 |
| | | | | | | of morphogenesis 2 | | | | | | | | |
| Hs.414164 | AB037845 | 57584 | chr10p12.1 | — | Hs.414164_at | Rho GTPase activating protein | ARHGAP21 | Amy | 0.468 | 0.913 | 0.231 | 0.888 | 0.045 | 0.755 |
| | | | | | | 21 | | | | | | | | |
| Hs.414362 | NM_016229 | 51700 | chr11p15.4 | 608342 | Hs.414362_at | cytochrome b5 reductase b5R.2 | CYB5R2 | Amy | 0.257 | 1.222 | 0.610 | 0.831 | 0.021 | 0.479 |
| Hs.414390 | NM_005861 | 10273 | chr16p13.3 | 607207 | Hs.414390_at | STIP1 homology and U-Box | STUB1 | Amy | 0.706 | 1.113 | 0.078 | 1.118 | 0.045 | 1.220 |
| | | | | | | containing protein 1 | | | | | | | | |
| Hs.414455 | AU146275 | 7716 | chr17q23.2 | 606747 | Hs.414455_at | zinc finger protein 161 | ZNF161 | Amy | 0.950 | 1.012 | 0.320 | 0.847 | 0.004 | 0.698 |
| Hs.414728 | AK022549 | 23446 | chr9q31.2 | 606105 | Hs.414728_at | choline transporter-like protein 1 | CTL1 | Amy | 0.462 | 1.115 | 0.797 | 0.937 | 0.013 | 0.580 |
| Hs.4147 | BC000687 | 23471 | chr8q13.3 | 605190 | Hs.4147_at | translocation associated | TRAM1 | Amy | 0.900 | 1.017 | 0.168 | 0.887 | 0.044 | 0.789 |
| | | | | | | membrane protein 1 | | | | | | | | |
| Hs.41502 | NM_024633 | 79686 | chr14q32.13 | — | Hs.41502_at | chromosome 14 open reading | C14orf139 | Amy | 0.742 | 0.954 | 0.869 | 0.964 | 0.025 | 0.693 |
| | | | | | | frame 139 | | | | | | | | |
| Hs.415240 | AL512757 | 81844 | chr7q22.1 | — | Hs.415240_at | tripartite motif-containing 56 | TRIM56 | Amy | 0.492 | 0.959 | 0.582 | 0.945 | 0.004 | 0.770 |
| Hs.416026 | NM_002971 | 6304 | chr3p23 | 602075 | Hs.416026_at | special AT-rich sequence | SATB1 | Amy | 0.554 | 1.049 | 0.552 | 0.959 | 0.045 | 1.276 |
| | | | | | | binding protein 1 (binds to | | | | | | | | |
| | | | | | | nuclear matrix/scaffold- | | | | | | | | |
| | | | | | | associating DNA's | | | | | | | | |
| Hs.417004 | NM_005620 | 6282 | chr1q21 | 603114 | Hs.417004_at | S100 calcium binding protein | S100A11 | Amy | 0.160 | 0.931 | 0.316 | 0.838 | 0.008 | 0.687 |
| | | | | | | A11 (calgizzarin) | | | | | | | | |
| Hs.418542 | AF418285 | 25953 | chr2q35 | — | Hs.418542_at | myofibrillogenesis regulator 1 | MR-1 | Amy | 0.701 | 1.059 | 0.330 | 1.090 | 0.005 | 1.254 |
| Hs.418692 | AF135266 | 26471 | chr16p11.2 | — | Hs.418692_at | p8 protein (candidate of | P8 | Amy | 0.830 | 1.014 | 0.963 | 0.994 | 0.023 | 0.756 |
| | | | | | | metastasis 1) | | | | | | | | |
| Hs.421457 | NM_003164 | 6811 | chr11q12.3 | 603189 | Hs.421457_at | syntaxin 5A | STX5A | Amy | 0.453 | 1.038 | 0.016 | 1.164 | 0.026 | 1.234 |
| Hs.42771 | BC015877 | 28513 | chr18q22-q23 | 603016 | Hs.42771_at | cadherin 19, type 2 | CDH19 | Amy | 0.821 | 0.986 | 0.356 | 0.887 | 0.029 | 0.673 |
| Hs.429643 | AY004175 | 23236 | chr20p12 | 607120 | Hs.429643_at | phospholipase C, beta 1 | PLCB1 | Amy | 0.309 | 1.173 | 0.479 | 0.946 | 0.002 | 1.283 |
| | | | | | | (phosphoinositide-specific) | | | | | | | | |
| Hs.429961 | NM_018475 | 55858 | chr4q12 | — | Hs.429961_at | TPA regulated locus | TPARL | Amy | 0.463 | 1.124 | 0.908 | 1.018 | 0.042 | 0.676 |
| Hs.430156 | NM_015993 | 51090 | chr16q13 | 600340 | Hs.430156_at | transmembrane 4 superfamily | TM4SF11 | Amy | 0.794 | 0.945 | 0.563 | 0.840 | 0.036 | 0.564 |
| | | | | | | member 11 (plasmolipin) | | | | | | | | |
| Hs.430606 | BC000105 | 1431 | chr12q13.2-q13.3 | 118950 | Hs.430606_at | citrate synthase | CS | Amy | 0.753 | 1.117 | 0.513 | 1.076 | 0.039 | 1.461 |
| Hs.432574 | U37689 | 5437 | chr3q28 | 606023 | Hs.432574_at | polymerase (RNA) II (DNA | POLR2H | Amy | 0.915 | 1.007 | 0.075 | 1.114 | 0.021 | 1.281 |
| | | | | | | directed) polypeptide H | | | | | | | | |
| Hs.432648 | | | | | Hs.432648_at | heat shock 70 kDa protein 2 | HSPA2 | Amy | 0.622 | 1.177 | 0.741 | 0.888 | 0.010 | 0.571 |
| Hs.432726 | AI953478 | 134359 | chr5q13.3 | — | Hs.432726_at | hypothetical protein FLJ35779 | FLJ35779 | Amy | 0.068 | 0.939 | 0.027 | 0.862 | 0.027 | 0.832 |
| Hs.432945 | AW194999 | 122416 | chr14q32.32 | — | Hs.432945_at | ankyrin repeat domain 9 | ANKRD9 | Amy | 0.865 | 1.013 | 0.229 | 1.093 | 0.039 | 1.236 |
| Hs.433159 | NM_000803 | 2350 | chr11q13.3-q13.5 | 136425 | Hs.433159_at | folate receptor 2 (fetal) | FOLR2 | Amy | 0.959 | 0.993 | 0.108 | 0.915 | 0.034 | 0.781 |
| Hs.433300 | BC020763 | 2207 | chr1q23 | 147139 | Hs.433300_at | Fc fragment of IgE, high affinity | FCER1G | Amy | 0.030 | 0.906 | 0.064 | 0.715 | 0.006 | 0.421 |
| | | | | | | I, receptor for, gamma | | | | | | | | |
| | | | | | | polypeptide | | | | | | | | |
| Hs.433328 | NM_019056 | 54539 | chrxp11.3 | 300403 | Hs.433328_at | neuronal protein 17.3 | P17.3 | Amy | 0.410 | 1.081 | 0.332 | 1.091 | 0.041 | 1.286 |
| Hs.433452 | AA121502 | 57493 | chr3q21.2 | — | Hs.433452_at | HEG homolog | HEG | Amy | 0.022 | 0.863 | 0.041 | 0.848 | 0.030 | 0.800 |
| Hs.433573 | AF073483 | 83638 | chr11q13.1 | — | Hs.433573_at | hypothetical protein p5326 | P5326 | Amy | 0.341 | 1.040 | 0.597 | 1.038 | 0.029 | 1.213 |
| Hs.433574 | N95026 | 23543 | chr22q13.1 | — | Hs.433574_at | RNA binding motif protein 9 | RBM9 | Amy | 0.401 | 1.146 | 0.491 | 1.043 | 0.016 | 1.321 |
| Hs.433732 | AI251890 | 1195 | chr2q33 | 601951 | Hs.433732_at | CDO-like kinase 1 | CLK1 | Amy | 0.783 | 0.949 | 0.106 | 0.837 | 0.004 | 0.612 |
| Hs.433753 | NM_004710 | 9144 | chr17q25.3 | 603926 | Hs.433753_at | synaptogyrin 2 | SYNGR2 | Amy | 0.122 | 1.103 | 0.635 | 0.944 | 0.003 | 0.727 |
| Hs.433839 | NM_001958 | 1917 | chr20q13.3 | 602959 | Hs.433839_at | eukaryotic translation elongation | EEF1A2 | Amy | 0.468 | 1.373 | 0.039 | 1.168 | 0.030 | 1.371 |
| | | | | | | factor 1 alpha 2 | | | | | | | | |
| Hs.434004 | AL527773 | 29 | chr17p13.3 | 600365 | Hs.434004_at | active BCR-related gene | ABR | Amy | 0.228 | 1.230 | 0.022 | 1.147 | 0.014 | 1.297 |
| Hs.434418 | AF036943 | 23040 | chr2p25.3 | — | Hs.434418_at | myelin transcription factor 1-like | MYT1L | Amy | 0.331 | 1.121 | 0.061 | 1.031 | 0.049 | 1.228 |
| Hs.434488 | BF590263 | 1462 | chr5q14.3 | 118661 | Hs.434488_at | chondroitin sulfate proteoglycan | CSPG2 | Amy | 0.311 | 0.875 | 0.288 | 0.845 | 0.022 | 0.693 |
| | | | | | | 2 (versican) | | | | | | | | |
| Hs.434502 | AW242125 | 159195 | chr10q22.2 | — | Hs.434502_at | chromosome 10 open reading | C10orf29 | Amy | 0.974 | 0.997 | 0.437 | 0.881 | 0.015 | 0.696 |
| | | | | | | frame 29 | | | | | | | | |
| Hs.435166 | NM_002296 | 3930 | chr1q42.1 | 600024 | Hs.435166_at | lamin B receptor | LBR | Amy | 0.914 | 1.016 | 0.617 | 0.931 | 0.015 | 0.653 |
| Hs.435295 | NM_018965 | 54209 | chr6p21.1 | 605086 | Hs.435295_at | triggering receptor expressed | TREM2 | Amy | 0.330 | 0.984 | 0.323 | 0.979 | 0.018 | 0.727 |
| | | | | | | on myeloid cells 2 | | | | | | | | |
| Hs.435326 | NM_004301 | 86 | chr3q26.33 | 604958 | Hs.435326_at | BAF53 | BAF53A | Amy | 0.510 | 0.917 | 0.378 | 0.783 | 0.007 | 0.602 |
| Hs.435670 | NM_002067 | 2767 | chr19p13.3 | 139313 | Hs.435670_at | guanine nucleotide binding | GNA11 | Amy | 0.586 | 1.047 | 0.121 | 1.077 | 0.003 | 1.233 |
| | | | | | | protein (G protein), alpha 11 | | | | | | | | |
| | | | | | | (Gq class) | | | | | | | | |
| Hs.435786 | AF465485 | 783 | chr10p12 | 600003 | Hs.435786_at | calcium channel, voltage- | CACNB2 | Amy | 0.055 | 1.297 | 0.080 | 1.244 | 0.043 | 1.516 |
| | | | | | | dependent, beta 2 subunit | | | | | | | | |
| Hs.435800 | AI520969 | 7431 | chr10p13 | 193060 | Hs.435800_at | vimentin | VIM | Amy | 0.130 | 0.871 | 0.925 | 0.983 | 0.007 | 0.784 |
| Hs.435976 | AW593887 | 56853 | chr18q12 | — | Hs.435976_at | bruno-like 4, RNA binding | BRUNOL4 | Amy | 0.263 | 1.050 | 0.697 | 1.027 | 0.013 | 1.212 |
| | | | | | | protein (Drosophila) | | | | | | | | |
| Hs.436066 | AF000425 | 7940 | chr6p21.3 | 109170 | Hs.436066_at | leukocyte specific transcript 1 | LST1 | Amy | 0.429 | 0.948 | 0.463 | 0.940 | 0.025 | 0.751 |
| Hs.436196 | BC036809 | 9639 | chr8p23 | 608136 | Hs.436196_at | Rho guanine nucleotide | ARHGEF10 | Amy | 0.870 | 0.992 | 0.540 | 0.944 | 0.020 | 0.768 |
| | | | | | | exchange factor (GEF) 10 | | | | | | | | |
| Hs.436325 | NM_004984 | 3798 | chr12q13.13 | 602821 | Hs.436325_at | kinesin family member 5A | KIF5A | Amy | 0.608 | 1.109 | 0.172 | 1.152 | 0.044 | 1.611 |
| Hs.436488 | AA102574 | 11177 | chr14q12-q13 | 605680 | Hs.436488_at | bromodomain adjacent to zinc | BAZ1A | Amy | 0.525 | 0.961 | 0.127 | 0.875 | 0.000 | 0.635 |
| | | | | | | finger domain, 1A | | | | | | | | |
| Hs.436494 | NM_021219 | 58494 | chr21q21.2 | 606870 | Hs.436494_at | junctional adhesion molecule 2 | JAM2 | Amy | 0.428 | 0.916 | 0.101 | 0.807 | 0.017 | 0.761 |
| Hs.436542 | AF142573 | 83690 | chr8q21.11 | — | Hs.436542_at | CocoaCrisp | LOC83690 | Amy | 0.349 | 1.109 | 0.785 | 0.972 | 0.000 | 0.651 |
| Hs.436596 | AL583171 | 10193 | chr12q13.2-q13.3 | — | Hs.436596_at | ring finger protein 41 | RNF41 | Amy | 0.221 | 1.158 | 0.077 | 1.187 | 0.033 | 1.212 |
| Hs.436617 | AV715391 | 115106 | chr18q21.1 | 608775 | Hs.436617_at | coiled-coil domain containing 5 | CCDC5 | Amy | 0.320 | 0.838 | 0.133 | 0.860 | 0.016 | 0.833 |
| | | | | | | (spindle associated) | | | | | | | | |
| Hs.436667 | AI986390 | 139728 | chrxq28 | — | Hs.436667_at | Similar to calcium/calmodulin- | MGC45419 | Amy | 0.139 | 1.178 | 0.187 | 1.141 | 0.011 | 1.295 |
| | | | | | | dependent protein kinase 1, | | | | | | | | |
| | | | | | | beta | | | | | | | | |
| Hs.436847 | AI141670 | 131408 | chr3q27.1 | — | Hs.436847_at | hypothetical protein MGC21688 | MGC21688 | Amy | 0.092 | 1.237 | 0.098 | 1.112 | 0.048 | 1.203 |
| Hs.437257 | BF308645 | 57580 | chr20q13.13 | 606905 | Hs.437257_at | phosphatidylinositol 3,4,5- | PREX1 | Amy | 0.931 | 0.985 | 0.769 | 0.945 | 0.019 | 0.766 |
| | | | | | | trisphosphate-dependent RAC | | | | | | | | |
| | | | | | | exchanger 1 | | | | | | | | |
| Hs.437277 | NM_014275 | 11282 | chr5q35 | 604561 | Hs.437277_at | mannosyl (alpha-1,3-)- | MGAT4B | Amy | 0.903 | 1.021 | 0.289 | 1.086 | 0.021 | 1.220 |
| | | | | | | glycoprotein beta-1,4-N- | | | | | | | | |
| | | | | | | acetylglucosaminyltransferase, | | | | | | | | |
| | | | | | | isoenzyme B | | | | | | | | |
| Hs.437362 | AB051515 | 85461 | chr2q24.2 | — | Hs.437362_at | KIAA1728 protein | KIAA1728 | Amy | 0.719 | 1.015 | 0.165 | 0.804 | 0.012 | 0.709 |
| Hs.437632 | AY029176 | 80333 | chr4p15.31 | 608182 | Hs.437632_at | Kv channel interacting protein 4 | KCNIP4 | Amy | 0.054 | 1.268 | 0.116 | 1.232 | 0.021 | 1.541 |
| Hs.437819 | BF058311 | 146059 | chr15q14 | 607465 | Hs.437819_at | congenital dyserythropoietic | CDAN1 | Amy | 0.196 | 0.877 | 0.642 | 0.962 | 0.026 | 0.828 |
| | | | | | | anemia, type I | | | | | | | | |
| Hs.438691 | NM_021971 | 29925 | chr3p21.31 | — | Hs.438691_at | GDP-mannose | GMPPB | Amy | 0.746 | 1.048 | 0.133 | 1.097 | 0.021 | 1.220 |
| | | | | | | pyrophosphorylase B | | | | | | | | |
| Hs.438720 | D55716 | 4176 | chr7q21.3-q22.1 | 600592 | Hs.438720_at | MCM7 minichromosome | MCM7 | Amy | 0.364 | 0.972 | 0.591 | 0.987 | 0.026 | 0.830 |
| | | | | | | maintenance deficient 7 (S. cerevisiae) | | | | | | | | |
| Hs.43910 | NM_006016 | 8763 | chr6q21 | 603356 | Hs.43910_at | CD164 antigen, sialomucin | CD164 | Amy | 0.875 | 0.959 | 0.303 | 1.095 | 0.015 | 0.748 |
| Hs.43913 | NM_006346 | 10464 | chr13q22.1 | 607532 | Hs.43913_at | progesterone-induced blocking | PIBF1 | Amy | 0.112 | 0.889 | 0.994 | 0.999 | 0.004 | 0.720 |
| | | | | | | factor 1 | | | | | | | | |
| Hs.439188 | AF479418 | 23063 | chr10q23.2 | — | Hs.439188_at | KIAA0261 | KIAA0261 | Amy | 0.345 | 1.065 | 0.847 | 0.989 | 0.020 | 0.815 |
| Hs.439190 | N30138 | 122786 | chr14q22.1 | — | Hs.439190_at | chromosome 14 open reading | C14orf31 | Amy | 0.762 | 0.958 | 0.314 | 0.912 | 0.006 | 0.754 |
| | | | | | | frame 31 | | | | | | | | |
| Hs.439463 | NM_001129 | 165 | chr7p13 | 602981 | Hs.439463_at | AE binding protein 1 | AEBP1 | Amy | 0.488 | 0.940 | 0.288 | 0.855 | 0.006 | 0.718 |
| Hs.439599 | NM_002372 | 4124 | chr5q21-q22 | 154582 | Hs.439599_at | mannosidase, alpha, class 2A, | MAN2A1 | Amy | 0.340 | 1.238 | 0.886 | 0.971 | 0.045 | 0.595 |
| | | | | | | member 1 | | | | | | | | |
| Hs.439671 | NM_005380 | 4681 | chr1p36.13-p36.11 | 600613 | Hs.439671_at | neuroblastoma, suppression of | NBL1 | Amy | 0.348 | 1.113 | 0.022 | 1.173 | 0.003 | 1.318 |
| | | | | | | tumorigenicity 1 | | | | | | | | |
| Hs.440379 | NM_014715 | 9743 | chr11q24-q25 | 608541 | Hs.440379_at | Rho GTPase-activating protein | RICS | Amy | 0.948 | 0.990 | 0.339 | 1.095 | 0.040 | 1.213 |
| Hs.44038 | NM_021255 | 57161 | chr14q21 | — | Hs.44038_at | pellino homolog 2 (Drosophila) | PELI2 | Amy | 0.183 | 0.916 | 0.152 | 0.798 | 0.046 | 0.786 |
| Hs.440401 | AK098125 | 54884 | chr2p11.2 | — | Hs.440401_at | hypothetical protein FLJ20296 | FLJ20296 | Amy | 0.194 | 1.123 | 0.768 | 0.966 | 0.003 | 0.751 |
| Hs.440497 | AL520102 | 8514 | chr1p36.3 | 601142 | Hs.440497_at | potassium voltage-gated | KCNAB2 | Amy | 0.300 | 1.181 | 0.033 | 1.143 | 0.042 | 1.251 |
| | | | | | | channel, shaker-related | | | | | | | | |
| | | | | | | subfamily, beta member 2 | | | | | | | | |
| Hs.440808 | AB011126 | 23048 | chr9q34 | 606191 | Hs.440808_at | formin binding protein 1 | FNBP1 | Amy | 0.589 | 1.048 | 0.560 | 0.929 | 0.043 | 0.764 |
| Hs.441281 | AF242529 | 29993 | chr6p21.3 | 606512 | Hs.441281_at | protein kinase C and casein | PACSIN1 | Amy | 0.078 | 1.160 | 0.035 | 1.131 | 0.012 | 1.287 |
| | | | | | | kinase substrate in neurons 1 | | | | | | | | |
| Hs.442733 | M63310 | 306 | chr4q13-q22 | 106490 | Hs.442733_at | annexin A3 | ANXA3 | Amy | 0.321 | 0.803 | 0.050 | 0.667 | 0.013 | 0.617 |
| Hs.44313 | NM_{—002908} | 5966 | chr2p13-p12 | 164910 | Hs.44313_at | v-rel reticuloendotheliosis viral | REL | Amy | 0.113 | 0.910 | 0.067 | 0.789 | 0.001 | 0.726 |
| | | | | | | oncogene homolog (avian) | | | | | | | | |
| Hs.443435 | NM_001797 | 1009 | chr16q22.1 | 600023 | Hs.443435_at | cadherin 11, type 2, OB- | CDH11 | Amy | 0.517 | 1.057 | 0.539 | 0.952 | 0.037 | 0.818 |
| | | | | | | cadherin (osteoblast) | | | | | | | | |
| Hs.443468 | AL133031 | 254251 | chr4p15.32 | — | Hs.443468_at | chromosome condensation | HCAP-G | Amy | 0.515 | 0.964 | 0.957 | 0.993 | 0.021 | 0.797 |
| | | | | | | protein G | | | | | | | | |
| Hs.443495 | AW235051 | 80777 | chr16q22.1 | — | Hs.443495_at | cytochrome b5 outer | CYB5-M | Amy | 0.081 | 1.113 | 0.172 | 1.072 | 0.035 | 1.224 |
| | | | | | | mitochondrial membrane | | | | | | | | |
| | | | | | | precursor | | | | | | | | |
| Hs.443836 | AK098048 | 1267 | chr17q21 | 123830 | Hs.443836_at | 2′,3′-cyclic nucleotide 3′ | CNP | Amy | 0.408 | 1.207 | 0.702 | 0.907 | 0.018 | 0.634 |
| | | | | | | phosphodiesterase | | | | | | | | |
| Hs.444327 | NM_004251 | 9367 | chrxp22.2 | 300284 | Hs.444327_at | RAB9A, member RAS | RAB9A | Amy | 0.630 | 1.067 | 0.208 | 1.225 | 0.008 | 0.766 |
| | | | | | | oncogene family | | | | | | | | |
| Hs.44439 | NM_016387 | 9306 | chr18q22.2 | 605118 | Hs.44439_at | suppressor of cytokine signaling 4 | SOCS4 | Amy | 0.769 | 0.925 | 0.444 | 0.888 | 0.020 | 0.720 |
| Hs.444445 | NM_003078 | 6604 | chr7q35-q36 | 601737 | Hs.444445_at | SWI/SNF related, matrix | SMARCD3 | Amy | 0.422 | 1.135 | 0.119 | 1.166 | 0.007 | 1.243 |
| | | | | | | associated, actin dependent | | | | | | | | |
| | | | | | | regulator of chromatin, | | | | | | | | |
| | | | | | | subfamily d, member 3 | | | | | | | | |
| Hs.444510 | NM_030984 | 6916 | chr7q34-q35 | 274180 | Hs.444510_at | thromboxane A synthase 1 | TBXAS1 | Amy | 0.346 | 1.038 | 0.537 | 1.029 | 0.004 | 0.829 |
| | | | | | | (platelet, cytochrome P450, | | | | | | | | |
| | | | | | | family 5, subfamily A) | | | | | | | | |
| Hs.444983 | | | | | Hs.444983_at | purinergic receptor P2Y, G- | P2RY12 | Amy | 0.340 | 0.795 | 0.106 | 0.783 | 0.019 | 0.344 |
| | | | | | | protein coupled, 12 | | | | | | | | |
| Hs.445489 | AF081583 | 58473 | chr11q13.5-q14.1 | 607651 | Hs.445489_at | pleckstrin homology domain | PLEKHB1 | Amy | 0.965 | 0.975 | 0.771 | 0.955 | 0.018 | 0.788 |
| | | | | | | containing, family B (evectins) | | | | | | | | |
| | | | | | | member 1 | | | | | | | | |
| Hs.446325 | AL353746 | 158038 | chr9p21.2-p21.1 | — | Hs.446325_at | hypothetical protein FLJ31810 | FLJ31810 | Amy | 0.674 | 1.061 | 0.082 | 1.139 | 0.018 | 1.406 |
| Hs.446471 | M28590 | 972 | chr5q32 | 142790 | Hs.446471_at | CD74 antigen (invariant | CD74 | Amy | 0.746 | 0.994 | 0.113 | 0.781 | 0.006 | 0.434 |
| | | | | | | polypeptide of major | | | | | | | | |
| | | | | | | histocompatibility complex, | | | | | | | | |
| | | | | | | class II antigen-associated) | | | | | | | | |
| Hs.446677 | | | | | Hs.446677_at | zinc finger protein 238 | ZNF238 | Amy | 0.252 | 1.260 | 0.137 | 1.161 | 0.034 | 1.432 |
| Hs.448805 | AF202640 | 51704 | chr16p12 | 605948 | Hs.448805_at | G protein-coupled receptor, | GPRC5B | Amy | 0.771 | 0.915 | 0.795 | 0.937 | 0.021 | 0.680 |
| | | | | | | family C, group 5, member B | | | | | | | | |
| Hs.449718 | AL521682 | 338773 | chr12q23.3 | — | Hs.449718_at | hypothetical protein LOC338773 | LOC338773 | Amy | 0.117 | 0.953 | 0.366 | 0.982 | 0.017 | 0.794 |
| Hs.45056 | NM_052904 | 114792 | chr6q16.1 | — | Hs.45056_at | KIAA1900 | KIAA1900 | Amy | 0.500 | 1.163 | 0.430 | 0.825 | 0.014 | 0.567 |
| Hs.456 | NM_000897 | 4056 | chr5q35 | 246530 | Hs.456_at | leukotriene C4 synthase | LTC4S | Amy | 0.398 | 1.038 | 0.087 | 0.842 | 0.035 | 0.774 |
| Hs.458291 | NM_000297 | 5311 | chr4q21-q23 | 173910 | Hs.458291_at | polycystic kidney disease 2 | PKD2 | Amy | 0.621 | 0.869 | 0.225 | 0.894 | 0.012 | 0.726 |
| | | | | | | (autosomal dominant) | | | | | | | | |
| Hs.458320 | AI937543 | 56941 | chr3q21.3 | — | Hs.458320_at | DC12 protein | DC12 | Amy | 0.327 | 1.053 | 0.275 | 1.075 | 0.042 | 1.241 |
| Hs.458335 | | | | | Hs.458335_at | chromosome 21 open reading | C21orf97 | Amy | 0.891 | 1.019 | 0.142 | 1.078 | 0.021 | 1.228 |
| | | | | | | frame 97 | | | | | | | | |
| Hs.458354 | NM_003247 | 7058 | chr6q27 | 188061 | Hs.458354_at | thrombospondin 2 | THBS2 | Amy | 0.825 | 0.934 | 0.201 | 0.729 | 0.033 | 0.537 |
| Hs.458482 | NM_004276 | 9478 | chr12q24.31 | 605563 | Hs.458482_at | calcium binding protein 1 | CABP1 | Amy | 0.916 | 1.038 | 0.778 | 1.033 | 0.010 | 1.551 |
| | | | | | | (calbrain) | | | | | | | | |
| Hs.459470 | NM_018639 | 55884 | chr12q24.23 | — | Hs.459470_at | WD repeat and SOCS box | WSB2 | Amy | 0.248 | 1.124 | 0.131 | 1.085 | 0.029 | 1.281 |
| | | | | | | containing protein 2 | | | | | | | | |
| Hs.459987 | AV712577 | 10541 | chr9q22.32 | — | Hs.459987_at | acidic (leucine-rich) nuclear | ANP32B | Amy | 0.564 | 1.085 | 0.513 | 0.929 | 0.032 | 0.762 |
| | | | | | | phosphoprotein 32 family, | | | | | | | | |
| | | | | | | member B | | | | | | | | |
| Hs.461300 | AK054714 | 126661 | chr1p34.1 | — | Hs.461300−_at | hypothetical protein LOC126661 | LOC126661 | Amy | 0.782 | 0.976 | 0.530 | 0.980 | 0.022 | 0.765 |
| Hs.46440 | NM_021094 | 6579 | chr12p12 | 602883 | Hs.46440_at | solute carrier organic anion | SLCO1A2 | Amy | 0.805 | 1.012 | 0.576 | 1.046 | 0.001 | 0.586 |
| | | | | | | transporter family, member 1A2 | | | | | | | | |
| Hs.47061 | AB018265 | 8408 | chr12q24.3 | 603168 | Hs.47061_at | unc-51-like kinase 1 (C. | ULK1 | Amy | 0.734 | 1.099 | 0.146 | 1.091 | 0.013 | 1.386 |
| | | | | | | elegans) | | | | | | | | |
| Hs.47357 | NM_003956 | 9023 | chr10q23 | 604551 | Hs.47357_at | cholesterol 25-hydroxylase | CH25H | Amy | 0.158 | 0.903 | 0.137 | 0.675 | 0.035 | 0.700 |
| Hs.47517 | NM_005619 | 6253 | chr19q13.32 | 603183 | Hs.47517_at | reticulon 2 | RTN2 | Amy | 0.076 | 1.208 | 0.059 | 1.161 | 0.022 | 1.336 |
| Hs.475848 | NM_017512 | 55556 | chr18p11.32 | 607427 | Hs.475848_at | rTS beta protein | HSRTSBETA | Amy | 0.476 | 1.093 | 0.656 | 1.089 | 0.028 | 0.614 |
| Hs.4766 | NM_014077 | 26017 | chr19pter-p13.3 | — | Hs.4766_at | DKFZP586O0120 protein | DKFZP586O | Amy | 0.246 | 1.261 | 0.201 | 1.111 | 0.038 | 1.205 |
| | | | | | | | 0120 | | | | | | | |
| Hs.479888 | NM_173808 | 257194 | chr1p31.1 | — | Hs.479888_at | neuronal growth regulator 1 | NEGR1 | Amy | 0.067 | 1.473 | 0.382 | 1.094 | 0.013 | 1.374 |
| Hs.484950 | | | | | Hs.484950_at | histone 1, H2ac | HIST1H2AC | Amy | 0.624 | 1.114 | 0.585 | 0.757 | 0.002 | 0.427 |
| Hs.48516 | NM_004048 | 567 | chr15q21-q22.2 | 109700 | Hs.48516_at | beta-2-microglobulin | B2M | Amy | 0.798 | 0.950 | 0.303 | 0.929 | 0.001 | 0.760 |
| Hs.4859 | AF367476 | 57018 | chr3q25.31 | — | Hs.4859_at | cyclin L1 | CCNL1 | Amy | 0.013 | 0.948 | 0.808 | 0.987 | 0.006 | 0.828 |
| Hs.48998 | AF169676 | 23768 | chr14q24-q32 | 604807 | Hs.48998_at | fibronectin leucine rich | FLRT2 | Amy | 0.970 | 0.997 | 0.946 | 1.004 | 0.008 | 1.213 |
| | | | | | | transmembrane protein 2 | | | | | | | | |
| Hs.4980 | NM_001290 | 9079 | chr4p16 | 603450 | Hs.4980_at | LIM domain binding 2 | LDB2 | Amy | 0.083 | 1.231 | 0.180 | 1.119 | 0.029 | 1.622 |
| Hs.498494 | | | | | Hs.498494_at | paired basic amino acid | PACE4 | Amy | 0.233 | 1.199 | 0.609 | 0.866 | 0.020 | 0.526 |
| | | | | | | cleaving system 4 | | | | | | | | |
| Hs.4993 | AB037734 | 57526 | chrXq13.3 | 300460 | Hs.4993_at | protocadherin 19 | PCDH19 | Amy | 0.988 | 1.001 | 0.117 | 1.084 | 0.014 | 1.293 |
| Hs.499659 | AK000478 | 23108 | chr17p13.3 | — | Hs.499659_at | KIAA1039 protein | KIAA1039 | Amy | 0.054 | 1.098 | 0.114 | 1.127 | 0.004 | 1.495 |
| Hs.500197 | NM_006695 | 10900 | chr17q21.31 | 605448 | Hs.500197_at | RaP2 interacting protein 8 | RPIP8 | Amy | 0.595 | 1.105 | 0.040 | 1.157 | 0.006 | 1.402 |
| Hs.508459 | NM_153456 | 266722 | chr13q32.1 | — | Hs.508459_at | heparan sulfate 6-O- | HS6ST3 | Amy | 0.349 | 1.039 | 0.245 | 1.096 | 0.039 | 1.242 |
| | | | | | | sulfotransferase 3 | | | | | | | | |
| Hs.509841 | NM_014989 | 22999 | chr6q12-q13 | 606629 | Hs.509841_at | regulating synaptic membrane | RIMS1 | Amy | 0.209 | 1.081 | 0.062 | 1.089 | 0.022 | 1.217 |
| | | | | | | exocytosis 1 | | | | | | | | |
| Hs.511745 | NM_006317 | 10409 | chr5p15.1-p14 | 605940 | Hs.511745_at | brain abundant, membrane | BASP1 | Amy | 0.150 | 1.152 | 0.084 | 1.094 | 0.040 | 1.294 |
| | | | | | | attached signal protein 1 | | | | | | | | |
| Hs.511922 | AF261715 | 219595 | chr11q14.3 | — | Hs.511922_at | prostate-specific membrane | PSMAL/GCP | Amy | 0.652 | 1.046 | 0.714 | 0.912 | 0.036 | 0.575 |
| | | | | | | antigen-like protein | III | | | | | | | |
| Hs.511936 | | | | | Hs.511936_at | ring finger protein 44 | RNF44 | Amy | 0.113 | 1.166 | 0.007 | 1.162 | 0.008 | 1.270 |
| Hs.511952 | AI458128 | 23466 | chr22q13.1 | — | Hs.511952_at | chromobox homolog 6 | CBX6 | Amy | 0.443 | 1.154 | 0.026 | 1.137 | 0.009 | 1.242 |
| Hs.512000 | NM_000407 | 2812 | chr22q11.21 | 138720 | Hs.512000_at | glycoprotein lb (platelet), beta | GP1BB | Amy | 0.318 | 1.100 | 0.209 | 1.137 | 0.047 | 1.236 |
| | | | | | | polypeptide | | | | | | | | |
| Hs.512319 | BC018336 | 374875 | chr19p13.3 | — | Hs.512319_at | short-chain | SCDR10 | Amy | 0.562 | 1.068 | 0.089 | 1.106 | 0.015 | 1.233 |
| | | | | | | dehydrogenase/reductase 10 | | | | | | | | |
| Hs.512651 | AL390158 | 56970 | chr17q21.31 | — | Hs.512651_at | hypothetical protein | DKFZp761G | Amy | 0.716 | 1.034 | 0.052 | 1.096 | 0.022 | 1.241 |
| | | | | | | DKFZp761G2113 | 2113 | | | | | | | |
| Hs.523532 | BC034024 | 758 | chr22q13.31 | 602112 | Hs.523532_at | chromosome 22 open reading | C22orf1 | Amy | 0.663 | 0.965 | 0.400 | 0.876 | 0.003 | 0.635 |
| | | | | | | frame 1 | | | | | | | | |
| Hs.528148 | AA736604 | 57471 | chr2q24.2 | — | Hs.528148_at | KIAA1189 protein | KIAA1189 | Amy | 0.417 | 1.412 | 0.846 | 0.934 | 0.022 | 0.599 |
| Hs.53066 | NM_012267 | 23640 | chr19q13.42 | — | Hs.53066_at | hsp70-interacting protein | HSPBP1 | Amy | 0.210 | 1.086 | 0.318 | 1.073 | 0.027 | 1.321 |
| Hs.54483 | NM_004688 | 9111 | chr2p24.3-q21.3 | 603525 | Hs.54483_at | N-myc (and STAT) interactor | NMI | Amy | 0.427 | 0.985 | 0.811 | 0.976 | 0.000 | 0.523 |
| Hs.5452 | NM_006676 | 10868 | chr9q34.11 | — | Hs.5452_at | ubiquitin specific protease 20 | USP20 | Amy | 0.160 | 1.086 | 0.037 | 1.060 | 0.004 | 1.211 |
| Hs.54697 | AI625739 | 23229 | chrxq11.2 | 300429 | Hs.54697_at | Cdc42 guanine nucleotide | ARHGEF9 | Amy | 0.421 | 1.220 | 0.050 | 1.165 | 0.021 | 1.342 |
| | | | | | | exchange factor (GEF) 9 | | | | | | | | |
| Hs.5509 | BC005926 | 2124 | chr17q11.2 | 158381 | Hs.5509_at | ecotropic viral integration site | EVI2B | Amy | 0.336 | 0.928 | 0.099 | 0.802 | 0.006 | 0.495 |
| | | | | | | 2B | | | | | | | | |
| Hs.55209 | BC030654 | 91833 | chr14q32.32 | — | Hs.55209_at | WD repeat domain 20 | WDR20 | Amy | 0.654 | 1.018 | 0.470 | 0.949 | 0.023 | 0.786 |
| Hs.552 | NM_001047 | 6715 | chr5p15 | 184753 | Hs.552_at | steroid-5-alpha-reductase, | SRD5A1 | Amy | 0.468 | 1.035 | 0.121 | 1.183 | 0.018 | 1.297 |
| | | | | | | alpha polypeptide 1 (3-oxo-5 | | | | | | | | |
| | | | | | | alpha-steroid delta 4- | | | | | | | | |
| | | | | | | dehydrogenase alpha 1) | | | | | | | | |
| Hs.56607 | BC006080 | 7462 | chr7q11.23 | 605719 | Hs.56607_at | Williams-Beuren syndrome | WBSCR5 | Amy | 0.489 | 0.967 | 0.228 | 0.901 | 0.005 | 0.712 |
| | | | | | | chromosome region 5 | | | | | | | | |
| Hs.5737 | BF115776 | 9917 | chr1p36.13-q41 | — | Hs.5737_at | family with sequence similarity | FAM20B | Amy | 0.345 | 1.229 | 0.076 | 1.135 | 0.003 | 1.231 |
| | | | | | | 20, member B | | | | | | | | |
| Hs.57856 | NM_012395 | 5218 | chr7q21-q22 | — | Hs.57856_at | PFTAIRE protein kinase 1 | PFTK1 | Amy | 0.062 | 1.213 | 0.987 | 1.001 | 0.004 | 1.273 |
| Hs.57937 | NM_145892 | 54715 | chr16p13.3 | 605104 | Hs.57937_at | ataxin 2-binding protein 1 | A2BP1 | Amy | 0.191 | 1.222 | 0.133 | 1.145 | 0.019 | 1.461 |
| Hs.58351 | AK090894 | 10351 | chr17q24 | — | Hs.58351_at | ATP-binding cassette, sub- | ABCA8 | Amy | 0.521 | 1.364 | 0.414 | 0.693 | 0.011 | 0.370 |
| | | | | | | family A (ABC1), member 8 | | | | | | | | |
| Hs.58617 | AL049383 | 9475 | chr2p24 | 604002 | Hs.58617_at | Rho-associated, coiled-coil | ROCK2 | Amy | 0.721 | 1.122 | 0.796 | 0.978 | 0.035 | 1.244 |
| | | | | | | containing protein kinase 2 | | | | | | | | |
| Hs.60177 | AL568422 | 22873 | chr13q32.1 | 608671 | Hs.60177_at | DAZ interacting protein 1 | DZIP1 | Amy | 0.407 | 1.158 | 0.031 | 1.143 | 0.015 | 1.273 |
| Hs.62180 | NM_018685 | 54443 | chr7p15-p14 | — | Hs.62180_at | anillin, actin binding protein | ANLN | Amy | 0.458 | 1.293 | 0.739 | 0.864 | 0.011 | 0.514 |
| | | | | | | (scraps homolog, Drosophila) | | | | | | | | |
| Hs.62661 | BC002666 | 2633 | chr1p22.2 | 600411 | Hs.62661_at | guanylate binding protein 1, | GBP1 | Amy | 0.943 | 1.004 | 0.384 | 0.927 | 0.004 | 0.555 |
| | | | | | | interferon-inducible, 67 kDa | | | | | | | | |
| Hs.6479 | BC006299 | 84315 | chr3p21.31 | — | Hs.6479_at | hypothetical protein MGC13272 | MGC13272 | Amy | 0.263 | 1.074 | 0.393 | 1.076 | 0.018 | 1.220 |
| Hs.65239 | AW026241 | 6330 | chr11q23.3 | 608256 | Hs.65239_at | sodium channel, voltage-gated, | SCN4B | Amy | 0.836 | 0.957 | 0.702 | 1.051 | 0.028 | 1.428 |
| | | | | | | type IV, beta | | | | | | | | |
| Hs.65848 | AL136594 | 84258 | chr19q13.33 | 600327 | Hs.65848_at | synaptotagmin III | SYT3 | Amy | 0.635 | 1.050 | 0.176 | 1.120 | 0.038 | 1.299 |
| Hs.66159 | AB037820 | 57574 | chr2q35 | 608208 | Hs.66159_at | KIAA1399 protein | KIAA1399 | Amy | 0.402 | 1.034 | 0.108 | 1.130 | 0.034 | 1.231 |
| Hs.66309 | BC004875 | 84272 | chr2p22.3 | — | Hs.66309_at | hypothetical protein MGC11061 | MGC11061 | Amy | 0.688 | 1.030 | 0.657 | 0.938 | 0.028 | 0.770 |
| Hs.6658 | BE221674 | 152404 | chr3q13.32 | 608351 | Hs.6658_at | immunoglobulin superfamily, | IGSF11 | Amy | 0.391 | 0.892 | 0.785 | 0.928 | 0.050 | 0.701 |
| | | | | | | member 11 | | | | | | | | |
| Hs.66708 | BC003570 | 9341 | chr1p36.23 | 603657 | Hs.66708_at | vesicle-associated membrane | VAMP3 | Amy | 0.926 | 0.981 | 0.936 | 1.014 | 0.038 | 0.762 |
| | | | | | | protein 3 (cellubrevin) | | | | | | | | |
| Hs.6820 | AF413522 | 219771 | chr10p11.21 | — | Hs.6820_at | chromosome 10 open reading | C10orf9 | Amy | 0.226 | 1.173 | 0.013 | 1.143 | 0.022 | 1.214 |
| | | | | | | frame 9 | | | | | | | | |
| Hs.6900 | AF070558 | 11342 | chr3q25.1 | — | Hs.6900_at | ring finger protein 13 | RNF13 | Amy | 0.914 | 1.022 | 0.956 | 0.994 | 0.015 | 0.707 |
| Hs.70327 | U36190 | 1397 | chr14q32.3 | 601183 | Hs.70327_at | cysteine-rich protein 2 | CRIP2 | Amy | 0.448 | 1.228 | 0.080 | 1.328 | 0.042 | 1.417 |
| Hs.70499 | NM_014210 | 2123 | chr17q11.2 | 158380 | Hs.70499_at | ecotropic viral integration site | EVI2A | Amy | 0.213 | 1.640 | 0.533 | 0.756 | 0.005 | 0.428 |
| | | | | | | 2A | | | | | | | | |
| Hs.72325 | BF694956 | 135114 | chr6q22.32 | — | Hs.72325_at | histidine triad nucleotide binding | HINT3 | Amy | 0.364 | 1.137 | 0.077 | 1.424 | 0.021 | 1.219 |
| | | | | | | protein 3 | | | | | | | | |
| Hs.72782 | AK023183 | 55783 | chr16q22.2 | — | Hs.72782_at | hypothetical protein FLJ11171 | FLJ11171 | Amy | 0.329 | 0.893 | 0.350 | 0.855 | 0.020 | 0.635 |
| Hs.74376 | NM_006334 | 10439 | chr9q34.3 | 605366 | Hs.74376_at | olfactomedin 1 | OLFM1 | Amy | 0.347 | 1.196 | 0.343 | 1.062 | 0.013 | 1.436 |
| Hs.74569 | AB020649 | 23207 | chr1p36.13 | — | Hs.74569_at | KIAA0842 protein | KIAA0842 | Amy | 0.513 | 0.937 | 0.303 | 1.052 | 0.036 | 1.297 |
| Hs.75082 | NM_001665 | 391 | chr11p15.5-p15.4 | 179505 | Hs.75082_at | ras homolog gene family, | ARHG | Amy | 0.058 | 0.839 | 0.191 | 0.828 | 0.010 | 0.747 |
| | | | | | | member G (rho G) | | | | | | | | |
| Hs.75236 | NM_021952 | 1996 | chr1p34 | 168360 | Hs.75236_at | ELAV (embryonic lethal, | ELAVL4 | Amy | 0.220 | 1.198 | 0.791 | 1.022 | 0.049 | 1.374 |
| | | | | | | abnormal vision, Drosophila)- | | | | | | | | |
| | | | | | | like 4 (Hu antigen D) | | | | | | | | |
| Hs.75256 | NM_002922 | 5996 | chr1q31 | 600323 | Hs.75256_at | regulator of G-protein signalling 1 | RGS1 | Amy | 0.181 | 0.870 | 0.320 | 0.554 | 0.042 | 0.280 |
| Hs.75438 | BC000576 | 5860 | chr4p15.31 | 261630 | Hs.75438_at | quinoid dihydropteridine | QDPR | Amy | 0.551 | 1.083 | 0.527 | 0.940 | 0.038 | 0.747 |
| | | | | | | reductase | | | | | | | | |
| Hs.75452 | NM_005345 | 3303 | chr6p21.3 | 140550 | Hs.75452_at | heat shock 70 kDa protein 1A | HSPA1A | Amy | 0.191 | 1.355 | 0.520 | 0.870 | 0.017 | 0.645 |
| Hs.75462 | BG339064 | 7832 | chr1q32 | 601597 | Hs.75462_at | BTG family, member 2 | BTG2 | Amy | 0.084 | 0.801 | 0.158 | 0.764 | 0.031 | 0.721 |
| Hs.75671 | NM_004603 | 6804 | chr7q11.23 | 186590 | Hs.75671_at | syntaxin 1A (brain) | STX1A | Amy | 0.061 | 1.140 | 0.090 | 1.097 | 0.034 | 1.297 |
| Hs.75794 | AW269335 | 1902 | chr9q31.3 | 602282 | Hs.75794_at | endothelial differentiation, | EDG2 | Amy | 0.982 | 0.996 | 0.941 | 0.984 | 0.029 | 0.687 |
| | | | | | | lysophosphatidic acid G-protein- | | | | | | | | |
| | | | | | | coupled receptor, 2 | | | | | | | | |
| Hs.76057 | AK096127 | 2582 | chr1p36-p35 | 606953 | Hs.76057_at | galactose-4-epimerase, UDP- | GALE | Amy | 0.157 | 1.223 | 0.799 | 1.023 | 0.028 | 1.338 |
| Hs.76224 | AI826799 | 2202 | chr2p16 | 601548 | Hs.76224_at | EGF-containing fibulin-like | EFEMP1 | Amy | 0.142 | 0.496 | 0.089 | 0.520 | 0.027 | 0.502 |
| | | | | | | extracellular matrix protein 1 | | | | | | | | |
| Hs.76289 | NM_000713 | 645 | chr19q13.1-q13.2 | 600941 | Hs.76289_at | biliverdin reductase B (flavin | BLVRB | Amy | 0.913 | 0.992 | 0.183 | 1.090 | 0.013 | 1.247 |
| | | | | | | reductase (NADPH)) | | | | | | | | |
| Hs.76364 | NM_004847 | 199 | chr6p21.3 | 601833 | Hs.76364_at | allograft inflammatory factor 1 | AIF1 | Amy | 0.302 | 0.930 | 0.118 | 0.883 | 0.042 | 0.805 |
| Hs.76507 | AF010312 | 9516 | chr16p13.3-p12 | 603795 | Hs.76507_at | lipopolysaccharide-induced TNF | LITAF | Amy | 0.830 | 1.017 | 0.921 | 1.017 | 0.018 | 0.717 |
| | | | | | | factor | | | | | | | | |
| Hs.76894 | AI656493 | 1635 | chr4q35.1 | 607638 | Hs.76894_at | dCMP deaminase | DCTD | Amy | 0.259 | 1.107 | 0.030 | 1.137 | 0.010 | 1.227 |
| Hs.76917 | BC040456 | 26269 | chr4q34.1 | 605649 | Hs.76917_at | F-box only protein 8 | FBXO8 | Amy | 0.566 | 0.923 | 0.409 | 0.894 | 0.041 | 0.782 |
| Hs.77422 | NM_002668 | 5355 | chrxp11.23 | 300112 | Hs.77422_at | proteolipid protein 2 (colonic | PLP2 | Amy | 0.426 | 0.961 | 0.480 | 0.917 | 0.046 | 0.807 |
| | | | | | | epithelium-enriched) | | | | | | | | |
| Hs.77646 | BC014479 | 54899 | chr3p14.3 | — | Hs.77646_at | PX domain containing | PXK | Amy | 0.293 | 1.328 | 0.980 | 1.007 | 0.034 | 0.542 |
| | | | | | | serine/threonine kinase | | | | | | | | |
| Hs.7778 | NM_018205 | 55222 | chr10q22.1 | — | Hs.7778_at | hypothetical protein FLJ10751 | FLJ10751 | Amy | 0.686 | 1.026 | 0.069 | 1.095 | 0.036 | 1.209 |
| Hs.77961 | D83043 | 3106 | chr6p21.3 | 142830 | Hs.77961_at | major histocompatibility | HLA-B | Amy | 0.843 | 0.915 | 0.275 | 0.927 | 0.008 | 0.724 |
| | | | | | | complex, class I, B | | | | | | | | |
| Hs.78224 | NM_002933 | 6035 | chr14q11.2 | 180440 | Hs.78224_at | ribonuclease, RNase A family, 1 | RNASE1 | Amy | 0.942 | 0.983 | 0.197 | 0.762 | 0.006 | 0.515 |
| | | | | | | (pancreatic) | | | | | | | | |
| Hs.7845 | NM_023939 | 65996 | chr19q13.43 | — | Hs.7845_at | hypothetical protein MGC2752 | MGC2752 | Amy | 0.681 | 1.010 | 0.384 | 1.064 | 0.012 | 1.202 |
| Hs.78746 | NM_002605 | 5151 | chr15q25.3 | 602972 | Hs.78746_at | phosphodiesterase 8A | PDE8A | Amy | 0.816 | 0.962 | 0.369 | 0.881 | 0.025 | 0.622 |
| Hs.78748 | NM_014747 | 9783 | chr1pter-p22.2 | — | Hs.78748_at | regulating synaptic membrane | RIMS3 | Amy | 0.121 | 1.216 | 0.035 | 1.120 | 0.023 | 1.394 |
| | | | | | | exocytosis 3 | | | | | | | | |
| Hs.78769 | NM_003249 | 7064 | chr19q13.3 | 601117 | Hs.78769_at | thimet oligopeptidase 1 | THOP1 | Amy | 0.887 | 0.987 | 0.336 | 1.067 | 0.019 | 1.489 |
| Hs.7886 | NM_020651 | 57162 | chr2p13.3 | — | Hs.7886_at | pellino homolog 1 (Drosophila) | PELI1 | Amy | 0.545 | 0.963 | 0.224 | 0.869 | 0.024 | 0.705 |
| Hs.78913 | AI033393 | 1524 | chr3p21.3 | 601470 | Hs.78913_at | chemokine (C-X3-C motif) | CX3CR1 | Amy | 0.571 | 0.931 | 0.083 | 0.772 | 0.009 | 0.475 |
| | | | | | | receptor 1 | | | | | | | | |
| Hs.78 | D13318 | 2551 | chr21q21.3 | 600609 | Hs.78_at | GA binding protein transcription | GABPA | Amy | 0.295 | 0.913 | 0.407 | 0.940 | 0.009 | 0.828 |
| | | | | | | factor, alpha subunit 60 kDa | | | | | | | | |
| Hs.79000 | NM_002045 | 2596 | chr3q13.1-q13.2 | 162060 | Hs.79000_at | growth associated protein 43 | GAP43 | Amy | 0.213 | 1.176 | 0.393 | 1.045 | 0.041 | 1.404 |
| Hs.79226 | NM_005103 | 9638 | chr11q24.2 | 604825 | Hs.79226_at | fasciculation and elongation | FEZ1 | Amy | 0.736 | 1.058 | 0.494 | 1.049 | 0.008 | 0.811 |
| | | | | | | protein zeta 1 (zygin I) | | | | | | | | |
| Hs.79299 | N66633 | 10184 | chr5q14.1 | — | Hs.79299_at | lipoma HMGIC fusion partner- | LHFPL2 | Amy | 0.567 | 1.038 | 0.291 | 0.890 | 0.036 | 0.814 |
| | | | | | | like 2 | | | | | | | | |
| Hs.7934 | BE222801 | 192683 | chr15q23 | — | Hs.7934_at | secretory carrier membrane | SCAMP5 | Amy | 0.642 | 1.036 | 0.165 | 1.079 | 0.035 | 1.203 |
| | | | | | | protein 5 | | | | | | | | |
| Hs.7946 | AI695017 | 57509 | chr8p22 | — | Hs.7946_at | mitochondrial tumor suppressor | MTSG1 | Amy | 0.993 | 1.003 | 0.878 | 0.963 | 0.035 | 0.724 |
| | | | | | | gene 1 | | | | | | | | |
| Hs.7989 | AB028968 | 23349 | chr9p13.2 | — | Hs.7989_at | KIAA1045 | KIAA1045 | Amy | 0.205 | 1.173 | 0.076 | 1.179 | 0.004 | 1.559 |
| Hs.80680 | NM_017458 | 9961 | chr16p13.1-P11.2 | 605088 | Hs.80680_at | major vault protein | MVP | Amy | 0.354 | 0.990 | 0.838 | 0.976 | 0.015 | 0.808 |
| Hs.80720 | AK074381 | 2649 | chr4q31.21 | 604439 | Hs.80720+_at | GRB2-associated binding | GAB1 | Amy | 0.999 | 1.000 | 0.464 | 0.893 | 0.002 | 0.620 |
| | | | | | | protein 1 | | | | | | | | |
| Hs.808 | AI591354 | 3185 | chr10q11.21-q11.22 | 601037 | Hs.808_at | heterogeneous nuclear | HNRPF | Amy | 0.386 | 0.942 | 0.984 | 1.002 | 0.008 | 0.725 |
| | | | | | | ribonucleoprotein F | | | | | | | | |
| Hs.80919 | AI768845 | 6856 | chr7q22.3 | — | Hs.80919_at | synaptophysin-like protein | SYPL | Amy | 0.964 | 0.990 | 0.700 | 0.944 | 0.001 | 0.655 |
| Hs.8117 | NM_018695 | 55914 | chr5q12.3 | 606944 | Hs.8117_at | erbb2 interacting protein | ERBB2IP | Amy | 0.766 | 0.922 | 0.290 | 0.860 | 0.003 | 0.706 |
| Hs.81424 | U67122 | 7341 | chr2q33 | 601912 | Hs.81424_at | ubiquitin-like 1 (sentrin) | UBL1 | Amy | 0.093 | 1.337 | 0.113 | 1.158 | 0.033 | 1.230 |
| Hs.82128 | NM_006670 | 7162 | chr6q14-q15 | 190920 | Hs.82128_at | trophoblast glycoprotein | TPBG | Amy | 0.725 | 1.065 | 0.606 | 0.913 | 0.022 | 1.267 |
| Hs.82163 | NM_000898 | 4129 | chrxp11.23 | 309860 | Hs.82163_at | monoamine oxidase B | MAOB | Amy | 0.685 | 0.938 | 0.311 | 1.080 | 0.047 | 0.775 |
| Hs.8217 | NM_006603 | 10735 | chrxq25 | 604359 | Hs.8217_at | stromal antigen 2 | STAG2 | Amy | 0.882 | 1.020 | 0.308 | 0.900 | 0.010 | 0.817 |
| Hs.82222 | NM_004636 | 7869 | chr3p21.3 | 601281 | Hs.82222_at | sema domain, immunoglobulin | SEMA3B | Amy | 0.702 | 0.966 | 0.642 | 0.941 | 0.017 | 0.745 |
| | | | | | | domain (Ig), short basic domain, | | | | | | | | |
| | | | | | | secreted, (semaphorin) 3B | | | | | | | | |
| Hs.82280 | W19676 | 6001 | chr10q25 | 602856 | Hs.82280_at | regulator of G-protein signalling | RGS10 | Amy | 0.522 | 0.940 | 0.207 | 0.865 | 0.019 | 0.592 |
| | | | | | | 10 | | | | | | | | |
| Hs.82318 | AB020707 | 10810 | chr13q12 | 605068 | Hs.82318_at | WAS protein family, member 3 | WASF3 | Amy | 0.767 | 1.049 | 0.018 | 1.138 | 0.032 | 1.203 |
| Hs.82353 | NM_006404 | 10544 | chr20q11.2 | 600646 | Hs.82353_at | protein C receptor, endothelial | PROCR | Amy | 0.370 | 0.913 | 0.591 | 0.974 | 0.031 | 0.743 |
| | | | | | | (EPCR) | | | | | | | | |
| Hs.82407 | | | | | Hs.82407_at | chemokine (C-X-C motif) ligand | CXCL16 | Amy | 0.556 | 1.029 | 0.173 | 0.844 | 0.004 | 0.636 |
| | | | | | | 16 | | | | | | | | |
| Hs.82568 | NM_000784 | 1593 | chr2q33-qter | 606530 | Hs.82568_at | cytochrome P450, family 27, | CYP27A1 | Amy | 0.156 | 1.090 | 0.366 | 0.900 | 0.001 | 0.748 |
| | | | | | | subfamily A, polypeptide 1 | | | | | | | | |
| Hs.82646 | BG537255 | 3337 | chr19p13.2 | 604572 | Hs.82646_at | DnaJ (Hsp40) homolog, | DNAJB1 | Amy | 0.539 | 1.100 | 0.834 | 1.020 | 0.034 | 0.825 |
| | | | | | | subfamily B, member 1 | | | | | | | | |
| Hs.82771 | NM_006296 | 7444 | chr2p16-p15 | 602169 | Hs.82771_at | vaccinia related kinase 2 | VRK2 | Amy | 0.190 | 1.091 | 0.855 | 0.966 | 0.015 | 0.635 |
| Hs.82927 | AI916249 | 271 | chr1p13.3 | 102771 | Hs.82927_at | adenosine monophosphate | AMPD2 | Amy | 0.360 | 1.048 | 0.146 | 1.104 | 0.004 | 1.247 |
| | | | | | | deaminase 2 (isoform L) | | | | | | | | |
| Hs.83077 | NM_001562 | 3606 | chr11q22.2-q22.3 | 600953 | Hs.83077_at | interleukin 18 (interferon- | IL18 | Amy | 0.620 | 0.923 | 0.460 | 0.904 | 0.045 | 0.768 |
| | | | | | | gamma-inducing factor) | | | | | | | | |
| Hs.83381 | NM_004126 | 2791 | chr7q31-q32 | 604390 | Hs.83381_at | guanine nucleotide binding | GNG11 | Amy | 0.043 | 0.838 | 0.074 | 0.803 | 0.027 | 0.797 |
| | | | | | | protein (G protein), gamma 11 | | | | | | | | |
| Hs.84665 | NM_006790 | 9499 | chr5q31 | 604103 | Hs.84665_at | titin immunoglobulin domain | TTID | Amy | 0.447 | 1.027 | 0.453 | 0.848 | 0.009 | 0.479 |
| | | | | | | protein (myotilin) | | | | | | | | |
| Hs.85155 | BE620915 | 677 | chr14q22-q24 | 601064 | Hs.85155_at | zinc finger protein 36, C3H type- | ZFP36L1 | Amy | 0.118 | 0.868 | 0.488 | 0.911 | 0.028 | 0.771 |
| | | | | | | like 1 | | | | | | | | |
| Hs.85226 | BC040833 | 3988 | chr10q23.2-q23.3 | 278000 | Hs.85226_at | lipase A, lysosomal acid, | LIPA | Amy | 0.793 | 1.024 | 0.644 | 0.964 | 0.011 | 0.708 |
| | | | | | | cholesterol esterase (Wolman | | | | | | | | |
| | | | | | | disease) | | | | | | | | |
| Hs.85618 | AW963951 | 256435 | chr1p31.1 | — | Hs.85618_at | sialyltransferase 7 ((alpha-N- | SIAT7C | Amy | 0.946 | 0.988 | 0.529 | 0.913 | 0.044 | 0.759 |
| | | | | | | acetylneuraminyl-2,3-beta- | | | | | | | | |
| | | | | | | galactosyl-1,3)-N-acetyl | | | | | | | | |
| | | | | | | galactosaminide alpha-2,6- | | | | | | | | |
| | | | | | | sialyltransferase) C | | | | | | | | |
| Hs.8594 | BC006316 | 57179 | chr5q35.2 | — | Hs.8594_at | KIAA1191 protein | KIAA1191 | Amy | 0.472 | 1.330 | 0.042 | 1.106 | 0.008 | 1.253 |
| Hs.87729 | NM_022783 | 64798 | chr8q24.12 | — | Hs.87729_at | hypothetical protein FLJ12428 | FLJ12428 | Amy | 0.893 | 0.981 | 0.270 | 0.808 | 0.036 | 0.628 |
| Hs.878 | NM_003104 | 6652 | chr15q158.3 | 182500 | Hs.878_at | sorbitol dehydrogenase | SORD | Amy | 0.847 | 1.010 | 0.217 | 1.168 | 0.025 | 0.791 |
| Hs.8813 | BC028028 | 6814 | chr1p13.3 | 608339 | Hs.8813_at | syntaxin binding protein 3 | STXBP3 | Amy | 0.649 | 0.962 | 0.300 | 0.898 | 0.013 | 0.747 |
| Hs.88778 | BC002511 | 873 | chr21q22.13 | 114830 | Hs.88778_at | carbonyl reductase 1 | CBR1 | Amy | 0.790 | 0.959 | 0.967 | 0.992 | 0.025 | 0.724 |
| Hs.8904 | NM_007268 | 11326 | chrxq12-q13.3 | 300353 | Hs.8904_at | Ig superfamily protein | Z39IG | Amy | 0.289 | 0.860 | 0.159 | 0.884 | 0.007 | 0.605 |
| Hs.89512 | R52647 | 491 | chr3p25.3 | 108733 | Hs.89512_at | ATPase, Ca++ transporting, | ATP2B2 | Amy | 0.327 | 1.142 | 0.096 | 1.166 | 0.032 | 1.275 |
| | | | | | | plasma membrane 2 | | | | | | | | |
| Hs.8986 | NM_000491 | 713 | chr1p36.3-p34.1 | 120570 | Hs.8986_at | complement component 1, q | C1QB | Amy | 0.242 | 0.948 | 0.112 | 0.772 | 0.009 | 0.459 |
| | | | | | | subcomponent, beta | | | | | | | | |
| | | | | | | polypeptide | | | | | | | | |
| Hs.90436 | NM_004890 | 9552 | chr17p13.2 | — | Hs.90436_at | sperm associated antigen 7 | SPAG7 | Amy | 0.773 | 0.981 | 0.186 | 1.083 | 0.011 | 1.273 |
| Hs.9082 | AU146949 | 53371 | chr4q21.1 | 607607 | Hs.9082_at | nucleoporin 54 kDa | NUP54 | Amy | 0.475 | 1.040 | 0.319 | 0.951 | 0.003 | 0.827 |
| Hs.91448 | NM_007026 | 11072 | chr17q12 | 606618 | Hs.91448_at | dual specificity phosphatase 14 | DUSP14 | Amy | 0.508 | 1.097 | 0.485 | 1.072 | 0.014 | 1.233 |
| Hs.914 | M27487 | 3113 | chr6p21.3 | 142880 | Hs.914_at | major histocompatibility | HLA-DPA1 | Amy | 0.146 | 0.888 | 0.131 | 0.668 | 0.004 | 0.381 |
| | | | | | | complex, class II, DP alpha 1 | | | | | | | | |
| Hs.92732 | BC002606 | 57595 | chrxq28 | — | Hs.92732_at | LU1 protein | KIAA1444 | Amy | 0.996 | 1.000 | 0.099 | 1.187 | 0.013 | 1.393 |
| Hs.9315 | NM_020190 | 56944 | chr1p13.2 | — | Hs.9315_at | HNOEL-iso protein | HNOEL-iso | Amy | 0.695 | 1.007 | 0.289 | 0.805 | 0.029 | 0.621 |
| Hs.9599 | NM_014251 | 10165 | chr7q21.3 | 603859 | Hs.9599_at | solute carrier family 25, member | SLC25A13 | Amy | 0.451 | 0.928 | 0.904 | 1.013 | 0.017 | 0.734 |
| | | | | | | 13 (citrin) | | | | | | | | |
| Hs.9622 | NM_018135 | 55168 | chr6p21.3 | — | Hs.9622_at | mitochondrial ribosomal protein | MRPS18A | Amy | 0.398 | 1.026 | 0.228 | 1.082 | 0.019 | 1.252 |
| | | | | | | S18A | | | | | | | | |
| Hs.9641 | NM_015991 | 712 | chr1p36.3-p34.1 | 120550 | Hs.9641_at | complement component 1, q | C1QA | Amy | 0.766 | 0.963 | 0.200 | 0.692 | 0.003 | 0.373 |
| | | | | | | subcomponent, alpha | | | | | | | | |
| | | | | | | polypeptide | | | | | | | | |
| Hs.96 | AI857639 | 5366 | chr18q21.32 | 604959 | Hs.96_at | phorbol-12-myristate-13- | PMAIP1 | Amy | 0.892 | 0.995 | 0.320 | 0.942 | 0.019 | 0.833 |
| | | | | | | acetate-induced protein 1 | | | | | | | | |
| Hs.9741 | AK024269 | 93643 | chr6p21.1 | — | Hs.9741_at | tight junction protein 4 | TJP4 | Amy | 0.756 | 0.967 | 0.884 | 1.017 | 0.013 | 0.736 |
| | | | | | | (peripheral) | | | | | | | | |
| Hs.97616 | NM_003025 | 6455 | chr19p13.3 | 601768 | Hs.97616_at | SH3-domain GRB2-like 1 | SH3GL1 | Amy | 0.266 | 1.090 | 0.085 | 1.155 | 0.013 | 1.229 |
| Hs.99272 | AI147740 | 116173 | chr14q11.2 | 607888 | Hs.99272_at | chemokine-like factor super | CKLFSF5 | Amy | 0.085 | 1.256 | 0.661 | 0.895 | 0.032 | 0.687 |
| | | | | | | family 5 | | | | | | | | |
| Hs.9927 | AI969112 | 55023 | chr6q14 | — | Hs.9927-_at | pleckstrin homology domain | PHIP | Amy | 0.328 | 0.885 | 0.928 | 1.009 | 0.020 | 0.828 |
| | | | | | | interacting protein | | | | | | | | |

TABLE 23

| | Repre- | | Chromo- | | | | | | | |
|---|---|---|---|---|---|---|---|---|---|---|
| UniGene | sentative | Locus | somal | | Probe set | | Gene | Platform, | | fold |
| ID | Public ID | Link | Location | OMIM | ID UG-1 | Gene Title | Symbol | Region | t-test | change |
| Hs.284137 | NM_024945 | 80010 | chr9q21.32 | — | Hs.284137_at | chromosome | C9orf76 | AnCg | 0.020725 | 0.816708 |
| | | | | | | 9 open | | | | |
| | | | | | | reading | | | | |
| | | | | | | frame 76 | | | | |
| Hs.407155 | NM_025097 | 80167 | chr4q28.2 | — | Hs.407155_at | hypothetical | FLJ21106 | AnCg | 0.026331 | 0.808572 |
| | | | | | | protein | | | | |
| | | | | | | FLJ21106 | | | | |
| Hs.170081 | NM_173675 | 285780 | chr6p25.1 | — | Hs.170081_at | hypothetical | FLJ33708 | AnCg | 0.047521 | 1.24261 |
| | | | | | | protein | | | | |
| | | | | | | FLJ33708 | | | | |
| Hs.114218 | NM_003506 | 8323 | chr8q22.3- | 603409 | Hs.114218_at | frizzled | FZD6 | AnCg | 0.03536 | 0.762731 |
| | | | q23.1 | | | homolog 6 | | | | |
| | | | | | | (Drosophila) | | | | |
| Hs.26312 | NM_006338 | 10446 | chr1q32.1 | 605492 | Hs.26312_at | leucine rich | LRRN5 | AnCg | 0.039771 | 1.201087 |
| | | | | | | repeat | | | | |
| | | | | | | neuronal 5 | | | | |
| Hs.408161 | AW007241 | 57611 | chr15q24.1 | — | Hs.408161_at | KIAA1465 | KIAA1465 | AnCg | 0.020208 | 1.261026 |
| | | | | | | protein | | | | |
| Hs.30581 | H05918 | 84623 | chr11q24 | 607761 | Hs.30581_at | kin of | KIRREL3 | AnCg | 0.02756 | 1.242475 |
| | | | | | | IRRE like 3 | | | | |
| | | | | | | (Drosophila) | | | | |
| Hs.285782 | NM_024993 | 80059 | chr2p12 | — | Hs.285782_at | leucine | LRRTM4 | AnCg | 0.049497 | 1.358254 |
| | | | | | | rich repeat | | | | |
| | | | | | | transmembrane | | | | |
| | | | | | | neuronal 4 | | | | |
| Hs.148932 | NM_020241 | 10501 | chr19p13.3 | — | Hs.148932_at | sema domain, | SEMA6B | AnCg | 0.023939 | 1.261144 |
| | | | | | | transmembrane | | | | |
| | | | | | | domain | | | | |
| | | | | | | (TM), and | | | | |
| | | | | | | cytoplasmic | | | | |
| | | | | | | domain, | | | | |
| | | | | | | (semaphorin) | | | | |
| | | | | | | 6B | | | | |
| Hs.247302 | NM_020648 | 57045 | chr18p11.3 | 605049 | Hs.247302_at | twisted | TWSG1 | AnCg | 0.028339 | 0.75457 |
| | | | | | | gastrulation | | | | |
| | | | | | | homolog 1 | | | | |
| | | | | | | (Drosophila) | | | | |
| Hs.128705 | | | | | Hs.128705_at | | | AnCg | 0.003369 | 0.629083 |
| Hs.134441 | | | | | Hs.134441_at | | | AnCg | 0.006414 | 0.74478 |
| Hs.143134 | AW207243 | — | — | — | Hs.143134_at | CDNA FLJ38181 | — | AnCg | 0.038675 | 1.25541 |
| | | | | | | fis, clone | | | | |
| | | | | | | FCBBF1000125 | | | | |
| Hs.146268 | AW001557 | — | — | — | Hs.146268_at | Clone DNA59613 | — | AnCg | 0.030621 | 1.325909 |
| | | | | | | phospholipase | | | | |
| | | | | | | inhibitor | | | | |
| | | | | | | (UNQ511) mRNA, | | | | |
| | | | | | | complete cds | | | | |
| Hs.162203 | | | | | Hs.162203_at | | | AnCg | 0.023928 | 0.814199 |
| Hs.170973 | | | | | Hs.170973_at | | | AnCg | 0.035994 | 1.320933 |
| Hs.170999 | AK025747 | 55137 | chr2q24.3 | 605295 | Hs.170999_at | fidgetin | FIGN | AnCg | 0.017672 | 0.771728 |
| Hs.191346 | | | | | Hs.191346_at | | | AnCg | 0.002449 | 0.734099 |
| Hs.213501 | | | | | Hs.213501_at | | | AnCg | 0.016879 | 1.363411 |
| Hs.250879 | BF982289 | 404217 | chr19p13.2 | — | Hs.250879_at | cortexin 1 | CTXN1 | AnCg | 0.011148 | 1.357262 |
| Hs.306834 | | | | | Hs.306834_at | | | AnCg | 0.041437 | 1.275879 |
| Hs.307559 | | | | | Hs.307559_at | | | AnCg | 0.015434 | 0.552768 |
| Hs.369984 | | | | | Hs.369984_at | | | AnCg | 0.037255 | 0.768352 |
| Hs.379010 | AK092432 | 8000 | chr8q24.2 | 602470 | Hs.379010_at | prostate stem | PSCA | AnCg | 0.024803 | 1.310589 |
| | | | | | | cell antigen | | | | |
| Hs.4204 | AA700440 | — | — | — | Hs.4204_at | CDNA FLJ30779 | — | AnCg | 0.043015 | 1.289724 |
| | | | | | | fis, clone | | | | |
| | | | | | | FEBRA2000815 | | | | |
| Hs.42294 | | | | | Hs.42294_at | | | AnCg | 0.005224 | 0.797537 |
| Hs.434124 | AK057562 | 149086 | chr1p35.2 | — | Hs.434124_at | hypothetical | LOC149086 | AnCg | 0.004279 | 0.577431 |
| | | | | | | protein | | | | |
| | | | | | | LOC149086 | | | | |
| Hs.435222 | | | | | Hs.435222_at | | | AnCg | 0.022825 | 1.722226 |
| Hs.437332 | | | | | Hs.437332_at | | | AnCg | 0.020915 | 1.200778 |
| Hs.438801 | | | | | Hs.438801_at | | | AnCg | 0.007572 | 1.279146 |
| Hs.446394 | NM_004327 | 613 | chr22q11.23 | 151410 | Hs.446394_at | breakpoint | BCR | AnCg | 0.013515 | 1.291847 |
| | | | | | | duster | | | | |
| | | | | | | region | | | | |
| Hs.446593 | BC029534 | — | — | — | Hs.446593_at | CDNA clone | — | AnCg | 0.018501 | 1.294104 |
| | | | | | | IMAGE: 6101590, | | | | |
| | | | | | | partial cds | | | | |
| Hs.448624 | | | | | Hs.448624_at | | | AnCg | 0.049625 | 1.259765 |
| Hs.452203 | AI939400 | 400999 | chr2q14.1 | — | Hs.452203_at | Hypothetical | — | AnCg | 0.009538 | 1.410325 |
| | | | | | | gene supported | | | | |
| | | | | | | by AK124342 | | | | |
| Hs.458379 | | | | | Hs.458379_at | | | AnCg | 0.00181 | 1.290973 |
| Hs.493302 | AK021913 | — | — | — | Hs.493302_at | CDNA FLJ11851 | — | AnCg | 0.004343 | 1.404944 |
| | | | | | | fis, clone | | | | |
| | | | | | | HEMBA1006744 | | | | |
| Hs.514146 | | | | | Hs.514146_at | | | AnCg | 0.017236 | 1.2257 |
| Hs.515369 | | | | | Hs.515369_at | | | AnCg | 0.04564 | 0.461191 |
| Hs.516311 | BF056746 | — | — | — | Hs.516311_at | MRNA; cDNA | — | AnCg | 0.017839 | 0.676076 |
| | | | | | | DKFZp686E10196 | | | | |
| | | | | | | (from clone | | | | |
| | | | | | | DKFZp686E10196); | | | | |
| | | | | | | complete cds | | | | |
| Hs.517410 | | | | | Hs.517410_at | | | AnCg | 0.001473 | 1.382544 |
| Hs.519758 | | | | | Hs.519758_at | | | AnCg | 0.019567 | 1.392608 |
| Hs.521215 | | | | | Hs.521215_at | | | AnCg | 0.000387 | 0.708137 |
| Hs.531424 | BF111846 | 399563 | — | — | Hs.531424_at | hypothetical | FLJ43806 | AnCg | 0.01124 | 1.265719 |
| | | | | | | protein | | | | |
| | | | | | | FLJ43806 | | | | |
| Hs.531897 | | | | | Hs.531897_at | | | AnCg | 0.013124 | 0.606227 |
| Hs.66095 | | | | | Hs.66095_at | | | AnCg | 0.00517 | 0.61691 |
| Hs.74832 | | | | | Hs.74832_at | | | AnCg | 0.021275 | 0.775137 |
| Hs.88558 | | | | | Hs.88558_at | | | AnCg | 0.036854 | 0.791453 |
| | | | | | _at | | | | | |
| | | | | | _at | | | | | |
| Hs.4863 | NM_030797 | 81553 | chr2p24.3- | — | Hs.4863_at | family with | FAM49A | DLPFC | 0.022942 | 1.382579 |
| | | | p24.2 | | | sequence | | | | |
| | | | | | | similarity 49, | | | | |
| | | | | | | member A/// | | | | |
| | | | | | | family with | | | | |
| | | | | | | sequence | | | | |
| | | | | | | similarity | | | | |
| | | | | | | 49, member A | | | | |
| Hs.127286 | BF109660 | 346887 | chr8q23.1 | — | Hs.127286_at | Similar to solute | — | DLPFC | 0.001526 | 1.35149 |
| | | | | | | carrier family | | | | |
| | | | | | | 16 (monocar- | | | | |
| | | | | | | boxylic acid | | | | |
| | | | | | | transporters), | | | | |
| | | | | | | member 14 | | | | |
| Hs.134228 | NM_020794 | 57554 | chr1p31.1 | — | Hs.134228_at | densin-180 | KIAA1365 | DLPFC | 0.049737 | 1.243024 |
| Hs.170357 | BC036602 | — | — | — | Hs.170357_at | Clone IMAGE: | — | DLPFC | 0.010211 | 1.725662 |
| | | | | | | 5274542, | | | | |
| | | | | | | mRNA | | | | |
| Hs.21374 | | | | | Hs.21374_at | | | DLPFC | 0.039593 | 1.316786 |
| Hs.235795 | AW043859 | — | — | — | Hs.235795_at | Clone IMAGE: | — | DLPFC | 0.037066 | 0.756002 |
| | | | | | | 5263020, | | | | |
| | | | | | | mRNA | | | | |
| Hs.307559 | | | | | Hs.307559_at | | | DLPFC | 0.011602 | 0.574393 |
| Hs.390616 | AF070581 | 5063 | chrXq22.3- | 300142 | Hs.390616_at | p21 (CDKN1A)- | PAK3 | DLPFC | 0.025615 | 1.449205 |
| | | | q23 | | | activated | | | | |
| | | | | | | kinase 3 | | | | |
| Hs.446340 | | | | | Hs.446340_at | | | DLPFC | 0.011087 | 1.544746 |
| Hs.46550 | | | | | Hs.46550_at | | | DLPFC | 0.02093 | 1.432785 |
| Hs.512343 | | | | | Hs.512343_at | | | DLPFC | 0.006425 | 1.268103 |
| Hs.519673 | | | | | Hs.519673_at | | | DLPFC | 0.04574 | 0.627049 |
| Hs.521800 | | | | | Hs.521800_at | | | DLPFC | 0.048286 | 1.279609 |
| Hs.7413 | | | | | Hs.7413_at | | | DLPFC | 0.032118 | 1.575143 |
| Hs.334854 | NM_024551 | 79602 | chr12p13.31 | 607946 | Hs.334854_at | adiponectin | ADIPOR2 | Amygdala | 0.003159 | 0.665698 |
| | | | | | | receptor 2 | | | | |
| Hs.442808 | BC002431 | 8704 | chr1p34- | 604013 | Hs.442808_at | UDP-Gal: | B4GALT2 | Amygdala | 0.023737 | 1.270982 |
| | | | p33 | | | betaGlcNAc | | | | |
| | | | | | | beta 1,4- | | | | |
| | | | | | | galactosyl- | | | | |
| | | | | | | transferase, | | | | |
| | | | | | | polypeptide 2 | | | | |
| Hs.380389 | NM_016014 | 51104 | chr9q21.13 | — | Hs.380389_at | chromosome | C9orf77 | Amygdala | 0.008226 | 0.66 |
| | | | | | | 9 open | | | | |
| | | | | | | reading | | | | |
| | | | | | | frame 77 | | | | |
| Hs.301478 | NM_024734 | 79789 | chr14q32.13 | — | Hs.301478_at | calmin | CLMN | Amygdala | 0.015269 | 0.75244 |
| | | | | | | (calponin- | | | | |
| | | | | | | like, trans- | | | | |
| | | | | | | membrane) | | | | |
| Hs.268764 | AW006648 | 342035 | chr15q21.2 | 608603 | Hs.268764_at | coliomin | COLM | Amygdala | 0.011039 | 0.648707 |
| Hs.511884 | AA501453 | 163786 | chr1p21.3 | — | Hs.511884_at | hypothetical | DKFZp761- | Amygdala | 0.049152 | 0.778291 |
| | | | | | | protein | A078 | | | |
| | | | | | | DKFZp761A078 | | | | |
| Hs.441044 | AA297258 | 10085 | chr5q14 | 606018 | Hs.441044_at | EGF-like | EDIL3 | Amygdala | 0.024605 | 0.744337 |
| | | | | | | repeats and | | | | |
| | | | | | | discoidin | | | | |
| | | | | | | I-like | | | | |
| | | | | | | domains 3 | | | | |
| Hs.30318 | NM_018252 | 55248 | chr1q32.3 | — | Hs.30318_at | hypothetical | FLJ 10874 | Amygdala | 0.029049 | 0.750136 |
| | | | | | | protein | | | | |
| | | | | | | FLJ10874 | | | | |
| Hs.310422 | NM_022771 | 64786 | chr12q21.1 | — | Hs.310422_at | TBC1 domain | TBC1D15 | Amygdala | 0.019059 | 0.738469 |
| | | | | | | family, | | | | |
| | | | | | | member 15 | | | | |
| Hs.135146 | BC025250 | 79828 | chr2q31.1 | — | Hs.135146_at | hypothetical | FLJ13984 | Amygdala | 0.028425 | 0.790096 |
| | | | | | | protein | | | | |
| | | | | | | FLJ13984 | | | | |
| Hs.523544 | NM_025032 | 80100 | chr1q21.2 | — | Hs.523544_at | hypothetical | FLJ21272 | Amygdala | 0.02064 | 0.56112 |
| | | | | | | protein | | | | |
| | | | | | | FLJ21272 | | | | |
| Hs.47122 | NM_003838 | 8790 | chr1p31.1 | 603609 | Hs.47122_at | fucose-1- | FPGT | Amygdala | 0.010068 | 0.753436 |
| | | | | | | phosphate | | | | |
| | | | | | | guanylyl- | | | | |
| | | | | | | transferase | | | | |
| Hs.287721 | NM_022873 | 2537 | chr1p35 | 147572 | Hs.287721_at | interferon, | G1P3 | Amygdala | 0.023733 | 0.786625 |
| | | | | | | alpha- | | | | |
| | | | | | | inducible | | | | |
| | | | | | | protein | | | | |
| | | | | | | (clone | | | | |
| | | | | | | IFI-6-16) | | | | |
| Hs.239155 | | | | | Hs.239155_at | | | Amygdala | 0.032254 | 0.787257 |
| Hs.438303 | NM_015340 | 23395 | chr3p21.3 | 604544 | Hs.438303_at | leucyl-tRNA | LARS2 | Amygdala | 0.036951 | 1.21561 |
| | | | | | | synthetase | | | | |
| | | | | | | 2, mito- | | | | |
| | | | | | | chondrial | | | | |
| Hs.129694 | NM_025168 | 55227 | chr6p12.1 | 608195 | Hs.129694_at | leucine | LRRC1 | Amygdala | 0.040074 | 0.721154 |
| | | | | | | rich repeat | | | | |
| | | | | | | containing 1 | | | | |
| Hs.116459 | NM_006122 | 4122 | chr15q26.1 | 600988 | Hs.116459_at | mannosidase, | MAN2A2 | Amygdala | 0.047118 | 0.828848 |
| | | | | | | alpha, class | | | | |
| | | | | | | 2A, member 2 | | | | |
| Hs.93121 | AB052917 | 23155 | chr1p13.3 | — | Hs.93121_at | Mid-1-related | MCLC | Amygdala | 0.03374 | 0.818216 |
| | | | | | | chloride | | | | |
| | | | | | | channel 1 | | | | |
| Hs.63236 | BG497783 | 128308 | chr1q42.13 | — | Hs.63236_at | mitochondrial | MRPL55 | Amygdala | 0.026502 | 1.240988 |
| | | | | | | ribosomal | | | | |
| | | | | | | protein L55 | | | | |
| Hs.436836 | NM_002462 | 4599 | chr21q22.3 | 147150 | Hs.436836_at | myxovirus | MX1 | Amygdala | 0.027157 | 0.797802 |
| | | | | | | (influenza | | | | |
| | | | | | | virus) | | | | |
| | | | | | | resistance 1, | | | | |
| | | | | | | interferon- | | | | |
| | | | | | | inducible | | | | |
| | | | | | | protein p78 | | | | |
| | | | | | | (mouse) | | | | |
| Hs.164682 | NM_000466 | 5189 | chr7q21- | 602136 | Hs.164682_at | peroxisome | PEX1 | Amygdala | 0.027517 | 0.739133 |
| | | | q22 | | | biogenesis | | | | |
| | | | | | | factor 1 | | | | |
| Hs.282702 | NM_006212 | 5208 | chr1q31 | 171835 | Hs.282702_at | 6-phospho- | PFKFB2 | Amygdala | 0.014018 | 0.791098 |
| | | | | | | fructo-2- | | | | |
| | | | | | | kinase/ | | | | |
| | | | | | | fructose- | | | | |
| | | | | | | 2,6-biphos- | | | | |
| | | | | | | phatase 2 | | | | |
| Hs.343329 | NM_002646 | 5287 | chr1q32 | 602838 | Hs.343329_at | phospho- | PIK3C2B | Amygdala | 0.049244 | 0.682849 |
| | | | | | | inositide-3- | | | | |
| | | | | | | kinase, | | | | |
| | | | | | | class 2, | | | | |
| | | | | | | beta | | | | |
| | | | | | | polypeptide | | | | |
| Hs.286073 | NM_003620 | 8493 | chr17q23.2 | 605100 | Hs.286073_at | protein phos- | PPM1D | Amygdala | 0.004151 | 0.827801 |
| | | | | | | phatase 1D | | | | |
| | | | | | | magnesium- | | | | |
| | | | | | | dependent, | | | | |
| | | | | | | delta isoform | | | | |
| Hs.152337 | AL551971 | 10196 | chr11p15.1 | 603190 | Hs.152337_at | HMT1 hnRNP | HRMT1L3 | Amygdala | 0.021309 | 0.775057 |
| | | | | | | methyl- | | | | |
| | | | | | | transferase- | | | | |
| | | | | | | like 3 | | | | |
| | | | | | | (S. cerevisiae) | | | | |
| Hs.442356 | BF439330 | 85358 | chr22q13.3 | 606230 | Hs.442356_at | SH3 and | SHANK3 | Amygdala | 0.026712 | 1.250287 |
| | | | | | | multiple | | | | |
| | | | | | | ankyrin | | | | |
| | | | | | | repeat | | | | |
| | | | | | | domains 3 | | | | |
| Hs.343603 | NM_003673 | 8557 | chr17q12 | 604488 | Hs.343603_at | titin-cap | TCAP | Amygdala | 0.03464 | 1.277915 |
| | | | | | | (telethonin) | | | | |
| Hs.48499 | NM_016516 | 51542 | chr2p13- | — | Hs.48499_at | vacuolar | VPS54 | Amygdala | 0.019562 | 0.795823 |
| | | | p14 | | | protein | | | | |
| | | | | | | sorting | | | | |
| | | | | | | 54 (yeast) | | | | |
| Hs.10359 | | | | | Hs.10359_at | | | Amygdala | 0.036402 | 0.615377 |
| Hs.105769 | | | | | Hs.105769_at | | | Amygdala | 0.025621 | 0.748727 |
| Hs.106148 | AL133577 | — | — | — | Hs.106148_at | MRNA; cDNA | — | Amygdala | 0.030866 | 0.823174 |
| | | | | | | DKFZp434G0972 | | | | |
| | | | | | | (from clone | | | | |
| | | | | | | DKFZp434G0972) | | | | |
| Hs.112592 | | | | | Hs.112592_at | | | Amygdala | 0.033801 | 0.649288 |
| Hs.117864 | | | | | Hs.117864_at | | | Amygdala | 0.006598 | 0.782827 |
| Hs.121806 | AU146685 | — | — | — | Hs.121806_at | CDNA FLJ11971 | — | Amygdala | 0.009175 | 0.663875 |
| | | | | | | fis, clone | | | | |
| | | | | | | HEMBB1001208 | | | | |
| Hs.124944 | | | | | Hs.124944_at | | | Amygdala | 0.001677 | 0.79208 |
| Hs.12867 | | | | | Hs.12867_at | | | Amygdala | 0.008073 | 0.711258 |
| Hs.135229 | AI674243 | 339162 | chr17q25.3 | — | Hs.135229_at | Similar to | — | Amygdala | 0.001917 | 1.371978 |
| | | | | | | ataxin 2 | | | | |
| | | | | | | binding | | | | |
| | | | | | | protein 1 | | | | |
| | | | | | | isoform gamma; | | | | |
| | | | | | | hexaribo- | | | | |
| | | | | | | nucleotide | | | | |
| | | | | | | binding | | | | |
| | | | | | | protein 1 | | | | |
| Hs.143821 | | | | | Hs.143821_at | | | Amygdala | 0.020925 | 0.588934 |
| Hs.145421 | AI939363 | — | — | — | Hs.145421_at | CDNA FLJ43322 | — | Amygdala | 0.003488 | 1.461045 |
| | | | | | | fis, clone | | | | |
| | | | | | | NT2RI2027975 | | | | |
| Hs.146268 | AW001557 | — | — | — | Hs.146268_at | Clone DNA59613 | — | Amygdala | 0.017818 | 1.314676 |
| | | | | | | phospholipase | | | | |
| | | | | | | inhibitor | | | | |
| | | | | | | (UNQ511) mRNA, | | | | |
| | | | | | | complete cds | | | | |
| Hs.155113 | | | | | Hs.155113_at | | | Amygdala | 0.00243 | 0.611274 |
| Hs.156672 | BE858593 | 344148 | chr2q21.2 | 608789 | Hs.156672_at | Nck-associated | NAP5 | Amygdala | 0.002186 | 0.586409 |
| | | | | | | protein 5 | | | | |
| Hs.161359 | | | | | Hs.161359_at | | | Amygdala | 0.023195 | 0.58472 |
| Hs.163893 | | | | | Hs.163893_at | | | Amygdala | 0.007779 | 0.639002 |
| Hs.164502 | BE783668 | 23025 | chr19p13.12 | — | Hs.164502_at | unc-13 | UNC13A | Amygdala | 0.003794 | 1.302452 |
| | | | | | | homolog A | | | | |
| | | | | | | (C. elegans) | | | | |
| Hs.170953 | | | | | Hs.170953_at | | | Amygdala | 0.001139 | 0.560582 |
| Hs.171939 | AI693178 | — | — | — | Hs.171939_at | MRNA; cDNA | — | Amygdala | 0.016426 | 1.337931 |
| | | | | | | DKFZp761L1121 | | | | |
| | | | | | | (from clone | | | | |
| | | | | | | DKFZp761L1121) | | | | |
| Hs.177502 | | | | | Hs.177502_at | | | Amygdala | 0.027013 | 0.71576 |
| Hs.178393 | | | | | Hs.178393_at | | | Amygdala | 0.004451 | 1.543364 |
| Hs.182606 | AI471969 | — | — | — | Hs.182606_at | Clone IMAGE: | — | Amygdala | 0.012741 | 1.483865 |
| | | | | | | 5301129, | | | | |
| | | | | | | mRNA | | | | |
| Hs.184454 | | | | | Hs.184454_at | | | Amygdala | 0.013842 | 1.347984 |
| Hs.187328 | | | | | Hs.187328_at | | | Amygdala | 0.004842 | 0.453853 |
| Hs.190334 | | | | | Hs.190334_at | | | Amygdala | 0.008215 | 0.726692 |
| Hs.191463 | | | | | Hs.191463_at | | | Amygdala | 0.026121 | 0.684305 |
| Hs.202121 | | | | | Hs.202121_at | | | Amygdala | 0.017193 | 1.466848 |
| Hs.205647 | | | | | Hs.205647_at | | | Amygdala | 0.007703 | 0.757727 |
| Hs.21349 | | | | | Hs.21349_at | | | Amygdala | 0.026684 | 1.583208 |
| Hs.221612 | | | | | Hs.221612_at | | | Amygdala | 0.013255 | 0.67585 |
| Hs.224794 | BQ002274 | — | — | — | Hs.224794_at | CDNA FLJ33375 | — | Amygdala | 0.002619 | 0.757862 |
| | | | | | | fis, clone | | | | |
| | | | | | | BRACE2006137 | | | | |
| Hs.22511 | | | | | Hs.22511_at | | | Amygdala | 0.010206 | 0.524585 |
| Hs.225161 | | | | | Hs.225161_at | | | Amygdala | 0.013364 | 0.569931 |
| Hs.23079 | H05023 | 401190 | chr5q12.3 | — | Hs.23079_at | hypothetical | DKFZp686- | Amygdala | 0.002046 | 1.921977 |
| | | | | | | protein | I0554 | | | |
| | | | | | | DKFZp686l0554 | | | | |
| Hs.23100 | NM_152530 | 27031 | chr3q22.1 | 604387 | Hs.23100_at | nephronoph- | NPHP3 | Amygdala | 0.025067 | 0.741499 |
| | | | | | | thisis 3 | | | | |
| | | | | | | (adolescent) | | | | |
| Hs.235795 | AW043859 | — | — | — | Hs.235795_at | Clone IMAGE: | — | Amygdala | 2.72E-05 | 0.35862 |
| | | | | | | 5263020, | | | | |
| | | | | | | mRNA | | | | |
| Hs.240443 | AU134977 | — | — | — | Hs.240443_at | Trophoblast- | — | Amygdala | 0.004325 | 0.636113 |
| | | | | | | derived | | | | |
| | | | | | | noncoding | | | | |
| | | | | | | RNA | | | | |
| Hs.255049 | | | | | Hs.255049_at | | | Amygdala | 0.001256 | 0.794556 |
| Hs.266457 | | | | | Hs.266457_at | | | Amygdala | 0.001958 | 0.723099 |
| Hs.266619 | | | | | Hs.266619_at | | | Amygdala | 0.023573 | 0.659342 |
| Hs.269099 | | | | | Hs.269099_at | | | Amygdala | 0.007654 | 0.588292 |
| Hs.270244 | | | | | Hs.270244_at | | | Amygdala | 0.021368 | 0.677287 |
| Hs.271609 | H21394 | — | — | — | Hs.271609_at | Full length | — | Amygdala | 0.034287 | 0.562163 |
| | | | | | | insert cDNA | | | | |
| | | | | | | YN68A11 | | | | |
| Hs.276976 | AW003140 | 401597 | chrXq13.1- | — | Hs.276976_at | Hypothetical | — | Amygdala | 0.035991 | 0.759303 |
| | | | q13.2 | | | gene supported | | | | |
| | | | | | | by AK125301 | | | | |
| Hs.278648 | AW836210 | — | — | — | Hs.278648_at | CDNA FLJ14085 | — | Amygdala | 0.002245 | 0.658288 |
| | | | | | | fis, clone | | | | |
| | | | | | | HEMBB1002534 | | | | |
| Hs.28170 | | | | | Hs.28170_at | | | Amygdala | 0.000576 | 0.595206 |
| Hs.284707 | BC033052 | — | — | — | Hs.284707_at | CDNA clone | — | Amygdala | 0.017452 | 0.824605 |
| | | | | | | IMAGE: 4770316, | | | | |
| | | | | | | partial cds | | | | |
| Hs.290550 | AI800515 | — | — | — | Hs.290550_at | Clone IMAGE: | — | Amygdala | 0.004158 | 0.646001 |
| | | | | | | 5288238, mRNA | | | | |
| Hs.290830 | AW952920 | — | — | — | Hs.290830_at | Full length | — | Amygdala | 0.002352 | 0.715007 |
| | | | | | | insert cDNA | | | | |
| | | | | | | clone YU27F12 | | | | |
| Hs.291967 | | | | | Hs.291967_at | | | Amygdala | 0.018922 | 0.703836 |
| Hs.292679 | | | | | Hs.292679_at | | | Amygdala | 0.009456 | 0.729281 |
| Hs.293334 | | | | | Hs.293334_at | | | Amygdala | 0.019492 | 0.730359 |
| Hs.293748 | BF447954 | — | — | — | Hs.293748_at | CDNA FLJ26063 | — | Amygdala | 0.006689 | 0.445111 |
| | | | | | | fis, clone | | | | |
| | | | | | | PRS04788 | | | | |
| Hs.293748 | BF447954 | — | — | — | Hs.293748_at | CDNA FLJ26063 | — | Amygdala | 0.005118 | 0.503619 |
| | | | | | | fis, clone | | | | |
| | | | | | | PRS04788 | | | | |
| Hs.293912 | AL137510 | — | — | — | Hs.293912_at | MRNA; cDNA | — | Amygdala | 0.001303 | 0.610613 |
| | | | | | | DKFZp761F052 | | | | |
| | | | | | | (from clone | | | | |
| | | | | | | DKFZp761F052) | | | | |
| Hs.296276 | | | | | Hs.296276_at | | | Amygdala | 0.001696 | 0.33197 |
| Hs.298014 | AU148154 | — | — | — | Hs.298014_at | CDNA FLJ14136 | — | Amygdala | 0.0023 | 0.625751 |
| | | | | | | fis, clone | | | | |
| | | | | | | MAMMA1002744 | | | | |
| Hs.298250 | | | | | Hs.298250_at | | | Amygdala | 0.017026 | 0.799761 |
| Hs.301237 | AU147177 | — | — | — | Hs.301237_at | CDNA FLJ12095 | — | Amygdala | 0.003289 | 0.627277 |
| | | | | | | fis, clone | | | | |
| | | | | | | HEMBB1002610 | | | | |
| Hs.304253 | | | | | Hs.304253_at | | | Amygdala | 0.004511 | 0.456269 |
| Hs.306329 | AL109684 | — | — | — | Hs.306329_at | MRNA full | — | Amygdala | 0.01041 | 0.741482 |
| | | | | | | length insert | | | | |
| | | | | | | cDNA clone | | | | |
| | | | | | | EUROIMAGE | | | | |
| | | | | | | 27080 | | | | |
| Hs.306458 | AL137424 | — | — | — | Hs.306458_at | MRNA; cDNA | — | Amygdala | 0.015341 | 0.744289 |
| | | | | | | DKFZp761G2123 | | | | |
| | | | | | | (from clone | | | | |
| | | | | | | DKFZp761G2123) | | | | |
| Hs.312469 | BF529886 | — | — | — | Hs.312469_at | CDNA FLJ45559 | — | Amygdala | 0.001127 | 1.675411 |
| | | | | | | fis, clone | | | | |
| | | | | | | BRTHA3003225 | | | | |
| Hs.317614 | BQ022804 | 143903 | chr11q23.2 | — | Hs.317614_at | layilin | LOC143903 | Amygdala | 0.001917 | 0.390905 |
| Hs.31841 | AI521765 | — | — | — | Hs.31841_at | CDNA FLJ33489 | — | Amygdala | 0.004573 | 0.691038 |
| | | | | | | fis, clone | | | | |
| | | | | | | BRAMY2003585 | | | | |
| Hs.332620 | BG545582 | — | — | — | Hs.332620_at | Clone IMAGE: | — | Amygdala | 0.011093 | 0.714316 |
| | | | | | | 4418644, | | | | |
| | | | | | | mRNA | | | | |
| Hs.337506 | AW611486 | — | — | — | Hs.337506_at | MRNA; cDNA | — | Amygdala | 0.002515 | 0.700655 |
| | | | | | | DKFZp566D053 | | | | |
| | | | | | | (from clone | | | | |
| | | | | | | DKFZp566D053) | | | | |
| Hs.348325 | BF692592 | — | — | — | Hs.348325_at | Clone IMAGE: | — | Amygdala | 0.022077 | 0.611878 |
| | | | | | | 4043849, | | | | |
| | | | | | | mRNA | | | | |
| Hs.349656 | AA885297 | 950 | chr4q21.1 | 602257 | Hs.349656_at | scavenger | SCARB2 | Amygdala | 0.049096 | 0.708297 |
| | | | | | | receptor | | | | |
| | | | | | | class B, | | | | |
| | | | | | | member 2 | | | | |
| Hs.352604 | AK054833 | — | — | — | Hs.352604_at | CDNA FLJ30271 | — | Amygdala | 0.006646 | 0.759894 |
| | | | | | | fis, clone | | | | |
| | | | | | | BRACE2002676 | | | | |
| Hs.356721 | NM_002136 | 3178 | chr12q13.1 | 164017 | Hs.356721_at | heterogeneous | HNRPA1 | Amygdala | 0.013735 | 0.77917 |
| | | | | | | nuclear ribo- | | | | |
| | | | | | | nucleoprotein | | | | |
| | | | | | | A1/// | | | | |
| | | | | | | heterogeneous | | | | |
| | | | | | | nuclear | | | | |
| | | | | | | ribonucleo- | | | | |
| | | | | | | protein A1 | | | | |
| Hs.36190 | | | | | Hs.36190_at | | | Amygdala | 0.002178 | 0.73084 |
| Hs.368747 | | | | | Hs.368747_at | | | Amygdala | 0.0235 | 0.690354 |
| Hs.370868 | | | | | Hs.370868_at | | | Amygdala | 0.003122 | 0.592345 |
| Hs.372914 | | | | | Hs.372914_at | | | Amygdala | 0.002545 | 0.722778 |
| Hs.375064 | BC030757 | — | — | — | Hs.375064_at | Clone IMAGE: | — | Amygdala | 0.020267 | 0.513317 |
| | | | | | | 4797534, | | | | |
| | | | | | | mRNA, | | | | |
| | | | | | | partial cds | | | | |
| Hs.378706 | AU147038 | — | — | — | Hs.378706_at | CDNA FLJ12064 | — | Amygdala | 0.00199 | 0.740046 |
| | | | | | | fis, clone | | | | |
| | | | | | | HEMBB1002232 | | | | |
| Hs.380331 | BC015390 | — | — | — | Hs.380331_at | CDNA FLJ41173 | — | Amygdala | 0.030727 | 2.243787 |
| | | | | | | fis, clone | | | | |
| | | | | | | BRACE2042394 | | | | |
| Hs.381882 | AL512701 | — | — | — | Hs.381882_at | CDNA FLJ39866 | — | Amygdala | 0.010168 | 0.73564 |
| | | | | | | fis, clone | | | | |
| | | | | | | SPLEN2015276 | | | | |
| Hs.383007 | | | | | Hs.383007_at | | | Amygdala | 0.014769 | 0.709824 |
| Hs.384620 | AF086073 | — | — | — | Hs.384620_at | Full length | — | Amygdala | 0.013717 | 0.562816 |
| | | | | | | insert cDNA | | | | |
| | | | | | | clone YZ55H04 | | | | |
| Hs.384626 | AF086037 | — | — | — | Hs.384626_at | Full length | — | Amygdala | 0.004341 | 0.655612 |
| | | | | | | insert cDNA | | | | |
| | | | | | | clone YW28D08 | | | | |
| Hs.386147 | | | | | Hs.386147_at | | | Amygdala | 0.02646 | 0.681543 |
| Hs.390616 | AF070581 | 5063 | chrXq22.3- | 300142 | Hs.390616_at | p21 (CDKN1A)- | PAK3 | Amygdala | 0.023157 | 1.472364 |
| | | | q23 | | | activated | | | | |
| | | | | | | kinase 3 | | | | |
| Hs.397085 | BC033548 | — | — | — | Hs.397085_at | Clone IMAGE: | — | Amygdala | 0.04571 | 0.743093 |
| | | | | | | 4825215, | | | | |
| | | | | | | mRNA | | | | |
| Hs.397369 | AU147851 | — | — | — | Hs.397369_at | CDNA FLJ11958 | — | Amygdala | 0.013779 | 0.697461 |
| | | | | | | fis, clone | | | | |
| | | | | | | HEMBB1000996 | | | | |
| Hs.400590 | AI819043 | — | — | — | Hs.400590_at | CDNA FLJ32589 | — | Amygdala | 0.005598 | 0.43 |
| | | | | | | fis, clone | | | | |
| | | | | | | SPLEN2000443 | | | | |
| Hs.40794 | AI368415 | 388135 | chr15q22.33 | — | Hs.40794_at | Similar to | — | Amygdala | 0.011758 | 1.26403 |
| | | | | | | RIKEN cDNA | | | | |
| | | | | | | 6030419C18 | | | | |
| | | | | | | gene | | | | |
| Hs.408264 | BU620691 | 283417/// | chr12q14.2/// | — | Hs.408264_at | hypothetical | FLJ32949/// | Amygdala | 0.017538 | 1.537381 |
| | | 349152 | chr7q22.1 | | | protein | FLJ36166 | | | |
| | | | | | | FLJ32949/// | | | | |
| | | | | | | hypothetical | | | | |
| | | | | | | protein | | | | |
| | | | | | | FLJ36166 | | | | |
| Hs.41688 | AI864441 | — | — | — | Hs.41688_at | CDNA FLJ42958 | — | Amygdala | 0.027645 | 1.30913 |
| | | | | | | fis, clone | | | | |
| | | | | | | BRSTN2010750 | | | | |
| Hs.4241 | | | | | Hs.4241_at | | | Amygdala | 0.005819 | 1.345413 |
| Hs.429581 | | | | | Hs.429581_at | | | Amygdala | 0.017093 | 0.606702 |
| Hs.429591 | | | | | Hs.429591_at | | | Amygdala | 0.008943 | 0.50843 |
| Hs.432924 | AW014647 | — | — | — | Hs.432924_at | Full length | — | Amygdala | 0.003946 | 1.420431 |
| | | | | | | insert cDNA | | | | |
| | | | | | | YI37C01 | | | | |
| Hs.433053 | | | | | Hs.433053_at | | | Amygdala | 0.012291 | 0.65199 |
| Hs.433923 | NM_001063 | 7018 | chr3q22.1 | 190000 | Hs.433923_at | transferrin | TF | Amygdala | 0.009177 | 0.416645 |
| Hs.436623 | | | | | Hs.436623_at | | | Amygdala | 0.01242 | 0.523161 |
| Hs.443287 | | | | | Hs.443287_at | | | Amygdala | 0.02899 | 0.755498 |
| Hs.443487 | | | | | Hs.443487_at | | | Amygdala | 0.004501 | 0.575491 |
| Hs.444181 | | | | | Hs.444181_at | | | Amygdala | 0.033273 | 0.705257 |
| Hs.444335 | | | | | Hs.444335_at | | | Amygdala | 0.000938 | 0.650602 |
| Hs.444555 | | | | | Hs.444555_at | | | Amygdala | 0.004951 | 1.288085 |
| Hs.444665 | AA430151 | — | — | — | Hs.444665_at | MRNA; cDNA | — | Amygdala | 0.021285 | 1.655944 |
| | | | | | | DKFZp686E1944 | | | | |
| | | | | | | (from clone | | | | |
| | | | | | | DKFZp686E1944) | | | | |
| Hs.461300 | AK054714 | 126661 | chr1p34.1 | — | Hs.461300_at | hypothetical | LOC126661 | Amygdala | 0.009085 | 0.822552 |
| | | | | | | protein | | | | |
| | | | | | | LOC126661 | | | | |
| Hs.466301 | | | | | Hs.466301_at | | | Amygdala | 0.003635 | 0.642776 |
| Hs.472323 | | | | | Hs.472323_at | | | Amygdala | 0.008676 | 0.644228 |
| Hs.501272 | | | | | Hs.501272_at | | | Amygdala | 0.008857 | 1.268105 |
| Hs.502810 | | | | | Hs.502810_at | | | Amygdala | 0.007839 | 0.546665 |
| Hs.504709 | | | | | Hs.504709_at | | | Amygdala | 0.008043 | 1.291178 |
| Hs.50495 | | | | | Hs.50495_at | | | Amygdala | 0.010546 | 0.720397 |
| Hs.508763 | BM353142 | — | — | — | Hs.508763_at | CDNA FLJ39845 | — | Amygdala | 0.002216 | 0.672116 |
| | | | | | | fis, clone | | | | |
| | | | | | | SPLEN2014452 | | | | |
| Hs.512151 | | | | | Hs.512151_at | | | Amygdala | 0.017229 | 0.367527 |
| Hs.513796 | | | | | Hs.513796_at | | | Amygdala | 0.004 | 1.441776 |
| Hs.514559 | | | | | Hs.514559_at | | | Amygdala | 0.040767 | 1.262803 |
| Hs.514909 | | | | | Hs.514909_at | | | Amygdala | 0.025037 | 0.714495 |
| Hs.514934 | | | | | Hs.514934_at | | | Amygdala | 0.02093 | 0.657218 |
| Hs.515369 | | | | | Hs.515369_at | | | Amygdala | 0.011008 | 0.360543 |
| Hs.515610 | | | | | Hs.515610_at | | | Amygdala | 6.24E−05 | 1.481497 |
| Hs.517410 | | | | | Hs.517410_at | | | Amygdala | 0.027833 | 1.464479 |
| Hs.517622 | | | | | Hs.517622_at | | | Amygdala | 0.003107 | 0.558423 |
| Hs.519673 | | | | | Hs.519673_at | | | Amygdala | 0.004901 | 0.430537 |
| Hs.519758 | | | | | Hs.519758_at | | | Amygdala | 0.004805 | 1.235818 |
| Hs.520047 | | | | | Hs.520047_at | | | Amygdala | 0.006288 | 0.256084 |
| Hs.522373 | | | | | Hs.522373_at | | | Amygdala | 0.002654 | 0.631341 |
| Hs.522551 | | | | | Hs.522551_at | | | Amygdala | 0.004278 | 1.593644 |
| Hs.524138 | | | | | Hs.524138_at | | | Amygdala | 0.026311 | 1.284691 |
| Hs.524947 | | | | | Hs.524947_at | | | Amygdala | 0.004034 | 0.658967 |
| Hs.525410 | | | | | Hs.525410_at | | | Amygdala | 0.002438 | 0.390568 |
| Hs.525566 | | | | | Hs.525566_at | | | Amygdala | 0.01019 | 0.573626 |
| Hs.526756 | CA776505 | — | — | — | Hs.526756_at | Full length | — | Amygdala | 0.003311 | 0.591593 |
| | | | | | | insert cDNA | | | | |
| | | | | | | clone YF43G08 | | | | |
| Hs.528308 | BC001387 | 11145 | chr11q12.3- | — | Hs.528308_at | HRAS-like | HRASLS3 | Amygdala | 0.00474 | 0.684806 |
| | | | q13.1 | | | suppressor 3 | | | | |
| Hs.528702 | AB000888 | 8611 | chr5q11 | 607124 | Hs.528702_at | phosphatidic | PPAP2A | Amygdala | 0.022873 | 0.783506 |
| | | | | | | acid phospha- | | | | |
| | | | | | | tase type 2A | | | | |
| Hs.529221 | | | | | Hs.529221_at | | | Amygdala | 0.00261 | 0.725799 |
| Hs.530015 | | | | | Hs.530015_at | | | Amygdala | 0.017647 | 0.649954 |
| Hs.530304 | | | | | Hs.530304_at | | | Amygdala | 0.035698 | 0.613183 |
| Hs.530540 | | | | | Hs.530540_at | | | Amygdala | 0.006211 | 0.675555 |
| Hs.530633 | BG112359 | — | — | — | Hs.530633_at | CDNA clone | — | Amygdala | 0.005983 | 0.59179 |
| | | | | | | MGC: 24463 | | | | |
| | | | | | | IMAGE: | | | | |
| | | | | | | 4082362, | | | | |
| | | | | | | complete cds | | | | |
| Hs.530863 | NM_020764 | 57524 | chr16p13.3 | — | Hs.530863_at | CASK | CASKIN1 | Amygdala | 0.009288 | 1.271367 |
| | | | | | | interacting | | | | |
| | | | | | | protein 1 | | | | |
| Hs.530988 | NM_016384 | — | — | — | Hs.530988_at | MRNA; cDNA | — | Amygdala | 0.014742 | 0.786987 |
| | | | | | | DKFZp779H233 | | | | |
| | | | | | | (from clone | | | | |
| | | | | | | DKFZp779H233) | | | | |
| Hs.6655 | AL355688 | — | — | — | Hs.6655_at | EST from clone | — | Amygdala | 0.044777 | 0.775312 |
| | | | | | | 208499, full | | | | |
| | | | | | | insert | | | | |
| Hs.71913 | | | | | Hs.71913_at | | | Amygdala | 0.036543 | 0.501381 |
| Hs.80720 | AK074381 | 2549 | chr4q31.21 | 604439 | Hs.80720_at | GRB2- | GAB1 | Amygdala | 0.001894 | 0.579585 |
| | | | | | | associated | | | | |
| | | | | | | binding | | | | |
| | | | | | | protein 1 | | | | |

TABLE 24

| | | | | | | | | code | |
|---|---|---|---|---|---|---|---|---|---|
| | | | | | | | | link | FC |
| | | | | | | | | normalized, | Code- |
| | Repre- | | Chromo- | | | | | p-value | link |
| UniGene | sentative | Locus | somal | | Probe | | Gene | (Diag- | Raw |
| ID | Public ID | Link | Location | OMIM | Name | Gene Title | Symbol | nostics) | log2 |
| Hs.194720 | AF098951 | 9429 | chr4q22 | 603756 | GE81445 | ATP-binding cassette, | ABCG2 | 0.001313 | 0.562347 |
| | | | | | | subfamily G (WHITE), | | | |
| | | | | | | member 2 | | | |
| Hs.234898 | NM_001093 | 32 | chr12q24.1 | 601557 | GE554953 | acetyl-Coenzyme A | ACACB | 0.000557 | 0.560812 |
| | | | | | | carboxylase beta | | | |
| Hs.287558 | NM_000700 | 301 | chr9q12-q21.2 | 151690 | GE59671 | annexin A1 | ANXA1 | 0.009335 | 2.078546 |
| Hs.268571 | NM_001645 | 341 | chr19q13.2 | 107710 | GE505140 | apolipoprotein C-I | APOC1 | 0.001325 | 2.509905 |
| Hs.40888 | AF193421 | 23237 | chr8q24.3 | — | GE57416 | activity-regulated | ARC | 0.012693 | 1.949471 |
| | | | | | | cytoskeleton- | | | |
| | | | | | | associated protein | | | |
| Hs.512643 | D90427 | 563 | chr7q22.1 | 194460 | GE59850 | alpha-2-glycoprotein | AZGP1 | 0.012211 | 0.437674 |
| | | | | | | 1, zinc | | | |
| Hs.171825 | BG326045 | 8553 | chr3p26 | 604256 | GE85322 | basic helix-loop- | BHLHB2 | 0.006207 | 1.836491 |
| | | | | | | helix domain con- | | | |
| | | | | | | taining, class B, 2 | | | |
| Hs.77311 | BC028229 | 10950 | chr21q21.1- | 605674 | GE81683 | BTG family, member 3 | BTG3 | 0.00466 | 2.027951 |
| | | | q21.2 | | | | | | |
| Hs.283683 | NM_020130 | 56892 | chr8p11.2 | 607702 | GE87019 | chromosome 8 open | C8orf4 | 0.001216 | 2.588593 |
| | | | | | | reading frame 4 | | | |
| Hs.192491 | NM_012113 | 23632 | chr1q21 | 604832 | GE86437 | carbonic anhydrase | CA14 | 0.000934 | 0.710337 |
| | | | | | | XIV | | | |
| Hs.446471 | M28590 | 972 | chr5q32 | 142790 | GE79594 | CD74 antigen (invariant | CD74 | 0.033704 | 2.330845 |
| | | | | | | polypeptide of major | | | |
| | | | | | | histocompatibility | | | |
| | | | | | | complex, class II | | | |
| | | | | | | antigen-associated) | | | |
| Hs.380627 | BC006171 | 54918 | chr3p22.3 | 607889 | GE85308 | chemokine-like factor | CKLFSF6 | 0.040862 | 1.424176 |
| | | | | | | super family 6 | | | |
| Hs.274127 | NM_016438 | 51751 | chr17q21.31 | — | GE80242 | CLST 11240 protein | CLST11240 | 0.000593 | 0.568223 |
| Hs.79187 | AY072911 | 1525 | chr21q21.1 | 602621 | GE81683 | coxsackie virus and | CXADR | 0.00466 | 2.027951 |
| | | | | | | adenovirus receptor | | | |
| Hs.26704 | BC005097 | 23191 | chr15q11 | 606322 | GE59548 | cytoplasmic FMR1 | CYFIP1 | 0.027249 | 2.226976 |
| | | | | | | interacting protein 1 | | | |
| Hs.165636 | NM_017594 | 54769 | chr9q22.2 | 607863 | GE846112 | DIRAS family, GTP- | DIRAS2 | 0.002726 | 3.180369 |
| | | | | | | binding RAS-like 2 | | | |
| Hs.356742 | NM_006442 | 10589 | chr11q13.3 | 602289 | GE81638 | DR1-associated pro- | DRAP1 | 0.005496 | 1.728007 |
| | | | | | | tein 1 (negative | | | |
| | | | | | | cofactor 2 alpha) | | | |
| Hs.420569 | NM_004089 | 1831 | chrxq22.3 | 602960 | GE56426 | delta sleep inducing | DSIPI | 0.000619 | 0.525127 |
| | | | | | | peptide, immunoreactor | | | |
| Hs.2128 | U16996 | 1847 | chr10q25 | 603069 | GE58962 | dual specificity | DUSP5 | 0.008311 | 2.29312 |
| | | | | | | phosphatase 5 | | | |
| Hs.23853 | BE386445 | 253461 | chr3q23 | — | GE79184 | hypothetical protein | FLJ35036 | 0.004127 | 1.472645 |
| | | | | | | FLJ35036 | | | |
| Hs.110571 | NM_015675 | 4616 | chr19p13.3 | 604948 | GE82030 | growth arrest and | GADD45B | 0.000325 | 3.730464 |
| | | | | | | DNA-damage-inducible, | | | |
| | | | | | | beta | | | |
| Hs.62661 | BC002666 | 2633 | chr1p22.2 | 600411 | GE81108 | guanylate binding | GBP1 | 0.007744 | 1.951445 |
| | | | | | | protein 1, interferon- | | | |
| | | | | | | inducible, 67 kDa | | | |
| Hs.46453 | NM_005291 | 2840 | chr2q21 | 603071 | GE60447 | G protein-coupled | GPR17 | 0.019726 | 0.314979 |
| | | | | | | receptor 17 | | | |
| Hs.75652 | NM_000851 | 2949 | chr1p13.3 | 138385 | GE57545 | glutathione S-trans- | GSTM5 | 0.008865 | 0.545885 |
| | | | | | | ferase M5 | | | |
| Hs.8821 | NM_021175 | 57817 | chr19q13.1 | 606464 | GE82598 | hepcidin antimicro- | HAMP | 0.003586 | 3.9895 |
| | | | | | | bial peptide | | | |
| Hs.352109 | NM_003518.3 | 8339 | 6p21.3 | 602798 | GE85033 | histone 1, H2bg | HIST1H2BG | 0.030363 | 1.766648 |
| Hs.70937 | NM_003536 | 8357 | chr6p22-p21.3 | 602818 | GE572087 | histone 1, H3h | HIST1H3H | 0.004033 | 2.255584 |
| Hs.3268 | X51757 | 3310 | chr1q23 | 140555 | GE59761 | heat shock 70 kDa | HSPA6 | 0.012769 | 2.047918 |
| | | | | | | protein 6 (HSP70B′) | | | |
| Hs.370873 | NM_005531 | 3428 | chr1q22 | 147586 | GE58028 | interferon, gamma- | IFI16 | 0.003499 | 2.056085 |
| | | | | | | inducible protein 16 | | | |
| Hs.14623 | NM_006332 | 10437 | chr19p13.1 | 604664 | GE81622 | interferon, gamma- | IFI30 | 0.004441 | 2.296817 |
| | | | | | | inducible protein 30 | | | |
| Hs.512234 | NM_000600.1 | 3569 | 7p21 | 147620 | GE59660 | interleukin 6 (inter- | IL6 | 0.00397 | 2.048871 |
| | | | | | | feron, beta 2) | | | |
| Hs.416385 | BE300521 | 3638 | chr7q36 | 602055 | GE85346 | insulin induced | INSIG1 | 0.034064 | 2.350952 |
| | | | | | | gene 1 | | | |
| Hs.80645 | NM_002198 | 3659 | chr5q31.1 | 147575 | GE59715 | interferon regu- | IRF1 | 0.002739 | 2.737088 |
| | | | | | | latory factor 1 | | | |
| Hs.78465 | BG491844 | 3725 | chr1p32-p31 | 165160 | GE57500 | v-jun sarcoma virus | JUN | 0.031737 | 2.110769 |
| | | | | | | 17 oncogene homolog | | | |
| | | | | | | (avian) | | | |
| Hs.283063 | NM_005574 | 4005 | chr11p13 | 180385 | GE79375 | LIM domain only 2 | LMO2 | 0.029926 | 1.606316 |
| | | | | | | (rhombotin-like 1) | | | |
| Hs.365706 | NM_000900 | 4256 | chr12p13.1- | 154870 | GE80964 | matrix Gla protein | MGP | 0.007283 | 2.151154 |
| | | | p12.3 | | | | | | |
| Hs.105547 | NM_015392 | 56654 | chr9q34.3 | 605798 | GE56276 | neural proliferation, | NPDC1 | 0.00336 | 1.65195 |
| | | | | | | differentiation and | | | |
| | | | | | | control, 1 | | | |
| Hs.94070 | AI765819 | 4958 | chr9q22.31 | — | GE53008 | osteomodulin | OMD | 0.020759 | 0.643537 |
| Hs.293464 | BC020691 | 10135 | chr7q22.3 | 608764 | GE86807 | pre-B-cell colony | PBEF1 | 0.00851 | 2.697207 |
| | | | | | | enhancing | | | |
| | | | | | | factor 1 | | | |
| Hs.51 | NM_002641 | 5277 | chrxp22.1 | 311770 | GE57051 | phosphatidylinositol | PIGA | 0.001235 | 2.017032 |
| | | | | | | glycan, class A | | | |
| | | | | | | (paroxysmal nocturnal | | | |
| | | | | | | hemoglobinuria) | | | |
| Hs.371003 | NM_016619 | 51316 | chr4q21.3 | 607515 | GE55372 | placenta-specific 8 | PLAC8 | 0.041597 | 2.088607 |
| Hs.307033 | AI983043 | 55041 | chr2q21.2 | — | GE544254 | pleckstrin homology | PLEKHB2 | 0.017474 | 1.277814 |
| | | | | | | domain containing, | | | |
| | | | | | | family B (evectins) | | | |
| | | | | | | member 2 | | | |
| Hs.348478 | NM_021105 | 5359 | chr3q23 | 604170 | GE62320 | phospholipid | PLSCR1 | 0.015981 | 2.015066 |
| | | | | | | scramblase 1 | | | |
| Hs.76556 | NM_014330 | 23645 | chr19q13.2 | — | GE81913 | protein phosphatase 1, | PPP1R15A | 0.010526 | 3.266182 |
| | | | | | | regulatory (inhibitor) | | | |
| | | | | | | subunit 15A | | | |
| Hs.381081 | NM_002800 | 5698 | chr6p21.3 | 177045 | GE60353 | proteasome (prosome, | PSMB9 | 0.00082 | 1.846565 |
| | | | | | | macropain) subunit, | | | |
| | | | | | | beta type, 9 (large | | | |
| | | | | | | multifunctional | | | |
| | | | | | | protease 2) | | | |
| Hs.436577 | NM_003469 | 7857 | chr2q35-q36 | 118930 | GE57887 | secretogranin II | SCG2 | 0.007024 | 3.218546 |
| | | | | | | (chromogranin C) | | | |
| Hs.89546 | NM_000450 | 6401 | chr1q22-q25 | 131210 | GE56057 | selectin E (endothelial | SELE | 0.006493 | 3.21028 |
| | | | | | | adhesion molecule 1) | | | |
| Hs.109051 | NM_031286 | 83442 | chr1p35-p34.3 | — | GE79881 | SH3 domain binding | SH3BGRL3 | 0.00726 | 1.712547 |
| | | | | | | glutamic acid-rich | | | |
| | | | | | | protein like 3///SH3 | | | |
| | | | | | | domain binding gluta- | | | |
| | | | | | | mic acid-rich protein | | | |
| | | | | | | like 3 | | | |
| Hs.435735 | NM_021095 | 8884 | chr2p23 | 604024 | GE56379 | solute carrier family | SLC5A6 | 0.001515 | 0.678555 |
| | | | | | | 5 (sodium-dependent | | | |
| | | | | | | vitamin transporter), | | | |
| | | | | | | member 6 | | | |
| Hs.380991 | NM_030751 | 150094 | chr21q22.3 | 605705 | GE62158 | SNF1-like kinase/// | SNF1LK | 0.019364 | 2.386375 |
| | | | | | | SNF1-like kinase | | | |
| Hs.398157 | NM_006622 | 10769 | chr5q12.1-q13.2 | 607023 | GE54551 | polo-like kinase 2 | PLK2 | 0.014722 | 1.853032 |
| | | | | | | (Drosophila) | | | |
| Hs.352018 | NM_000593 | 6890 | chr6p21.3 | 170260 | GE79181 | transporter 1, ATP- | TAP1 | 0.000286 | 2.297921 |
| | | | | | | binding cassette, sub- | | | |
| | | | | | | family B (MDR/TAP) | | | |
| Hs.78824 | AL833389 | 7075 | chr1p34-p33 | 600222 | GE59865 | tyrosine kinase with | TIE | 0.000246 | 0.467508 |
| | | | | | | immunoglobulin and | | | |
| | | | | | | epidermal growth factor | | | |
| | | | | | | homology domains | | | |
| Hs.211600 | AI738896 | 7128 | chr6q23 | 191163 | GE61999 | tumor necrosis factor, | TNFAIP3 | 0.019227 | 2.354308 |
| | | | | | | alpha-induced protein 3 | | | |
| Hs.114412 | NM_004786 | 9352 | chr18q21.2 | 603049 | GE539832 | thioredoxin-like 1 | TXNL1 | 0.000351 | 1.632106 |
| Hs.17917 | AL574194 | 10894 | chr11p15 | 605702 | GE58413 | extracellular link | XLKD1 | 0.049184 | 2.328181 |
| | | | | | | domain containing 1 | | | |
| Hs.268571 | NM_001645 | 341 | chr19q13.2 | 107710 | GE505140 | apolipoprotein C-1 | APOC1 | p < 0.05, | 2.509905 |
| | | | | | | | | 3 batches | |
| Hs.47546 | AW968493 | 55780 | chr6q27 | — | GE87817 | chromosome 6 open | C6orf70 | p < 0.05, | 0.682264 |
| | | | | | | reading frame 70 | | 3 batches | |
| Hs.140944 | NM_012135 | 26240 | chr6p25-pter | — | GE60346 | DNA segment on chromo- | D6S2654E | p < 0.05, | 0.848706 |
| | | | | | | some 6(unique) 2654 | | 3 batches | |
| | | | | | | expressed sequence | | | |
| Hs.18788 | NM_016246 | 51171 | chr19q13.33 | — | GE58712 | dehydrogenase/reductase | DHRS10 | p < 0.05, | 1.344068 |
| | | | | | | (SDR family) member 10 | | 3 batches | |
| Hs.494204 | AW299245 | 91298 | chr12q21.33 | — | GE56729 | hypothetical protein | DKFZp434 | p < 0.05, | 0.544692 |
| | | | | | | DKFZp434N2030 | N2030 | 3 batches | |
| Hs.76591 | BF116183 | 23197 | chr5q35.2 | — | GE53562 | expressed in T-cells | ETEA | p < 0.05, | 0.851599 |
| | | | | | | and eosinophils in | | 3 batches | |
| | | | | | | atopic dermatitis | | | |
| Hs.287629 | NM_025027 | 80095 | chr19q13.4 | — | GE479292 | zinc finger protein | ZNF606 | p < 0.05, | 0.765441 |
| | | | | | | 606 | | 3 batches | |
| Hs.135569 | AL831857 | 84913 | chr2p11.2 | — | GE83540 | atonal homolog 8 | ATOH8 | p < 0.05, | 0.684805 |
| | | | | | | (Drosophila) | | 3 batches | |
| Hs.89519 | T85841.1 | 54726 | 4q31.21 | | GE53713 | HIV-1 induced protein | HSHIN1 | p < 0.05, | 0.763166 |
| | | | | | | HIN-1 | | 3 batches | |
| Hs.26745 | AF151078 | 51259 | chr11q13.1 | — | GE82136 | HSPC244 | MGC: 13379 | p < 0.05, | 0.716451 |
| | | | | | | | | 3 batches | |
| Hs.315167 | L11372 | 79075 | chr8q24.12 | — | GE63328 | defective in sister | DCC1 | p < 0.05, | 0.606126 |
| | | | | | | chromatid cohesion | | 3 batches | |
| | | | | | | homolog 1 | | | |
| | | | | | | (S. cerevisiae) | | | |
| Hs.404 | BC030550 | 4300 | chr9p22 | 159558 | GE81406 | myeloid/lymphoid or | MLLT3 | p < 0.05, | 0.727899 |
| | | | | | | mixed-lineage leukemia | | 3 batches | |
| | | | | | | (trithorax homolog, | | | |
| | | | | | | Drosophila); trans- | | | |
| | | | | | | located to, 3 | | | |
| Hs.196585 | NM_015485 | 25950 | chr1p21.3 | — | GE88260 | RWD domain containing 3 | RWDD3 | p < 0.05, | 0.784195 |
| | | | | | | | | 3 batches | |
| Hs.78824 | AL833389 | 7075 | chr1p34-p33 | 600222 | GE59865 | tyrosine kinase with | TIE | p < 0.05, | 0.467508 |
| | | | | | | immunoglobulin and | | 3 batches | |
| | | | | | | epidermal growth | | | |
| | | | | | | factor homology | | | |
| | | | | | | domains | | | |
| Hs.179526 | NM_006472.1 | 10628 | 1q21.2 | 606599 | GE58805 | thioredoxin interacting | TXNIP | p < 0.05, | 0.433622 |
| | | | | | | protein | | 3 batches | |
| Hs.76561 | AA084273 | 342908 | chr19q13.32 | — | GE85622 | zinc finger protein 404 | ZNF404 | p < 0.05, | 0.557599 |
| | | | | | | | | 3 batches | |
| Hs.355957 | NM_001029 | 6231 | chr12q13 | 603701 | GE80976 | ribosomal protein S26 | RPS26 | p < 0.05, | 1.749582 |
| | | | | | | | | 3 batches | |
| NULL | AP003480.1 | | | | GE86003 | | | p < 0.05. | 1.648659 |
| | | | | | | | | 3 batches | |
| Hs.299123 | BF593518.1 | | | | GE607429 | | | p < 0.05, | 1.529494 |
| | | | | | | | | 3 batches | |
| Hs.334931 | BC008122.1 | | | | GE748859 | | | p < 0.05, | 1.441456 |
| | | | | | | | | 3 batches | |
| Hs.475880 | BE072907.1 | | | | GE573233 | | | p < 0.05, | 1.323119 |
| | | | | | | | | 3 batches | |
| Hs.48797 | N94759.1 | | | | GE557728 | | | p < 0.05, | 0.901628 |
| | | | | | | | | 3 batches | |
| Hs.49658 | BC013872 | 57212 | chr1p36.32 | — | GE53221 | KIAA0495 | KIAA0495 | p < 0.05, | 0.750704 |
| | | | | | | | | 3 batches | |
| Hs.518925 | BM699227.1 | | | | GE88534 | | | p < 0.05, | 0.737717 |
| | | | | | | | | 3 batches | |
| Hs.273099 | AK023774.1 | | | | GE572395 | | | p < 0.05, | 0.672578 |
| | | | | | | | | 3 batches | |
| Hs.433156 | NM_005162 | 91445 | chr22 | — | GE61190 | hypothetical protein | FLJ38628 | p < 0.05, | 0.662088 |
| | | | | | | FLJ38628 | | 3 batches | |
| Hs.433156 | NM_005162 | 91445 | chr22 | — | GE61190 | hypothetical protein | FLJ38628 | p < 0.05, | 0.662088 |
| | | | | | | FLJ38628 | | 3 batches | |
| Hs.314413 | AI289609.1 | | | | GE754401 | | | p < 0.05, | 0.644233 |
| | | | | | | | | 3 batches | |
| NULL | NM_199050.1 | | | | GE56267 | | | p < 0.05, | 0.60657 |
| | | | | | | | | 3 batches | |
| Hs.133536 | AI379149.1 | | | | GE633524 | | | p < 0.05, | 0.592899 |
| | | | | | | | | 3 batches | |
| Hs.377159 | BM713079.1 | | | | GE826874 | | | p < 0.05, | 0.576892 |
| | | | | | | | | 3 batches | |
| NULL | INCYTE | | | | GE87335 | | | p < 0.05, | 0.571783 |
| | UNIQUE | | | | | | | 3 batches | |
| Hs.278081 | AV718725.1 | | | | GE500797 | | | p < 0.05, | 0.382559 |
| | | | | | | | | 3 batches | |

TABLE 25
Schizophrenia Cohort 2: AnCg, DLPFC, Amygdala.

| | number of |
|---|---|
| Pathway | probe sets |
| Apoptosis | 55 |
| Blood group glycolipid biosynthesis-neolactoseries | 3 |
| Cell_cycle1-5 TGF_Beta_Signaling_Pathway | 27 |
| Electron_Transport_Chain | 1 |
| G_Protein_Signaling MAPK_Cascade | 5 |
| G13_Signaling_Pathway Integrin- | 4 |
| mediated_cell_adhesion Wnt_signaling | |
| Glycerolipid metabolism | 3 |
| GPCRs_Class_A_Rhodopsin-like Peptide_GPCRs | 6 |
| GPCRs_Class_B_Secretin-like | 8 |
| GPCRs_Other | 3 |
| Hypertrophy_model | 2 |
| Integrin-mediated_cell_adhesion | 7 |
| Nuclear_Receptors | 4 |
| Ovarian_Infertility_Genes | 3 |
| Phosphatidylinositol signaling system | 16 |
| Prostaglandin and leukotriene metabolism | 1 |

1. A method for determining whether a subject has or is predisposed for a mental disorder, the method comprising the steps of:
(i) obtaining a biological sample from a subject;
(ii) contacting the sample with a reagent that selectively associates with a polynucleotide or polypeptide encoded by a nucleic acid that hybridizes under stringent conditions to a nucleotide sequence of Tables 1 and 22-24; and
(iii) detecting the level of reagent that selectively associates with the sample, thereby determining whether the subject has or is predisposed for a mental disorder.

2. The method of claim 1, wherein the reagent is an antibody.

3. The method of claim 1, wherein the reagent is a nucleic acid.

4. The method of claim 1, wherein the reagent associates with a polynucleotide.

5. The method of claim 1, wherein the regent associates with a polypeptide.

6. The method of claim 1, wherein the level of reagent that associates with the sample is different from a level associated with humans without a mental disorder.

7. The method of claim 1, wherein the biological sample is obtained from amniotic fluid.

8. The method of claim 1, wherein the mental disorder is a psychotic disorder.

9. The method of claim 6, wherein the level of reagent that associates with the sample is higher than a level associated with humans without a mental disorder.

10. The method of claim 8, wherein the psychotic disorder is schizophrenia.

11. A method of identifying a compound for treatment of a mental disorder, the method comprising the steps of:
(i) contacting the compound with a polypeptide, the polypeptide encoded by a polynucleotide that hybridizes under stringent conditions to a nucleic acid sequence comprising a nucleotide sequence of Tables 1 and 22-24; and
(ii) determining the functional effect of the compound upon the polypeptide, thereby identifying a compound for treatment of a mental disorder.

12. The method of claim 11, wherein the contacting step is performed in vitro.

13. The method of claim 11, wherein the polypeptide is expressed in a cell and the cell is contacted with the compound.

14. The method of claim 11, wherein the mental disorder is a psychotic disorder.

15. The method of claim 14, wherein the psychotic disorder is schizophrenia.

16. The method of claim 14, further comprising administering the compound to an animal and determining the effect on the animal.

17. The method of claim 16, wherein the determining step comprises testing the animal's mental function.

18. A method of identifying a compound for treatment of a mental disorder in a subject, the method comprising the steps of:
(i) contacting the compound to a cell, the cell comprising a polynucleotide that hybridizes under stringent conditions to a nucleotide sequence of Tables 1 and 22-24; and
(ii) selecting a compound that modulates expression of the polynucleotide, thereby identifying a compound for treatment of a mental disorder.

19. The method of claim 18, wherein the expression of the polynucleotide is enhanced.

20. The method of claim 18, wherein the expression of the polynucleotide is decreased.

21. The method of claim 18, further comprising administering the compound to an animal and determining the effect on the animal.

22. The method of claim 21, wherein the determining step comprises testing the animal's mental function.

23. The method of claim 18, wherein the mental disorder is a psychotic disorder.

24. The method of claim 23, wherein the psychotic disorder is schizophrenia

25. A method of treating a mental disorder in a subject, the method comprising the step of administering to the subject a therapeutically effective amount of a compound identified using the method of claim 11 or claim 18.

26. The method of claim 25, wherein the mental disorder is a psychotic disorder.

27. The method of claim 25, wherein the compound is a small organic molecule.

28. The method of claim 26, wherein the psychotic disorder is schizophrenia.

29. A method of treating mental disorder in a subject, the method comprising the step of administering to the subject a therapeutically effective amount of a polypeptide, the polypeptide encoded by a polynucleotide that hybridizes under stringent conditions to a nucleotide sequence of Tables 1 and 22-24.

30. The method of claim 29, wherein the mental disorder is schizophrenia.

31. A method of treating mental disorder in a subject, the method comprising the stepof administering to the subject a therapeutically effective amount of a nucleic acid, wherein the nucleic acid hybridizes under stringent conditions to a nucleotide sequence of Tables 1 and 22-24.

32. The method of claim 31, wherein the mental disorder is schizophrenia.
